(19) United States
(12) Patent Application Publication
(10) Pub. No.: US 2013/0203061 A1
(43) Pub. Date: Aug. 8, 2013

(54) METHODS AND SYSTEMS FOR ISOLATING, STORING, AND ANALYZING VESICLES

(71) Applicants: Michael Klass, Oro Valley, AZ (US); David Spetzler, Scottsdale, AZ (US); Traci Pawlowski, Phoenix, AZ (US)

(72) Inventors: Christine Kuslich, Paradise Valley, AZ (US); Michael Klass, Oro Valley, AZ (US); David Spetzler, Scottsdale, AZ (US); Traci Pawlowski, Phoenix, AZ (US)

(21) Appl. No.: 13/512,017

(22) PCT Filed: Nov. 30, 2010
(86) PCT No.: PCT/US10/58461

§ 371 Date: Dec. 3, 2012

Related U.S. Application Data
(60) Provisional application No. 61/265,350, filed on Nov. 30, 2009.
(60) Provisional application No. 61/265,343, filed on Nov. 30, 2009.
(60) Provisional application No. 61/265,341, filed on Nov. 30, 2009.
(60) Provisional application No. 61/265,348, filed on Nov. 30, 2009.
(60) Provisional application No. 61/266,937, filed on Dec. 4, 2009.

(51) Int. Cl. G01N 33/574; C12Q 1/68

(52) U.S. Cl. CPC G01N 33/57484; C12Q 1/6886
USPC 435/6.12; 436/501

(57) ABSTRACT

Provided herein are methods and systems for isolating, storing, and analyzing a vesicle from a sample The vesicle can be isolated using one or more lectins that bind to a vesicle One or more additional binding agents, such as a non lectin binding agent can also be used to isolate or analyze a vesicle

### CROSS-REFERENCE

This application claims the benefit of priority to U.S. Provisional Application Nos. 61/266,937, filed Dec. 4, 2009; 61/265,350, filed Nov. 30, 2009; 61/265,343, filed Nov. 30, 2009; 61/265,348, filed Nov. 30, 2009; and 61/265,341, filed Nov. 30, 2009, of which each is incorporated herein by reference in its entirety.

### BACKGROUND

Adequate sensitivity and specificity of a diagnostic assay is critical for disease detection, prognostic prediction, monitoring, and therapeutic decisions. Biomarkers for conditions and diseases such as cancer include biological molecules such as proteins, peptides, lipids, RNAs, DNA and modifications thereof. Their detection in many cases relies on assaying samples from a patient's tissue to identify the condition or disease. Methods to obtain these tissues of interest for analysis can be invasive, costly and pose complication risks for the patient. On the other hand, use of bodily fluids to isolate or detect biomarkers often significantly dilutes a biomarker resulting in readouts that lack requisite sensitivity. Additionally, most biomarkers are produced in low or moderate amounts in non-diseased tissues which can result in problems with adequate specificity.

The identification of specific biomarkers, such as DNA, RNA and proteins, can provide bio-signatures that are used for the diagnosis, prognosis, or theranosis of conditions or diseases. Vesicles present in a biological sample provide a source of biomarkers, e.g., the markers can be biological molecules that are present within a vesicle or on the surface of a vesicle. Characteristics of vesicles (e.g., size, surface antigens, cell-of-origin) can also provide a diagnostic, prognostic or theranostic readout. Thus, biomarkers associated with vesicles and characteristics of a vesicle can be detected to provide a diagnosis, prognosis, or theranosis.

Vesicles have been found in a number of body fluids, including blood plasma, breast milk, bronchioalveolar lavage fluid and urine. Vesicles also take part in the communication between cells, as transport vehicles for proteins, RNAs, DNAs, viruses, and prions. Vesicles secreted by cancer or other diseased cells, can be assessed to aid in diagnosis and individualized treatment decisions. Vesicles can also be used to identify and monitor physiological processes, e.g., pregnancy.

The present invention provides methods and systems for characterizing a phenotype by analyzing a vesicle. The vesicle can be isolated using one or more lectins.

### SUMMARY

Provided herein are methods, compositions and devices for isolating and analyzing a vesicle. In an aspect, the invention provides a method for determining a bio-signature of a vesicle comprising: contacting a vesicle from a biological sample obtained from a subject with one or more lectins; and determining a bio-signature of the vesicle.

In another aspect, the invention provides a method for isolating a vesicle comprising: contacting a vesicle from a biological sample obtained from a subject with one or more lectins; contacting the vesicle with one or more non-lectin binding agents; and determining a bio-signature of the vesicle.

In still another aspect, the invention provides a method for isolating of a plurality of vesicles comprising: applying the plurality of vesicles to a plurality of substrates, wherein each substrate is coupled to one or more lectins, and each subset of the plurality of substrates comprises a different lectin or combination of lectins than another subset of the plurality of substrates; and capturing at least a subset of the plurality of vesicles bound to the one or more lectins. In some embodiments, the method further comprises determining a bio-signature for each of the captured vesicles.

The methods of the invention can be used for characterizing a phenotype for the subject based on the bio-signature. The phenotype can be a cancer. Characterizing includes providing a diagnosis, prognosis, or theranosis, a determination of drug efficacy, monitoring the status of the subject's response or resistance to a treatment or selection of a treatment for the cancer. In some embodiments, the subject is non-responsive to a current therapeutic being administered to the subject. For example, the therapeutic can be a cancer therapeutic. In some embodiments, the characterizing comprises differentiating prostate cancer (PCa) and benign prostatic hyperplasia (BPH).

Characterizing the cancer can be performed by comparing the bio-signature to one or more reference values. The one or more reference values can be derived from the bio-signature identified in a different subject or group of subjects. In addition, the one or more reference values can be derived from the bio-signature identified in the subject over a time course. For example, the biosignature in the subject is followed over time, wherein a change in the biosignature can indicate an occurrence of cancer, a worsening cancer, an improving cancer, a remission, an effective treatment or an ineffective treatment. In some embodiments, lack of change in the biosignature over time may indicate these events.

The bio-signature may comprise an expression level, presence, absence, mutation, copy number variation, truncation, duplication, insertion, modification, sequence variation, or molecular association of one or more biomarkers. The biomarkers may be derived from any biological entity which provides informative information for characterizing the phenotype. For example, the one or more biomarkers can be a nucleic acid, peptide, protein, lipid, antigen, carbohydrate, a proteoglycan, or a combination thereof.

In some embodiments, the one or more biomarkers are detected using microarray analysis, PCR, hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), image cytometry, qRT-PCR, real-time PCR, PCR, flow cytometry, mass spectrometry, or a combination thereof.

In some embodiments, the bio-signature determined using the subject methods comprises a level or presence of one or more general vesicle biomarkers. In some embodiments, the bio-signature determined using the subject methods comprises a level or presence of one or more cell-of-origin specific biomarkers. In some embodiments, the bio-signature determined using the subject methods comprises a level or presence of one or more disease specific biomarkers.

The biomarker can be used in any appropriate combination. The bio-signature determined using the subject methods may comprise a level or presence of one or more general vesicle biomarkers and a level or presence of one or more cell-of-origin biomarkers. The bio-signature may also comprise a level or presence of one or more general vesicle biomarkers, and a level or presence of one or more disease specific biomarkers. The bio-signature may also comprise a level or presence of one or more general vesicle biomarkers, a level or presence of one or more cell-of-origin biomarkers, and a level or presence of one or more disease specific biomarkers.

Illustrative general vesicle biomarkers comprise CD63, CD9, CD81, CD82, CD37, CD53, or Rab-5b. An illustrative bio-signature comprises a level or presence of one or more of CD9, CD63 and CD81; a level or presence of one or more of PSMA (prostate specific membrane antigen, sometimes referred to as PSM) and PCSA (prostate cell surface antigen); and a level or presence of one or more of B7H3 and EpCam. The biomarkers can be detected on the surface of the vesicle. The biosignature can be used for a diagnosis, prognosis or theranosis of prostate cancer.

The one or more lectins used in the methods of the invention can include without limitation Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), Griffonia (Bandeiraea) Simplicifolia Lectin II (GS-II), or a combination thereof.

The one or more lectins can be in solution or can be bound to a substrate. The substrate can be a planar substrate or a particle. Vesicles captured by substrate bound lectins can be subsequently disassociated from the substrate. The vesicles can also be released from soluble lectins.

In some embodiments, the methods of the invention further comprise passing the biological sample through one or more porous membranes. Passing the biological sample through one or more porous membranes can be performed prior to contacting the vesicle with the one or more lectins. Alternately, passing the biological sample through one or more porous membranes can be performed subsequent to contacting the vesicle with the one or more lectins. The sample can also be passed through a membrane before and after contacting the vesicle with the one or more lectins.

The non-lectin binding agent used by the methods of the invention can be selected from the group consisting of: DNA, RNA, monoclonal antibodies, polyclonal antibodies, Fabs, Fab′, single chain antibodies, synthetic antibodies, DNA aptamers, RNA aptamers, peptoids, zDNA, peptide nucleic acids (PNAs), locked nucleic acids (LNAs), synthetic occurring chemical compounds, naturally occurring chemical compounds, dendrimers, and combinations thereof.

In some embodiments of the subject methods, the vesicle is isolated by size exclusion chromatography, density gradient centrifugation, differential centrifugation, nanomembrane ultrafiltration, or combinations thereof. These isolation steps can be performed prior to contacting the vesicle with the one or more lectins. Alternately, these isolation steps can be performed subsequent to contacting the vesicle with the one or more lectins. The isolation steps can be performed before and after contacting the vesicle with the one or more lectins.

The vesicle or plurality of vesicles can be a cell-of-origin specific vesicle. The cell-of-origin can be a tumor or cancer cell. In various embodiments, the cell-of-origin is a lung, pancreas, stomach, intestine, bladder, kidney, ovary, testis, skin, colorectal, breast, prostate, brain, esophagus, liver, placenta, or fetal cell.

The biological sample used in the subject methods may comprise a bodily fluid. For example, the bodily fluid can be without limitation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the bodily fluid comprises blood. In some embodiments, the bodily fluid comprises sera. In some embodiments, the bodily fluid comprises plasma. In some embodiments, the bodily fluid comprises urine.

In an aspect, the invention provides a composition comprising a vesicle and a preservation buffer. In some embodiments, the preservation buffer comprises a fixative. The fixative can be selected from the group consisting of: diazolidinyl urea, imidazolidinyl urea, dimethylol-5,5-dimethylhydantoin, dimethylol urea, 2-bromo-2-nitropropane-1,3-diol, 5-hydroxymethoxymethyl-1-aza-3,7-dioxabicyclo(3.3.0)octane and 5-hydroxymethyl-1-aza-3,7-dioxabicyclo(3.3.0)octane and 5-hydroxypoly[methyleneoxy]methyl-1-aza-3,7-dioxabicyclo(3.3.0)octane, sodium hydroxymethyl glycinate and mixtures thereof. In some embodiments, the preservation buffer comprises from about 1 to about 20 percent, about 10 percent, about 4 to about 6 percent, or about 5 percent by weight imidazolidinyl urea. The preservation buffer can include a mixture of imidazolidinyl urea and diazolidinyl urea. The preservation buffer may comprise a total concentration of imidazolidinyl urea and diazolidinyl urea from about 4 percent to about 10 percent by weight. The weight ratio of imidazolidinyl urea to diazolidinyl urea can be from about 10:1 to about 1:10.

In some embodiments, the preservation buffer comprises a protease inhibitor. For example, the protease inhibitor can be phenylmethylsulfonyl fluoride.

In an embodiments, the preservation buffer comprises an additive selected from the group consisting of polyethylene glycol (PEG), ethylenediaminetetraacetic acid (EDTA), phosphate buffered saline and mixtures thereof. The preservation buffer may contain from about 0.001 to about 0.2 percent by weight EDTA. The preservation buffer may contain up to about 1 percent by weight PEG. The preservation buffer may also comprise 0.3% phosphate buffered saline and ethylene diaminetetraacetic acid, 0.3% polyethylene glycol and 3% imidazolidinyl urea.

The preservation buffer can be formulated to prevent degradation of the vesicle at room temperature. In some embodiments, the preservation buffer prevents degradation of the vesicle at room temperature for at least about 12, 24, 36, 48, 60, 72, 84, or 96 hours.

In some embodiments, the vesicle stored in the preservation buffer is derived from a cancer cell. The cancer cell can be a lung, pancreas, stomach, intestine, bladder, kidney, ovary, testis, skin, colorectal, breast, prostate, brain, esophagus, or liver cell.

In a related aspect, the invention provides a method for storing a vesicle comprising: contacting a vesicle from a biological sample obtained from a subject with a lectin; and storing the vesicle in a composition comprising a preservation buffer. The preservation buffer can be a preservation buffer described above.

In other aspects, the invention provides a composition comprising a vesicle, a lectin, and a label. The invention further provides a composition comprising a vesicle, a lectin, and a non-lectin binding agent.

In some embodiments, the vesicle in the compositions is derived from a cancer cell. The cancer cell can be a lung, pancreas, stomach, intestine, bladder, kidney, ovary, testis, skin, colorectal, breast, prostate, brain, esophagus, or liver cell.

The lectin in the compositions can bind a vesicle proteoglycan or a fragment thereof. As a non-limiting example, the lectin can bind high mannose glycoproteins. Illustrative lectins for inclusion in the compositions include Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), and Griffonia (Bandeiraea) Simplicifolia Lectin II (GS-II).

In some embodiments, the non-lectin binding agent in the composition binds an vesicle component, wherein the binding agent is selected from the group consisting of: DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), synthetic chemical compounds, naturally occurring chemical compounds, dendrimers, and combinations thereof. In some embodiments, the non-lectin binding agent is an antibody. In some embodiments, the non-lectin binding agent is an aptamer. The antibody or aptamer can binds a tumor antigen, a cell-of-origin specific antigen, or a general vesicle antigen. Appropriate tumor antigens include without limitation B7H3 or EpCam. Appropriate cell-of-origin antigens include without limitation PSMA or PCSA. Appropriate general vesicle antigens include without limitation CD63, CD9, CD81, CD82, CD37, CD53, or Rab-5b, e.g., one, two or three of CD9, CD63 and CD81. When a composition comprises more than one non-lectin binding agent, a combination of antibodies and/or aptamers can be included.

In some embodiments, the non-lectin binding agent is attached to a label. The non-lectin binding agent can be attached directly to the label. Alternately, the non-lectin binding agent can be attached indirectly to the label. Similarly, the lectin in the composition can be attached to the label. The lectin can be attached directly to the label. Alternately, the lectin can be attached indirectly to the label. Labels for use with the compositions of the invention include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal, a nanoparticle, a quantum dot, a gold particle, a silver particle and a radioactive label.

In some embodiments, the lectin in the composition is attached to a substrate. The substrate can be a planar substrate or a particle. The substrate can be made of various materials, including without limitation agarose, aminocelite, resins, silica, polysaccharide, plastic or proteins. Silica based substrates include without limitation glass beads, sand, and diatomaceous earth. Polysaccharide substrates include without limitation dextran, cellulose and agarose. Protein based substrates include without limitation gelatin. Plastics include without limitation polystyrenes, polysuflones, polyesters, polyurethanes, polyacrylates and their activated and native amino and carboxyl derivatives. In some embodiments, the substrate is a bead. The bead may comprise an intrinsic label, such as a fluorescent label. The bead can also be magnetic.

The lectin in the composition can be attached to the substrate by a linker. In some embodiments, the linker is cleavable. In some embodiments, the linker comprises gluteraldehyde, C2 to C18 dicarboxylates, diamines, dialdehydes, dihalides, or mixtures thereof.

In another aspect, the invention provides a composition comprising a substantially enriched population of vesicles, wherein the enriched population of vesicles comprises vesicles with a substantially identical glycosylation pattern. For example, the vesicles with a substantially identical glycosylation pattern may comprise at least 30% of the total vesicle population of the composition, e.g., at least 40%, 50%, 60%, 70%, 80%, or at least 90% of the population of the composition.

In some embodiments, the enriched population is at least two fold enriched compared to the original composition, e.g., a biological sample from which the enriched composition is derived. That is, the concentration of enriched population of vesicles in the composition is at least two times the concentration of a population of a plurality of vesicles with the glycosylation pattern in a biological sample from which the composition was derived.

In some embodiments, the enriched population of vesicles comprises cell-of-origin specific vesicles. For example, the cell-of-origin can be a tumor or cancer cell. Alternately, the cell-of-origin can be a lung, pancreas, stomach, intestine, bladder, kidney, ovary, testis, skin, colorectal, breast, prostate, brain, esophagus, liver, placenta, or fetal cell.

In still another aspect, the invention provides a device for isolating a vesicle comprising: a chamber comprising a lectin configured to capture a vesicle; and a chamber comprising a non-lectin binding agent configured to capture a vesicle. The non-lectin binding agent can be attached to a substrate.

In some embodiments, the lectin and the non-lectin binding agent are present in the same chamber of the device. In other embodiments, the lectin is present in a first chamber and the non-lectin binding agent is present in a second chamber of the device. The first chamber and the second chamber can be in fluid communication. In some embodiments, the device is configured and arranged such that a biological sample flows through the first chamber prior to the second chamber. In other embodiments, the device is configured and arranged such that a biological sample flows through the second prior to the first chamber.

The non-lectin binding agent within the device can be selected from the group consisting of: DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), synthetic or naturally occurring chemical compounds, dendrimers, and combinations thereof.

The device can also be configured to include an additional chamber, wherein the chamber comprises an additional binding agent. The additional binding agent can be a lectin or non-lectin binding agent as described herein.

In still another aspect, the invention provides a device for isolating a vesicle comprising: a chamber comprising a lectin configured to capture the vesicle; and a porous membrane configured to permit another vesicle to pass through. The porous membrane can be a hollow fiber membrane. The porous membrane may exclude substantially all cells from passing through the pores. In some embodiments, the porous membrane of the device has pores less than about 700 nm in diameter. In some embodiments, the porous membrane has pores with an inside diameter of about 0.3 mm and an outside diameter of about 0.5 mm.

The device can be configured and arranged such that the chamber comprises the porous membrane, wherein the lectin is disposed within an extrachannel space of the chamber proximate to an exterior surface of the porous membrane.

The device can be configured so that a cartridge surrounds at least one porous membrane, the porous membrane having a lumen, and the cartridge and the at least one porous membrane defining an extralumenal space there between, wherein the device comprises an inlet port and an outlet port in fluid communication with the lumen, and at least one port in fluid communication with the extralumenal space, wherein the device is configured for a vesicle of a biological sample to pass through the lumen and through the porous membrane into the extralumenal space while preventing a cellular portion of the biological sample passed through the lumen to pass through the porous membrane into the extralumenal space. In some embodiments, the chamber is external to the cartridge. In other embodiments, the chamber is internal to the cartridge. In some embodiments, the extralumenal space is the chamber.

The device may further comprise a chamber comprising a non-lectin binding agent. The chamber comprising the non-lectin binding agent can be the same chamber comprising the lectin. Alternately, the chamber comprising the non-lectin binding agent can be a different chamber than the chamber comprising the lectin.

In some embodiments, the device comprises a pump configured to pump a biological sample into the device at an assisted flow rate, the assisted flow rate being selected to increase a clearance rate of the device by at least two times over a clearance rate of the device without the pump.

The lectin in any of the devices of the invention may comprise Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), and Griffonia (Bandeiraea) Simplicifolia Lectin II (GS-II). In some embodiments, the lectin in the device is attached to a substrate. The substrate can be a planar substrate or a particle. The substrate can be made of various materials, including without limitation agarose, aminocelite, resins, silica, polysaccharide, plastic or proteins. Silica based substrates include without limitation glass beads, sand, and diatomaceous earth. Polysaccharide substrates include without limitation dextran, cellulose and agarose. Protein based substrates include without limitation gelatin. Plastics include without limitation polystyrenes, polysuflones, polyesters, polyurethanes, polyacrylates and their activated and native amino and carboxyl derivatives. In some embodiments, the substrate is a bead. The bead may comprise an intrinsic label, such as a fluorescent label. The bead can also be magnetic.

The lectin in the device can be attached to the substrate by a linker. In some embodiments, the linker is cleavable. In some embodiments, the linker comprises gluteraldehyde, C2 to C18 dicarboxylates, diamines, dialdehydes, dihalides, or mixtures thereof.

In a related aspect, the invention provides a device configured for isolating of a plurality of vesicles comprising: a plurality of substrates, wherein each substrate is coupled to one or more lectins, and each subset of the plurality of substrates comprises a different lectin or combination of lectins than another subset of the plurality of substrates.

In another aspect, the invention provides a method of characterizing a cancer in a subject comprising: identifying in a single assay a bio-signature of one or more vesicles in a biological sample from the subject, wherein the identifying comprises: determining the presence of level or one or more general vesicle protein biomarkers; determining the presence of level or one or more cell-specific protein biomarkers; and determining the presence of level or one or more disease-specific protein biomarkers; and comparing said presence or levels in the biological sample to a reference to determine whether the presence or levels indicate that the subject may be predisposed to or afflicted with the cancer, thereby characterizing the cancer. The characterizing can be determining the presence or absence of cancer. In some embodiments, the one or more general vesicle protein biomarkers comprise CD9, CD63, CD81, or a combination thereof; the one or more cell-specific protein biomarkers comprise PSMA, PCSA, or both; and the one or more disease-specific protein biomarkers comprise EpCam, B7H3, or both. The cancer can be but is not limited to prostate cancer. For example, the characterizing can include differentiating prostate cancer and benign prostatic hyperplasia (BPH). In such cases, one or more of the protein biomarkers can be selected from Table 1 herein as it relates to prostate cancer or benign prostatic hyperplasia (BPH).

The sample used in the methods of characterizing a cancer can be a bodily fluid. The volume of the sample can be less than 2 mL. The bodily fluid can be peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood. In some embodiments, the bodily fluid comprises blood. In some embodiments, the bodily fluid comprises sera. In some embodiments, the bodily fluid comprises plasma. In some embodiments, the bodily fluid comprises urine.

The one or more vesicles can have a diameter of about 30 nm to about 800 nm, e.g., about 30 nm to about 200 nm.

The one or more vesicles can be isolated using one or more of size exclusion chromatography, density gradient centrifugation, differential centrifugation, nanomembrane ultrafiltration, immunoabsorbent capture, affinity purification, and microfluidic separation. Combinations of the techniques can be used. The vesicles can be isolated prior to identifying the biosignature. Alternately, the biological sample is not enriched for vesicles prior to determining the bio-signature.

The general vesicle protein biomarkers used in the methods of characterizing a cancer can be CD63, CD9, CD81, CD82, CD37, CD53, or Rab-5b. The one or more disease-specific protein biomarkers can be a-biomarker for a tumor or cancer cell. The one or more cell-specific protein biomarkers can be a biomarker for a lung, pancreas, stomach, intestine, bladder, kidney, ovary, testis, skin, colorectal, breast, prostate, brain, esophagus, liver, placenta, or fetal cell.

In some embodiments of the method of characterizing a cancer, the determining comprises measuring an expression level, presence, absence, mutation, truncation, insertion, modification, sequence variation or molecular association of the protein biomarkers. The characterizing may further comprise one or more of determining an amount of vesicles, a temporal evaluation of a variation in vesicle half-life, a temporal evaluation of circulating vesicle half-life, a temporal evaluation of vesicle metabolic half-life, or determining a vesicle activity.

The one or more of the protein biomarkers can be associated with a clinically distinct tumor type or subtype of cancer. A variety of useful biomarkers can be assessed. For example, one or more of the protein biomarkers can be selected from Table 1 herein. In some embodiments, the one or more of the protein biomarkers are selected from the group consisting of: CD9, PSCA, TNFR, CD63, MFG-E8, EpCam, Rab, CD81, STEAP, PCSA, 5T4, PSMA, CD59, CD66 and B7H3. In some embodiments, the bio-signature comprises at least two biomarkers selected from the group consisting of: EpCam, CD9, PCSA, CD63, CD81, PSMA and B7H3. For example, the one or more general vesicle protein biomarkers comprise CD9, CD63 and CD81; the one or more cell-specific protein biomarkers comprise PSMA and PCSA; and the one or more disease-specific protein biomarkers comprise B7H3. These markers can be used to characterize a prostate cancer.

The bio-signature identified by the subject method may comprise one or more binding agents. The binding agent can be without limitation an antigen, DNA molecule, RNA molecule, antibody, antibody fragment, aptamer, peptoid, zDNA, peptide nucleic acid (PNA), locked nucleic acids (LNA), lectin, peptide, dendrimer or chemical compound. The binding agent can be selected from Table 2 herein.

The invention contemplates various approaches to detecting the biomarkers. In an embodiments, detecting one or more of the protein biomarkers comprises: capturing the one or more vesicles with one or more primary antibodies; detecting the captured one or more vesicles with one or more detection antibodies; allowing an enzyme linked secondary antibody to react with the one or more detection antibodies; adding a detection reagent; and detecting a reaction between the reagent and the enzyme linked secondary antibody. In other embodiments, detecting one or more of the protein biomarkers comprises: capturing the one or more vesicles with one or more primary binding agents; and detecting the captured one or more vesicles with one or more detection binding agents. The one or more primary binding agents may comprise without limitation an antibody to a protein or antigen selected from the group consisting of: Rab 5b, CD63, caveolin-1, CD9, PSCA, TNFR, CD63, MFG-E8, EpCam, Rab, CD81, STEAP, PCSA, 5T4, PSMA, CD59, CD66, B7H3 and fragments thereof. The one or more detection binding agents may comprise without limitation an antibody to a protein or antigen selected from the group consisting of: Rab 5b, CD63, caveolin-1, CD9, PSCA, TNFR, CD63, MFG-E8, EpCam, Rab, CD81, STEAP, PCSA, 5T4, PSMA, CD59, CD66, B7H3 and fragments thereof. The one or more primary binding agents can be attached to one or more substrates. The one or more substrates can be an array, well or particle. In some embodiments, the one or more substrates comprise a magnetic bead. In some embodiments, the one or more substrates comprise a fluorescently labeled bead. The particle can be intrinsically labeled. The particle can also be labeled with more than one label.

Characterizing the cancer according to the methods of the inventions can include a diagnosis, prognosis, determination of drug efficacy, monitoring the status of the subject's response or resistance to a treatment or selection of a treatment for the cancer. In some embodiments, the subject is non-responsive to a current therapeutic being administered to the subject. For example, the therapeutic can be a cancer therapeutic.

In some embodiments, characterizing the cancer comprises comparing the bio-signature to one or more reference values. The one or more reference values can be derived from the bio-signature identified in a different subject or group of subjects. The one or more reference values can also be derived from the bio-signature identified in the subject over a time course. For example, the biosignature in the subject is followed over time, wherein a change in the biosignature can indicate an occurrence of cancer, a worsening cancer, an improving cancer, a remission, an effective treatment or an ineffective treatment. In some embodiments, lack of change in the biosignature over time may indicate these events.

In an aspect, the invention provides a method for characterizing a prostate disorder in a sample from a subject comprising: determining a first amount of vesicles in the sample from the subject by capturing vesicles in the sample with an anti-PCSA antibody attached to a substrate and detecting the anti-PCSA captured vesicles using one or more of an anti-CD9 antibody, an anti-CD63 antibody and an anti-CD81 antibody; and characterizing the prostate disorder by comparing the first amount of vesicles to one or more reference values.

In another aspect, the invention provides a method for characterizing a prostate disorder in a sample from a subject comprising: determining a first amount of vesicles in the sample from the subject by capturing vesicles in the sample with an anti-B7H3 antibody attached to a substrate and detecting the anti-B7H3 captured vesicles using one or more of an anti-CD9 antibody, an anti-CD63 antibody and an anti-CD81 antibody; and characterizing the prostate disorder by comparing the first amount of vesicles to one or more reference values.

In still another aspect, the invention provides a method for characterizing a prostate disorder in a sample from a subject comprising: determining a first amount of vesicles in the sample by capturing vesicles in the sample with an anti-PSMA antibody attached to a substrate and detecting the anti-PSMA captured vesicles using one or more of an anti-CD9 antibody, an anti-CD63 antibody and an anti-CD81 antibody; and characterizing the prostate disorder by comparing the first amount of vesicles to one or more reference values.

In yet another aspect, the invention provides a method for characterizing a prostate disorder in a sample from a subject comprising: determining a first amount of vesicles in the sample by capturing vesicles in the sample with an anti-PCSA antibody attached to a substrate and detecting the anti-PCSA captured vesicles using one or more of an anti-CD9 antibody, an anti-CD63 antibody and an anti-CD81 antibody; determining a second amount of vesicles in the sample by capturing vesicles in the sample with an anti-B7H3 antibody attached to a substrate and detecting the anti-B7H3 captured vesicles using one or more of an anti-CD9 antibody, an anti-CD63 antibody and an anti-CD81 antibody; determining a third amount of vesicles in a sample by capturing vesicles in the sample with an anti-PSMA antibody attached to a substrate and detecting the anti-PSMA captured vesicles using one or more of an anti-CD9 antibody, an anti-CD63 antibody and an anti-CD81 antibody; and characterizing the prostate disorder by comparing the first, second, and third amount of vesicles to one or more reference values. The method can further comprise determining a fourth amount of vesicles in the sample from the subject by capturing membrane vesicles with an anti-EpCam antibody attached to a substrate and detecting the vesicles using one or more of an anti-CD9 antibody, an anti-CD63 antibody and an anti-CD81 antibody. The determining steps, including the optional determining step with the anti-EpCam antibody, can be carried out in a single assay, thus providing a multiplex assay.

In the methods of characterizing a prostate disorder, the characterizing may comprise a diagnosis, prognosis, determination of drug efficacy, monitoring the status of, or selection of a treatment for the prostate disorder.

The substrate used in the methods of characterizing a prostate disorder can be a bead. The vesicles can be detecting using flow cytometry.

The anti-CD9 antibody, the anti-CD63 antibody and the anti-CD81 antibody can each comprise a fluorescent label. The label can be different for each of these antibodies or the same for all of these antibodies. The vesicles detected in each step can be detected using the anti-CD9 antibody, the anti-CD63 antibody and the anti-CD81 antibody.

The one or more reference values comprise an amount of vesicles identified in a different subject or group of subjects. Alternately, the one or more reference values comprise an amount of vesicles identified in the subject over a time course.

The sample used in the methods of characterizing a prostate disorder can be a bodily fluid. The bodily fluid can be urine, semen, blood, plasma or serum. In some embodiments, the bodily fluid comprises plasma.

The prostate disorder can be prostate cancer or benign prostatic hyperplasia (BPH).

### INCORPORATION BY REFERENCE

A10 publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a method of identifying a bio-signature of a vesicle to characterize a disease by isolating a vesicle using a lectin. FIG. 1B depicts a method of using a lectin and a non-lectin binding agent to isolate a vesicle, wherein a bio-signature for the vesicle is determined and used to characterize a phenotype.

FIG. 2 is a flow chart of an exemplary method disclosed herein.

FIG. 3 illustrates assessing vesicles from normal and cancer subjects using a single capture agent and single detection agent. The capture agent is an antibody for EpCam and the detection agent detects A) CD81, B) EpCam, or C) CD9.

FIG. 4 illustrates methods of characterizing a phenotype by assessing vesicle biosignatures. (A) is a schematic of a planar substrate coated with a capture antibody, which captures vesicles expressing that protein. The capture antibody is for a vesicle protein that is specific or not specific for vesicles derived from diseased cells (“disease vesicle”). The detection antibody binds to the captured vesicle and provides a fluorescent signal. The detection antibody can detect an antigen that is generally associated with vesicles, or is associated with a cell-of-origin or a disease, e.g., a cancer. (B) is a schematic of a bead coated with a capture antibody, which captures vesicles expressing that protein. The capture antibody is for a vesicle protein that is specific or not specific for vesicles derived from diseased cells (“disease vesicle”). The detection antibody binds to the captured vesicle and provides a fluorescent signal. The detection antibody can detect an antigen that is generally associated with vesicles, or is associated with a cell-of-origin or a disease, e.g., a cancer. (C) is an example of a screening scheme that can be performed by multiplexing using the beads as shown in (B). (D) presents illustrative schemes for capturing and detecting vesicles to characterize a phenotype. (E) presents illustrative schemes for assessing vesicle payload to characterize a phenotype.

FIG. 5 illustrates multiple detectors can increase the signal of vesicle detection. (A) Median intensity values are plotted as a function of purified vesicle concentration from the VCaP cell line when labeled with a variety of prostate specific PE conjugated antibodies. Vesicles captured with EpCam (left graphs) or PCSA (right graphs) and the various proteins detected by the detector antibody are listed to the right of each graph. In both cases the combination of CD9 and CD63 gives the best increase in signal over background (bottom graphs depicting percent increase). The combination of CD9 and CD63 gave about 200% percent increase over background. (B) further illustrates prostate cancer/prostate vesicle-specific marker multiplexing improves detection of prostate cancer cell derived vesicles. Median intensity values are plotted as a function of purified vesicle concentration from the VCaP cell line when labeled with a variety of prostate specific PE conjugated antibodies. Vesicles captured with PCSA (left) and vesicles captured with EpCam (right) are depicted. In both cases the combination of B7H3 and PSMA gives the best increase in signal over background.

FIG. 6 is a schematic of protein expression patterns. Different proteins are typically not distributed evenly or uniformly on a vesicle shell. Vesicle-specific proteins, e.g., CD9, CD63 or CD81, are typically more common, while cancer-specific proteins, e.g., CD66 or EpCam are less common. Capture of a vesicle can be more accomplished using a more common, less cancer-specific protein, and cancer-specific proteins used in the detection phase. Capture of a vesicle can also be more accomplished using a less common, cancer-specific protein and using more common, less cancer-specific proteins used in the detection phase to increase the signal of the captured vesicles.

FIG. 7 illustrates a method of depicting results using a bead based method of detecting vesicles from a subject. (A) For an individual patient, a graph of the bead enumeration and signal intensity using a screening scheme as depicted in FIG. 4B, where ˜100 capture beads are used for each capture/detection combination assay per patient. For a given patient, the output shows number of beads detected vs. intensity of signal. The number of beads captured at a given intensity is an indication of how frequently a vesicle expresses the detection protein at that intensity. The more intense the signal for a given bead, the greater the expression of the detection protein. (B) is a normalized graph obtained by combining normal patients into one curve and cancer patients into another, and using bio-statistical analysis to differentiate the curves. Data from each individual is normalized to account for variation in the number of beads read by the detection machine, added together, and then normalized again to account for the different number of samples in each population.

FIG. 8 illustrates prostate cancer bio-signatures. (A) is a histogram of intensity values collected from a multiplexing experiment using the Luminex platform, where beads were functionalized with CD63 antibody, incubated with vesicles purified from patient plasma, and then labeled with a phycoerythrin (PE) conjugated EpCam antibody. The darker shaded bars (blue) represent the population from 12 normal subjects and the lighter shaded bars (green) are from 7 stage 3 prostate cancer patients. (B) is a normalized graph for each of the histograms shown in (A), as described in FIG. 7. The distributions are of a Gaussian fit to intensity values from the Luminex results of (A) for both prostate patient samples and normal samples. (C) is an example of one of the prostate bio-signatures shown in (B), the CD63 versus CD63 bio-signature (upper graph) where CD63 is used as the detector and capture antibody. The lower three panels show the results of flow cytometry on three prostate cancer cell lines (VCaP, LNcap, and 22RV1). Points above the horizontal line indicate beads that captured vesicles with CD63 that contain B7H3. Beads to the right of the vertical line indicate beads that have captured vesicles with CD63 that have PSMA. Those beads that are above and to the right of the lines have all three antigens. CD63 is a surface protein that is associated with vesicles, PSMA is surface protein that is associated with prostate cells, and B7H3 is a surface protein that is associated with aggressive cancers (specifically prostate, ovarian, and non-small-cell lung). The combination of all three antigens together identifies vesicles that are from cancer prostate cells. The majority of CD63 expressing prostate cancer vesicles also have prostate-specific membrane antigen, PSMA, and B7H3 (implicated in regulation of tumor cell migration and invasion and an indicator of aggressive cancer as well as clinical outcome). (D) is a prostate cancer vesicle topography. The upper panels show the results of capturing and labeling with CD63, CD9, and CD81 in various combinations. Almost all points are in the upper right quadrant indicating that these three markers are highly coupled. That is, if a vesicle has one of these markers, it typically has all three. The lower row depicts the results of capturing cell line vesicles with B7H3 and labeling with CD63 and PSMA. Both VCaP and 22RV1 show that most vesicles captured with B7H3 also have CD63, and that there are two populations, those with PSMA and those without. The presence of B7H3 may be an indication of how aggressive the cancer is, as LNcap does not have a high amount of B7H3 containing vesicles (not many spots with CD63). LnCap is an earlier stage prostate cancer analogue cell line.

FIG. 9 depicts a table of the sensitivity and specificity for different prostate signatures. “Vesicle” lists the threshold value or reference value of vesicle levels, “Prostate” lists the threshold value or reference value used for prostate vesicles, “Cancer-1,” “Cancer-2,” and “Cancer-3” lists the threshold values or reference values for the three different bio-signatures for prostate cancer, the “QC-1” and “QC-2” columns list the threshold values or reference values for quality control, or reliability, and the last four columns list the specificities and sensitivities for benign prostate hyperplasia (BPH).

FIG. 10 illustrates (A) the sensitivity and specificity, and the confidence level, for detecting prostate cancer using antibodies to the listed proteins listed as the detector and capture antibodies. CD63, CD9, and CD81 are general vesicle markers and EpCam is a cancer marker. The individual results are depicted in (B) for EpCam versus CD63, with 99% confidence, 100% (n=8) cancer patient samples were different from the Generalized Normal Distribution and with 99% confidence, 77% (n=10) normal patient samples were not different from the Generalized Normal Distribution; (C) for CD81 versus CD63, with 99% confidence, 90% (n=5) cancer patient samples were different from the Generalized Normal Distribution; with 99% confidence, 77% (n=10) normal patient samples were not different from the Generalized Normal Distribution; (D) for CD63 versus CD63, with 99% confidence, 60% (n=5) cancer patient samples were different from the Generalized Normal Distribution; with 99% confidence, 80% (n=10) normal patient samples were not different from the Generalized Normal Distribution; (E) for CD9 versus CD63, with 99% confidence, 90% (n=5) cancer patient samples were different from the Generalized Normal Distribution; with 99% confidence, 77% (n=10) normal patient samples were not different from the Generalized Normal Distribution.

FIG. 11 is a schematic for A) a vesicle PCa assay, which leads to a B) decision tree.

FIG. 12A illustrates the ability of a vesicle bio-signature to discriminate between normal prostate and PCa samples. Cancer markers included EpCam and B7H3. General vesicle markers included CD9, CD81 and CD63. Prostate specific markers included PCSA. The test was found to be 98% sensitive and 95% specific for PCa vs normal samples. FIG. 12B illustrates mean fluorescence intensity (MFI) on the Y axis for vesicle markers of FIG. 12A in normal and prostate cancer patients.

FIG. 13A illustrates improved sensitivity of the vesicle assays of the invention versus conventional PCa testing. FIG. 13B illustrates improved specificity of the vesicle assays of the invention versus conventional PCa testing.

FIG. 14 illustrates discrimination of BPH samples from normals and PCa samples using CD63.

FIG. 15 illustrates the ability of a vesicle bio-signature to discriminate between normal prostate and PCa samples. Cancer markers included EpCam and B7H3. General vesicle markers included CD9, CD81 and CD63. Prostate specific markers included PCSA. The test was found to be 98% sensitive and 84% specific for PCa vs normal & BPH samples.

FIG. 16 illustrates improved specificity of the vesicle assays of the invention for PCa versus conventional testing even when BPH samples are included.

FIG. 17 illustrates ROC curve analysis of the vesicle assays of the invention versus conventional testing.

FIG. 18 illustrates a correlation between general vesicle (e.g. vesicle “MV”) levels, levels of prostate-specific MVs and MVs with cancer markers.

FIG. 19A is a schematic for a vesicle PCa assay, which leads to a decision tree as shown in FIG. 19B. FIG. 19C shows the results of a vesicle detection assay for prostate cancer following the decision tree versus detection using elevated PSA levels. FIG. 19D shows the results of a vesicle detection assay for prostate cancer following the decision tree on a cohort of 933 PCa and non-PCa patient samples. FIG. 19E shows an ROC curve corresponding to the data shown in FIG. 19D.

FIG. 20 illustrates the use of cluster analysis to set the MFI threshold for vesicle biomarkers of prostate cancer. A) Raw and log transformed data for 149 samples. The raw data is plotted in the left column and the transformed data in the right. B) Cluster analysis on PSMA vs B7H3 using log transformed data as input. The circles (normals) and x's (cancer) show the two clusters found. The open large circles show the point that was used as the center of the cluster. Blue lines show the chosen cutoff for each parameter. C) Cluster analysis on PCSA vs B7H3 using log transformed data as input. The circles (normals) and x's (cancer) show the two clusters found. The open large circles show the point that was used as the center of the cluster. Blue lines show the chosen cutoff for each parameter. D) Cluster analysis on PSMA vs PCSA using log transformed data as input. The circles and x's show the two clusters found. The open large red circles show the point that was used as the center of the cluster. Blue lines show the chosen cutoff for each parameter. E) The thresholds determined in B-D) were applied to the larger set of data containing 313 samples, and resulted in a sensitivity of 92.8% and a specificity of 78.7%.

### DETAILED DESCRIPTION OF THE INVENTION

Disclosed herein are methods and systems for isolating, storing, and analyzing a vesicle. The vesicle can be analyzed, such as by determining a bio-signature of the vesicle, which can be used to characterize a phenotype of an individual or subject.

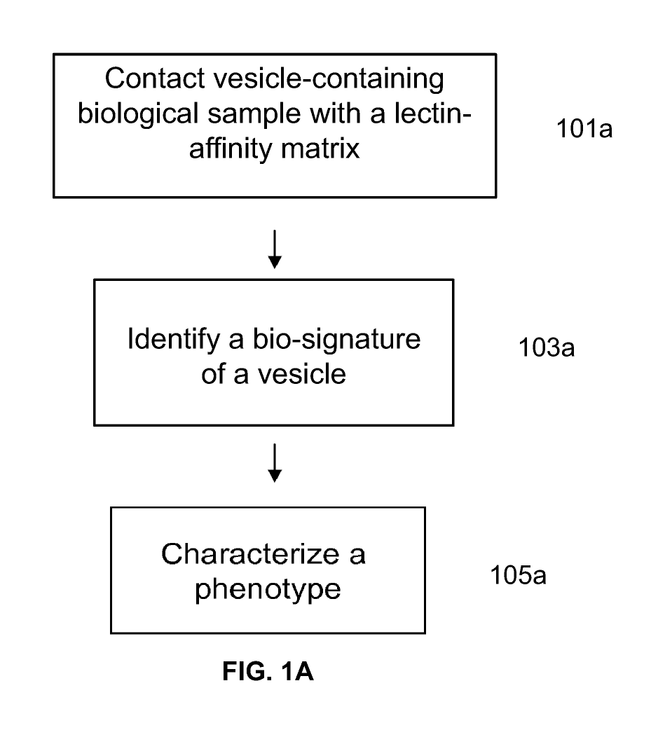

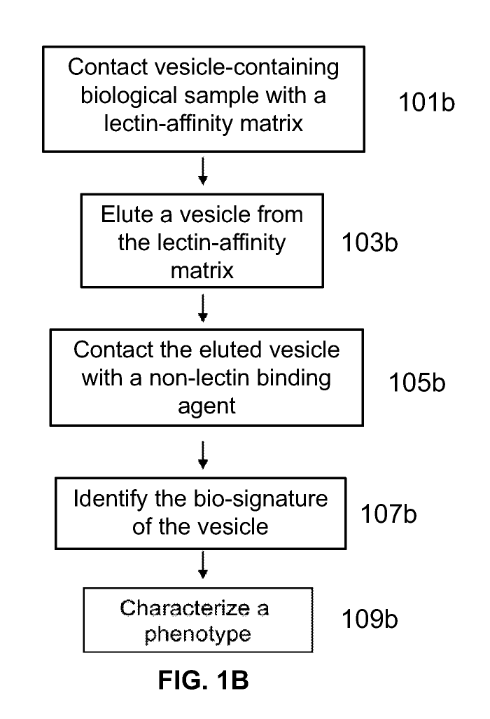

A method of characterizing a phenotype by analyzing a vesicle is as depicted in FIG. 1A. In step 101a, a vesicle-containing biological sample is contacted with a lectin (such as a lectin-affinity matrix). At step 103a, a bio-signature is identified for the vesicle and at step 105a, a phenotype is characterized based on the bio-signature. In another embodiment, the method is as depicted in FIG. 1B, wherein at step 101b, a vesicle-containing biological sample is contacted with a lectin (such as a lectin-affinity matrix). At step 103b, the vesicle is eluted from the lectin (such as from the lectin-affinity matrix). At step 105b, the eluted vesicle is contacted with a non-lectin binding agent (for example, an antibody to a tumor antigen). At step 107b, a bio-signature is identified for the vesicle and at step 109b, a phenotype is characterized based on the bio-signature.

### Vesicles

Products and methods of the invention are directed to assaying one or more vesicles. A vesicle, as used herein, is a vesicle that is shed from cells. Vesicles are also referred to generally as membrane vesicles. Vesicles or membrane vesicles include without limitation the following types or species: microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phopholipid vesicle, liposomal vesicle, argosome, texasome, secresome, tolerosome, melanosome, oncosome, or exocytosed vehicle. Unless otherwise specified, methods that make use of a species of vesicle can be applied to other types of vesicles. Vesicles comprise spherical structures with a lipid bilayer similar to cell membranes which surrounds an inner compartment which can contain soluble components, sometimes referred to as the payload. In some embodiments, the methods of the invention make use of exosomes, which are small secreted vesicles of about 40-100 nm in diameter. For a review of membrane vesicles, including types and characterizations, see Thery et al., Nat Rev Immunol. 2009 August; 9(8):581-93. Some properties of different types of vesicles include those in Table 1:

TABLE 1
Vesicle Properties

| | | | | Membrane | Exosome-like | Apoptotic |
|---|---|---|---|---|---|---|
| Feature | Exosomes | Microvesicles | Ectosomes | particles | vesicles | vesicles |
| Size | 50-100 nm | 100-1,000 nm | 50-200 nm | 50-80 nm | 20-50 nm | 50-500 nm |
| Density in | 1.13-1.19 g/ml | | | 1.04-1.07 g/ml | 1.1 g/ml | 1.16-1.28 g/ml |
| sucrose | | | | | | |
| EM | Cup shape | Irregular | Bilamellar round | Round | Irregular shape | Heterogeneous |
| appearance | | shape, electron | structures | | | |
| | | dense | | | | |
| Sedimentation | 100,000 g | 10,000 g | 160,000-200,000 g | 100,000-200,000 g | 175,000 g | 1,200 g, |
| | | | | | | 10,000 g, |
| | | | | | | 100,000 g |
| Lipid | Enriched in | Expose PPS | Enriched in | | No lipid rafts | |
| composition | cholesterol, | | cholesterol and | | | |
| | sphingomyelin | | diacylglycerol; | | | |
| | and ceramide; | | expose PPS | | | |
| | contains lipid | | | | | |
| | rafts; expose PPS | | | | | |
| Major protein | Tetraspanins | Integrins, | CR1 and | CD133; no | TNFRI | Histones |
| markers | (CD63, CD9), | selectins and | proteolytic | CD63 | | |
| | Alix, TSG101 | CD40 ligand | enzymes; no | | | |
| | | | CD63 | | | |
| Intracellular | Internal | Plasma | Plasma | Plasma | | |
| origin | compartments | membrane | membrane | membrane | | |
| | (endosomes) | | | | | |

Abbreviations:
phosphatidylserine (PPS);
electron microscopy (EM)

Vesicles can be released into the extracellular environment from cells. Cells releasing vesicles include without limitation cells that originate from, or are derived from, the ectoderm, endoderm, or mesoderm. The cells may have undergone genetic, environmental, and/or any other variations or alterations. For example, the cell can be tumor cells or cells having various genetic mutations. A vesicle can be created intracellularly when a segment of the cell membrane spontaneously invaginates and is ultimately exocytosed (see for example, Keller et al., Immunol. Lett. 107 (2): 102-8 (2006)). A vesicle can have a diameter of greater than 10, 20, or 30 nm. They can have a diameter of about 30-1000 nm, about 30-800 nm, about 30-200 nm, or about 30-100 nm. In some embodiments, the vesicle has a diameter of less than 10,000 nm, 1000 nm, 800 nm, 500 nm, 200 nm, 100 nm or 50 nm.

Vesicles include shed membrane bound particles that are derived from either the plasma membrane or an internal membrane. Vesicles also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). A circulating tumor-derived vesicle is a vesicle shed into circulation or bodily fluids from tumor cells. When such vesicle is an exosome, it may be defined as a circulating-tumor derived exosome (CTE). In some instances, a vesicle can be derived from a specific cell of origin. CTE, as with a cell-of-origin specific vesicle, typically have one or more unique biomarkers that permit isolation of the CTE or cell-of-origin specific vesicle, e.g., from a bodily fluid and sometimes in a specific manner.

Vesicles can be directly assayed from a biological sample. The level or amount of vesicles in the sample, the bio-signature of one or more vesicles in the sample, or both, can be determined without prior isolation, purification, or concentration of the biological sample or vesicle. Alternatively, the vesicle in the sample may be isolated, purified, or concentrated from a sample prior to analysis.

### Samples

A vesicle can be isolated from a biological sample obtained from the subject. A subject or patient can include, but is not limited to, mammals such as bovine, avian, canine, equine, feline, ovine, porcine, or primate animals (including humans and non-human primates). A subject may also include mammals of importance due to being endangered, such as Siberian tigers; or economic importance, such as animals raised on farms for consumption by humans, or animals of social importance to humans such as animals kept as pets or in zoos. Examples of such animals include but are not limited to: carnivores such as cats and dogs; swine including pigs, hogs and wild boars; ruminants or ungulates such as cattle, oxen, sheep, giraffes, deer, goats, bison, camels or horses. Also included are birds that are endangered or kept in zoos, as well as fowl and more particularly domesticated fowl, i.e. poultry, such as turkeys and chickens, ducks, geese, guinea fowl. Also included are domesticated swine and horses (including race horses). In addition, any animal species connected to commercial activities are also included such as those animals connected to agriculture and aquaculture and other activities in which disease monitoring, diagnosis, and therapy selection are routine practice in husbandry for economic productivity and/or safety of the food chain.

The subject can have a pre-existing disease or condition, such as cancer. Alternatively, the subject may not have any known pre-existing condition. The subject may also be non-responsive to an existing or past treatment, such as a treatment for cancer.

The biological sample obtained from the subject may be any bodily fluid. For example, the biological sample can be peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation which may be of fetal or maternal origin.

The biological sample may also be a tissue sample or biopsy, from which vesicles may be obtained. For example, if the sample is a solid sample, cells from the sample can be cultured and vesicle product induced.

The biological sample may be obtained through a third party, such as a party not performing the analysis of the vesicle. For example, the sample may be obtained through a clinician, physician, or other health care manager of a subject from which the sample is derived. In some embodiments, the biological sample is obtained by the same party analyzing the vesicle.

The volume of the biological sample used for analyzing a vesicle can be in the range of between 0.1-20 mL, such as less than about 20, 15, 10, 9, 8, 7, 6, 5, 4, 3, 2, 1 or 0.1 mL. In some embodiments, the sample is about 0.1, 0.2, 0.3, 0.4, 0.5, 0.6, 0.7, 0.8, 0.9, 1.0, 1.5, 2, 3, 4, 5, 6, 7, 8, 9, 10, 11, 12, 13, 14, 15, 16, 17, 18, 19, or 20 mL. In some embodiments, the sample is about 1,000, 900, 800, 700, 600, 500, 400, 300, 250, 200, 150, 100, 75, 50, 25 or 10 μl. For example, a small volume sample could be obtained by a prick or swab.

In some embodiments, analysis of one or more vesicles in a biological sample is used to determine whether an additional biological sample should be obtained for analysis. For example, analysis of one or more vesicles in a serum sample can be used to determine whether a biopsy should be obtained. Similarly, analysis of one or more vesicles in a plasma sample can be used to determine whether a biopsy should be obtained.

### Binding Agents

A vesicle can also be isolated using one or more binding agents. A binding agent is an agent that binds to a vesicle component, or vesicle biomarker, which can be any component present in a vesicle or on the vesicle. The vesicle component can be a nucleic acid (e.g. RNA or DNA), protein, peptide, polypeptide, antigen, lipid, carbohydrate, or proteoglycan. The binding agent can be a capture agent, such that a capture agent captures the vesicle by binding to a vesicle target, such as carbohydrate or glycoprotein. The capture agent can be coupled to a substrate and used to isolate the vesicle, such as described herein. A vesicle can be isolated using one or more binding agents for a vesicle glycoprotein or carbohydrate. For example, the capture agent or binding agent can be a lectin.

A binding agent can be a lectin, nucleic acid (e.g. DNA, RNA), monoclonal antibody, polyclonal antibody, Fab, Fab′, single chain antibody, synthetic antibody, aptamer (DNA/RNA), peptoid, zDNA, peptide nucleic acid (PNA), locked nucleic acid (LNA), synthetic or naturally occurring chemical compound (including but not limited to a drug or labeling reagent), dendrimer, or any combination thereof. For example, the binding agent can be a lectin and used to isolate a vesicle.

In some instances, a single binding agent is used to isolate or detect a vesicle. In other instances, a combination of different binding agents is used to isolate or detect a vesicle. For example, at least 2, 3, 4, 5, 6, 7, 8, 9, 10, 11, 12, 13, 14, 15, 16, 17, 18, 19, 20, 25, 50, 75 or 100 different binding agents may be used to isolate or detect a vesicle from a biological sample. The one or more different binding agents for a vesicle can form a vesicle bio-signature in whole or in part, as further described below. Different binding agents can be used for multiplex analysis. In some embodiments, isolation or detection of more than one population of vesicles is performed by isolating or detecting each vesicle population with a different binding agent. Different binding agents can be bound to different particles, wherein the different particles are labeled. The particles can be differently labeled in order to distinguish particles. In another embodiment, an array comprising different binding agents is used for multiplex analysis, wherein the different binding agents are differentially labeled or can be ascertained based on the location of the binding agent on the array. Multiplexing can be accomplished up to the resolution capability of the labels or detection method, as described below.

The binding agent can be a binding agent that binds vesicle “housekeeping proteins,” or general vesicle biomarkers, such as CD63, CD9, CD81, CD82, CD37, CD53, or Rab-5b. Tetraspanins, a family of membrane proteins, can be used as general vesicle markers. The tetraspanins include CD151, CD53, CD37, CD82, CD81, CD9 and CD63. The binding agent can also be an agent that binds to vesicles derived from specific cell types, such as tumor cells (e.g. binding agent for EpCam) or specific cell-of-origins. The binding agent can be specific for a tumor antigen. For example, the binding agent used to isolate a vesicle may be a binding agent for an antigen selected from Table 2.

TABLE 2
Exemplary cancers by lineage, group comparisons of cells/tissue, and specific disease states and
antigens specific to those cancers, group cell/tissue comparisons and specific disease states.

| Cancer Lineage, | | |
|---|---|---|
| Group | | |
| Comparison, | | |
| Disease State | Antigens | References |
| Breast | BCA-225 | Cerani et al., 1985 |
| Breast | BCA-225 | Mesa-Tejada et al., 1988 |
| Breast | BCA-225 | Loy et al., 1991 |
| Breast | BCA-225 | Ma et al., 1993 |
| Breast | hsp70 | Wolfers et al. 2001 Nat Med 793: 297 |
| Breast | MART-1 | Wolfers et al. 2001 Nat Med 793: 297 |
| Breast | ER | Oldenhuis C N et al., Eur J Cancer. 2008 May; 44(7): 946-53. Epub |
| | | 2008 Apr. 7; Payne S J et al., Histopathology. 2008 January; 52(1): 82-90 |
| Breast | Class III b-tubulin | Galmarini C M et al., Clin Cancer Res. 2008 Jul. 15; 14(14): 4511-6 |
| Breast | VEGFA | Linderholm B K et al., Cancer Res. 2001 Mar. 1; 61(5): 2256-60 |
| Breast | HER2/neu (for | De Laurentiis M et al., Ann Oncol. 2005 May; 16 Suppl 4: iv7-13. |
| | Her2 + BC) | |
| Breast | GPR30 | Filardo E J et al., Steroids. 2008 October; 73(9-10): 870-3. |
| Breast | ErbB4(JM) | Määttä J A et al., Mol Biol Cell. 2006 January; 17(1): 67-79. |
| | isoform | |
| Breast | MPR8 | Bera T K et al., Molecular Medicine 7(8): 509-516, 2001 |
| Breast | MISIIR | Jamie N Bakkum-Gamez et al., Gynecologic oncology (Gynecol |
| | | Oncol) Vol. 108 Issue 1 Pg. 141-8 |
| Ovarian | CA125 (0C125)# | Bast et al., 1981 |
| Ovarian | CA125 | Dabawat S, et al., 1983 |
| Ovarian | CA125 | Davis H et al., 1986 |
| Ovarian | CA125 | Nouwen E, et al., 1986 |
| Ovarian | CA125 | Quirk J, et al., 1988 |
| Ovarian | CA-125 | Fukazawa I et al., 1988 |
| Ovarian | VEGFA | Osada R et al., Hum Pathol. 2006 November; 37(11): 1414-25. |
| Ovarian | VEGFR2 | Chen B Y et al., Zhonghua Zhong Liu Za Zhi. 2005 January; 27(1): 33-7 |
| Ovarian | HER2 | Steffensen K D et al., Int J Oncol. 2008 July; 33(1): 195-204 |
| Ovarian | MISIIR | Jamie N Bakkum-Gamez et al., Gynecologic oncology (Gynecol |
| | | Oncol) Vol. 108 Issue 1 Pq. 141-8 |
| Lung | CYFRA 21-1 | Kulpa J, et al., C Clin Chem 48: 1931-1937 (2002) |
| Lung | TPA-M | Kulpa J, et al., supra. |
| Lung | TPS | Kulpa J, et al., supra. |
| Lung | CEA | Kulpa J, et al., supra. |
| Lung | SCC-Ag | Kulpa J, et al., supra. |
| Lung | XAGE-1b | Kikuchi et al., Cancer Immunity, 8: 13 (2008) |
| Lung | HLA class I | Kikuchi et al., supra. |
| Lung | TA-MUC1 | Kuemmel et al., Lung Cancer Jun. 6, 2008 |
| Lung | KRAS | Zhang Z et al., Cancer Biol Ther. 2006 November; 5(11): 1481-6 |
| Lung | hENT1 | Oguri T et al., Cancer Lett. 2007 Oct. 18; 256(1): 112-9. |
| Lung | kinin B1 receptor | Chee J et al., Biol Chem. 2008 September; 389(9): 1225-33. |
| Lung | kinin B2 receptor | Chee J et al., Biol Chem. 2008 September; 389(9): 1225-33. |
| Lung | TSC403 | Ozaki K et al., CANCER RESEARCH 58, 3499-3503, Aug. 15, |
| | | 1998 |
| Lung | HTI56 | Dobbs L G et al., JHC Volume 47(2): 129-137, 1999 |
| Lung | DC-LAMP | Salaun B et al., American Journal of Pathology. 2004; 164: 861-871 |
| Colon | CEA | Park et al., 2002 |
| Colon | MUC2 | Park et al., 2002 |
| Colon | GPA33 | Huber et al., 2005 |
| Colon | CEACAM5 | Huber et al., 2005 |
| Colon | ENFB1 | Huber et al., 2006 |
| Colon | CCSA-3 | Leman et al., 2007 |
| Colon | CCSA-4 | Leman et al., 2008 |
| Colon | ADAM 10 | Choi et al., 2007, J Ethnopharmacol 110(1): 49-55. |
| Colon | CD44 | Choi et al., 2007, J Ethnopharmacol 110(1): 49-55. |
| Colon | NG2 | Choi et al., 2007, J Ethnopharmacol 110(1): 49-55. |
| Colon | ephrin-B1 | Choi et al., 2007, J Ethnopharmacol 110(1): 49-55. |
| Colon | plakoglobin | Choi et al., 2007, J Ethnopharmacol 110(1): 49-55. |
| Colon | galectin-4 | Choi et al., 2007, J Ethnopharmacol 110(1): 49-55. |
| Colon | RACK1 | Choi et al., 2007, J Ethnopharmacol 110(1): 49-55 |
| Colon | tetraspanin-8 | Choi et al., 2007, J Ethnopharmacol 110(1): 49-55 |
| Colon | FasL | Choi et al., 2007, J Ethnopharmacol 110(1): 49-55 |
| Colon | A33 | Choi et al., 2007, J Ethnopharmacol 110(1): 49-55 |
| Colon | CEA | Choi et al., 2007, J Ethnopharmacol 110(1): 49-55 |
| Colon | EGFR | Choi et al., 2007, J Ethnopharmacol 110(1): 49-55 |
| Colon | dipeptidase 1 | Choi et al., 2007, J Ethnopharmacol 110(1): 49-55 |
| Colon | PTEN | Frattini et al., 2007 |
| Colon | Na(+)-dependent | Wang Y et al., Pediatr Res. 1994 October; 36(4): 514-21. |
| | glucose transporter | |
| Colon | UDP- | Gong Q H et al., Pharmacogenetics 11: 357-368(2001). |
| | glucuronosyltransferase | |
| | 1A | |
| Benign Prostatic | KIA1 | Ueda T, et al., 1996 |
| Hyperplasia | | |
| Benign Prostatic | Intact Fibronectin | Janković M M, Kosanović M M, Dis Markers. 2008; 25(1): 49-58. |
| Hyperplasia | | |
| Prostate | PSA | Nurmikko P et al., 2000 |
| Prostate | TMPRSS2 | Wilson S et al., Biochem J. 2005 Jun. 15; 388(Pt 3): 967-72. |
| Prostate | FASLG | Huber et al., 2005, Gastroenterol Nurs 28(6): 510-1. |
| Prostate | TNFSF10 | Huber et al., 2005, Gastroenterol Nurs 28(6): 510-1 |
| Prostate | PSMA | Pinto J T et al., Clin Cancer Res. 1996 September; 2(9): 1445-51. |
| Prostate | NGEP | Das S et al., Cancer Res. 2007 Feb. 15; 67 (4): 1594-1601 |
| Prostate | IL-7R1 | Haudenschild D R et al., Prostate. 2006 Sep. 1; 66(12): 1268-74. |
| Prostate | CSCR4 | Chinni S R et al., Mol Cancer Res. 2008 March; 6(3): 446-57. |
| Prostate | CysLT1R | Matsuyama M et al., Oncol Rep. 2007 JuI; 18(1): 99-104. |
| Prostate | TRPM8 | Bidaux G et al., J Clin Invest. 2007 June; 117(6): 1647-57. |
| Prostate | Kv1.3 | Prevarskaya N et al., Cell Death Differ. 2007 July; 14(7): 1295-304. |
| Prostate | TRPV6 | Prevarskaya N et al., Cell Death Differ. 2007 July; 14(7): 1295-304. |
| Prostate | TRPM8 | Prevarskaya N et al., Cell Death Differ. 2007 July; 14(7): 1295-304. |
| Prostate | PSGR | Xu L L et al., Cancer Res. 2000 Dec. 1; 60(23): 6568-72. |
| Prostate | MISIIR | Bakkum-Gamez J. N. et al., Gynecol Oncol Vol. 108 Issue 1 Pg. |
| | | 141-8 |
| Melanoma | TYRP1 | Mears et al., 2004 |
| Melanoma | SILV | Mears et al., 2004 |
| Melanoma | MLANA | Mears et al., 2004 |
| Melanoma | MCAM | Mears et al., 2004 |
| Melanoma | CD63 | Azorsa et al. 1991 |
| Melanoma | CD63 | Barrio et al. 1998 |
| Melanoma | CD63 | Demetrick et al., 1992 |
| Melanoma | CD63 | Mete et al., 2005 |
| Melanoma | CD63 | Kwon et al., 2007 |
| Melanoma | Alix | Mears et al., 2004, Proteomics 4(12): 4019-31. |
| Melanoma | hsp70 | Mears et al., 2004, Proteomics 4(12): 4019-31 |
| Melanoma | moesin | Mears et al., 2004, Proteomics 4(12): 4019-31. |
| Melanoma | p120 catenin | Mears et al., 2004, Proteomics 4(12): 4019-31 |
| Melanoma | PGRL | Mears et al., 2004, Proteomics 4(12): 4019-31 |
| Melanoma | syntaxin-binding | Mears et al., 2004, Proteomics 4(12): 4019-31 |
| | protein 1 & 2 | |
| Melanoma | DUSP1 | |
| Brain | PRMT8 | Lee et al., 2005 |
| Brain | BDNF | Binder and Scharfman, 2004 |
| Brain | EGFR | Hicke et al., J. Biol. Chem. 276, 48644-48654, 2001; Daniels et al., |
| | | PNAS 100, 15416-15421, 2003 |
| Brain | DPPX | Kim et al., J. Biochem, 2001, Vol. 129, No. 2 289-295 |
| Brain | Elk | Lhotak V et al., MOLECULAR AND CELLULAR BIOLOGY, |
| | | May 1991, p. 2496-2502 |
| Brain | Densin-180 | Apperson M L et al., Journal of Neuroscience Volume 16, Number |
| | | 21, Issue of Nov. 1, 1996 pp. 6839-6852 |
| Brain | BAI2 | Shiratsuchi T et al., Cytogenet Cell Genet. 1997; 79(1-2): 103-8. |
| Brain | BAI3 | Shiratsuchi T et al., Cytogenet Cell Genet. 1997; 79(1-2): 103-8. |
| Psoriasis | flt-1 | Detmar M, et al., 1994 |
| Psoriasis | VPF receptors | Detmar M, et al., 1994 |
| Psoriasis | kdr | Detmar M, et al., 1994 |
| CVD | FATP6 | Gimeno R E et al., J Biol Chem. 2003 May 2; 278(18): 16039-44. |
| Hematological | CD44 | Liu J and Jiang G, II Mol Immunol. 2006 October; 3(5): 359-65. |
| malignancies | | |
| Hematological | CD58 | Kroger N, et al., 1997 |
| malignancies | | |
| Hematological | CD31 | Kroger N, et al., 1998 |
| malignancies | | |
| Hematological | CD11a | Kroger N, et al., 1999 |
| malignancies | | |
| Hematological | CD49d | Kroger N, et al., 2000 |
| malignancies | | |
| Hematological | GARP | Wang R et al., PLoS ONE. 2008 Jul. 16; 3(7): e2705. |
| malignancies | | |
| Hematological | BTS | Suenaga T et al., Eur J Immunol. 2007 November; 37(11): 3197-207. |
| malignancies | | |
| Hematological | Raftlin | Saeki K et al., The EMBO Journal (2003) 22, 3015-3026 |
| malignancies | | |
| Hepatocellular | HBxAg | Wang W, et al., 1991 |
| Carcinoma | | |
| Hepatocellular | HBsAg | Wang W, et al., 1991 |
| Carcinoma | | |
| Hepatocellular | NLT | Simonson G D et al., Journal of Cell Science 107, 1065-1072 (1994) |
| Carcinoma | | |
| Cervical Cancer | MCT-1 | Pinheiro C, et al., 2008 |
| Cervical Cancer | MCT-2 | Pinheiro C, et al., 2008 |
| Cervical Cancer | MCT-4 | Pinheiro C, et al., 2008 |
| Head and Neck | EGFR | Sheikh Ali M A et al., Cancer Sci. 2008 August; 99(8): 1589-94 |
| Cancer | | |
| Head and Neck | EphB4 | Yavrouian E J et al., Arch Otolaryngol Head Neck Surg. 2008 |
| Cancer | | September; 134(9): 985-91. |
| Head and Neck | EphrinB2 | Yavrouian E J et al., Arch Otolaryngol Head Neck Surg. 2008 |
| Cancer | | September; 134(9): 985-91. |
| Endometrial | AlphaV Beta6 | Hecht J L et al., Appl Immunohistochem Mol Morphol. 2008 Aug. |
| Cancer | integrin | 11. |
| Autoimmune | Tim-2 | Chakravarti S, et al., 2005 |
| Disease | | |
| Irritable Bowel | II-16 | Seegert D, et al., 2001 |
| Disease | | |
| Irritable Bowel | 5-HT | Kerckhoffs A P et al., Neurogastroenterol Motil. 2008 |
| Disease | | August; 20(8): 900-7. |
| Irritable Bowel | II-1beta | Seegert D, et al., 2001 |
| Disease | | |
| Irritable Bowel | II-12 | Seegert D, et al., 2001 |
| Disease | | |
| Irritable Bowel | TNF-alpha | Seegert D, et al., 2001 |
| Disease | | |
| Irritable Bowel | interferon gamma | Seegert D, et al., 2001 |
| Disease | | |
| Irritable Bowel | II-6 | Seegert D, et al., 2001 |
| Disease | | |
| Irritable Bowel | Rantes | Seegert D, et al., 2001 |
| Disease | | |
| Irritable Bowel | MCP-1 | Seegert D, et al., 2001 |
| Disease | | |
| Diabetes | IL-6 | Pradhan A, et al., 2001 |
| Diabetes | CRP | Pradhan A, et al., 2001 |
| Diabetes | RBP4 | Lee S J et al., Anal Chem. 2008 Apr. 15; 80(8): 2867-73. |
| Barrett's | p53 | Hamelin R, et al., 1994 |
| Esophagus | | |
| Barrett's | MUC1 | Burjonrappa S C et al., Indian J Cancer. 2007 January-March; 44(1): 1-5. |
| Esophagus | | |
| Barrett's | MUC6 | Glickman J N et al., Am J Surg Pathol. 2003 October; 27(10): 1357-65 |
| Esophagus | | |
| Fibromyalgia | neopterin | Bonaccorso S, et al., 1997 |
| Fibromyalgia | gp130 | Maes M et al., 1999 |
| Stroke | S-100 | Missler U, et al., 1997 |
| Stroke | Neuron specific | Missler U, et al., 1997 |
| | enolase | |
| Stroke | PARK7 | Allard L, et al., 2005 |
| Stroke | NDKA | Allard L, et al., 2005 |
| Stroke | ApoC-I | Allard L, et al., 2005 |
| Stroke | ApoC-III | Allard L, et al., 2003 |
| Stroke | SAA | Allard L, et al., 2003 |
| Stroke | AT-III fragment | Allard L, et al., 2003 |
| Stroke | Lp-PLA2 | http://www.doctorslounge.com/neurology/news/stroke_lp- |
| | | pla2_crp.shtml; Gorelick P B, Am J Cardiol. 2008 Jun. |
| | | 16; 101(12A): 34F-40F |
| Stroke | hs-CRP | http://www.doctorslounge.com/neurology/news/stroke_lp- |
| | | pla2_crp.shtml |
| Multiple | B7 | Ferrante P, et al., 1998 |
| Sclerosis | | |
| Multiple | B7-2 | Ferrante P, et al., 1998 |
| Sclerosis | | |
| Multiple | CD-95(fas) | Ferrante P, et al., 1998 |
| Sclerosis | | |
| Multiple | Apo-1/Fas | Ferrante P, et al., 1998 |
| Sclerosis | | |
| Parkinsons | PARK2 | Shimura H, eat al., 2000 |
| Disease | | |
| Parkinsons | Ceruloplasmin | Shi M et al., Neurobiol Dis. 2008 Sep. 26. |
| Disease | | |
| Parkinsons | VDBP | Zhang et al., 2008 Am. J. Clin. Pathol. 129, 526-9., |
| Disease | | |
| Parkinsons | tau | Zhang et al., 2008 Am. J. Clin. Pathol. 129, 526-9; Mollenhauer B |
| Disease | | et al., Dement Geriatr Cogn Disord; 2006; 22(3): 200-8; Davidsson P |
| | | and Sjögren M, Dis Markers. 2005; 21(2): 81-92. |
| Parkinsons | DJ-1 | Waragai et al., 2007 Neurosci. Lett. 425, 18-22 & Waragai et at |
| Disease | | 2006 Biochem. Biophys. Res. Commun. 345, 967-72 |
| Rheumatic | Citrulinated fibrin | Skriner et al., 2006 |
| Disease | a-chain | |
| Rheumatic | CD5 antigen-like | Skriner et al., 2006 |
| Disease | fibrinogen | |
| | fragment D | |
| Rheumatic | CD5 antigen-like | Skriner et al., 2006 |
| Disease | fibrinogen | |
| | fragment B | |
| Rheumatic | TNFalpha | Anderson A K et al., Arthritis Res Ther. 2008; 10(2): 204. Epub 2008 |
| Disease | | Mar. 14. |
| Alzheimers | APP695 | Rebeck G, et al., 2001 |
| Disease | | |
| Alzheimers | APP751 | Rebeck G, et al., 2001 |
| Disease | | |
| Alzheimers | APP770 | Rebeck G, et al., 2001 |
| Disease | | |
| Alzheimers | BACE1 | Hebert S S et al., 2008. Proc Natl Acad Sci U.S.A., 105(17): 6415- |
| Disease | | 20 |
| Alzheimers | Cystatin C | Simonsen et al., 2008 Neurobiol. Aging. 29, 961-8 |
| Disease | | |
| Alzheimers | Amyloid Beta | Simonsen et al., 2008 Neurobiol. Aging. 29, 961-8 |
| Disease | | |
| Alzheimers | t-Tau | Simonsen et al., 2008 Neurobiol. Aging. 29, 961-8 |
| Disease | | |
| Alzheimers | Complement factor | Hye et al., 2006 Brain. 129, 3042-50 |
| Disease | H | |
| Alzheimers | alpha-2- | Hye et al., 2006 Brain. 129, 3042-50 |
| Disease | macroglobulin | |
| Alzheimers | APOE4 | Albert M S, Proc Natl Acad Sci USA. 1996 Nov. 26; 93(24): 13547- |
| Disease | | 51. |
| Prion Diseases | PrPSc | Takemura K et al., Exp Biol Med (Maywood) February; 231(2)204-14, |
| | | 2006 |
| Prion Diseases | 14-3-3 zeta | Kubler E et at, British Medical Bulletin 66: 267-279, 2003 |
| Prion Diseases | S-100 | Kubler E et al, British Medical Bulletin 66: 267-279, 2003 |
| Prion Diseases | AQP-4 | Kubler E et al, British Medical Bulletin 66: 267-279, 2003 |
| Chronic | Chemokine | White F A et al., Proc Natl Acad Sci USA. 2007 Dec. |
| Neuropathic Pain | receptor (CCR2/4) | 18; 104(51): 20151-8 |
| Peripheral | OX42 (rodent) | Blackbeard J et al., J Neurosci Methods. 2007 Aug. 30; 164(2): 207- |
| Neuropathic Pain | | 17 |
| Peripheral | ED9 (rodent) | Blackbeard J et al., J Neurosci Methods. 2007 Aug. 30; 164(2): 207- |
| Neuropathic Pain | | 17 |
| Schizophrenia | ATP5B | Altar C A, Neuropsychopharmacology. 2008 Oct. 15. |
| Schizophrenia | ATP5H | Altar C A, Neuropsychopharmacology. 2008 Oct. 15. |
| Schizophrenia | ATP6V1B | Altar C A, Neuropsychopharmacology. 2008 Oct. 15. |
| Schizophrenia | DNM1 | Altar C A, Neuropsychopharmacology. 2008 Oct. 15. |
| GIST | PDGFRA | Yang J et al., ncer. 2008 Oct. 1; 113(7): 1532-43 |
| GIST | c-kit | Yang J et al., ncer. 2008 Oct. 1; 113(7): 1532-43 |
| GIST | NHE-3 | Kulaksiz H et al., Cell Tissue Res. 2001 March; 303(3): 337-43. |
| Renal Cell | HIF1alpha | Rathmell W K, Chen S, Expert Rev Anticancer Ther. 2008 |
| Carcinoma | | January; 8(1): 63-73. |
| Renal Cell | VEGF | Rathmell W K, Chen S, Expert Rev Anticancer Ther. 2008 |
| Carcinoma | | January; 8(1): 63-74. |
| Renal Cell | PDGFRA | Rathmell W K, Chen S, Expert Rev Anticancer Ther. 2008 |
| Carcinoma | | January; 8(1): 63-74. |
| Cirrhosis | NLT | Simonson G D et al., Journal of Cell Science 107, 1065-1072 (1994) |
| Cirrhosis | HBsAg | Wang, W. et al., 1991 |
| Esophageal | CaSR | Justinich C J et al., Am J Physiol Gastrointest Liver Physiol. 2008 |
| cancer | | January; 294(1): G120-9. |
| Influenza | Hemmaglutanin | Verma R K and Jain Amita, FEMS Immunol Med Microbiol 51 |
| | | (2007) 453-461 |
| Influenza | Neurominidase | Verma R K and Jain Amita, supra. |
| TB | Antigen 60 | Verma R K and Jain Amita, supra. |
| TB | HSP antigen | Verma R K and Jain Amita, supra. |
| TB | Lipoarabinomannan | Verma R K and Jain Amita, supra. |
| | antigen | |
| TB | Antigen of | Verma R K and Jain Amita, supra. |
| | acylated trehalose | |
| | family | |
| TB | DAT antigen | Verma R K and Jain Amita, supra. |
| TB | Sulfolipid antigen | Verma R K and Jain Amita, supra. |
| TB | TAT antigen | Verma R K and Jain Amita, supra. |
| TB | Trehalose 6,6- | Verma R K and Jain Amita, supra. |
| | dimycolate (cord- | |
| | factor) antigen | |
| HIV | Gp41 | Phogat S et al., J Intern Med. 2007 July; 262(1): 26-43. |
| HIV | gp120 | Phogat S et al., J Intern Med. 2007 July; 262(1): 26-43. |
| Autism | VIP | Nelson K B et al Annals of Neurology 2001, 49: 597-606.. |
| Autism | PACAP | Nelson K B et al Annals of Neurology 2001, 49: 597-606. |
| Autism | CGRP | Nelson K B et al Annals of Neurology 2001, 49: 597-606. |
| Autism | NTS | Nelson K B et al Annals of Neurology 2001, 49: 597-606. |
| Asthma | YKL-40 | Scot, I., Thorax 2008; 63: 365, A New Biomarker in Asthma |
| Asthma | S-nitrosothiols | Holgate, S T., Lancet. 1998 May 2; 351(9112): 1317-9. |
| Asthma | SCCA2 | Izuhara, K., Allergol Int. 2006 December; 55 (4): 361-7. |
| Asthma | PAI | Izuhara, K., Allergol Int. 2006 December; 55 (4): 361-7. |
| Asthma | amphiregulin | Izuhara, K., Allergol Int. 2006 December; 55 (4): 361-7. |
| Asthma | Periostin | Izuhara, K., Allergol Int. 2006 December; 55 (4): 361-7. |
| Lupus | TNFR | Suh C H and Kim H A, Expert Rev Mol Diagn. 2008 March; 8(2): 189- |
| | | 98 |
| Vulnerable | Alpha v Beta 3 | Burtea C et al., Cardiovasc Res. 2008 Apr. 1; 78(1): 148-57. |
| plaque | integrin | |
| Vulnerable | MMP9 | Blankenberg S et al., 2003 Circulation 107: 1579-1585. |
| plaque | | |

The binding agent can be for an antigen such as 5T4, B7H3, caveolin, CD63, CD9, E-Cadherin, MFG-E8, PSCA, PSMA, Rab-5B, STEAP, TNFR1, CD81, EpCam, CD59, or CD66. One or more binding agents, such as one or more binding agents for two or more of the antigens, can be used for isolating a vesicle. The binding agent used can be selected based on the desire of isolating vesicles derived from particular cell types, or cell-of-origin specific vesicles.

The binding agent for a vesicle can also be selected from those listed in Table 3.

TABLE 3
Exemplary cancers by lineage, group comparisons of cells/tissue, and specific disease states and binding
agents specific to those cancers, group cell/tissue comparisons and specific disease states

| Cancer Lineage, | | |
|---|---|---|
| Group | | |
| Comparison, | | |
| Other | | |
| Significant | | |
| Disease State | Binding Agents | Reference(s) |
| Breast | Herceptin (Trastuzumab) | Adams G P, Weiner L M, Nat Biotechnol. 2005 |
| | | September; 23(9): 1147-57. |
| Breast | CCND1 PNA | Tian et al, NAR 24(5-7): 1085-91, 2005; Tian et al., Ann NY |
| | | Acad Sci 1059, 106-44, 2005 |
| Breast | MYC PNA | Tian et at, NAR 24(5-7): 1085-91, 2005; Tian et al., Ann NY |
| | | Acad Sci 1059, 106-44, 2005 |
| Breast | IGF-1 PNA | Tian et al, J. of Nucl Med 48(10), 1699-707, 20007 |
| Breast | MYC PNA | Tian et al., Bioconjug Chem 16)1)70-9, 2005 |
| Breast | SC4 aptamer (Ku) | Zhang et al. 2004 |
| Breast | All-7 aptamer (ERB2) | Kunz et al., MolecularCancer Research(4) 983998, 2006 |
| Breast | Galectin -3 binding agent | Cancer Invest 26(6)615-23, 2008 |
| Breast | mucin-type O-glycans | Cancer Invest 26(6)615-23, 2008 |
| | binding agent | |
| Breast | L-PHA binding agent | Abbott et al., J Proteome Res 7(4)1470-80, 2008 |
| Breast | Galectin-9 binding agent | Yamaguchi et al., Breast J 5(2), 2006 |
| Breast | ER | Payne S J et al., Histopathology. 2008 January; 52(1): 82-90. |
| Breast | PR | Payne S J et al., Histopathology. 2008 January; 52(1): 82-90. |
| Ovarian | (90)Y-muHMFG1 | Oei et al 2008 |
| | binding agent | |
| Ovarian | OC125 (anti-CA125 | Matsuoka et al 1987 |
| | antibody) | |
| Ovarian | monoclonal antibodies | Kosmas et al, Oncology 55 (5), 435-446, 1998 |
| | (HMFG1, HMFG2, | |
| | H317, and H17E2), | |
| | Hu2PLAP | |
| Lung | SCLC specific aptamer | Chen et at, Chem Med Chem (3)991-1001, 2008 |
| | HCA12 | |
| Lung | SCLC specific aptamer | Chen et al, Chem Med Chem (3)991-1001, 2008 |
| | HCC03 | |
| Lung | SCLC specific aptamer | Chen et al, Chem Med Chem (3)991-1001, 2008 |
| | HCH07 | |
| Lung | SCLC specific aptamer | Chen et at, Chem Med Chem (3)991-1001, 2008 |
| | HCH01 | |
| Lung | A-p50 aptamer (NF-KB) | Mi et al., Mol Ther 16(1)66-73, 2008 |
| Lung | Cetuximab | Rossi A et al., Rev Recent Clin Trials. 2008 September; 3(3): 217-27 |
| Lung | Panitumumab | Rossi A et al., Rev Recent Clin Trials. 2008 September; 3(3): 217-27 |
| Lung | Bevacizumab | Gettinger S et al., Semin Respir Crit Care Med. 2008 |
| | | June; 29(3): 291-301 |
| Lung | L19 antibody | Pedretti et al., Lung Cancer Sep. 15, 2008 |
| Lung | F16 antibody | Pedretti et al., Lung Cancer Sep. 15, 2008 |
| Lung | anti-CD45 (anti-ICAM-1 | Brooks et al., Int J Cancer 2438(10)2438-45, 2008 |
| | antibody, aka UV3) | |
| Lung | L2G7 Ab (anti-HGF | Stabile et al., Mol Cancer Ther 7(7)1913-22, 2008 |
| | antibody) | |
| Colon | angiopoietin 2 specific | Sarraf-Yazdi et al., J SURG Res 146(1)16-23, 2008. |
| | aptamer | |
| Colon | beta-catenin aptamer | Lee et al., Cancer Research 66(21)10560-6, 2006. |
| Colon | TCF1 aptamer | Choi et al., Mol Caner Therapy (9)2428-34, 2006. |
| Colon | anti-Derlin1 antibody | Ran et al., Clin Cancer Res 14(206538-45, 2008 |
| Colon | anti-RAGE antibody | Turovskaya et al., Carcinogenesis 29(10)2035-2043, 2008. |
| Colon | monoclonal antibody | Turovskaya et al., Carcinogenesis 29(10)2035-2043, 2008. |
| | gb3.1 | |
| Colon | Galectin-3 binding agent | Greco et al., Glycobiology 14(9)783-92, 2004. |
| Colon | Cetuximab | Giuliani F, Colucci G et al., Int J Biol Markers. 2007 January- |
| | | March; 22(l Suppl 4): S62-70 |
| Colon | Panitumumab | Chua Y J, Cunningham D, Clin Colorectal Cancer. 2005 |
| | | November; 5 Suppl 2: S81-8. |
| Colon | Matuzumab | Chua Y J, Cunningham D, Clin Colorectal Cancer. 2005 |
| | | November; 5 Suppl 2: S81-8. |
| Colon | Bevacizumab | Majer M et al., Anticancer Agents Med Chem. 2007 |
| | | September; 7(5): 492-503 |
| Colon | Mac-2 binding agent | Lotz M M et al., Proc Natl Acad Sci USA. 1993 90(18): |
| | | 8319-23, “Mitogen-activated protein kinases p42mapk and |
| | | p44mapk are required for fibroblast proliferation.” |
| Adenoma versus | Complement C3 | Qui et al, J of Proteome Res 7(4)1693-1703, 2008 |
| CRC | | |
| Adenoma versus | histidine-rich | Qui et al, J of Proteome Res 7(4)1693-1703, 2008 |
| CRC | glycoprotein binding | |
| | agent | |
| Adenoma versus | kininogen-1 binding | Qui et al, J of Proteome Res 7(4)1693-1703, 2008 |
| CRC | agent | |
| Adenoma versus | Galectin-3 binding agent | Schoeppner H L et al., Cancer. 1995 Jun. 15; 75(12): 2818-26. |
| CRC | | |
| Adenoma with | Galectin-3 binding agent | Schoeppner H L et al., Cancer. 1995 Jun. 15; 75(12): 2818-26. |
| low grade versus | | |
| high grade | | |
| dysplasia | | |
| CRC versus | anti-ODC monoclonal | Hu H Y et al., World J Gastroenterol. 2005 Apr. |
| normal | antibody | 21; 11(15): 2244-8. |
| CRC versus | anti-CEA monoclonal | Zhang H Z et al., Cancer Res. 1989 Oct. 15; 49(20): 5766-73. |
| normal | antibody | |
| CRC versus | Mac-2 binding agent | Lotz M M et al., Proc Natl Acad Sci USA. 1993 Apr |
| normal | | 15; 90(8): 3466-70. |
| Prostate | PSA binding agent | Nurmikko P et al., 2000, Clin Chem 46(10): 1610-8. |
| Prostate | PSMA binding agent | Aggarwal S et al., Cancer Res. 2006 Sep. 15; 66(18): 9171-7. |
| Prostate | TMPRSS2 binding agent | Wilson S et al., Biochem J. 2005 Jun. 15; 388(Pt 3): 967-72. |
| Prostate | monoclonal antibody | Sawant et al., J Drug Target 16(7)601-4, 2008. |
| | 5D4 | |
| Prostate | XPSM-A9 | Lupold et al., Cancer Research 62(14): 4029-4033, 2002. |
| Prostate | XPSM-A10 | Lupold et al., Cancer Research 62(14): 4029-4033, 2002. |
| Prostate | Galectin-3 binding agent | Califice et al., Int J Oncol 25(4)983-92, 2004 |
| Prostate | E-selectin binding agent | Bhaskar et al., Cancer Research 63(19(6387-94, 2003. |
| Prostate | Galectin-1 binding agent | van den Brule et al., J Pathology 193(1)80-7, 2001 |
| Prostate | E4 (IgG2a kappa) | Nilsson S et al., Cancer Biother Radiopharm. 1997 |
| | | December; 12(6): 395-403. |
| Melanoma | Tremelimumab (anti- | Camacho L H, Expert Opin Investig Drugs 17(3)371-85, 2008. |
| | CTLA4 antibody) | |
| Melanoma | 1pilimumumab (anti- | Lens M et al., Recent Patents Anticancer Drug Discovery |
| | CTLA4 antibody) | Jun3(2)105-13, 2008. |
| Melanoma | CTLA-4 aptamers | Santulli-Marotto et al., Cancer Research 63(21)7483-9, 2003. |
| Melanoma | STAT-3 pep tide | Nagel Wolfrum et al., Molecular Cancer Research 2: 170-182, |
| | aptamers | 2004 |
| Melanoma | Galectin-1 binding agent | Mathieu et al., J Invest Dermatol 127(10)2399-410, 2007, Le |
| | | Mercier et al., J Neuropathol Exp Neurol. 67(5)456-69, 2008. |
| Melanoma | Galectin-3 binding agent | Prieto et al., Clin Cancer Res 12(22)6709-15, 2006; |
| | | Vereecken et al., Arch Dermatol Res 296(8)353-8, 2005 |
| Melanoma | PNA | Dore et al., Pigment Cell Res 7(6)461-4, 1994. |
| Pancreatic | H38-15 (HGF aptamer) | Saito T and Tomida M, DNA Cell Biol. 2005 October; 24(10): 624- |
| | | 33. |
| Pancreatic | H38-21(HGF aptamer) | Saito T and Tomida M, DNA Cell Biol. 2005 October; 24(10): 624- |
| | | 33. |
| Pancreatic | Matuzumab | Kleepspeies A et al., Clin Cancer Res. 2008 Sep. |
| | | 1; 14(17): 5426-36 |
| Pancreatic | Cetuximanb | Burris H 3rd et al., Oncologist. 2008 March; 13(3): 289-98. |
| Pancreatic | Bevacizumab | Burris H 3rd et al., Oncologist. 2008 March; 13(3): 289-98. |
| Brain | aptamer 111.1 (pigpen) | Blank Met al., JBC May 11; 276(19)16464-8, 2001 |
| Brain | TTA1 (Tenascin-C ) | Hicke et al., J. Biol. Chem. 276, 48644-48654, 2001; Daniels |
| | aptamer | et al., PNAS 100, 15416-15421, 2003 |
| Psoriasis | E-selectin binding agent | Rottman J B et al., Lab Invest. 2001 March; 81(3): 335-47. |
| Psoriasis | ICAM-1 binding agent | Rottman J B et al., Lab Invest. 2001 March; 81(3): 335-47. |
| Psoriasis | VLA-4 binding agent | Rottman J B et al., Lab Invest. 2001 March; 81(3): 335-47. |
| Psoriasis | VCAM-1 binding agent | Rottman J B et al., Lab Invest. 2001 March; 81(3): 335-47. |
| Psoriasis | alphaEbeta7 binding | Rottman J B et al., Lab Invest. 2001 March; 81(3): 335-47. |
| | agent | |
| Cardiovascular | RB007 (factor IXA | Chan M Y et al., Circulation. 2008 Jun. 3; 117(22): 2865-74. |
| Disease | aptamer) | Epub 2008 May 27 |
| Cardiovascular | ARC1779 (anti VWF) | Gilbert J C et al., Circulation. 2007 Dec. 4; 116(23): 2678-86. |
| Disease | aptamer | Epub 2007 Nov. 19 |
| Cardiovascular | LOX1 binding agent | Dunn S et al., Biochem J. 2008 Jan. 15; 409(2): 349-55. |
| Disease | | |
| Hematological | anti-CD20 | Ravandi F et al., Clin Cancer Res. 2003 February; 9(2): 535. |
| malignancies | | |
| Hematological | anti-CD52 | Ravandi F et al., Clin Cancer Res. 2003 February; 9(2): 535. |
| malignancies | | |
| B-Cell Chronic | Rituximab | Robak T, Leuk Lymphoma. 2004 February; 45(2): 205-19. |
| Lymphocytic | | |
| Leukemias | | |
| B-Cell Chronic | Alemtuzumab | Robak T, Leuk Lymphoma. 2004 February; 45(2): 205-19. |
| Lymphocytic | | |
| Leukemias | | |
| B-Cell Chronic | Apt48 (BCL6) | Chattopadhyay et al 2006, J Assoc Physicians India 54: 547. |
| Lymphocytic | | |
| Leukemias | | |
| B-Cell Chronic | R0-60 aptamer | Wu C C et al., Hum Gene Ther. 2003 Jun 10; 14(9): 849-60. |
| Lymphocytic | | |
| Leukemias | | |
| B-Cell Chronic | D-R15-8 aptamer | Wu C C et al., Hum Gene Ther. 2003 Jun 10; 14(9): 849-60. |
| Lymphocytic | | |
| Leukemias | | |
| B-cell lymphoma | Ibritumomab | Cheson B D, Leonard J P, N Engl J Med. 2008 Aug. |
| | | 7; 359(6): 613-26. |
| B-cell lymphoma | Tositumomab | Cheson B D, Leonard J P, supra. |
| B-cell lymphoma | Anti-CD20 Antibodies | Cheson B D, Leonard J P, supra. |
| B-cell lymphoma | Alemtuzumab | Cheson B D, Leonard J P, supra. |
| B-cell lymphoma | Galiximab | Cheson B D, Leonard J P, supra. |
| B-cell lymphoma | Anti-CD40 Antibodies | Cheson B D, Leonard J P, supra. |
| B-cell lymphoma | Epratuzumab | Cheson B D, Leonard J P, supra.. |
| B-cell lymphoma | Lumiliximab | Cheson B D, Leonard J P, supra. |
| B-cell lymphoma | Monoclonal antibody | Cheson B D, Leonard J P, supra. |
| | Hu1D10 | |
| B-cell | Galectin-3 binding agent | D'Haene N et al., Int J Immunopathol Pharmacol. 2005 July- |
| lymphoma- | | September; 18(3): 431-43. |
| DLBCL | | |
| B-cell lymphoma | Apt48 | Chattopadhyay A et al., Oncogene. 2006 Apr. 6; 25(15): 2223- |
| | | 33. |
| Burkitt's | TD05 aptamer | Mallikaratchy P et al., Mol Cell Proteomics December; 6(12)2230- |
| lymphoma | | 8, 2007. |
| Burkitt's | IgM monoclonal | Wiels J et al., Cancer Res. 1984 January; 44(1): 129-33. |
| lymphoma | antibody (38-13) | |
| Cervical Cancer | Galectin-9 binding agent | Liang et al., Clin Oncol 134(8)899-907, 2008. |
| Cervical Cancer | HPVE7 aptamer | Nauenburg S et al., FASEB J. 2001 March; 15(3): 592-4. Epub |
| | | 2001 Jan. 19. |
| Endometrial | Galectin-1 binding agent | Mylonas I et al., Anticancer Res. 2007 JulyAugust; 27(4A): 1975- |
| Cancer | | 80. |
| Head and Neck | (111)In-cMAb U36 | Sandstrom K et al., Tumour Biol. 2008; 29(3): 137-44. |
| Cancer | | |
| Head and Neck | anti-LOXL4 antibody | Weise J B et al., Eur J Cancer. 2008 June; 44(9): 1323-31. |
| Cancer | | |
| Head and Neck | U36 monoclonal | Verel I et al., Int J Cancer. 2002 May 20; 99(3): 396-402. |
| Cancer | antibody | |
| Head and Neck | BIWA-1 monoclonal | Verel I et al., Int J Cancer. 2002 May 20; 99(3): 396-402. |
| Cancer | antibody | |
| Head and Neck | BIWA-2 monoclonal | Verel I et al., Int J Cancer. 2002 May 20; 99(3): 396-402. |
| Cancer | antibody | |
| Head and Neck | BIWA-4 monoclonal | Verel I et al., Int J Cancer. 2002 May 20; 99(3): 396-402. |
| Cancer | antibody | |
| Head and Neck | BIWA-8 monoclonal | Verel I et al., Int J Cancer. 2002 May 20; 99(3): 396-402. |
| Cancer | antibody | |
| Irritable Bowel | ACCA (anti-glycan | Li X et al., World J Gastroenterol. 2008 Sep. 7; 14(33): 5115- |
| Disease | antibody) | 24. |
| Irritable Bowel | ALCA(anti-glycan | Li X et al., World J Gastroenterol. 2008 Sep. 7; 14(33): 5115- |
| Disease | antibody) | 24. |
| Irritable Bowel | AMCA (anti-glycan | Li X et al., World J Gastroenterol. 2008 Sep. 7; 14(33): 5115- |
| Disease | antibody) | 24. |
| Diabetes | RBP4 aptamer | Lee S J et al., Anal Chem. 2008 Apr. 15; 80(8): 2867-73. |
| Fibromyalgia | L-selectin binding agent | Macedo J A et al., J Neuroimmunol. 2007 August; 188(1-2): 159- |
| | | 66,. |
| Multiple | Natalizumab (Tysabri) | Goodin D S et al., Neurology. 2008 Sep. 2; 71(10): 766-73. |
| Sclerosis | | |
| Rheumatic | Rituximab (anti-CD20 | Anderson A K et al., Arthritis Res Ther. 2008; 10(2): 204. Epub |
| Disease | antibody) | 2008 Mar. 14. |
| Rheumatic | Keliximab (anti-CD4 | Anderson A K et al., Arthritis Res Ther. 2008; 10(2): 204. Epub |
| Disease | antibody) | 2008 Mar. 14. |
| Alzheimers | TH14-BACE1 aptamers | Rentmeister A et al., RNA. 2006 September; 12(9): 1650-60. Epub |
| Disease | | 2006 Aug. 3 |
| Alzheimers | S10-BACE1 aptamers | Rentmeister A et al., RNA. 2006 September; 12(9): 1650-60. Epub |
| Disease | | 2006 Aug. 3 |
| Alzheimers | anti-Abeta monoclonal | Geylis V et al., Autoimmun Rev. 2006 January; 5(1): 33-9. Epub |
| Disease | antibody | 2005 Aug. 1. Review. |
| Alzheimers | Bapineuzumab (AAB- | Hock C et al., Neuron. 2003 May 22; 38(4): 54754 |
| Disease | 001) - Elan | |
| Alzheimers | LY2062430 (anti- | Irena Melnikova, Nature Reviews Drug Discovery 6, 341-342 |
| Disease | amyloid beta Ab)-Eli | (May 2007) |
| | Lilly | |
| Alzheimers | BACE1-Anti sense | Faghihi M A et al., Nat Med. 2008 July; 14(7): 723-30. Epub |
| Disease | | 2008 Jun 29 |
| Prion Diseases | rhuPrP © aptamer | Takemura K et al., Exp Biol Med (Maywood) February; 231(2)204- |
| | | 14, 2006 |
| Prion Diseases | DP7 aptamer | Proske D et al., Chembiochem. 2002 Aug. 2; 3(8): 717-25. |
| Prion Diseases | Thioaptamer 97 | King D J et al., J Mol Biol. 2007 Jun. 15; 369(4): 1001-14. Epub |
| | | 2007 Feb. 9 |
| Prion Diseases | SAF-93 aptamer | Rhie A et al., J Biol Chem. 2003 Oct. 10; 278(41): 39697-705. |
| | | Epub 2003 Aug. 5 |
| Prion Diseases | 15B3 (anti-PrPSc | Korth C et al., Nature 390: 74-77, 1997 |
| | antibody) | |
| Prion Diseases | monoclonal anti PrPSc | Jones M et al., Brain Pathol. 2008 May 26. |
| | antibody P1: 1 | |
| Prion Diseases | 1.5D7, 1.6F4 antibodies | Cordes H, J Immunol Methods September 15; 337(2)106-20, 2008 |
| Prion Diseases | monoclonal antibody | Krasemann S et al., Mol Med. 1996 November; 2(6): 725-34 |
| | 14D3 | |
| Prion Diseases | monoclonal antibody | Krasemann S et al., Mol Med. 1996 November; 2(6): 725-34 |
| | 4F2 | |
| Prion Diseases | monoclonal antibody | Krasemann S et al., Mol Med. 1996 November; 2(6): 725-34 |
| | 8G8 | |
| Prion Diseases | monoclonal antibody | Krasemann S et al., Mol Med. 1996 November; 2(6): 725-34 |
| | 12F10 | |
| Sepsis | HA-1A monoclonal | Cross A S and Opal S Journal of Endotoxin Research, Vol. 1, |
| | antibody | No. 1, 57-69 (1994) |
| Sepsis | E-5 monoclonal antibody | Cross A S and Opal S Journal of Endotoxin Research, Vol. 1, |
| | | No. 1, 57-69 (1994) |
| Sepsis | TNF-alpha monoclonal | Abraham E et al., JAMA Vol. 273 No. 12, Mar. 22, 1995 |
| | antibody | |
| Sepsis | Afelimomab | Vincent J L Int J Clin Pract. 2000 April; 54(3): 190-3 |
| Sepsis | E-selectin binding agent | Tsokos M et al., Int Journal of Legal Medicine, Volume 113, |
| | | Number 6: 338-342, 2000 |
| Schizophrenia | L-selectin binding agent | Iwata Y et al., Schizophr Res. 2007 January; 89(1-3): 154-60. Epub |
| | | 2006 Oct. 17 |
| Schizophrenia | N-CAM binding agent | Vawter M P et al., Exp Neurol. 1998 February; 149(2): 424-32 |
| Depression | GPlb binding agent | Walsh M T et al., Life Sci. 2002 May 17; 70(26): 3155-65 |
| GIST | anti-DOG1 antibody | Espinosa F et al., Am J Surg Pathol February; 32(2)210-8, 2008 |
| Esophageal | CaSR binding agent | Justinich C J et al., Am J Physiol Gastrointest Liver Physiol. |
| cancer | | 2008 January; 294(1): G120-9. |
| Gastric cancer | Calpain nCL-2 binding | Hata et al., J. Biol. Chem., Vol. 281, Issue 16, 11214-11224, |
| | agent | Apr. 21, 2006 |
| Gastric cancer | drebrin binding agent | Keon B H et al., Journal of Cell Science, Vol 113, Issue 2 325- |
| | | 336 |
| Osteoarthritis | DDR-2 binding agent | Xu et al., Arthritis Rheum. 2007 August; 56(8): 2663-73. |
| COPD | CXCR3 binding agent | Freeman C M et al., Am J Pathol. 2007 September; 171(3): 767-76. |
| COPD | CCR5 binding agent | Freeman C M et al., Am J Pathol. 2007 September; 171(3): 767-76. |
| COPD | CXCR6 binding agent | Freeman C M et al., Am J Pathol. 2007 September; 171(3): 767-76. |
| Asthma | VIP binding agent | Nelson K B et al Annals of Neurology 2001, 49: 597-606. |
| Asthma | PACAP binding agent | Nelson K B et al Annals of Neurology 2001, 49: 597-606.. |
| Asthma | CGRP binding agent | Nelson K B et al Annals of Neurology 2001, 49: 597-606.. |
| Asthma | NT3 binding agent | Nelson K B et al Annals of Neurology 2001, 49: 597-606.. |
| Asthma | YKL-40 binding agent | Scot, I., Thorax 2008; 63: 365, A New Biomarker in Asthma |
| Asthma | S-nitrosothiols | Holgate, S T., Lancet. 1998 May 2; 351(9112): 1317-9. |
| Asthma | SCCA2 binding agent | Izuhara, K., Allergol Int. 2006 December; 55 (4): 3617. |
| Asthma | PAI binding agent | Izuhara, K., Allergol Int. 2006 December; 55 (4): 3617. |
| Asthma | amphiregulin binding | Izuhara, K., Allergol Int. 2006 December; 55 (4): 3617. |
| | agent | |
| Asthma | Periostin binding agent | Izuhara, K., Allergol Int. 2006 December; 55 (4): 3617. |
| Vulnerable | Gd-DTPA-g-mimRGD | Burtea C et al., Cardiovasc Res. 2008 Apr. 1; 78(1): 148-57. |
| plaque | (Alpha v Beta 3 integrin | |
| | binding peptide) | |
| Vulnerable | MMP-9 binding agent | Blankenberg S et al., 2003 Circulation 107: 1579-1585. |
| plaque | | |

The binding agents can be used to detect the vesicles, such as for detecting cell-of-origin specific vesicles. A binding agent or multiple binding agents can themselves form a binding agent profile that provides a bio-signature for a vesicle. One or more binding agents can be selected from Table 2. For example, if a vesicle population is detected or isolated using two, three, four or more binding agents in a differential detection or isolation of a vesicle from a heterogeneous population of vesicles, the particular binding agent profile for the vesicle population provides a bio-signature for the particular vesicle population. The vesicle can be detected using any number of binding agents in a multiplex fashion. Thus, the binding agent can also be used to form a bio-signature for a vesicle. The bio-signature can be used to characterize a phenotype.

Lectins

The binding agent can be a lectin. Lectins are proteins that bind selectively to polysaccharides and glycoproteins and are widely distributed in plants and animals. For example, lectins such as those derived from Galanthus nivalis in the form of Galanthus nivalis agglutinin (“GNA”), Narcissus pseudonarcissus in the form of Narcissus pseudonarcissus agglutinin (“NPA”) and the blue green algae Nostoc ellipsosporum called “cyanovirin” (Boyd et al. Antimicrob Agents Chemother 41(7): 1521 1530, 1997; Hammar et al. Ann N Y Acad Sci 724: 166 169, 1994; Kaku et al. Arch Biochem Biophys 279(2): 298 304, 1990) can be used to isolate a vesicle. These lectins can bind to glycoproteins having a high mannose content (Chervenak et al. Biochemistry 34(16): 5685 5695, 1995). High mannose glycoprotein refers to glycoproteins having mannose-mannose linkages in the form of α-1→3 or α-1→6 mannose-mannose linkages.

Other examples of lectins that can be used include, but not be limited to, Lens culimaris agglutinin-A (LCA), which specifically binds to proteins modified with fucose; wheat germ agglutinin (WGA), which has preferential binding to N-acetylglucosamine; concanavalin A (Con A), which recognizes α-linked mannose; and Griffonia (Bandeiraea) Simplicifolia Lectin II (GS-II), which binds to α- or β-linked N-acetylglucosamine residues.

One or more lectins can also be employed to isolate a vesicle. In some instances, a combination of lectins may be employed to isolate a vesicle. For example, at least 2, 3, 4, 5, 6, 7, 8, 9, 10, 11, 12, 13, 14, 15, 16, 17, 18, 19, 20, 25, 50, 75 or 100 different lectins may be used to isolate a vesicle from a biological sample.

Different lectins can also be used for multiplexing. For example, isolation of more than one population of vesicles (for example, vesicles from specific cell types) can be performed by isolating each vesicle population with a different lectin. Different lectins can be bound to different particles, wherein the different particles are labeled. Each particle can be bound to a lectin or combination of lectins. In another embodiment, an array comprising different lectins can be used for multiplex analysis, wherein the different lectins are differentially labeled or can be ascertained based on the location of the binding agent on the array. Multiplexing can be accomplished up to the resolution capability of the labels or detection method.

Methods and devices for using lectins to capture vesicles are described in International Patent Applications PCT/US2009/066626, entitled “AFFINITY CAPTURE OF CIRCULATING BIOMARKERS” and filed Dec. 3, 2009, and PCT/US2007/006101, entitled “EXTRACORPOREAL REMOVAL OF MICROVESICULAR PARTICLES” and filed Mar. 9, 2007, each of which applications is incorporated by reference herein in its entirety.

Non-Lectin Binding Agents

One or more lectins can be used with one or more non-lectin binding agents to isolate a vesicle. For example, at least 2, 3, 4, 5, 6, 7, 8, 9, 10, 11, 12, 13, 14, 15, 16, 17, 18, 19, 20, 25, 50, 75 or 100 lectin and non-lectin binding agents may be used to isolate a vesicle from a biological sample. A non-lectin binding agent can be DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), synthetic or naturally occurring chemical compounds (including but not limited to drugs, labeling reagents), dendrimers, or combinations thereof. The binding agent can be an agent that binds one or more lectins. Lectin capture can be applied to the isolation of the biomarker cathepsin D since it is a glycosylated protein capable of binding the lectins Galanthus nivalis agglutinin (GNA) and concanavalin A (ConA).

The non-lectin binding agent can be an antibody. For example, a vesicle may be isolated using one or more antibodies specific for one or more antigens present on the vesicle as well as a lectin specific for one or more glycoproteins present on the vesicle. As an example, a vesicle can have CD63 on its surface, and an antibody, or capture antibody, for CD63 can be used to isolate the vesicle. The antibody can be used along with one or more lectins that bind the vesicle to capture the vesicle. As another example, a vesicle derived from a tumor cell can express EpCam and/or B7H3. The vesicle can be isolated using an antibody for EpCam and/or B7H3, and optionally a lectin that binds the vesicle. Other antibodies for isolating vesicles can include an antibody, or capture antibody, to CD9, PSCA, TNFR, CD63, B7H3, MFG-E8, EpCam, Rab, CD81, STEAP, PCSA, PSMA, or 5T4. In some embodiments, one or more antibodies and one or more lectins can be used simultaneously to isolate a vesicle.

The antibodies disclosed herein can be immunoglobulin molecules or immunologically active portions of immunoglobulin molecules, i.e., molecules that contain an antigen binding site that specifically binds an antigen and synthetic antibodies. The immunoglobulin molecules can be of any class (e.g., IgG, IgE, IgM, IgD or IgA) or subclass of immunoglobulin molecule. Antibodies according to the invention include without limitation polyclonal, monoclonal, bispecific, synthetic, humanized and chimeric antibodies, single chain antibodies, Fab fragments and F(ab′)₂ fragments, Fv or Fv′ portions, fragments produced by a Fab expression library, anti-idiotypic (anti-Id) antibodies, or epitope-binding fragments of any of the above. An antibody, or generally any molecule, binds specifically to an antigen (or other molecule) if the antibody binds preferentially to the antigen versus other molecules. In some embodiments, antibodies used with the invention have less than 30%, 20%, 10%, 5% or 1% cross-reactivity with another molecule that may be present in the sample, e.g., other vesicle surface markers. In some embodiments, antibodies that cross react with multiple markers are used to bind vesicles. For example, an antibody that cross reacts with related members of a surface protein family can be used to bind vesicles displaying various members of that family.

The binding agent can be a polypeptide or peptide. The term polypeptide is used in its broadest sense and may include a protein, peptide, a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the invention can be chemically synthesized using standard techniques. The polypeptides may comprise D-amino acids (which are resistant to L-amino acid-specific proteases), a combination of D- and L-amino acids, β amino acids, or various other designer or non-naturally occurring amino acids (e.g., β-methyl amino acids, Cα-methyl amino acids, and Nα-methyl amino acids, etc.) to convey special properties. Synthetic amino acids may include ornithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the α-carbons, as in amino acids. The terms polypeptides and peptides are used interchangeably throughout this application unless otherwise stated.

A combination of one or more lectins with one or more non-lectin binding agents can also be used for multiplexing. For example, isolation of more than one population of vesicles (for example, vesicles from specific cell types) can be performed by isolating each vesicle population with a different binding agent or combination of binding agents. Different binding agents or binding agent combinations can be bound to different particles, wherein the different particles are labeled.

For example, a subset of particles can be used to isolate more than one population of vesicles. Each particle in a subset of particles is linked to a lectin, whereas in another subset of particles, each particle is linked to another binding agent, such as an antibody. The lectin binds one population of vesicles, whereas the antibody binds another population of vesicles. In some embodiments, the subset of particles can each be linked to more than one binding agent, such as a combination of different lectins or a combination of one or more lectins with one or more non-lectin binding agents.

In another embodiment, an array comprising different lectins and binding agents can be used for multiplex analysis, wherein the different lectins and binding agents are differentially labeled or can be ascertained based on the location of the binding agent on the array. Multiplexing can be accomplished up to the resolution capability of the labels or detection method.

A binding agent, such as an antibody or lectin, for isolating vesicles is preferably contacted with the biological sample comprising the vesicles of interest for a time sufficient for the binding agent to bind to a component of the vesicle. In one embodiment, an antibody is contacted with a biological sample for various intervals ranging from seconds to days, including but not limited to, about 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 45 minutes, 1 hour, 2 hours, 3 hours, 5 hours, 7 hours, 10 hours, 15 hours, 1 day, 3 days, 7 days or 10 days. The time can be selected to provide for efficient binding without allowing degradation of the binding agent system or vesicles.

### Flow Cytometry

In some embodiments, isolation or detection of a vesicle is performed using flow cytometry. Flow cytometry can be used for sorting particles such as a bead or microsphere suspended in a stream of fluid. As particles pass through the cytometer they can be selectively charged then deflected into separate paths of flow. It is therefore possible to separate populations from an original mix, such as a biological sample, often with a high degree of accuracy and speed.

Flow cytometry allows simultaneous multiparametric analysis of the physical and/or chemical characteristics of single cells or other entities flowing through an optical/electronic detection apparatus. A beam of light of a single frequency (color), e.g., a laser light, is directed onto a hydrodynamically focused stream of fluid. A number of detectors are aimed at the point where the stream passes through the light beam; one in line with the light beam (Forward Scatter or FSC) and several perpendicular to it (Side Scatter or SSC) and one or more fluorescent detectors. Each suspended particle passing through the beam scatters the light in some way, and fluorescent chemicals in the particle may be excited into emitting light at a lower frequency than the light source. This combination of scattered and fluorescent light is picked up by the detectors, and by analyzing fluctuations in brightness at each detector (one for each fluorescent emission peak), it is possible to deduce various facts about the physical and chemical structure of each individual particle. FSC correlates with the cell size and SSC depends on the inner complexity of the particle, such as shape of the nucleus, the amount and type of cytoplasmic granules or the membrane roughness. Some flow cytometers use only light scatter for measurement.

Flow cytometers can analyze several thousand particles every second in “real time” and can actively separate out and isolate particles having specified properties. They offer high-throughput automated quantification, and separation, of the set parameters for a high number of single cells during each analysis session. Flow cytometers can have multiple lasers and fluorescence detectors, allowing multiple labels to be used to more precisely specify a target population by their phenotype. Thus, a flow cytometer, such as a multicolor flow cytometer, can be used to detect one or more vesicles with a single or multiple fluorescent labels or colors. In some embodiments, the flow cytometer can also sort or isolate different vesicle populations, such as by size or by different markers.

The flow cytometer may have one or more lasers, such as 1, 2, 3, 4, 5, 6, 7, 8, 9, 10 or more lasers. In some embodiments, the flow cytometer can detect more than one color or fluorescent label, such as at least 2, 3, 4, 5, 6, 7, 8, 9, 10, 11, 12, 13, 14, 15, 16, 17, 18, 19, or 20 different colors or fluorescent labels. For example, the flow cytometer can have at least 2, 3, 4, 5, 6, 7, 8, 9, 10, 11, 12, 13, 14, 15, 16, 17, 18, 19, or 20 fluorescence detectors.

Examples of commercially available flow cytometers that can be used to detect or analyze one or more vesicles and/or to sort or separate different populations of vesicles include without limitation the MoFlo™ XDP Cell Sorter (Beckman Coulter, Brea, Calif.), MoFlo™ Legacy Cell Sorter (Beckman Coulter, Brea, Calif.), BD FACSAria™ Cell Sorter (BD Biosciences, San Jose, Calif.), BD™ LSRII (BD Biosciences, San Jose, Calif.), and BD FACSCalibur™ (BD Biosciences, San Jose, Calif.). Use of multicolor or multi-fluor cytometers can be used in multiplex analysis of vesicles, as further described below. In some embodiments, the flow cytometer can sort, and thereby collect or sort more than one population of vesicles based one or more characteristics. In embodiments wherein different populations of vesicles differ in size, vesicles within each population can be differentially detected or sorted based on size. In another embodiment, two different populations of vesicles are differentially labeled to allow for detection or sorting. Size and label can be used together for detection and sorting.

The data resulting from flow-cytometers can be plotted in one dimension to produce histograms, in two dimensions as dot plots, or in three dimensions with newer software. The regions on these plots can be sequentially separated by a series of subset extractions which are termed gates. Specific gating protocols exist for diagnostic and clinical purposes especially in relation to hematology. The plots are often made on logarithmic scales. Because the emission spectra of different fluorescent dyes can overlap, signals at the detectors can be compensated electronically as well as computationally. Fluorophores for labeling biomarkers may include those described in Ormerod, Flow Cytometry 2nd ed., Springer-Verlag, New York (1999), and in Nida et al., Gynecologic Oncology 2005; 4 889-894 which are incorporated herein by reference.

### Multiplexing

Different vesicle populations can be isolated or detected using different binding agents, e.g., using the binding agents disclosed herein. The different binding agents can be used for multiplexing different vesicle populations. Multiplexing refers to simultaneously measuring multiple analytes in a single assay. As a non-limiting example, one or more lectins and/or one or more vesicle protein markers can be detected simultaneously in a single assay. Each population in a biological sample can be labeled with a different label, such as a fluorophore, quantum dot, radioactive label or the like. The label can be directly conjugated to a binding agent or indirectly used to detect a binding agent that binds a vesicle. The number of populations detected in a multiplexing assay is dependent on the resolution capability of the labels and the summation of signals, as more than two differentially labeled vesicle populations that bind two or more affinity elements can produce summed signals.

Multiplexing can be performed on multiple populations of vesicles. Multiplexing of more than 2, 3, 4, 5, 6, 7, 8, 9, 10, 11, 12, 13, 14, 15, 16, 17, 18, 19, 20, 25, 50, 75 or 100 different vesicle populations can be performed. In some embodiments, one population of vesicles specific to a cell-of-origin is assayed together with a second population of vesicles specific to a different cell-of-origin, where each population is labeled with a different label. In another embodiment, a population of vesicles with a particular biomarker or bio-signature is multiplex assayed along a second population of vesicles with a different biomarker or bio-signature. These embodiments can be extended to differentiate a plurality of vesicles having different characteristics.

In one embodiment, multiplex analysis is performed by contacting a plurality of vesicles comprising more than one population of vesicles to a plurality of substrates in a single assay. The substrate may comprise beads. Each bead is coupled to one or more capture agents. The plurality of beads is divided into subsets, where beads with the same capture agent or combination of capture agents form a subset of beads, such that each subset of beads has a different capture agent or combination of capture agents than another subset of beads. The beads are used to capture vesicles that comprise a component that binds to the capture agent. The different subsets of beads can be used to capture different populations of vesicles. The captured vesicles can be analyzed, e.g., by detecting one or more vesicle characteristic such as size or biomarkers.

Flow cytometry can be used in combination with a particle-based or bead based assay. Multiparametric immunoassays or other high throughput detection assays using beads coated with cognate ligands and reporter molecules with specific activities consistent with high sensitivity automation can be used. As described, beads in a subset can be differentially labeled from every other subset. In a particle based assay system, a binding agent or capture agent for a vesicle, such as a capture antibody, is immobilized on addressable beads or microspheres. Each binding agent for each individual binding assay (such as an immunoassay when the binding agent is an antibody) can be coupled to a distinct type of microsphere (i.e., microbead) and the binding assay reaction takes place on the surface of the microspheres. Microspheres can be distinguished by different labels. For example, a microsphere with a specific capture agent would have a different signaling label as compared to another microsphere with a different capture agent. Microspheres can be dyed with discrete fluorescence intensities such that the fluorescence intensity of a microsphere with a specific binding agent is different than that of another microsphere with a different binding agent. Vesicles bound by the differing capture agents can be detected by via the differing labels.

A microsphere can be labeled or dyed with at least 2 different labels or dyes. In some embodiments, a microsphere is labeled with at least 3, 4, 5, 6, 7, 8, 9, or 10 different labels. Different microspheres in a plurality of microspheres can have more than one label or dye, wherein various subsets of the microspheres have various ratios and combinations of the labels or dyes permitting detection of different microspheres with different binding agents. For example, the various ratios and combinations of labels and dyes can permit different fluorescent intensities. Alternatively, the various ratios and combinations maybe used to generate different detection patterns to identify the different binding agents. The microspheres can be labeled or dyed externally or may have intrinsic fluorescence or signaling labels. In some embodiments, beads are loaded separately with appropriate binding agent. Vesicles are isolated based on the different binding agents on the differentially labeled microspheres to which the different binding agents are coupled.

In another embodiment, multiplex analysis is performed using a planar substrate, wherein the substrate comprises a plurality of capture agents. The plurality of capture agents can capture one or more populations of vesicles, and one or more biomarkers of the captured vesicles detected. The planar substrate can be a microarray or other substrate as further described herein.

### Substrates

A binding agent can be linked directly or indirectly to a solid surface or substrate. A solid surface or substrate includes physically separable solids to which a binding agent can be directly or indirectly attached. These surfaces or substrates include without limitation surfaces provided by microarrays, wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, TEFLON™, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the substrate with a biological sample.

In some embodiments, an antibody used to isolate a vesicle is bound to a solid substrate of a well, such as a well of a commercially available plate (e.g. from Nunc, Milan Italy). Such plates are known in the art, e.g., 96 and 384 well plates. Each well can be coated with an antibody. In some embodiments, the antibody used to isolate a vesicle is bound to a solid substrate in an array. The array can have a predetermined spatial arrangement of molecule interactions, binding islands, biomolecules, zones, domains or spatial arrangements of binding islands or binding agents deposited within discrete boundaries. The term array may be used herein to refer to multiple arrays arranged on a surface, such as would be the case with a surface bearing multiple copies of an array. Such surfaces bearing multiple arrays may also be referred to as multiple arrays or repeating arrays.

Arrays typically contain addressable moieties that can detect the presence of an entity, e.g., a vesicle in the sample via a binding event. An array may be referred to as a microarray. Arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein—protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. In a non-limiting example, a protein array can be used to detect vesicles having certain proteins on their surface. Antibody arrays comprise antibodies spotted onto the protein chip that are used as capture molecules to detect proteins or other biological materials from a sample, e.g., from cell or tissue lysate solutions. For example, antibody arrays can be used to detect vesicle-associated biomarkers from bodily fluids, e.g., serum or urine. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Cellular arrays can also be used to capture vesicles due to the similarity between a vesicle and cellular membrane. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

A binding agent can also be bound to particles such as beads or microspheres. For example, an antibody specific for a component of a vesicle can be bound to a particle, and the antibody-bound particle is used to isolate a vesicle from a biological sample. In some embodiments, the microspheres may be magnetic or fluorescently labeled. In addition, a binding agent for isolating vesicles can be a solid substrate itself. In some embodiments, latex beads, such as aldehyde/sulfate beads (Interfacial Dynamics, Portland, Oreg.) are used.

Binding agents bound to magnetic beads can be used to isolate a vesicle. In a non-limiting example, consider that a biological sample such as serum from a patient is collected for prostate cancer screening. The sample can be incubated with anti-PSMA or anti-PCSA coupled to magnetic microbeads and isolated, thereby capturing vesicles of prostate epithelial cell origin. In an embodiment, a low-density microcolumn can be placed in the magnetic field of a MACS Separator and the column is then washed with a buffer solution such as Tris-buffered saline. The magnetic immune complexes can then be applied to the column and unbound, non-specific material discarded. The PSMA or PCSA selected vesicle can be recovered by removing the column from the separator and placing it on a collection tube. A buffer can be added to the column and the magnetically labeled vesicle can be released by applying the plunger supplied with the column. The isolated vesicle can be diluted in IgG elution buffer and the complex can then be centrifuged to separate the microbeads from the vesicle. The pelleted isolated cell-of-origin specific vesicle can be resuspended in buffer such as phosphate-buffered saline and quantitated. Alternatively, due to the strong adhesion force between the antibody captured cell-of-origin specific vesicle and the magnetic microbeads, a proteolytic enzyme such as trypsin can be used for the release of captured vesicles without the need for centrifugation. The proteolytic enzyme can be incubated with the antibody captured cell-of-origin specific vesicles for at least a time sufficient to release the vesicles. One of skill will appreciate that this approach can be applied to isolating other specific vesicles by using binding agents that recognize different biomarkers.

A binding agent attached directly or indirectly to a solid surface or substrate can be used to capture a vesicle. The capture vesicle can be released from the substrate and analyzed or subjected to further isolation or concentration methods. Alternatively, the captured vesicle can be analyzed while still attached to the substrate.

A binding agent, such as an antibody specific to an antigen listed in Table 1, a binding agent listed in Table 2, a lectin binding agent, can be labeled to allow for its detection. Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as ³H, ¹¹C, ¹⁴C, ¹⁸F, ³²F, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available at probes.invitrogen.com/handbook.

A binding agent can be labeled directly, e.g., via a covalent bond. Binding agents can also be indirectly labeled, such as when a label is attached to the binding agent through a binding system. In a non-limiting example, consider an antibody labeled through biotin-streptavidin. Alternatively, an antibody is not labeled, but is later contacted with a second antibody that is labeled after the first antibody is bound to an antigen of interest. For example, various enzyme-substrate labels are available or disclosed (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include without limitation horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Lectin-Attached Substrates

One or more lectins can be attached to any substrate such as, but not limited to, agarose, aminocelite, resins, silica, polysaccharide, plastic and proteins. The silica can be glass beads, sand, diatomaceous earth, or a combination thereof. In some embodiments, a lectin is attached to a polysaccharide, such as dextran, cellulose, agarose, or a combination thereof. Yet in other embodiments, the lectin is attached to a protein, such as gelatin. A lectin can also be attached to a plastic, such as a plastic selected from the group consisting of polystyrenes, polysuflones, polyesters, polyurethanes, polyacrylates and their activated and native amino and carboxyl derivatives.

Any number of different polymers can be used as a substrate. To obtain a reactive polyacrylic acid polymer, for example, carbodiimides can be used (Valuev et al., 1998, Biomaterials, 19:41-3). Once the polymer has been activated, the lectins can be attached directly or via a linker to form in either case an affinity matrix. Suitable linkers include, but are not limited to, avidin, strepavidin, biotin, protein A, and protein G. The lectins may also be directly bound to coupling agents such as bifunctional reagents, or may be indirectly bound. A lectin can be bound to a substrate by a linker, such as a linker selected from the group, but consisting of gluteraldehyde, C2 to C18 dicarboxylates, diamines, dialdehydes, dihalides, and mixtures thereof. Furthermore, the linker can be a cleavable linker. For example, linkers used to couple peptides or amino acids to a substrate can also be used to attach a lectin.

The linker can be cleavable, such as a chemically cleavable moiety selected from an acid-cleavable moiety, a base-cleavable moiety, and a nucleophile-cleavable moiety. The cleavable linker moiety may be cleavable by a number of different mechanisms. The chemically cleavable linkage can comprise a modified base, a modified sugar, a disulfide bond, a chemically cleavable group incorporated into the phosphate backbone, or a chemically cleavable linker. For example, the cleavable linkage can comprise a cleavable linker moiety cleavable by acid, base, oxidation, reduction, heat, light, metal ion catalyzed, displacement, or elimination chemistry. In certain embodiments, the cleavable linker moiety may be cleaved by light, i.e., photocleavable, or the cleavable linker moiety may be chemically cleavable, e.g., acid- or base-labile. In such embodiments, the cleavable linker moiety comprises either a photocleavable moiety or chemically cleavable moiety.

Some examples of these linkages are described in PCT WO 96/37630, incorporated herein by reference. Chemically cleavable groups that may be incorporated include dialkoxysilane, 3′-(S)-phosphorothioate, 5′-(S)-phosphorothioate, 3′-(N)-phosphoroamidate, 5′-(N)-phosphoroamidate, cyanoether, aminocarbamate, dithioacetal, disulfide, and the like. In further embodiments the chemically cleavable linkage may be a modified sugar, such as ribose. Alternatively, the linkage may be a disulfide bond. Photocleavable or photolabile moieties that may be employed may include, but are not limited to: o-nitroarylmethine and arylaroylmethine, as well as derivatives thereof, and the like.

The substrate can be used as an affinity matrix to isolate a vesicle. The affinity matrix can be used in chromatography methods. In some embodiments, a lectin affinity matrix is prepared using Cyanogen Bromide to covalently couple a lectin to agarose. For example, Cyanogen bromide (CNBr) activated agarose can be used for direct coupling using a method, or modified method, as described in Cuatrecasas, et al (Cuatracasas et al. Proc Natl Acad Sci USA 61(2): 636-643, 1968). A lectin affinity matrix can also be prepared by coupling one or more lectins with glass beads via Schiff's base and reduction with cyanoborohydride. The silica lectin affinity matrix can be prepared by a modification of the method of Hermanson (Hermanson. Bioconjugate Techniques: 785, 1996).

A lectin can be covalently coupled to aminocelite using glutaraldehyde Aminocelite can be prepared by reaction of celite (silicate containing diatomaceous earth) by overnight reaction in an aqueous solution of aminopropyl triethoxysilane. The aminated celite can be washed free of excess reagent with water and ethanol and dried overnight to yield an off white powder. The powder can then be suspended in glutaraldehyde, the excess glutaraldehyde removed by filtration and washing with water until no detectable aldehyde remained in the wash using Schiff's reagent. The filter cake can then be resuspended borohydride coupling buffer containing one or more lectins, such as GNA, and the reaction allowed to proceed. At the end of the reaction, unreacted lectins can be washed off and the unreacted aldehyde aminated with ethanolamine.

Lectin affinity columns and chromatography medium for binding a vesicle is also commercially available. For example, agarose bound lectins wheat Germ Agglutinin, Elderberry lectin, and Maackia amurensis lectin can be purchased from Vector Laboratories (Burlingame, Calif., USA). Additional chromatography medium is commercially available. Candidate resins with lectin can be evaluated for their ability to bind a vesicle using any suitable method including, but not limited to, those described herein. Samples can be loaded onto the column and incubated to allow for binding. In some embodiments, non-specifically bound components can be removed by washing the column with binding buffer.

One or more lectins can also be attached to a substrate, such as a particle. For example, a lectin can be bound to particles, such as beads or microspheres. The microspheres may be magnetic or fluorescently labeled. The microspheres or nanospheres may comprise plastics (including acrylics, polystyrene, copolymers of styrene or other materials, polypropylene, polyethylene, polybutylene, polyurethanes, TEFLON™, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole). The particle may be intrinsically or extrinsically labeled. For example, the particle may be intrinsically dyed or contain a metal core, such as gold or silver core, such as commercially available from Luminex (Austin, Tex.) or Oxonica, Inc. (Mountain View, Calif.).

The one or more lectins can also be attached to a planar substrate, such as an array or microarray. The array can have a predetermined spatial arrangement of molecule interactions, binding islands, biomolecules, zones, domains or spatial arrangements of binding islands or binding agents deposited within discrete boundaries. Further, the term array may be used herein to refer to multiple arrays arranged on a surface, such as would be the case where a surface bore multiple copies of an array. Such surfaces bearing multiple arrays may also be referred to as multiple arrays or repeating arrays.

Non-Lectin Binding Agent Attached Substrates

As described herein, a binding agent, such as a non-lectin binding agent, can be attached directly or indirectly to a solid surface or substrate. The non-lectin binding agent can be used in combination with a lectin in isolating a vesicle. The non-lectin and lectin binding agent can be attached to the same substrate, or to different substrates. For example, a single substrate may comprise both a lectin and a non-lectin binding agent. Alternatively, a lectin and a non-lectin binding agent, such as an antibody, are each linked to a different substrate. For example, a lectin can be attached to an agarose resin and an antibody attached to a particle.

A binding agent can also be bound to particles such as beads or microspheres. For example, an antibody specific for a vesicle component can be bound to a particle, and the antibody-bound particle is used to isolate vesicles from a biological sample. In some embodiments, the microspheres may be magnetic or fluorescently labeled, such as described herein. The microspheres may be magnetic or fluorescently labeled. The microspheres or nanospheres may comprise plastics (including acrylics, polystyrene, copolymers of styrene or other materials, polypropylene, polyethylene, polybutylene, polyurethanes, TEFLON™, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole). The particle may be intrinsically or extrinsically labeled. For example, the particle may be intrinsically dyed or contain a metal core, such as gold or silver core, such as commercially available from Luminex (Austin, Tex.) or Oxonica, Inc. (Mountain View, Calif.). Other labels are described herein.

The binding agent may be linked to a solid surface or substrate, such as arrays, particles, wells and other substrates described above. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

### Devices

Also provided herein is a device for isolating vesicles. The device can be a microfluidic or nanofluidic device. The device can be disposable. The device can capture a vesicle using one or more lectins. The device for isolating a vesicle can comprise one or more chambers. The device can comprise a chamber comprising one or more lectins configured to capture a vesicle. The chamber can comprise a single type of lectin or a plurality of different types of lectins. The lectin can be a lectin that binds high mannose glycoproteins present on a vesicle. The lectin can be, but not limited to, Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), or Griffonia (Bandeiraea) Simplicifolia Lectin II (GS-II).

The chamber can comprise one or more lectins bound to a substrate, such as those described herein. For example, the substrate can be a planar substrate or a particle. The substrate can be selected from the group consisting of agarose, aminocelite, resins, silica, polysaccharide, plastic and proteins. For example, the substrate can comprise glass beads, sand, diatomaceous earth, or any combination thereof. The polysaccharide can comprise dextran, cellulose, agarose or any combination thereof. The protein substrate can comprise gelatin. In some embodiments, the substrate is a plastic is selected from the group consisting of polystyrenes, polysuflones, polyesters, polyurethanes, polyacrylates and their activated and native amino and carboxyl derivatives. The one or more lectins can be attached to the substrate by a linker, such as avidin, strepavidin, biotin, protein A, and protein G. The lectins may also be directly bound to using coupling agents such as bifunctional reagents, or may be indirectly bound. A lectin can be bound to a substrate by a linker, such as a linker selected from the group, but consisting of gluteraldehyde, C2 to C18 dicarboxylates, diamines, dialdehydes, dihalides, and mixtures thereof. Furthermore, the linker can be a cleavable linker. For example, linkers used to couple peptides or amino acids to a substrate can also be used to attach a lectin. In some embodiments, the linker is cleavable, such as a chemically cleavable moiety selected from an acid-cleavable moiety, a base-cleavable moiety, and a nucleophile-cleavable moiety. For example, the cleavable linkage can comprise a cleavable linker moiety cleavable by acid, base, oxidation, reduction, heat, light, metal ion catalyzed, displacement, or elimination chemistry.

The chamber comprising one or more lectins can be a column. The lectin-attached substrate can be filled or packed in a column. For example, a filter cartridge (such as commercially available from Glen Research, Silverton, Va.) can be prepared with a lectin resin, sealed and equilibrated.

In other embodiments, the device comprises one or more porous membranes. The porous membrane can be a hollow fiber membrane. The membrane can be formed by any number of polymers known to the art, for example, polysulfone, polyethersulfone, polyamides, polyimides, cellulose acetate, and polyacrylamide. In some embodiments, the membrane can have pores less than about 10,000, 5,000, 2000, 1000, 1500, 1000, 900, or 800 nm, such as less than 700 nm, in diameter. In some embodiments, the pores are less than about 600, 500, 400, 300, 200, 100, 30 nm. In some embodiments, the pores have an inside diameter of about 0.3 mm and an outside diameter of about 0.5 mm. In some embodiments, the porous membrane can exclude substantially all cells from passing through its pores.

The one or more porous membrane can be in a chamber with the one or more lectins. For example, the lectin can be disposed within a space or an extrachannel space (see for example, U.S. Pat. No. 7,226,429) of the chamber proximate to an exterior surface of the one or more porous membranes. A solution containing lectins can be loaded on to the device through a port leading to the extrachannel space. The lectins can immobilize to the exterior of the membrane and unbound lectins can be collected from a port leading out of the extrachannel space, by washing with saline or other solutions.

In some embodiments, a cartridge surrounds the one or more porous membranes. For example, a porous membrane can have a lumen and the cartridge and the porous membrane define an extralumenal space there between. The device can further comprise an inlet port and an outlet port in fluid communication with the lumen, and at least one port in fluid communication with the extralumenal space, wherein the device is configured for a vesicle of a biological sample to pass through the lumen and through the porous membrane into the extralumenal space while preventing a cellular portion of the biological sample passed through the lumen to pass through said porous membrane into said extralumenal space.

In some embodiments, the chamber comprising the one or more lectins is external to the cartridge. In other embodiments, the chamber is internal to the cartridge. In yet other embodiments, the extralumenal space is the chamber. Devices of this general type are disclosed in U.S. Pat. Nos. 4,714,556, 4,787,974, 6,528,057, 7,226,429, the disclosures of which are incorporated herein by reference.

In one embodiment of the presently disclosed device, a biological sample passes through the lumen of a hollow fiber membrane that is in contact, on the non-biological sample wetted side of the membrane, with immobilized lectins, which form a means to accept and immobilize vesicles. Thus, the device retains vesicles bound by lectin while allowing other components to pass through the lumen. In some embodiments, the device further comprises one or more additional binding agents, such as non-lectin binding agents. The non-lectin binding agent, such as an antibody to a tumor, can be immobilized along with the lectins and thus can also accept and immobilize vesicles.

The device comprising a lectin configured to capture a vesicle can further comprise one or more additional binding agents. For example, the device can comprise a chamber comprising a lectin configured to capture a vesicle and one or more additional binding agents that is present in the same or different chamber. The one or more additional binding agent can be a non-lectin binding agent. In some embodiments, the one or more additional binding agent is present in a different chamber than the chamber comprising one or more lectins.

The additional binding agent can be a different type of lectin, or a non-lectin binding agent selected from the group consisting of: DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), synthetic or naturally occurring chemical compounds, dendrimers, and combinations thereof. The additional binding agent can also be attached to a substrate, such as those disclosed herein.

In some embodiments, the one or more lectins is present in a first chamber and the additional one or more non-binding agents, such as a non-lectin binding agent is present in a second chamber of the device. The first chamber and the second chamber can be in fluid communication, such that a biological sample flows through the first chamber prior to the second chamber. In some embodiments, the biological sample flows through the second chamber prior to the first chamber.

For example, the first chamber can comprise one or more lectins and the second chamber an antibody. A biological sample comprising a vesicle can flow through said first chamber, wherein the vesicles are captured in the chamber. The vesicles can then be released and flow through the second chamber and be captured by the antibody.

The device can further comprise additional chambers with the same or different binding agents. The chambers can be columns.

The device can also further comprising a pump configured to pump a biological sample into said device at an assisted flow rate, the assisted flow rate being selected to increase a clearance rate of said device by at least two times over a clearance rate of said device without said pump.

In some embodiments, the device is configured to isolate a plurality of vesicles, such as different populations of vesicles. The device comprises a plurality of substrates, wherein each substrate is coupled to one or more lectins, and each subset of the plurality of substrates comprises a different lectin or combination of lectins than another subset of said plurality of substrates.

In other embodiments, the device further comprises a component for size exclusion size exclusion chromatography, density gradient centrifugation, differential centrifugation, nanomembrane ultrafiltration, or combinations thereof. The chamber or column can be in fluid communication with the chamber comprising the one or more lectins configured to capture a vesicle. For example, a biological sample can flow through the chamber comprising the one or more lectins configured to capture a vesicle prior to the component in the device for size exclusion size exclusion chromatography, density gradient centrifugation, differential centrifugation, nanomembrane ultrafiltration, or combinations thereof. Alternatively, the biological sample can flow through the chamber comprising the one or more lectins configured to capture a vesicle subsequent to the biological sample flowing through the component in the device for size exclusion size exclusion chromatography, density gradient centrifugation, differential centrifugation, nanomembrane ultrafiltration, or combinations thereof.

### Isolation of Vesicles

Also provided herein is a method for isolating or capturing a vesicle using one or more lectins. The isolation can be performed using one or more of the devices disclosed herein. The method comprises contacting a vesicle with a lectin. The vesicle can be from an in vitro sample, such as from a biological sample obtained from a subject with a lectin. In some embodiments, the method comprises contacting a vesicle with a lectin and a non-lectin binding agent. The method for isolating or capturing a vesicle using one or more lectins can further comprise analyzing the vesicle. The captured vesicle can be directly assayed or analyzed while still attached to the substrate. Alternatively, the vesicle can be analyzed after being released from the substrate. Analysis of the vesicle can comprise determining a bio-signature of the vesicle. The bio-signature can be used to characterize a phenotype. In some embodiments, the method of contacting a vesicle from a biological sample obtained from a subject with a lectin further comprises storing the vesicle in a preservation buffer.

A vesicle can be captured or isolated from a sample by contacting the vesicle with a lectin. The lectin can be bound to a substrate, such as described above. For example, the substrate can be a planar substrate or a particle. The substrate can be selected from the group consisting of agarose, aminocelite, resins, silica, polysaccharide, plastic and proteins. In some embodiments, a device comprising one or more lectins, further disclosed below, can be used to capture or isolate the one or more vesicles.

The method of isolating a vesicle can further comprise releasing the vesicle from the substrate. The vesicle can released from the substrate and the lectin, by eluting the vesicle with sugars, such as sugars the selected lectin used for capturing the vesicle is specifically or preferentially binds to. Buffers for eluting glycoproteins from lectins, such as from lectin affinity columns are known in the art and can be used for eluting a vesicle from a lectin, such as described in US20090136960. Buffers, and their respective lectin columns, can also be obtained commercially (such as AffiSep™ Lectin Columns & Kits, available from Galab Technologies, Germany and Qproteome Total Glycoprotein Kit from Qiagen, Valencia, Calif.). For example, elution of bound material from Lentil Lectin Sepharose 4B, commercially available from GE Healthcare (Piscataway, N.J.) can be achieved using a gradient of alpha-D-methyl-mannoside or alpha-D-methyl-glucoside. Glucose or mannose can also be used. Elution of tightly bound materials can also be facilitated by including 1% deoxycholate (or other detergents) in the elution buffers.

On another embodiment, vesicles captured by agarose bound lectins Wheat Germ Agglutinin, (WGA) Elderberry lectin, (SNA), and Maackia amurensis lectin, (MAL) can be released from an elution buffer comprising glucosamine. Alternatively, the vesicle glycoprotein can be cleaved, or if a cleavable linker was used to attach the lectin to the substrate, the linker can be cleaved. For example, if the cleavable linkage comprises a cleavable linker moiety cleavable by acid, base, oxidation, reduction, heat, light, metal ion catalyzed, displacement, or elimination chemistry, the respective cleaving agent can be used to release the vesicle from the substrate. The released vesicle can still be bound to the lectin, and then analyzed. Alternatively, the vesicle released from the substrate but still bound to the lectin can then have the lectin removed.

The method of isolating a vesicle can also comprise passing the biological sample through one or more porous membranes. Passing the biological sample through one or more porous membranes can be performed prior to contacting the vesicle with one or more lectins. Alternatively, passing the biological sample through one or more porous membranes can be subsequent to contacting the vesicle with one or more lectins. In some embodiments, the biological sample is collected and subjected to another passing through of one or more porous membranes.

In some embodiments, the method of isolating a vesicle comprises contacting a vesicle with a lectin and a non-lectin binding agent. The isolation of a vesicle can comprise contacting a vesicle with a lectin and a non-lectin binding agent concurrently or sequentially. For example, a vesicle can first be contacted with a lectin prior to being contacted with a non-lectin binding agent, such as an antibody to a tumor antigen. Alternatively, a vesicle can be contacted with a non-lectin binding agent, such as an antibody to a tumor antigen prior to being contacted with a lectin. The methods can further comprising contacting the vesicle with one or more additional binding agents, concurrently or sequentially.

Yet in other embodiments, the method comprises isolating a plurality of vesicles comprising: applying said plurality of vesicles to a plurality of substrates, wherein each substrate is coupled to one or more lectins, and each subset of said plurality of substrates comprises a different lectin or combination of lectins than another subset of said plurality of substrates; and capturing at least a subset of said plurality of vesicles bound to said one or more lectins. The method of can comprising determining a bio-signature for each of said captured vesicles.

The method of isolating a vesicle can further comprise one or more additional steps prior to, or subsequent to, contacting a vesicle with one or more binding agents, such as one or more lectins or one or more binding agents.

For example, a vesicle may be concentrated or isolated from a biological sample using size exclusion chromatography, density gradient centrifugation, differential centrifugation, nanomembrane ultrafiltration, or combinations thereof prior to contacting a vesicle with one or more binding agents, such as a lectin.

Size exclusion chromatography, such as gel permeation columns, centrifugation or density gradient centrifugation, and filtration methods can be used. For example, vesicles can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

In other embodiments, non-vesicle components can be removed prior to contacting a vesicle with one or more lectins. Highly abundant proteins, such as albumin and immunoglobulin, may hinder isolation of vesicles from a biological sample. For example, vesicles may be isolated from a biological sample using a system that utilizes multiple antibodies that are specific to the most abundant proteins found in blood. Such a system can remove up to several proteins at once, thus unveiling the lower abundance species such as cell-of-origin specific vesicles. The isolation of vesicles from a biological sample may also be enhanced by high abundant protein removal methods as described in Chromy et al. J Proteome Res 2004; 3:1120-1127.

In some embodiments, prior to lectin affinity chromatography, high abundance serum proteins are removed (e.g., using the ProtromeLab IgY-12 proteome partitioning kit (Beckman Coulter, Fullerton, Calif.)). This column enables removal of albumin, IgG, α1-antitrpsin, IgA, IgM, transferring, haptoglobin, al-acid glycoprotein, α2-macroglobin, HDL (apolipoproteins A-I and A-II) and fibrinogen in a single step.

Isolation or enrichment of vesicles from biological samples can also be enhanced by use of sonication (for example, by applying ultrasound), or the use of detergents, other membrane-active agents, or any combination thereof. For example, ultrasonic energy can be applied to a potential tumor site, and without being bound by theory, release of vesicles from the tissue can be increased, allowing an enriched population of vesicles that can be analyzed or assessed from a biological sample using one or more methods disclosed herein.

### Characterizing a Phenotype

A In an aspect of the invention, a phenotype of a subject is characterized by analyzing a biological sample and determining the presence, level, amount, or concentration of one or more populations of vesicles in the sample. In embodiments, characterization includes determining an absolute presence or absence, a quantitative level, or a relative level compared to a standard, e.g., the level of all vesicles present, the level of a housekeeping marker, and/or the level of a spiked-in marker. In some embodiments, vesicles are purified or concentrated from a sample prior to determining the amount of vesicles. Unless otherwise specified, “purified” or “isolated” as used herein refer to partial or complete purification or isolation. In other embodiments, vesicles are directly assessed from a sample, without prior purification or concentration. The detected vesicles can be cell-of-origin specific vesicles or vesicles with a specific bio-signature. Bio-signature include specific pattern of biomarkers, e.g., patterns of biomarkers indicative of a phenotype that is desirable to detect, such as a disease phenotype. The detected amount of vesicles can be used when characterizing a phenotype, such as a diagnosis, prognosis, theranosis, or prediction of responder/non-responder status. In some embodiments, the detected amount is used to determine a physiological or biological state, such as pregnancy or the stage of pregnancy. The detected amount of vesicles can also be used to determine treatment efficacy, stage of a disease or condition, or progression of a disease or condition. For example, the amount of one or more vesicles can be proportional or inversely proportional to an increase in disease stage or progression. The detected amount of vesicles can also be used to monitor progression of a disease or condition or to monitor a subject's response to a treatment.

The vesicles can be evaluated by comparing the level of vesicles with a reference level or value of vesicles. The reference value can be particular to physical or temporal endpoint. For example, the reference value can be from the same subject from whom a sample is assessed, or the reference value can be from a representative population of samples, e.g., samples from normal subjects without the disease. Therefore, a reference value provides a threshold measurement that can be compared to the readout for a vesicle population assayed in a given sample. Such reference values may be set according to data pooled from groups of sample corresponding to a particular cohort, including but not limited to age (e.g., newborns, infants, adolescents, young, middle-aged adults, seniors and adults of varied ages), racial/ethnic groups, normal versus diseased subjects, smoker v. non-smoker, subjects receiving therapy versus untreated subjects, different time points of treatment for a particular individual or group of subjects similarly diagnosed or treated or combinations thereof. Determining vesicle levels at different time points of treatment for a particular individual can provide a method for monitoring the individual's response to the treatment or progression of a disease or condition for which the individual is being treated.

A reference value may be based on samples assessed from the same subject so to provide individualized tracking. In some embodiments, frequent testing of vesicles in samples from a subject provides better comparisons to the reference values previously established for that subject. Such time course measurements are used to allow a physician to more accurately assess the subject's disease stage or progression and therefore inform a better decision for treatment. In some cases, the variance of vesicle levels is reduced when comparing a subject's own vesicle levels over time, thus allowing a individualized threshold to be defined for the subject, e.g., a threshold at which a diagnosis is made. Temporal intrasubject variation allows each individual to serve as their own longitudinal control for optimum analysis of disease or physiological state. As an illustrative example, consider that the level of vesicles derived from prostate cells is measured in a subject's blood over time. A spike in the level of prostate-derived vesicles in the subject's blood can indicate hyperproliferation of prostate cells, e.g., due to prostate cancer.

In some embodiments, reference values are established for unaffected individuals of varying ages, ethnic backgrounds and sexes by determining the amount of vesicles of interest in the unaffected individuals. The reference value for a reference population can be used as a baseline for detection of one or more vesicle populations in a test subject. If a sample from a subject has a level or value that is similar to the reference, the subject might be determined to not have the disease, or of having a low risk of developing a disease.

In other embodiments, reference values or levels are established for individuals with a particular phenotype by determining the amount of one or more populations of vesicles in an individual with the phenotype, e.g., a disease or a response to therapy. In an embodiment, an index of values is generated for a particular phenotype. Different disease stages can have different values, determined from individuals with the different disease stages. A subject's value can be compared to the index and a diagnosis or prognosis of the disease can be determined, e.g., the disease stage or progression wherein the subject's levels most closely correlate with the index. In other embodiments, an index of values is generated for therapeutic efficacies. For example, the level of vesicles of individuals with a particular disease can be generated and correlated with treatments that were effective for the individual. The levels can be used to generate values of which is a subject's value is compared, and a treatment or therapy can be selected for the individual, e.g., by predicting from the levels whether the subject is likely to be a responder or non-responder for a treatment.

In some embodiments, a reference value is determined for individuals without a phenotype, by isolating or detecting vesicles linked to the phenotype. As a non-limiting example, individuals with varying stages of colorectal cancer and noncancerous polyps can be surveyed using the same techniques described for unaffected individuals and the levels of circulating vesicles for each group can be determined. In some embodiments, the levels are defined as means±standard deviations from at least two separate experiments performed in at least triplicate. Comparisons between these groups can be made using statistical tests to determine statistical significance of distinguishing vesicle biosignatures. In some embodiments, statistical significance is determined using a parametric statistical test. The parametric statistical test can comprise, without limitation, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than 0.05, 0.01, 0.005, 0.001, 0.0005, or 0.0001. The p-values can also be corrected for multiple comparisons, e.g., using a Bonferroni correction, a modification thereof, or other technique known to those in the art, e.g., the Hochberg correction, Holm-Bonferroni correction, \check{S}idák correction, Dunnett's correction or Tukey's multiple comparisons. In some embodiments, an ANOVA is followed by Tukey's correction for post-test comparing of the biomarkers from each population.

Reference values can also be established for disease recurrence monitoring (or exacerbation phase in MS), for therapeutic response monitoring, or for predicting responder/non-responder status.

In some embodiments, a reference value is determined using an artificial vesicle, also referred to herein as a synthetic vesicle. Methods for manufacturing artificial vesicles are known to those of skill in the art, e.g., using liposomes. Artificial exosomes can be manufactured using methods disclosed in US20060222654 and U.S. Pat. No. 4,448,765, which are incorporated herein by reference in its entirety. Artificial vesicles can be constructed with known markers to facilitate capture and/or detection. In some embodiments, artificial vesicles are spiked into a bodily sample prior to processing. The level of intact synthetic vesicle can be tracked during processing, e.g., using filtration or other isolation methods disclosed herein, to provide a control for the amount of vesicles in the initial versus processed sample. Similarly, artificial vesicles can be spiked into a sample before or after any processing steps. In some embodiments, artificial vesicles are used to calibrate equipment used for isolation and detection of vesicles.

Artificial vesicle can be produced and used a control to test the viability of an assay, such as a bead-based assay. The artificial vesicle can bind to both the beads and to the detection antibodies. Thus, the artificial vesicle contains the amino acid sequence/conformation that each of the antibodies binds. The artificial vesicle can comprise a purified protein or a synthetic peptide sequence to which the antibody binds. The artificial vesicle could be a bead, e.g., a polystyrene bead, that is capable of having biological molecules attached thereto. If the bead has an available carboxyl group, then the protein or peptide could be attached to the bead via an available amine group, such as using carbodiimide coupling.

In another embodiment, the artificial vesicle can be a polystyrene bead coated with avidin and a biotin is placed on the protein or peptide of choice either at the time of synthesis or via a biotin-maleimide chemistry. The proteins/peptides to be on the bead can be mixed together in ratio specific to the application the artificial vesicle is being used for, and then conjugated to the bead. These artificial vesicles can then serve as a link between the capture beads and the detection antibodies, thereby providing a control to show that the components of the assay are working properly.

The value can be a quantitative or qualitative value. The value can be a direct measurement of the level of vesicles (example, mass per volume), or an indirect measure, such as the amount of a specific biomarker. The value can be a quantitative, such as a numerical value. In other embodiments, the value is qualitative, such as no vesicles, low level of vesicles, medium level, high level of vesicles, or variations thereof.

The reference value can be stored in a database and used as a reference for the diagnosis, prognosis, theranosis, disease stratification, disease monitoring, treatment monitoring or prediction of non-responder/responder status of a disease or condition based on the level or amount of vesicles, such as total amount of vesicles, or the amount of a specific population of vesicles, such as cell-of-origin specific vesicles or vesicles with a specific bio-signature. In an illustrative example, consider a method of determining a diagnosis for a cancer. Vesicles from reference subjects with and without the cancer are assessed and stored in the database. The reference subjects provide biosignature indicative of the cancer or of another state, e.g., a healthy state. A sample from a test subject is then assayed and the vesicle biosignature are compared against those in the database. If the subject's biosignature correlates more closely with reference values indicative of cancer, a diagnosis of cancer may be made. Conversely, if the subject's biosignature correlates more closely with reference values indicative of a healthy state, the subject may be determined to not have the disease. One of skill will appreciate that this example is non-limiting and can be expanded for assessing other phenotypes, e.g., other diseases, prognosis, theranosis, disease stratification, disease monitoring, treatment monitoring or prediction of non-responder/responder status, and the like.

Many analytical techniques are available to assess vesicles. In some embodiments, vesicle levels are characterized using mass spectrometry, flow cytometry, immunocytochemical staining, Western blotting, electrophoresis, chromatography or x-ray crystallography in accordance with procedures known in the art. For example, vesicles can be characterized and quantitatively measured using flow cytometry as described in Clayton et al., Journal of Immunological Methods 2001; 163-174, which is herein incorporated by reference in its entirety. Vesicle levels may be determined using binding agents as described above. For example, a binding agent to vesicles can be labeled and the label detected and used to determine the amount of vesicles in a sample. The binding agent can be bound to a substrate, such as arrays or particles, such as described above. Alternatively, the vesicles may be labeled directly.

In some embodiments, electrophoretic tags or eTags are used to determine the amount of vesicles of interest. eTags are small fluorescent molecules linked to nucleic acids or antibodies and are designed to bind one specific nucleic acid sequence or protein, respectively. After the eTag binds its target, an enzyme is used to cleave the bound eTag from the target. The signal generated from the released eTag, called a “reporter,” is proportional to the amount of target nucleic acid or protein in the sample. The eTag reporters can be identified by capillary electrophoresis. The unique charge-to-mass ratio of each eTag reporter—that is, its electrical charge divided by its molecular weight—makes it show up as a specific peak on the capillary electrophoresis readout. Thus, by targeting a specific biomarker of a vesicle with an eTag, the amount or level of vesicles is determined.

The vesicle level can determined from a heterogeneous population of vesicles, such as the total population of vesicles in a sample. Alternatively, the vesicles level is determined from a homogenous population, or substantially homogenous population of vesicles, such as the level of specific cell-of-origin vesicles, such as vesicles from prostate cancer cells. In yet other embodiments, the level is determined for vesicles with a particular biomarker or combination of biomarkers, such as a biomarker specific for prostate cancer. Determining the level vesicles can be performed in conjunction with determining the biomarker or combination of biomarkers of a vesicle. Alternatively, determining the amount of vesicle may be performed prior to or subsequent to determining the biomarker or combination of biomarkers of the vesicles.

The amount of vesicles in a sample can be assayed in a multiplexed manner. Multiplex analysis can be used for determining the amount of more than one population of vesicles, such as different cell-of-origin specific vesicles with different biomarkers or combination of biomarkers.

### Specificity and Sensitivity

Performance of a diagnostic or related test is typically assessed using statistical measures. The performance of the characterization can be assessed by measuring sensitivity, specificity and related measures. For example, a level of vesicles of interest can be assayed to characterize a phenotype, such as detecting a disease. The sensitivity and specificity of the assay to detect the disease is determined.

A true positive is a subject with a characteristic, e.g., a disease or disorder, correctly identified as having the characteristic. A false positive is a subject without the characteristic that the test improperly identifies as having the characteristic. A true negative is a subject without the characteristic that the test correctly identifies as not having the characteristic. A false negative is a person with the characteristic that the test improperly identifies as not having the characteristic. The ability of the test to distinguish between these classes provides a measure of test performance.

The specificity of a test is defined as the number of true negatives divided by the number of actual negatives (i.e., sum of true negatives and false positives). Specificity is a measure of how many subjects are correctly identified as negatives. A specificity of 100% means that the test recognizes all actual negatives—for example, all healthy people will be recognized as healthy. A lower specificity indicates that more negatives will be determined as positive.

The sensitivity of a test is defined as the number of true positives divided by the number of actual positives (i.e., sum of true positives and false negatives). Specificity is a measure of how many subjects are correctly identified as positives. A sensitivity of 100% means that the test recognizes all actual positives—for example, all sick people will be recognized as sick. A lower sensitivity indicates that more positives will be missed by being determined as negative.

The accuracy of a test is defined as the number of true positives and true negatives divided by the sum of all true and false positives and all true and false negatives. It provides one number that combines sensitivity and specificity measurements.

Sensitivity, specificity and accuracy are determined at a particular discrimination threshold value. For example, a common threshold for prostate cancer (PCa) detection is 4 ng/mL of prostate specific antigen (PSA) in serum. A level of PSA equal to or above the threshold is considered positive for PCa and any level below is considered negative. As the threshold is varied, the sensitivity and specificity will also vary. For example, as the threshold for detecting cancer is increased, the specificity will increase because it is harder to call a subject positive, resulting in fewer false positives. At the same time, the sensitivity will decrease. A receiver operating characteristic curve (ROC curve) is a graphical plot of the true positive rate (i.e., sensitivity) versus the false positive rate (i.e., 1-specificity) for a binary classifier system as its discrimination threshold is varied. The ROC curve shows how sensitivity and specificity change as the threshold is varied. The Area Under the Curve (AUC) of an ROC curve provides a summary value indicative of a test's performance over the entire range of thresholds. The AUC is equal to the probability that a classifier will rank a randomly chosen positive sample higher than a randomly chosen negative sample. An AUC of 0.5 indicates that the test has a 50% chance of proper ranking, which is equivalent to no discriminatory power (a coin flip also has a 50% chance of proper ranking). An AUC of 1.0 means that the test properly ranks (classifies) all subjects. The AUC is equivalent to the Wilcoxon test of ranks.

A vesicle characteristic or bio-signature can be used to characterize a phenotype with at least 50, 51, 52, 53, 54, 55, 56, 57, 58, 59, 60, 61, 62, 63, 64, 65, 66, 67, 68, 69, or 70% sensitivity, such as with at least 71, 72, 73, 74, 75, 76, 77, 78, 79, 80, 81, 82, 83, 84, 85, 86, or 87% sensitivity. In some embodiments, the phenotype is characterized with at least 87.1, 87.2, 87.3, 87.4, 87.5, 87.6, 87.7, 87.8, 87.9, 88.0, or 89% sensitivity, such as at least 90% sensitivity. The phenotype can be characterized with at least 91, 92, 93, 94, 95, 96, 97, 98, 99 or 100% sensitivity.

A vesicle characteristic or bio-signature can be used to characterize a phenotype of a subject with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

A vesicle characteristic or bio-signature can be used to characterize a phenotype of a subject, e.g., based on vesicle level or other characteristic, with at least 50% sensitivity and at least 60, 65, 70, 75, 80, 85, 90, 95, 99, or 100% specificity; at least 55% sensitivity and at least 60, 65, 70, 75, 80, 85, 90, 95, 99, or 100% specificity; at least 60% sensitivity and at least 60, 65, 70, 75, 80, 85, 90, 95, 99, or 100% specificity; at least 65% sensitivity and at least 60, 65, 70, 75, 80, 85, 90, 95, 99, or 100% specificity; at least 70% sensitivity and at least 60, 65, 70, 75, 80, 85, 90, 95, 99, or 100% specificity; at least 75% sensitivity and at least 60, 65, 70, 75, 80, 85, 90, 95, 99, or 100% specificity; at least 80% sensitivity and at least 60, 65, 70, 75, 80, 85, 90, 95, 99, or 100% specificity; at least 85% sensitivity and at least 60, 65, 70, 75, 80, 85, 90, 95, 99, or 100% specificity; at least 86% sensitivity and at least 60, 65, 70, 75, 80, 85, 90, 95, 99, or 100% specificity; at least 87% sensitivity and at least 60, 65, 70, 75, 80, 85, 90, 95, 99, or 100% specificity; at least 88% sensitivity and at least 60, 65, 70, 75, 80, 85, 90, 95, 99, or 100% specificity; at least 89% sensitivity and at least 60, 65, 70, 75, 80, 85, 90, 95, 99, or 100% specificity; at least 90% sensitivity and at least 60, 65, 70, 75, 80, 85, 90, 95, 99, or 100% specificity; at least 91% sensitivity and at least 60, 65, 70, 75, 80, 85, 90, 95, 99, or 100% specificity; at least 92% sensitivity and at least 60, 65, 70, 75, 80, 85, 90, 95, 99, or 100% specificity; at least 93% sensitivity and at least 60, 65, 70, 75, 80, 85, 90, 95, 99, or 100% specificity; at least 94% sensitivity and at least 60, 65, 70, 75, 80, 85, 90, 95, 99, or 100% specificity; at least 95% sensitivity and at least 60, 65, 70, 75, 80, 85, 90, 95, 99, or 100% specificity; at least 96% sensitivity and at least 60, 65, 70, 75, 80, 85, 90, 95, 99, or 100% specificity; at least 97% sensitivity and at least 60, 65, 70, 75, 80, 85, 90, 95, 99, or 100% specificity; at least 98% sensitivity and at least 60, 65, 70, 75, 80, 85, 90, 95, 99, or 100% specificity; at least 99% sensitivity and at least 60, 65, 70, 75, 80, 85, 90, 95, 99, or 100% specificity; or substantially 100% sensitivity and at least 60, 65, 70, 75, 80, 85, 90, 95, 99, or 100% specificity.

A vesicle characteristic or bio-signature can be used to characterize a phenotype of a subject with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97% accuracy, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% accuracy.

In some embodiments, a vesicle characteristic or bio-signature is used to characterize a phenotype of a subject with an AUC of at least 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, or 0.97, such as with at least 0.971, 0.972, 0.973, 0.974, 0.975, 0.976, 0.977, 0.978, 0.978, 0.979, 0.980, 0.981, 0.982, 0.983, 0.984, 0.985, 0.986, 0.987, 0.988, 0.989, 0.99, 0.991, 0.992, 0.993, 0.994, 0.995, 0.996, 0.997, 0.998, 0.999 or 1.00.

Furthermore, the confidence level for determining the specificity, sensitivity, accuracy or AUC, may be determined with at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% confidence.

Other related performance measures include positive and negative likelihood ratios [positive LR=sensitivity/(1−specificity); negative LR=(1−sensitivity)/specificity]. Such measures can also be used to gauge test performance according to the methods of the invention.

### Classification

Vesicles biosignatures can be used to classify a sample. For example, a sample can be classified as, or predicted to be, a responder or non-responder to a given treatment for a given disease or disorder. Many statistical classification techniques are known to those of skill in the art. In supervised learning approaches, a group of samples from two or more groups are analyzed with a statistical classification method. Biomarkers can be discovered that can be used to build a classifier that differentiates between the two or more groups. A new sample can then be analyzed so that the classifier can associate the new with one of the two or more groups. Commonly used supervised classifiers include without limitation the neural network (multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with the invention include quadratic classifiers, k-nearest neighbor, boosting, decision trees, random forests, neural networks, pattern recognition, Bayesian networks and Hidden Markov models. One of skill will appreciate that these or other classifiers, including improvements of any of these, are contemplated within the scope of the invention.

Classification using supervised methods is generally performed by the following methodology:

In order to solve a given problem of supervised learning (e.g. learning to recognize handwriting) one has to consider various steps:

1. Gather a training set. These can include, for example, samples that are from a subject with or without a disease or disorder, subjects that are known to respond or not respond to a treatment, subjects whose disease progresses or does not progress, etc. The training samples are used to “train” the classifier.

2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality; but should be large enough to accurately predict the output. The features might include a set of biomarkers such as those derived from vesicles as described herein.

3. Determine the structure of the learned function and corresponding learning algorithm. A learning algorithm is chosen, e.g., artificial neural networks, decision trees, Bayes classifiers or support vector machines. The learning algorithm is used to build the classifier.

4. Build the classifier. The learning algorithm is run the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier is determined as described above, it can be used to classify a sample, e.g., that of a subject who is being analyzed by the methods of the invention. As an example, a classifier can be built using data for levels of vesicles of interest in reference subjects with and without a disease as the training and test sets. Vesicle levels found in a sample from a test subject are assessed and the classifier is used to classify the subject as with or without the disease.

Unsupervised learning approaches can also be used with the invention. Clustering is an unsupervised learning approach wherein a clustering algorithm correlates a series of samples without the use the labels. The most similar samples are sorted into “clusters.” A new sample could be sorted into a cluster and thereby classified with other members that it most closely associates. Many clustering algorithms are known to those of skill in the art.

### Cell-of-Origin and Disease-Specific Vesicles

The binding agents disclosed herein can be used to isolate or detect a vesicle, such as a cell-of-origin vesicle or vesicle with a specific bio-signature. In one embodiment, binding agents are used to isolate or detect a heterogeneous population of vesicles from a sample. In one embodiment, the binding agents are used to isolate or detect a homogeneous population of vesicles from a heterogeneous population of vesicles. The homogeneous population can be cell-of-origin specific vesicles or other populations of vesicles with specific bio-signatures.

A homogeneous population of vesicles, such as cell-of-origin specific vesicles, can be analyzed to characterize a phenotype for a subject. Cell-of-origin specific vesicles are vesicles derived from specific cell types, which include without limitation cells of a defined tissue, defined organ, tumor of interest or other diseased tissue of interest, circulating tumor or diseased cells, or cells of maternal or fetal origin. In some embodiments, the vesicles are derived from tumor cells or lung, pancreas, stomach, intestine, bladder, kidney, ovary, testis, skin, colorectal, breast, prostate, brain, esophagus, liver, placenta, or fetal cells. The isolated vesicle can also be from a particular sample type, such as vesicle from urine, blood, semen, feces, saliva, other bodily fluids, or solid tissue.

A cell-of-origin specific vesicle from a biological sample can be isolated using one or more binding agents that are specific for vesicles for that cell-of-origin. In one embodiment, the binding agents recognize surface antigens on the surface of the vesicles, e.g., surface proteins. In an embodiment, vesicles for analysis of a disease or condition are isolated using one or more binding agents specific for biomarkers for that disease or condition. The disease include cancers, neurological disorders, cardiovascular disorders, immune disorders (e.g., autoimmune diseases), infectious disorders (e.g., microbial or viral diseases).

A vesicle can be concentrated prior to isolation or detection of a cell-of-origin specific vesicle, such as through centrifugation, chromatography, or filtration, as described above. This step or steps can produce a heterogeneous population of vesicles prior to isolation of cell-of-origin specific vesicles. Alternatively, the vesicle is not concentrated, or the biological sample is not enriched for a vesicle, prior to isolation of a cell-of-origin vesicle. An example of the later case includes direct capture from a bodily fluid such as blood.

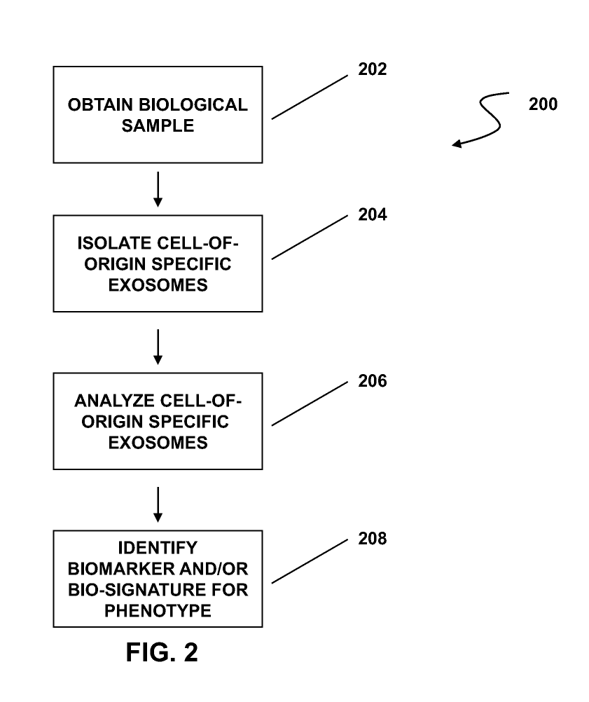

FIG. 2 illustrates a flowchart which depicts one method 200 for isolating or identifying a cell-of-origin specific vesicle. First, a biological sample is obtained from a subject in step 202. The sample can be obtained from a third party or from the same party performing the vesicle analysis. Next, cell-of-origin specific vesicles are isolated from the biological sample in step 204. The isolated cell-of-origin specific vesicles are then analyzed in step 206 and a biomarker or bio-signature for a particular phenotype is identified in step 208. The method may be applied to measure any appropriate phenotype. In some embodiments, prior to step 204, vesicles are concentrated or isolated from a biological sample to produce a homogeneous population of vesicles. For example, a heterogeneous population of vesicles may be isolated using centrifugation, chromatography, filtration, or other methods as described above, prior to use of one or more binding agents specific for isolating or identifying vesicles derived from specific cell types.

A cell-of-origin specific vesicle can be isolated from a biological sample of a subject using one or more binding agents that bind with high specificity to the cell-of-origin specific vesicle. In some embodiments, a single binding agent is used to isolate a cell-of-origin specific vesicle. In other embodiments, a combination of binding agents is used to isolate a cell-of-origin specific vesicle. In some embodiments, at least 2, 3, 4, 5, 6, 7, 8, 9, 10, 11, 12, 13, 14, 15, 16, 17, 18, 19, 20, 25, 50, 75, or 100 different binding agents are used to isolate a cell-of-origin vesicle. A population of vesicles having the same binding agent profile can be identified by using a single or a plurality of binding agents.

One or more binding agents can be selected based on their specificity for a target antigen(s) that is specific to a cell-of-origin, e.g., a cell-of-origin that is related to a tumor, autoimmune disease, cardiovascular disease, neurological disease, infection or other disease or disorder. The cell-of-origin can be from a cell that is informative for a diagnosis, prognosis, disease stratification, theranosis, prediction of responder/non-responder status, disease monitoring, treatment monitoring and the like as related to such diseases and disorders. The cell-of-origin can also be from a cell useful to discover biomarkers for use thereto. Non-limiting examples of antigens which may be used singularly, or in combination, to isolate a cell-of-origin specific vesicle, disease specific vesicle, or tumor specific vesicle, as listed in Table 1 and are also described herein. The antigen can comprise membrane bound antigens which are accessible to binding agents, e.g., surface proteins or fragments thereof. In some embodiments, the antigen is a biomarker related to characterizing a phenotype, e.g., a disease marker. In some embodiments, the antigen is a biomarker specific to a cell-of-origin, e.g., a cell derived from the prostate, lung, breast, or GI tract. In some embodiments, the antigen is a biomarker specific to a class of vesicles, e.g., exosomes.

A number of exemplary binding agents useful for binding to vesicles associated with cancer, autoimmune diseases, cardiovascular diseases, neurological diseases, infection or other disease or disorders are presented in U.S. patent application Ser. No. 12/591,226, filed Nov. 12, 2009 and entitled “Methods and Systems of Using Exosomes for Determining Phenotypes,” which application is hereby incorporated by reference in its entirety.

One of skill will appreciate that any applicable antigen that can be used to isolate an informative vesicle is contemplated by the invention. Binding agents, e.g., antibodies, aptamers and lectins, can be chosen that recognize surface antigens and/or fragments thereof, as outlined herein. The binding agents can recognize antigens specific to the desired cell type or location and/or recognize biomarkers associated with the desired cells. The cells can be, e.g., tumor cells, other diseased cells, cells that serve as markers of disease such as activated immune cells, etc. One of skill will appreciate that binding agents for any cells of interest can be useful for isolating vesicles associated with those cells. One of skill will further appreciate that the binding agents disclosed herein can be used for detecting vesicles of interest. As a non-limiting example, a binding agent to a vesicle biomarker can be labeled directly or indirectly in order to detect vesicles bound by one of more of the same or different binding agents.

The binding agents are chosen to characterize the phenotype of interest. For example, a vesicle derived from a prostate cancer cell can be isolated using a binding agent, e.g., an antibody or aptamer, that is specific for an antigen associated with vesicles from a cell of prostate cancer origin, including without limitation PSA, TMPRSS2, FASLG, TNFSF10, PSMA, PCSA, NGEP, Il-7RI, CSCR4, CysLT1R, TRPM8, Kv1.3, TRPV6, TRPM8, PSGR, MISIIR, galectin-3, PCA3, TMPRSS2:ERG, or a combination thereof. Any appropriate antigens that are specific for vesicles derived from prostate cancer cells can be used for isolation thereof. Similarly, a vesicle derived from a benign prostatic hyperplasia (BPH) cell can be isolated using a binding agent, e.g., an antibody or aptamer, which is specific for an antigen associated with vesicles from a cell associated with BPH including, but not limited to, KIA1, intact fibronectin, or a combination thereof. Any appropriate antigens that are specific for vesicles derived from cells associated with BPH can be used for isolation thereof.

One of skill will appreciate that binding agents for biomarkers of vesicles associated with other cells of interest can be used similarly, including those disclosed in U.S. patent application Ser. No. 12/591,226, filed Nov. 12, 2009 and entitled “Methods and Systems of Using Exosomes for Determining Phenotypes,” which application is hereby incorporated by reference in its entirety. Likewise, additional markers for the cell types can be useful for isolating those vesicles, either individually, in combination with one or more markers listed above, or in combination with other markers. Cell-specific binding agents can be used in combination with vesicle specific binding agents to isolate vesicles from a given origin. As a non-limiting illustrative example, vesicle binding agents can be used in combination with breast cancer-specific binding agents to detect or isolate vesicles of breast cancer origin.

A cell-of-origin specific vesicle can be isolated using novel binding agents, e.g., using methods such as described herein. A cell-of-origin specific vesicle can also be isolated from a biological sample using isolation methods based on cellular binding partners or binding agents of such vesicles. Such cellular binding partners include without limitation peptides, proteins, RNA, DNA, apatmers, lectins, cells or serum-associated proteins. Useful binding partners bind in a recognizable manner to desired vesicles when one or more specific biomarkers are present. Isolation or detection of a cell-of-origin specific vesicle can be carried out with a single binding partner or binding agent, or a combination of binding partners or binding agents whose singular application or combined application results in cell-of-origin specific isolation or detection. Non-limiting examples of such binding agents are provided in Table 2. As a non-limiting illustrative example, a vesicle for characterizing breast cancer can be isolated with one or more binding agents including estrogen, progesterone, trastuzumab, CCND1, MYC PNA, IGF-1 PNA, MYC PNA, SC4 aptamer (Ku), AII-7 aptamer (ERB2), Galectin-3, mucin-type O-glycans, L-PHA, and/or Galectin-9. In some embodiments, one or more of these are used along with antibodies that recognize breast cancer markers as described above.

In various embodiments, binding agents are used for isolating or detecting cell-of-origin specific vesicles based on: i) detection of binding to antigens specific for cell-of-origin specific vesicles; ii) the absence of detection of markers specific for cell-of-origin specific vesicles; or iii) detection of expression levels of biomarkers specific for cell-of-origin specific vesicles. In an embodiment, a heterogeneous population of vesicles is applied to a surface coated with specific binding agents designed to identify the cell-of-origin characteristics of the vesicles. Various binding agents, e.g., antibodies or aptamers, can be arrayed on a solid surface or substrate wherein the heterogeneous population of vesicles is allowed to contact the solid surface or substrate for a sufficient time to allow binding events to take place. The presence or absence of binding events at given locations on the array surface or substrate can identify the presence or absence of vesicle populations that are specific to a given cell-of-origin. That is, binding events signal the presence of a vesicle having an antigen recognized by the bound antibody or aptamer. Conversely, lack of binding events signal that the absence of vesicles having an antigen recognized by the bound antibody or aptamer.

A cell-of-origin specific vesicle can be enriched or isolated using one or more binding agents using a magnetic capture method, fluorescence activated cell sorting (FACS) or laser cytometry as described herein. Magnetic capture methods include, but are not limited to, the use of magnetically activated cell sorter (MACS) microbeads or magnetic columns. Examples of immunoaffinity and magnetic particle methods that can be used are found in U.S. Pat. No. 4,551,435, 4,795,698, 4,925,788, 5,108,933, 5,186,827, 5,200,084 or 5,158,871. A cell-of-origin specific vesicle can also be isolated following the general methods described in U.S. Pat. No. 7,399,632, by using combination of antigens specific to a vesicle.

Any other appropriate method for isolating or otherwise enriching the cell-of-origin specific vesicles with respect to a biological sample can be used according to the present invention. As described herein, size exclusion chromatography such as gel permeation columns, centrifugation or density gradient centrifugation, and filtration methods can be used in combination with the other antigen selection methods described herein. The cell-of-origin specific vesicles may also be isolated following the methods described in Koga et al., Anticancer Research, 25:3703-3708 (2005), Taylor et al., Gynecologic Oncology, 110:13-21 (2008), Nanjee et al., Clin Chem, 2000; 46:207-223 or U.S. Pat. No. 7,232,653.

Vesicles can be isolated and/or detected to provide diagnosis, prognosis, disease stratification, theranosis, prediction of responder or non-responder status, disease monitoring, treatment monitoring and the like. In one embodiment, vesicles are isolated from cells having a disease or disorder, e.g., cells derived from a malignant cell, a site of autoimmune disease, cardiovascular disease, neurological disease, or infection. In some embodiments, the isolated vesicles are derived from cells related to such diseases and disorders. The isolated vesicles are also useful to discover novel biomarkers. By identifying biomarkers associated with vesicles, isolated vesicles can be assessed for characterizing a phenotype as described herein.

### Bio-Signature

A vesicle bio-signature from a subject can be used to characterize a phenotype of the subject. A bio-signature can include the level of one or more biomarkers. A biosignature of a vesicle of interest can include particular antigens or biomarkers that are present on the vesicle. A bio-signature can also include one or more antigens or biomarkers that are carried as payload within the vesicle. A bio-signature can comprise a combination of one or more antigens or biomarkers that are present on the vesicle with one or more biomarkers that are detected in the vesicle. A biosignature can further comprise other information about a vesicle aside from its biomarkers. Such information can include vesicle size, circulating half-life, metabolic half-life, and specific activity in vivo or in vitro. A biosignature can comprise the biomarkers or other characteristics used to build a classifier.

Vesicles can be purified or concentrated prior to determining the bio-signature of the vesicle. For example, a cell-of-origin specific vesicle can be isolated and its bio-signature determined. Alternatively, the bio-signature of the vesicle can be directly assayed from a sample, without prior purification or concentration. The bio-signature can be used to determine a diagnosis, prognosis, or theranosis of a disease or condition or similar measures described herein. A bio-signature can also be used to determine treatment efficacy, stage of a disease or condition, or progression of a disease or condition, or responder/non-responder status. Furthermore, a bio-signature may be used to determine a physiological state, such as pregnancy.

A characteristic of a vesicle in and of itself can be assessed to determine a bio-signature. The characteristic can be used to diagnose, detect or determine a disease stage or progression, the therapeutic implications of a disease or condition, or characterize a physiological state. Such characteristics include without limitation the level or amount of vesicles, vesicle size, temporal evaluation of the variation in vesicle half-life, circulating vesicle half-life, metabolic half-life of a vesicle, or activity of a vesicle.

Biomarkers included in a biosignature may include one or more proteins or peptides (e.g., providing a protein signature), nucleic acids (e.g. RNA signature as described, or a DNA signature), lipids (e.g. lipid signature), or combinations thereof. In some embodiments, the bio-signature can also comprise the type or amount of drug or drug metabolite present in a vesicle, (e.g., providing a drug signature), as such drug may be taken by a subject from which the biological sample is obtained, resulting in a vesicle carrying the drug or metabolites of the drug.

A bio-signature can also correspond to an expression level, presence, absence, mutation, variant, copy number variation, truncation, duplication, modification, or molecular association of one or more biomarkers associated with the vesicle. A genetic variant, or nucleotide variant, refers to changes or alterations to a gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant may occur in transcriptional regulatory regions, untranslated regions of mRNA, exons, introns, or exon/intron junctions. The genetic variant may or may not result in stop codons, frame shifts, deletions of amino acids, altered gene transcript splice forms or altered amino acid sequence.

In an embodiment, nucleic acid payload within the vesicle is assessed for nucleotide variants. The nucleic acid biomarker may comprise the RNA content of a vesicle, such that the signature includes analysis of one or more RNA species, e.g., mRNA, miRNA, snoRNA, snRNA, rRNAs, tRNAs, siRNA, hnRNA, shRNA, or a combination thereof. Therefore, a vesicle can be assayed to determine a RNA signature. Similarly, DNA payload can be assessed to form a DNA signature.

An RNA signature or DNA signature can also include a mutational, epigenetic modification, or genetic variant analysis of the RNA or DNA present in the vesicle. Epigenetic modifications include patterns of DNA methylation. See, e.g., Lesche R. and Eckhardt F., DNA methylation markers: a versatile diagnostic tool for routine clinical use. Curr Opin Mol Ther. 2007 June; 9(3):222-30, which is incorporated herein by reference in its entirety. A bio-signature of a vesicle can comprise one or more miRNA signatures combined with one or more additional signatures including, but not limited to, an mRNA signature, DNA signature, protein signature, peptide signature, antigen signature, or any combination thereof. For example, the bio-signature can comprise one or more miRNA biomarkers with one or more DNA biomarkers, one or more mRNA biomarkers, one or more snoRNA biomarkers, one or more protein biomarkers, one or more peptide biomarkers, one or more antigen biomarkers, one or more antigen biomarkers, one or more lipid biomarkers, or any combination thereof.

A bio-signature can comprise a combination of one or more antigens or binding events with more or more binding agents, such as listed in Tables 1 and 2, or those described in U.S. patent application Ser. No. 12/591,226, filed Nov. 12, 2009 and entitled “Methods and Systems of Using Exosomes for Determining Phenotypes,” which application is hereby incorporated by reference in its entirety. The bio-signature can further comprise one or more other biomarkers, such as, but not limited to, miRNA, DNA (e.g. single stranded DNA, complementary DNA, or noncoding DNA), or mRNA. For example, the bio-signature of a vesicle can comprise a combination of one or more antigens, such as shown in Table 1, one or more binding agents, such as shown in Table 2, and one or more biomarkers for a condition or disease of interest such as those described in U.S. patent application Ser. No. 12/591,226. The bio-signature can comprise one or more biomarkers, for example miRNA, with one or more antigens specific for a cancer cell (for example, as shown in Table 1). The biosignature can be derived from surface markers on the vesicle and/or payload markers from within the vesicle (e.g., miRNA payload).

In some embodiments, a vesicle has a bio-signature that is specific to the cell-of-origin and is used to derive disease-specific or biological state specific diagnostic, prognostic or therapy-related bio-signatures representative of the cell-of-origin. In other embodiments, a vesicle has a bio-signature that is specific to a given disease or physiological condition that is different from the bio-signature of the cell-of-origin for use in the diagnosis, prognosis, staging, therapy-related determinations or physiological state characterization. Biosignatures can also comprise a combination of cell-of-origin specific and non-specific vesicles.

Vesicle biosignatures can be used to evaluate diagnostic criteria such as presence of disease, disease staging, disease monitoring, disease stratification, or surveillance for detection, metastasis or recurrence or progression of disease. The bio-signature of a vesicle can also be used clinically in making decisions concerning treatment modalities including therapeutic intervention. The bio-signature of a vesicle can further be used clinically to make treatment decisions, including whether to perform surgery or what treatment standards should be utilized along with surgery (e.g., either pre-surgery or post-surgery). As an illustrative example, a vesicle biosignature that indicates an aggressive form of cancer may call for a more aggressive surgical procedure and/or more aggressive therapeutic regimen to treat the patient.

A bio-signature can be used in therapy related diagnostics to provide tests useful to diagnose a disease or choose the correct treatment regimen, such as provide a theranosis. Theranostics includes diagnostic testing that provides the ability to affect therapy or treatment of a diseased state. Theranostics testing provides a theranosis in a similar manner that diagnostics or prognostic testing provides a diagnosis or prognosis, respectively. As used herein, theranostics encompasses any desired form of therapy related testing. Therapy related tests can be used to predict and assess drug response in individual subjects, i.e., to provide personalized medicine. Therapy related tests are also useful to select a subject for treatment who is particularly likely to benefit from the treatment or to provide an early and objective indication of treatment efficacy in an individual subject. Thus, a vesicle signature may indicate that treatment should be altered to select a more promising treatment, thereby avoiding the great expense of delaying beneficial treatment and avoiding the financial and morbidity costs of administering an ineffective drug(s).

Therapy related diagnostics are also useful in clinical diagnosis and management of a variety of diseases and disorders, which include, but are not limited to cardiovascular disease, cancer, infectious diseases, sepsis, neurological diseases, central nervous system related diseases, endovascular related diseases, and autoimmune related diseases. Therapy related diagnostics also aid in the prediction of drug toxicity, drug resistance or drug response. Therapy related tests may be developed in any suitable diagnostic testing format, which include, but are not limited to, e.g., immunohistochemical tests, clinical chemistry, immunoassay, cell-based technologies, nucleic acid tests or body imaging methods. Therapy related tests can further include but are not limited to, testing that aids in the determination of therapy, testing that monitors for therapeutic toxicity, or response to therapy testing. Thus, a bio-signature of a vesicle can be used to predict or monitor a subject's response to a treatment. A bio-signature of a vesicle or the amount of vesicles with a particular bio-signature can be determined at different time points for a subject after initiating, removing, or altering a particular treatment.

In some embodiments, a determination or prediction as to whether a subject is responding to a treatment is made based on a change on the amount of vesicles, amount of vesicles with a particular bio-signature, or the bio-signature detected for one or more vesicles. In another embodiment, a subject's condition is monitored by determining a bio-signature of a vesicle or the amount of vesicles, such as vesicles with a particular bio-signature, at different time points. The progression, regression, or recurrence of a condition is determined. Response to therapy can also be measured over a time course. Thus, the invention provides a method of monitoring a status of a disease or other medical condition in a subject, comprising isolating or detecting a vesicle fraction from a biological sample from the subject, detecting the overall amount of vesicles or the amount of vesicles with a particular bio-signature, or detecting the bio-signature of one or more vesicles (such as the presence, absence, or expression level of a biomarker). The vesicle biosignatures are used to monitor the status of the disease or condition.

In some embodiments, a bio-signature is used to determine whether a particular disease or condition is resistant to a drug. If a subject is drug resistant, a physician need not waste valuable time with such drug treatment. To obtain early validation of a drug choice or treatment regimen, a bio-signature is determined for a vesicle obtained from a subject. The bio-signature is used to assess whether the particular subject's disease has the biomarker associated with drug resistance. Such a determination enables doctors to devote critical time as well as the patient's financial resources to effective treatments.

In some embodiments, a vesicle bio-signature is used to assess whether a subject is afflicted with disease, is at risk for developing disease or to assess the stage or progression of the disease. In illustrative examples, a bio-signature is used to assess whether a subject has prostate cancer by detecting one or more of the general vesicle markers CD9, CD63 and CD81; one or more prostate epithelial markers including PCSA or PSMA; and one or more cancer markers such as B7H3 and/or EpCam. Higher levels of the markers in a sample from a subject than in a control individual without prostate cancer can indicate the presence of PCa in the subject. In another illustrative example, a bio-signature is used to determine a stage of a disease or condition as described in U.S. patent application Ser. No. 12/591,226.

In some embodiments, characterizing a phenotype comprises determining the amount of vesicles, such a heterogeneous population of vesicles, and the amount of one or more homogeneous population of vesicles, such as a population of vesicles with the same bio-signature. In an embodiment, determination of the total amount of vesicles in a sample (i.e. not cell-type specific) and determining the presence of one or more cell-of-origin specific vesicles are used to characterize a phenotype. Threshold values, or reference values or amounts can be determined based on comparisons of normal subjects and subjects with the phenotype of interest, as further described herein, and criteria based on the threshold or reference values determined. The different criteria can be used to characterize a phenotype.

One criterion for characterizing a phenotype comprises the amount of a heterogeneous population of vesicles in a sample. In one embodiment, general vesicle markers, such as tetraspanins such as CD9, CD81, and CD63, are used to determine the amount of vesicles in a sample. The expression level of CD9, CD81, CD63, or a combination thereof can be detected and if the level is greater than a threshold level, the criterion is met. In another embodiment, the criterion is met if a level of CD9, CD81 and/or CD63, is lower than a threshold value or reference value. In another embodiment, the criterion is based on whether the amount of vesicles is higher than a threshold or reference value. Another criterion is based on the amount of vesicles with a specific bio-signature. If the amount of vesicles with the specific bio-signature is lower than a threshold or reference value, the criterion is met. In another embodiment, if the amount of vesicles with the specific bio-signature is higher than a threshold or reference value, the criterion is met. A criterion can also be based on the amount of vesicles derived from a particular cell type. If the amount is lower than a threshold or reference value, the criterion is met. In another embodiment, if the amount is higher than a threshold value, the criterion is met.

In a non-limiting example, consider that vesicles from prostate cells are determined by detecting the biomarker PCSA or PSCA, and that a criterion is met if the level of detected PCSA or PSCA is greater than a threshold level. The threshold can be the level of the same markers in a sample from a control cell line or control subject. Another criterion can be based on whether the amount of vesicles derived from a cancer cell or comprising one or more cancer specific biomarkers. For example, the biomarkers B7H3, EpCam, or both, can be determined and a criterion met if the level of detected B7H3 and/or EpCam is greater than a threshold level or within a pre-determined range. If the amount is lower, or higher, than a threshold or reference value, the criterion is met. A criterion can also be the reliability of the result, such as meeting a quality control measure or value. A detected amount of B7H3 and/or EpCam in a test sample that is above the amount of these markers in a control sample may indicate the presence of a cancer in the test sample.

A phenotype for a subject can be characterized based on meeting any number of useful criteria. In some embodiments, at least 1, 2, 3, 4, 5, 6, 7, 8, 9, or 10 criteria are used. For example, for the characterizing of a cancer, a number of different criteria can be used when the subject is diagnosed with a cancer: 1) if the amount of vesicles in a sample from a subject is higher than a reference value; 2) if the amount of a cell type specific vesicles (i.e. vesicles derived from a specific tissue or organ) is higher than a reference value; or 3) if the amount of vesicles with one or more cancer specific biomarkers is higher than a reference value. The method can further include a quality control measure, such that the results are provided for the subject if the samples meet the quality control measure. In some embodiments, if the criteria are met but the quality control is questionable, the subject is reassessed.

A bio-signature can be determined by comparing the amount of vesicles, the structure of a vesicle, or any other informative characteristic of a vesicle. Vesicle structure can be assessed using transmission electron microscopy, see for example, Hansen et al., Journal of Biomechanics 31, Supplement 1: 134-134(1) (1998), or scanning electron microscopy. Various combinations of methods and techniques or analyzing one or more vesicles can be used to determine a phenotype for a subject.

A characteristic of a vesicle can include without limitation the presence or absence, copy number, expression level, or activity level of a biomarker. Other vesicle characteristics include the presence of a mutation (e.g., mutations which affect activity of a transcription or translation product, such as substitution, deletion, or insertion mutations), variant, or post-translation modification of a biomarker. Post-translational modification of a protein biomarker include without limitation acylation, acetylation, phosphorylation, ubiquitination, deacetylation, alkylation, methylation, amidation, biotinylation, gamma-carboxylation, glutamylation, glycosylation, glycyation, hydroxylation, covalent attachment of heme moiety, iodination, isoprenylation, lipoylation, prenylation, GPI anchor formation, myristoylation, farnesylation, geranylgeranylation, covalent attachment of nucleotides or derivatives thereof, ADP-ribosylation, flavin attachment, oxidation, palmitoylation, pegylation, covalent attachment of phosphatidylinositol, phosphopantetheinylation, polysialylation, pyroglutamate formation, racemization of proline by prolyl isomerase, tRNA-mediation addition of amino acids such as arginylation, sulfation, the addition of a sulfate group to a tyrosine, or selenoylation of the biomarker.

The methods described herein can be used to identify a bio-signature that is associated with a disease, condition or physiological state. The bio-signature can also be used to determine if a subject is afflicted with cancer or is at risk for developing cancer. A subject at risk of developing cancer can include those who may be predisposed or who have pre-symptomatic early stage disease.

A bio-signature can also be used to provide a diagnostic or theranostic determination for other diseases including but not limited to autoimmune diseases, inflammatory bowel diseases, cardiovascular disease, neurological diseases such as Alzheimer's disease, Parkinson's disease, Multiple Sclerosis, infectious disease such as sepsis, pancreatitis or other disease, conditions or symptoms listed in as disclosed in U.S. patent application Ser. No. 12/591,226.

The bio-signature can also be used to identify a given pregnancy state from the peripheral blood, umbilical cord blood, or amniotic fluid (e.g. miRNA signature specific to Down's Syndrome) or adverse pregnancy outcome such as pre-eclampsia, pre-term birth, premature rupture of membranes, intrauterine growth restriction or recurrent pregnancy loss. The bio-signature can also be used to indicate the health of the mother or the health of the fetus at all developmental stages, the pre-implantation embryo or a newborn.

A bio-signature can be used for pre-symptomatic diagnosis. Furthermore, the bio-signature can be utilized to detect disease, determine disease stage or progression, determine the recurrence of disease, identify treatment protocols, determine efficacy of treatment protocols or evaluate the physiological status of individuals related to age and environmental exposure.

Monitoring a vesicle bio-signature can be used to identify toxic exposures in a subject including, but not limited to, situations of early exposure or exposure to an unknown or unidentified toxic agent. Without being bound by any one specific theory for mechanism of action, vesicles can shed from damaged cells and in the process compartmentalize specific contents of the cell including both membrane components and engulfed cytoplasmic contents. Cells exposed to toxic agents/chemicals may increase vesicle shedding to expel toxic agents or metabolites thereof, thus resulting in increased vesicle levels. Thus, monitoring vesicle levels, vesicle bio-signature, or both, allows assessment of an individual's response to toxic agent(s).

Furthermore, a vesicle can be used to identify states of drug-induced toxicity or the organ injured, by detecting one or more specific antigen, binding agent, biomarker, or any combination thereof of the vesicle. The level of vesicles, changes in the bio-signature of a vesicle, or both, can be used to monitor an individual for acute, chronic, or occupational exposures to any number of toxic agents including, but not limited to, drugs, antibiotics, industrial chemicals, toxic antibiotic metabolites, herbs, household chemicals, and chemicals produced by other organisms, either naturally occurring or synthetic in nature.

In some embodiments, a bio-signature is used to identify conditions or diseases, including cancers of unknown origin, also known as cancers of unknown primary (CUP). For example, a vesicle may be isolated from a biological sample as previously described to arrive at a heterogeneous population of vesicles. The heterogeneous population of vesicles can then be applied to surfaces coated with specific binding agents designed to identify antigen specific characteristics of the vesicle population that are specific to a given cell-of-origin. Further, as described above, the bio-signature of a vesicle can correlate with the cancerous state of cells. Compounds that inhibit cancer in a subject may cause a change, e.g., a change in bio-signature of a vesicle, which can be monitored by serial isolation of vesicles over time and course of treatment. The level of vesicles or changes in the level of vesicles with a specific bio-signature can be monitored to concomitantly monitor treatment efficacy.

In an aspect, characterizing a phenotype of a subject comprises a method of determining whether the subject is likely to respond or not respond to a therapy. The methods of the invention also include determining new biosignatures useful in predicting whether the subject is likely to respond or not. One or more subjects that respond to a therapy (responders) and one or more subjects that do not respond to the same therapy (non-responders) can have their vesicles interrogated. Interrogation can be performed to identify vesicle biosignatures that classify a subject as a responder or non-responder to the treatment of interest. In some aspects, the presence, quantity, and payload of a vesicle are assayed. The payload of a vesicle includes, for example, internal proteins, nucleic acids such as miRNA, lipids or carbohydrates.

A biosignature indicative of responder/non-responder status can be used for theranosis. A sample from subjects with known or determinable responder/non-responder status may be analyzed for one or more of the following: amount of vesicles, amount of a unique subset or species of vesicles, biomarkers in such vesicles, biosignature of such vesicles, etc. In one instance, vesicles such as microvesicles or exosomes from responders and non-responders are analyzed for the presence and/or quantity of one or more miRNAs, such as miR-122 or miR-141. A difference in biosignatures between responders and non-responders can be used for theranosis. In another embodiment, vesicles are obtained from subjects having a disease or condition. Vesicles are also obtained from subjects free of such disease or condition. The vesicles from both groups of subjects are assayed for unique biosignatures that are associated with all subjects in that group but not in subjects from the other group. Such biosignatures or biomarkers can then used as a diagnostic for the presence or absence of the condition or disease, or to classify the subject as belonging on one of the groups (those with/without disease, aggressive/non-aggressive disease, responder/non-responder, etc).

In an aspect, characterizing a phenotype of a subject comprises a method of staging a disease. The methods of the invention also include determining new biosignatures useful in staging. In an illustrative example, vesicles are assayed from patients having a stage I cancer and patients having stage II or stage III of the same cancer. In some embodiments, vesicles are assayed in patients with metastatic disease. A difference in biosignatures or biomarkers between vesicles from each group of patient is identified (e.g., vesicles from stage III cancer may have an increased expression of one or more genes or miRNAs), thereby identifying a biosignature or biomarker that distinguishes different stages of a disease. Such biosignature can then be used to stage patients having the disease.

In some instances, a biosignature is determined by assaying vesicles from a subject over a period of time, e.g., daily, semiweekly, weekly, biweekly, semimonthly, monthly, bimonthly, semiquarterly, quarterly, semiyearly, biyearly or yearly. For example, the biosignatures in patients on a given therapy can be monitored over time to detect signatures indicative of responders or non-responders for the therapy. Similarly, patients with differing stages of disease have their vesicles interrogated over time. The payload or physical attributes of the vesicles in each point in time can be compared. A temporal pattern can thus form a biosignature that can then be used for theranosis, diagnosis, prognosis, disease stratification, treatment monitoring, disease monitoring or making a prediction of responder/non-responder status. As an illustrative example only, an increasing amount of a biomarker (e.g., miR 122) in vesicles over a time course is associated with metastatic cancer, as opposed to a stagnant amounts of the biomarker in vesicles over the time course that are associated with non-metastatic cancer. A time course may last over at least 1 week, 2 weeks, 3 weeks, 4 weeks, 1 month, 6 weeks, 8 weeks, 2 months, 10 weeks, 12 weeks, 3 months, 4 months, 5 months, 6 months, 7 months, 8 months, 9 months, 10 months, 11 months, 12 months, one year, 18 months, 2 years, or at least 3 years.

The level of vesicles, level of vesicles with a specific bio-signature, or a bio-signature of a vesicle can be used to assess the efficacy of a therapy for a condition. In an embodiment, vesicles are used to assess the efficacy of a cancer treatment, e.g., chemotherapy, radiation therapy, surgery, or any other therapeutic approach useful for treating cancer in a subject. In addition, a bio-signature can be used in a screening assay to identify candidate or test compounds or agents (e.g., proteins, peptides, peptidomimetics, peptoids, small molecules or other drugs) that have a modulatory effect on the bio-signature of a vesicle. Compounds identified via such screening assays may be useful, for example, for modulating, e.g., inhibiting, ameliorating, treating, or preventing conditions or diseases.

In one embodiment, the invention provides a screening method for drug development. A bio-signature for a vesicle is obtained from a patient who is undergoing successful treatment for a particular disease, e.g., a cancer. Cells from a patient with the disease but not being treated with the same treatment can are cultured and vesicles from the cultures obtained for determining bio-signatures. The cells are treated with test compounds and the bio-signature of the vesicles from the cultures are compared to the bio-signature of the vesicles obtained from the patient undergoing successful treatment. Bio-signatures that are similar to those of the patient undergoing successful treatment indicate a successful treatment and the corresponding test compounds can be selected for further studies.

The bio-signature of a vesicle can be used to monitor the influence of an agent (e.g., drug compounds) on the bio-signature in clinical trials. Monitoring the level of vesicles, changes in the bio-signature of a vesicle, or both, can also be used in a method of assessing the efficacy of a test compound, such as a test compound for inhibiting cancer cells. A vesicle biosignature of individuals who respond to the drug can also be used as a diagnostic predict responder/non-responder status of new patients.

In addition to diagnosing or confirming the presence of or risk for developing a disease, condition or a syndrome, the methods and compositions disclosed herein also provide a system for optimizing the treatment of a subject having such a disease, condition or syndrome. The vesicle bio-signature of a vesicle can be used to determine the effectiveness of a particular therapeutic intervention (pharmaceutical or non-pharmaceutical) and to alter the intervention to 1) reduce the risk of developing adverse outcomes, 2) enhance the effectiveness of the intervention or 3) identify resistant states. Accordingly, the real-time treatment of a subject can be improved by identifying the bio-signature of a vesicle to guide treatment selection.

Tests that identify the level of vesicles, the bio-signature of a vesicle, or both, can be used to identify which patients are most suited to a particular therapy, and provide feedback on how well a drug is working, so as to optimize treatment regimens. For example, in pregnancy-induced hypertension and associated conditions, therapy-related diagnostics can flexibly monitor changes in important parameters (e.g., cytokine and/or growth factor levels) over time, to optimize treatment.

Within the clinical trial setting of investigational agents as defined by the FDA, MDA, EMA, USDA, and EMEA, therapy-related diagnostics as determined by a bio-signature disclosed herein, can provide key information to optimize trial design, monitor efficacy, and enhance drug safety. For instance, for trial design, therapy-related diagnostics can be used for patient stratification, determination of patient eligibility (inclusion/exclusion), creation of homogeneous treatment groups, and selection of patient samples that are optimized to a matched case control cohort. Such therapy-related diagnostic can therefore provide the means for patient efficacy enrichment, thereby minimizing the number of individuals needed for trial recruitment. For efficacy, therapy-related diagnostics can be useful for monitoring therapy and assessing efficacy criteria. Alternatively, for safety, therapy-related diagnostics can be used to prevent adverse drug reactions or avoid medication error and monitor compliance with the therapeutic regimen.

In some embodiments, the invention provides a method of identifying responder and non-responders to a treatment undergoing clinical trials, comprising detecting vesicle levels and/or biosignatures in subjects enrolled in the clinical trial, and identifying vesicles levels and/or biosignatures that distinguish between responders and non-responders. In a further embodiment, the vesicle levels and/or biosignatures are measured in a drug naive subject and used to predict whether the subject will be a responder or non-responder. The prediction can be based upon whether the vesicle levels and/or biosignatures of the drug naive subject correlate more closely with the clinical trial subjects identified as responders, thereby predicting that the drug naive subject will be a responder. Conversely, if the vesicle levels and/or biosignatures of the drug naive subject correlate more closely with the clinical trial subjects identified as non-responders, the methods of the invention can predict that the drug naive subject will be a non-responder. The prediction can therefore be used to stratify potential responders and non-responders to the treatment. In some embodiments, the prediction is used to guide a course of treatment, e.g., by helping treating physicians decide whether to administer the drug. In some embodiments, the prediction is used to guide selection of patients for enrollment in further clinical trials. In a non-limiting example, vesicle levels and/or biosignatures that predict responder/non-responder status in Phase II trials can be used to select patients for a Phase III trial, thereby increasing the likelihood of response in the Phase III patient population. One of skill will appreciate that the method can be adapted to identify vesicles levels and/or biosignatures to stratify subjects on criteria other than responder/non-responder status. In one embodiment, the criterion is treatment safety. Therefore the method is followed as above to identify subjects who are likely or not to have adverse events to the treatment. In a non-limiting example, vesicle levels and/or biosignatures that predict safety profile in Phase II trials can be used to select patients for a Phase III trial, thereby increasing the treatment safety profile in the Phase III patient population.

Vesicle biosignatures, which can include biomarkers, vesicle levels or other vesicle characteristics, can be used to monitor drug efficacy, determine response or resistance to a given drug, or both, thereby enhancing drug safety. An illustrative example, in colon cancer vesicles are typically shed from colon cancer cells and can be isolated from the peripheral blood and used to isolate one or more biomarkers, e.g., KRAS mRNA which can then be sequenced to detect KRAS mutations. In the case of mRNA biomarkers, the mRNA can be reverse transcribed into cDNA and sequenced (e.g., by Sanger sequencing or high throughput sequencing methods) to determine if there are mutations present that confer resistance to a drug (e.g., resistance to cetuximab or panitumimab). In another example, vesicles that are specifically shed from lung cancer cells are isolated from a biological sample and used to isolate a lung cancer biomarker, e.g., EGFR mRNA. The EGFR mRNA is processed to cDNA and sequenced to determine if there are EGFR mutations present that show resistance or response to specific drugs or treatments for lung cancer.

One or more bio-signatures can be grouped so that information obtained about the set of bio-signatures in a particular group provides a reasonable basis for making a clinically relevant decision, such as but not limited to a diagnosis, prognosis, or management of treatment, such as treatment selection.

As in many diagnostic settings, it is often desirable to use the fewest number of markers sufficient to make a correct medical judgment. Fewer markers can avoid statistical overfitting of a classifier and can prevent a delay in treatment pending further analysis as well inappropriate use of time and resources.

Also disclosed herein are methods of conducting retrospective analysis on samples (e.g., serum and tissue biobanks) for the purpose of correlating qualitative and quantitative properties, such as bio-signatures of vesicles, with clinical outcomes in terms of disease state, disease stage, progression, prognosis; therapeutic efficacy or selection; or physiological conditions. Furthermore, methods and compositions disclosed herein are useful for conducting prospective analysis on a sample (e.g., serum and/or tissue collected from individuals in a clinical trial) for the purpose of correlating vesicle bio-signatures with clinical outcomes in terms of disease state, disease stage, progression, prognosis; therapeutic efficacy or selection; or physiological conditions can also be performed.

Vesicle bio-signatures can be determined based on a surface marker profile of a vesicle or contents of a vesicle, in addition to characteristics of the vesicle such as level, size or morphology. A bio-signature of a vesicle can comprise at least 2, 3, 4, 5, 6, 7, 8, 9, 10, 11, 12, 13, 14, 15, 16, 17, 18, 19, 20, 25, 30, 40, 50, 75, or 100 characteristics. In one embodiment, a bio-signature with more than one characteristic, such as at least 2, 3, 4, 5, 6, 7, 8, 9, 10, 11, 12, 13, 14, 15, 16, 17, 18, 19, 20, 25, 30, 40, 50, 75, or 100 characteristics, may provide higher sensitivity and/or specificity in characterizing a phenotype. In some embodiments, assessing a plurality of characteristics provides increased sensitivity and/or specificity as compared to assessing fewer characteristics.

The bio-signatures can also be used to build a classifier to classify a sample as belonging to a group, such as belonging to a group having a disease or not, a group having an aggressive disease or not, or a group of responders or non-responders. In one embodiment, a vesicle classifier is used to determine whether a subject has an aggressive or non-aggressive prostate cancer. This can help a physician to determine whether to watch the PCa, i.e., prescribe “watchful waiting,” or perform a prostatectomy. In another embodiment, a vesicle classifier is used to determine whether a breast cancer patient is likely to respond or not to tamoxifen, thereby helping the physician to determine whether or not to treat the patient with tamoxifen or another drug.

A bio-signature can comprise one or more biomarkers. The biomarker can be any component present within a vesicle or on a vesicle's surface. These biomarkers include without limitation a nucleic acid (e.g. RNA (mRNA, miRNA, etc.) or DNA), protein, peptide, polypeptide, antigen, lipid, carbohydrate, or proteoglycan.

The bio-signature can include the presence or absence, expression level, mutational state, genetic variant state, or any modification (such as epigenetic modification or post-translation modification) of a biomarker (e.g. any one or more biomarker listed in Table 1). The expression level of a biomarker can be compared to a control or reference, to determine the overexpression or underexpression (or upregulation or downregulation) of a biomarker in a sample. In some embodiments, the control or reference level comprises the amount of a same biomarker, such as a miRNA, in a control sample from a subject that does not have or exhibit the condition or disease. In another embodiment, the control of reference levels comprises that of a housekeeping marker whose level is minimally affected, if at all, in different biological settings such as diseased versus non-diseased states. In yet another embodiment, the control or reference level comprises that of the level of the same marker in the same subject but in a sample taken at a different time point. Other types of controls are described herein.

Nucleic acid biomarkers include any RNA or DNA species detectably associated with vesicles. For example, the biomarker can be mRNA, miRNA, small nucleolar RNAs (snoRNA), small nuclear RNAs (snRNA), ribosomal RNAs (rRNA), heterogeneous nuclear RNA (hnRNA), ribosomal RNAS (rRNA), siRNA, transfer RNAs (tRNA), or shRNA. The DNA can be double-stranded DNA, single stranded DNA, complementary DNA, or noncoding DNA. miRNAs are short ribonucleic acid (RNA) molecules which average about 22 nucleotides long. miRNAs act as post-transcriptional regulators that bind to complementary sequences in the three prime untranslated regions (3′ UTRs) of target messenger RNA transcripts (mRNAs), which can result in gene silencing. One miRNA may act upon 1000s of mRNAs. miRNAs play multiple roles in negative regulation, e.g., transcript degradation and sequestering, translational suppression, and may also have a role in positive regulation, e.g., transcriptional and translational activation. By affecting gene regulation, miRNAs can influence many biologic processes. Different sets of expressed miRNAs are found in different cell types and tissues.

Biomarkers for use with the invention include a polypeptide, peptides or protein, which terms are used interchangeably throughout unless otherwise noted. In some embodiments, the protein biomarker comprises its modification state, truncations, mutations, expression level (such as overexpression or underexpression as compared to a reference level), and/or post-translational modifications, such as described above. In a non-limiting example, a biosignature for a disease can include a protein having a certain post-translational modification that is more prevalent in vesicles associated with the disease than without.

A bio-signature may include a number of the same type of biomarkers (e.g., two different mRNAs, each corresponding to a different gene) or one or more of different types of biomarkers (e.g. mRNAs, miRNAs, proteins, peptides, ligands, and antigens).

The one or more biomarkers can be detected using a probe. A probe can comprise an oligonucleotide, such as DNA or RNA, an aptamer, monoclonal antibody, polyclonal antibody, Fabs, Fab′, single chain antibody, synthetic antibody, peptoid, zDNA, peptide nucleic acid (PNA), locked nucleic acid (LNA), lectin, synthetic or naturally occurring chemical compound (including but not limited to a drug or labeling reagent), dendrimer, or a combination thereof. The probe can be directly detected, for example by being directly labeled, or be indirectly detected, such as through a labeling reagent. The probe can selectively recognize a biomarker. For example, a probe that is an oligonucleotide can selectively hybridize to a miRNA biomarker.

In aspects, the invention provides for the diagnosis, theranosis, prognosis, disease stratification, disease staging, treatment monitoring or predicting responder/non-responder status of a disease or disorder in a subject. The invention comprises assessing vesicles from a subject, including assessing biomarkers present on the vesicles and/or assessing payload within the vesicles, such as protein, nucleic acid or other biological molecules. Any appropriate biomarker that can be assessed using a vesicle and that relates to a disease or disorder can be used the carry out the methods of the invention. Furthermore, any appropriate technique to assess a vesicle as described herein can be used.

As an illustrative example, benign prostatic hyperplasia (BPH) specific biomarkers from a vesicle can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, and can be used to create a BPH specific bio-signature. The protein, ligand, or peptide that can be assessed in a vesicle can include, but is not limited to, intact fibronectin.

The invention also provides an isolated vesicle comprising one or more BPH specific biomarkers, such as listed in Table 1 for BPH. A composition comprising the isolated vesicle is also provided. Accordingly, in some embodiments, the composition comprises a population of vesicles comprising one or more BPH specific biomarkers, such as listed in Table 1 for BPH. The composition can comprise a substantially enriched population of vesicles, wherein the population of vesicles is substantially homogeneous for BPH specific vesicles or vesicles comprising one or more BPH specific biomarkers, such as listed in Table 1 for BPH.

One or more BPH specific biomarkers, such as listed in Table 1 for BPH, can also be detected by one or more systems disclosed herein, for characterizing a BPH. For example, a detection system can comprise one or more probes to detect one or more BPH specific biomarkers, such as listed in Table 1 for BPH, of one or more vesicles of a biological sample. One of skill will appreciate that numerous other vesicle associated biomarkers can be used to create a biosignature for BPH in addition to those specifically described here, e.g., those disclosed in U.S. patent application Ser. No. 12/591,226.

Similarly, prostate cancer (PCa) specific biomarkers from a vesicle can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in Table 1, and can be used to create a prostate cancer specific bio-signature. For example, a bio-signature for prostate cancer can comprise miR-9, miR-21, miR-141, miR-370, miR-200b, miR-210, miR-155, or miR-196a. In some embodiments, the bio-signature can comprise one or more overexpressed miRs, such as, but not limited to, miR-202, miR-210, miR-296, miR-320, miR-370, miR-373, miR-498, miR-503, miR-184, miR-198, miR-302c, miR-345, miR-491, miR-513, miR-32, miR-182, miR-31, miR-26a-1/2, miR-200c, miR-375, miR-196a-1/2, miR-370, miR-425, miR-425, miR-194-1/2, miR-181a-1/2, miR-34b, let-7i, miR-188, miR-25, miR-106b, miR-449, miR-99b, miR-93, miR-92-1/2, miR-125a, or miR-141, or any combination thereof.

The bio-signature can also comprise one or more underexpressed miRs such as, but not limited to, let-7a, let-7b, let-7c, let-7d, let-7g, miR-16, miR-23a, miR-23b, miR-26a, miR-92, miR-99a, miR-103, miR-125a, miR-125b, miR-143, miR-145, miR-195, miR-199, miR-221, miR-222, miR-497, let-7f, miR-19b, miR-22, miR-26b, miR-27a, miR-27b, miR-29a, miR-29b, miR-30_{—}5p, miR-30c, miR-100, miR-141, miR-148a, miR-205, miR-520h, miR-494, miR-490, miR-133a-1, miR-1-2, miR-218-2, miR-220, miR-128a, miR-221, miR-499, miR-329, miR-340, miR-345, miR-410, miR-126, miR-205, miR-7-1/2, miR-145, miR-34a, miR-487, or let-7b, or any combination thereof. The bio-signature can comprise upregulated or overexpressed miR-21, downregulated or underexpressed miR-15a, miR-16-1, miR-143 or miR-145, or any combination thereof.

The one or more mRNAs that may be analyzed can include, but are not limited to, AR, PCA3, or any combination thereof and can be used as specific biomarkers from a vesicle for prostate cancer.

The protein, ligand, or peptide that can be assessed in a vesicle can include, but is not limited to, FASLG, HSP60, PSMA, PCSA or TNESF10 or any combination thereof. Antibodies for binding PSMA are found in U.S. Pat. Nos. 6,207,805 and 6,512,096. Furthermore, a vesicle isolated or assayed can be prostate cancer cell specific, or derived from prostate cancer cells. Furthermore, the snoRNA that can be used as an vesicle biomarker for prostate cancer can include, but is not limited to, U50. Examples of prostate cancer bio-signatures are further described below.

The invention also provides an isolated vesicle comprising one or more prostate cancer specific biomarkers, such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in Table 1 for prostate cancer. In some embodiments, the isolated vesicle is EpCam+, CK+, CD45−. A composition comprising the isolated vesicle is also provided. Accordingly, in some embodiments, the composition comprises a population of vesicles comprising one or more prostate cancer specific biomarkers such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in Table 1 for prostate cancer. In some embodiments, the composition comprises a population of vesicles that are EpCam+, CK+, CD45−. The composition can comprise a substantially enriched population of vesicles, wherein the population of vesicles is substantially homogeneous for prostate cancer specific vesicles or vesicles comprising one or more prostate cancer specific biomarkers, such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in Table 1 for prostate cancer. In one embodiment, the composition can comprise a substantially enriched population of vesicles that are EpCam+, CK+, CD45−.

One or more prostate cancer specific biomarkers, such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in Table 1 for prostate cancer can also be detected by one or more systems disclosed herein, for characterizing a prostate cancer. In some embodiments, the biomarkers EpCam, CK (cytokeratin), and CD45 are detected by one or more of systems disclosed herein, for characterizing prostate cancer, such as determining the prognosis for a subject's prostate cancer, or the therapy-resistance of a subject. For example, a detection system can comprise one or more probes to detect one or more prostate cancer specific biomarkers, such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in Table 1 for prostate cancer, of one or more vesicles of a biological sample. In one embodiment, the detection system can comprise one or more probes to detect EpCam, CK, CD45, or a combination thereof.

One of skill will appreciate that numerous other vesicle associated biomarkers can be used to create a biosignature for PCa in addition to those specifically described here, e.g., those disclosed in U.S. patent application Ser. No. 12/591,226.

### Biomarker Detection

A bio-signature can be detected qualitatively or quantitatively. Analysis of a vesicle can comprise detecting the level of vesicles in combination with determining the bio-signature of the vesicles. Determining the level, amount, or concentration of vesicles can be performed in conjunction with determining the bio-signature of the vesicle. In some embodiments, the level of vesicles with a particular biomarker is determined and used to characterize a phenotype. In other embodiments, determining the amount of vesicles is performed prior to or subsequent to determining the biomarkers of the vesicles. The results of methods of detecting biosignatures can be used to develop a database of information useful for informing diagnostic and therapeutic decision making, e.g., what biomarkers are differentially present in subjects that respond or not to a given therapy.

In some embodiments, methods for analyzing biomarkers of tissues or cells are used to analyze the biomarkers associated with or contained in vesicles. For example, a biomarker can be detected by microarray analysis, polymerase chain reaction (PCR) (including PCR-based methods such as real time polymerase chain reaction (RT-PCR), quantitative real time polymerase chain reaction (Q-PCR/qPCR) and the like), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or combinations thereof. A biomarker, such as a nucleic acid, can be amplified prior to detection. A biomarker can also be detected by immunoblot, immunoprecipitation, enzyme-linked immunosorbent assay (ELISA; EIA), radioimmunoassay (RIA), flow cytometry, or electron microscopy (EM).

Biosignatures can be detected using capture agents and detection agents, as described herein. A capture agent can comprise an antibody or other entity which recognizes a vesicle and is useful for capturing, e.g., isolating, the vesicle. A detection agent can comprise an antibody or other entity which recognizes a vesicle and is useful for detecting a vesicle. In some embodiments, the detection agent is labeled and the label is detected, thereby detecting the vesicle. In many cases, the antigen or other vesicle-moiety that is recognized by the capture and detection agents are interchangeable. As a non-limiting example, consider a vesicle having a cell-of-origin specific antigen on its surface and a cancer-specific antigen on its surface. In one instance, the vesicle can be captured using an antibody to the cell-of-origin specific antigen, e.g., by tethering the capture antibody to a substrate, and then the vesicle is detected using an antibody to the cancer-specific antigen, e.g., by labeling the detection antibody with a fluorescent dye and detecting the fluorescent radiation emitted by the dye. In another instance, the vesicle can be captured using an antibody to the cancer specific antigen, e.g., by tethering the capture antibody to a substrate, and then the vesicle is detected using an antibody to the cell-of-origin specific antigen, e.g., by labeling the detection antibody with a fluorescent dye and detecting the fluorescent radiation emitted by the dye.

In some embodiments, a same biomarker is recognized by both a capture agent and a detection agent. This scheme can be used depending on the setting. In one embodiment, the biomarker is sufficient to detect the vesicle of interest, e.g., to capture cell-of-origin specific vesicles. In other embodiments, the biomarker is multifunctional, e.g., having both cell-of-origin specific and cancer specific properties. The biomarker can be used in concert with other biomarkers for capture and detection as well.

One method of detecting a biomarker comprises purifying or isolating a heterogeneous population of vesicles from a biological sample, as described above, and performing a sandwich assay. A vesicle in the population can be captured with a capture agent. The capture agent can be a capture antibody, such as a primary antibody. The capture antibody can be bound to a substrate, for example an array, well, or particle. The captured or bound vesicle can be detected with a detection agent, such as a detection antibody. For example, the detection antibody can be for an antigen of the vesicle. The detection antibody can be directly labeled and detected. Alternatively, the detection agent can be indirectly labeled and detected, such as through an enzyme linked secondary antibody that can react with the detection agent. A detection reagent or detection substrate can be added and the reaction detected, such as described in PCT Publication No. WO2009092386. In an illustrative example wherein the capture agent binds Rab-5b and the detection agent binds or detects CD63 or caveolin-1, the capture agent can be an anti-Rab 5b antibody and the detection agent can be an anti-CD63 or anti-caveolin-1 antibody. In some embodiments, the capture agent binds CD9, PSCA, TNFR, CD63, B7H3, MFG-E8, EpCam, Rab, CD81, STEAP, PCSA, PSMA, or 5T4. For example, the capture agent can be an antibody to CD9, PSCA, TNFR, CD63, B7H3, MFG-E8, EpCam, Rab, CD81, STEAP, PCSA, PSMA, or 5T4. The detection agent can be an agent that binds or detects CD63, CD9, CD81, B7H3, or EpCam, such as a detection antibody to CD63, CD9, CD81, B7H3, or EpCam. Various combinations of capture and/or detection agents can be used in concert. In an embodiment, the capture agents comprise PCSA, PSMA, B7H3 and optionally EpCam. The detection agents can be one or more tetraspanin such CD9, CD63 and CD81. Increasing numbers of such general vesicle markers can improve the detection signal in some cases.

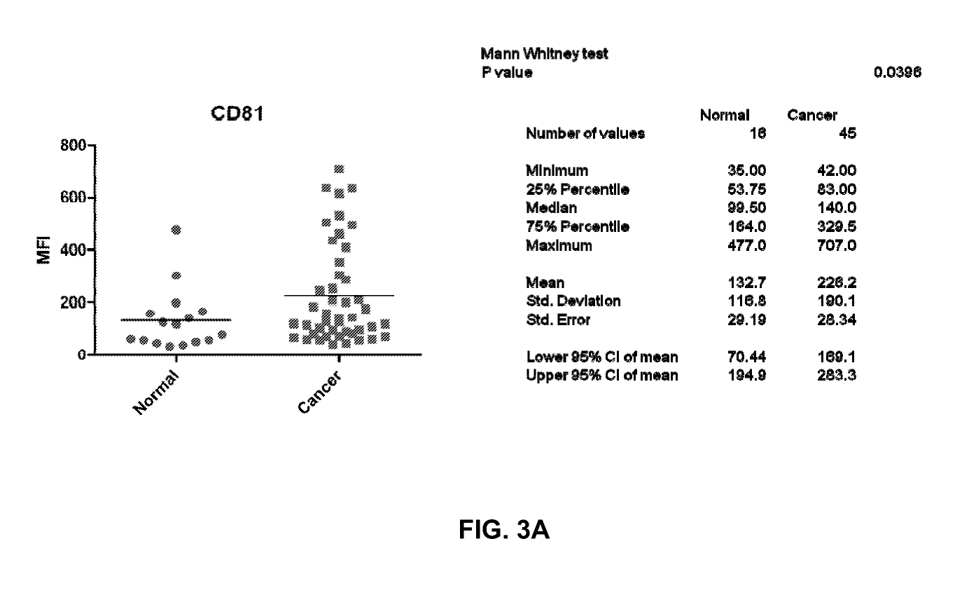

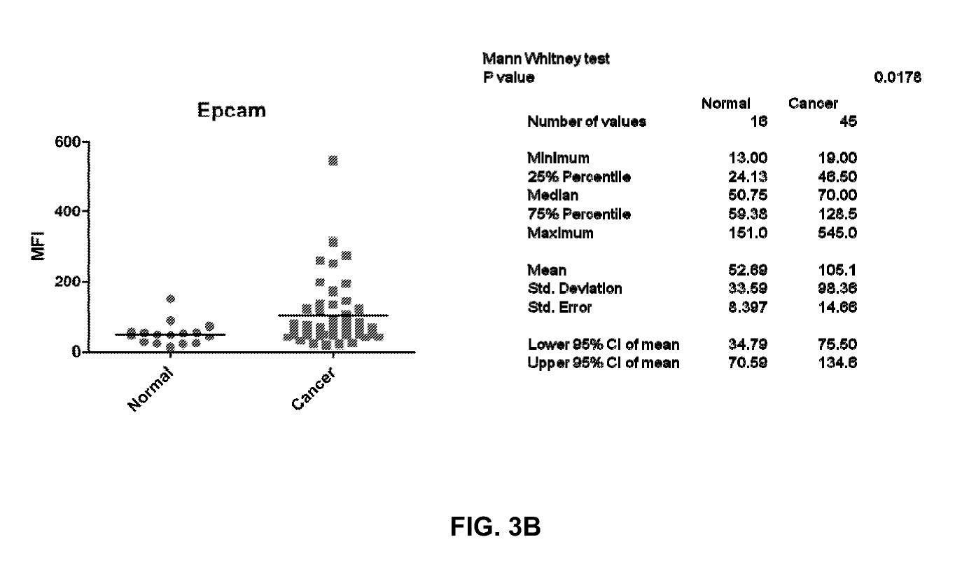

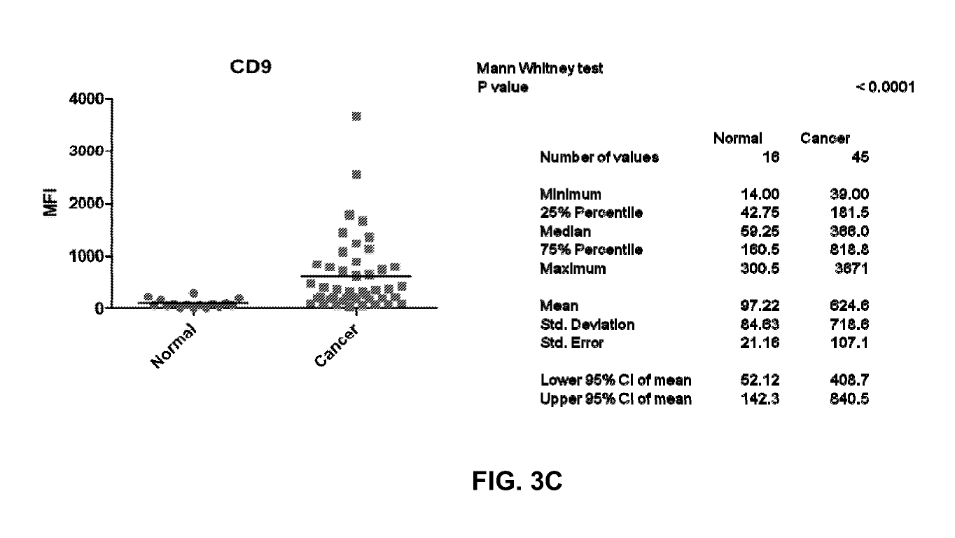

In some embodiments, the capture agent binds or targets EpCam, and the one or more biomarkers detected on the vesicle are CD9 and/or CD63. In one embodiment, the capture agent binds or targets EpCam, and the one or more biomarkers detected on the vesicle are CD9, EpCam and/or CD81. See, e.g., FIG. 3, which illustrates assessing vesicles from normal and cancer subjects using a single capture agent and single detection agent, using a capture agent that is an antibody for EpCam and detection agent that detects A) CD81, B) EpCam, or C) CD9. The single capture agent can be selected from PCSA, PSMA, B7H3, CD81, CD9 and CD63.

In other embodiments, the capture agent targets PCSA, and the one or more biomarkers detected on the captured vesicle are B7H3 and/or PSMA. In other embodiments, the capture agent targets PSMA, and the one or more biomarkers detected on the captured vesicle are B7H3 and/or PCSA. In other embodiments, the capture agent targets B7H3, and the one or more biomarkers detected on the captured vesicle are PSMA and/or PCSA. In yet other embodiments, the capture agent targets CD63 and the one or more biomarkers detected on the vesicle are CD81, CD83, CD9 and/or CD63. The different capture agent and biomarker combinations disclosed herein can be used to characterize a phenotype, such as detecting, diagnosing or prognosing a disease, e.g., a cancer. In some embodiments, vesicles are analyzed to characterize prostate cancer using a capture agent targeting EpCam and detection of CD9 and CD63; a capture agent targeting PCSA and detection of B7H3 and PSMA; or a capture agent of CD63 and detection of CD81. In other embodiments, vesicles are used to characterize colon cancer using capture agent targeting CD63 and detection of CD63, or a capture agent targeting CD9 coupled with detection of CD63. One of skill will appreciate that targets of capture agents and detection agents can be used interchangeably. In an illustrative example, consider a capture agent targeting PCSA and detection agents targeting B7H3 and PSMA. Because all of these markers are useful for detecting PCa derived vesicles, B7H3 or PSMA could be targeted by the capture agent and PCSA could be recognized by a detection agent. For example, in some embodiments, the detection agent targets PCSA, and one or more biomarkers used to capture the vesicle comprise B7H3 and/or PSMA. In other embodiments, the detection agent targets PSMA, and the one or more biomarkers used to capture the vesicle comprise B7H3 and/or PCSA. In other embodiments, the detection agent targets B7H3, and the one or more biomarkers used to capture the vesicle comprise PSMA and/or PCSA. In some embodiments, the invention provides a method of detecting prostate cancer cells in bodily fluid using capture agents and/or detection agents to PSMA, B7H3 and/or PCSA. The bodily fluid can comprise blood, including serum or plasma. The bodily fluid can comprise ejaculate or sperm. In further embodiments, the methods of detecting prostate cancer further use capture agents and/or detection agents to CD81, CD83, CD9 and/or CD63. Additional agents can improve the test performance, e.g., improving test accuracy or AUC, either by providing additional biological discriminatory power and/or by reducing experimental noise.

Techniques of detecting biomarkers for use with the invention include the use of a planar substrate such as an array (e.g., biochip or microarray), with molecules immobilized to the substrate as capture agents that facilitate the detection of a particular bio-signature of a vesicle. The array can be provided as part of a kit for assaying one or more vesicles. A molecule that identifies the biomarkers of interest, such as the antigens in Table 1, can be included in an array for detection and diagnosis of diseases including presymptomatic diseases. In some embodiments, an array comprises a custom array comprising biomolecules selected to specifically identify biomarkers of interest. Customized arrays can be modified to detect biomarkers that increase statistical performance, e.g., additional biomolecules that identifies a bio-signature which lead to improved cross-validated error rates in multivariate prediction models (e.g., logistic regression, discriminant analysis, or regression tree models). In some embodiments, customized array(s) are constructed to study the biology of a disease, condition or syndrome and profile vesicles that are shed in defined physiological states. Markers for inclusion on the customized array be chosen based upon statistical criteria, e.g., having a desired level of statistical significance in differentiating between phenotypes or physiological states. In some embodiments, standard significance of p-value=0.05 is chosen to exclude or include biomolecules on the microarray. The p-values can be corrected for multiple comparisons. As an illustrative example, nucleic acids extracted from samples from a subject with or without a disease can be hybridized to a high density microarray that binds to thousands of gene sequences. Vesicle derived nucleic acids whose levels are significantly different between the samples with or without the disease can be selected as biomarkers to distinguish samples as having the disease or not. A customized array can be constructed to detect the selected biomarkers. In some embodiments, customized arrays comprise low density microarrays, which refer to arrays with lower number of addressable binding agents, e.g., tens or hundreds instead of thousands. Low density arrays can be formed on a substrate. In some embodiments, customizable low density arrays use PCR amplification in plate wells, e.g., TaqMan® Gene Expression Assays (Applied Biosystems by Life Technologies Corporation, Carlsbad, Calif.).

A planar array generally contains addressable locations (e.g., pads, addresses, or micro-locations) of biomolecules in an array format. The size of the array will depend on the composition and end use of the array. Arrays can be made containing from 2 different molecules to many thousands. Generally, the array comprises from two to as many as 100,000 or more molecules, depending on the end use of the array and the method of manufacture. A microarray generally comprises at least one biomolecule that identifies or captures a biomarker present in a bio-signature of a specific cell-of-origin vesicle. In some arrays, multiple substrates are used, either of different or identical compositions. Accordingly, planar arrays may comprise a plurality of smaller substrates.

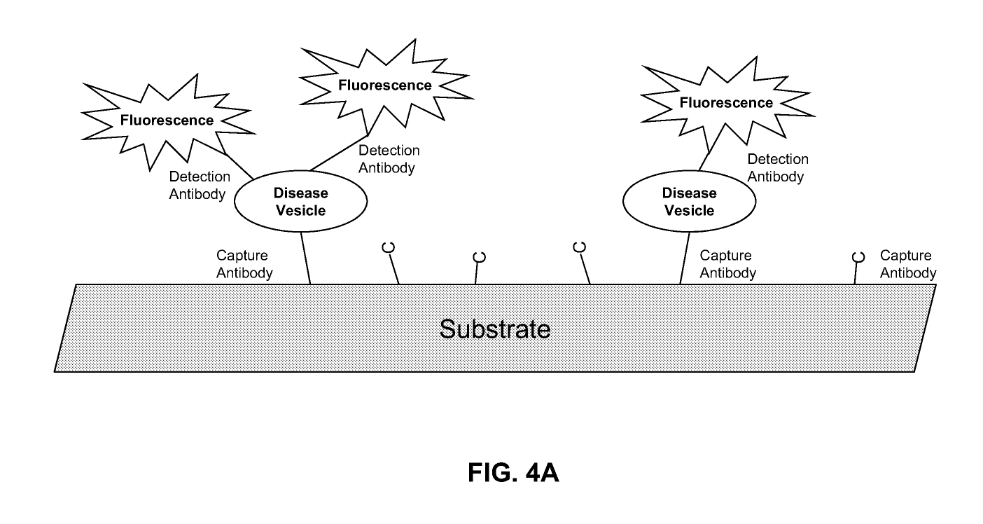

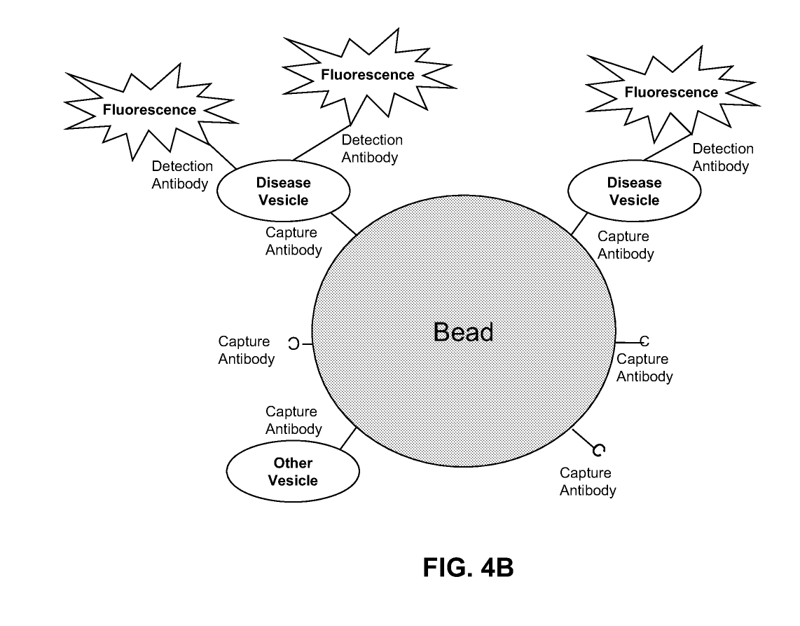

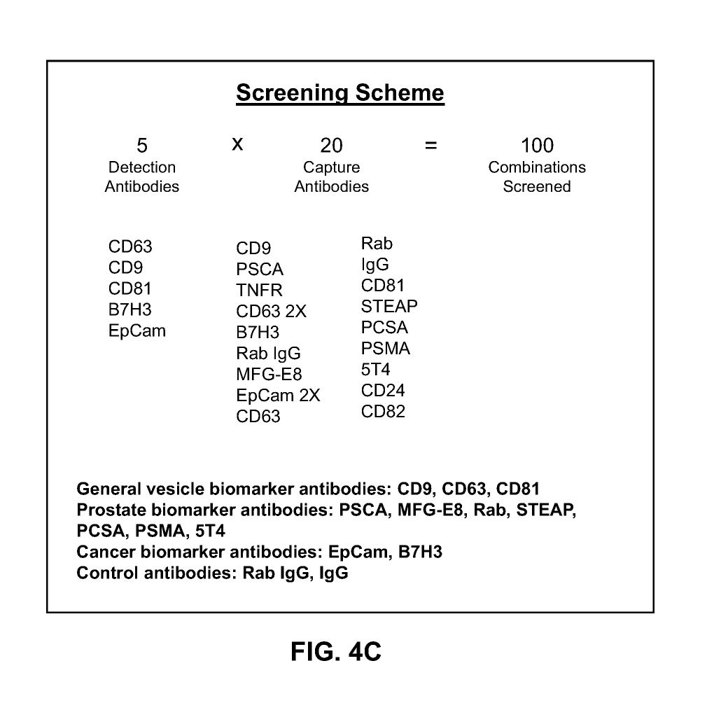

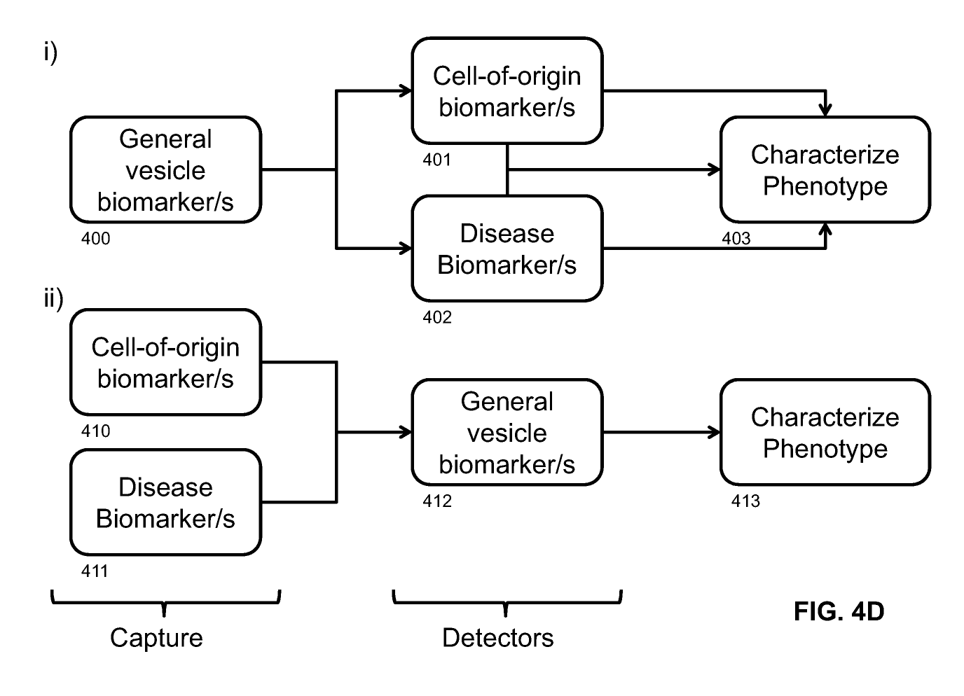

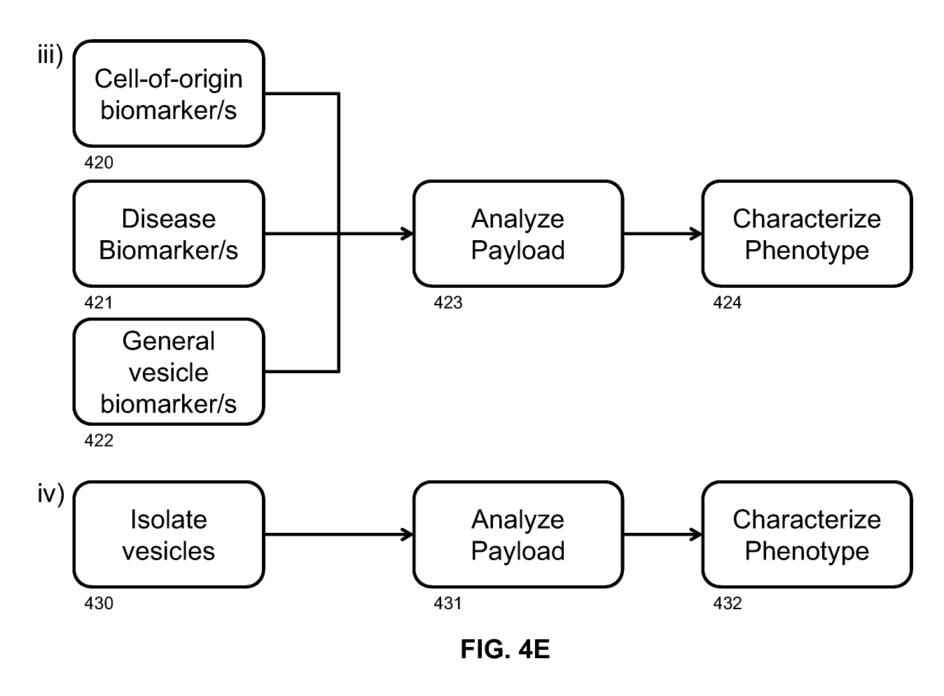

The present invention can make use of many types of arrays for detecting a biomarker, e.g., a biomarker associated with a vesicle biosignature. Useful arrays or microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). These arrays are described in more detail above. In some embodiments, microarrays comprise biochips that provide high-density immobilized arrays of recognition molecules (e.g., antibodies), where biomarker binding is monitored indirectly (e.g., via fluorescence). FIG. 4A shows an illustrative configuration in which capture antibodies against a vesicle antigen of interest are tethered to a surface. The captured vesicles are then detected using detector antibodies against the same or different vesicle antigens of interest. The capture antibodies can be substituted with tethered aptamers as available and desirable. Fluorescent detectors are shown. Other detectors can be used similarly, e.g., enzymatic reaction, detectable nanoparticles, radiolabels, and the like. In other embodiments, an array comprises a format that involves the capture of proteins by biochemical or intermolecular interaction, coupled with detection by mass spectrometry (MS).

An array or microarray that can be used to detect one or more biomarkers of a vesicle bio-signature can be made according to the methods described in U.S. Pat. Nos. 6,329,209; 6,365,418; 6,406,921; 6,475,808; and 6,475,809, and U.S. patent application Ser. No. 10/884,269, each of which is herein incorporated by reference in its entirety. Custom arrays to detect specific selections of sets of biomarkers described herein can be made using the methods described in these patents. Commercially available microarrays can also be used to carry out the methods of the invention, including without limitation those from Affymetrix (Santa Clara, Calif.), Illumina (San Diego, Calif.), Agilent (Santa Clara, Calif.), Exiqon (Denmark), or Invitrogen (Carlsbad, Calif.). Custom and/or commercial arrays include arrays for detection proteins, nucleic acids, and other biological molecules and entities (e.g., cells, vesicles, virii) as described herein.

In some embodiments, molecules to be immobilized on an array comprise proteins or peptides. One or more types of proteins may be immobilized on a surface. In certain embodiments, the proteins are immobilized using methods and materials that minimize the denaturing of the proteins, that minimize alterations in the activity of the proteins, or that minimize interactions between the protein and the surface on which they are immobilized.

Array surfaces useful may be of any desired shape, form, or size. Non-limiting examples of surfaces include chips, continuous surfaces, curved surfaces, flexible surfaces, films, plates, sheets, or tubes. Surfaces can have areas ranging from approximately a square micron to approximately 500 cm². The area, length, and width of surfaces may be varied according to the requirements of the assay to be performed. Considerations may include, for example, ease of handling, limitations of the material(s) of which the surface is formed, requirements of detection systems, requirements of deposition systems (e.g., arrayers), or the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within microwell plates having any number of wells. In such embodiments, the bottoms of the wells may serve as surfaces for the formation of arrays, or arrays may be formed on other surfaces and then placed into wells. In certain embodiments, such as where a surface without wells is used, binding islands may be formed or molecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer necessary.

In some embodiments, the immobilized molecules can bind to one or more vesicles present in a biological sample contacting the immobilized molecules. In some embodiments, the immobilized molecules modify or are modified by molecules present in the one or more vesicles contacting the immobilized molecules. Contacting the sample typically comprises overlaying the sample upon the array.

Modifications or binding of molecules in solution or immobilized on an array can be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces are measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as atomic force microscopy (AFM), scanning force microscopy (SFM) or scanning electron microscopy (SEM); and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8):363-369 (1999), and references cited therein; Lakowicz J R, Principles of Fluorescence Spectroscopy, 2nd Edition, Plenum Press (1999), or Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, each of which is herein incorporated by reference in its entirety.

Microarray technology can be combined with mass spectroscopy (MS) analysis and other tools. Electrospray interface to a mass spectrometer can be integrated with a capillary in a microfluidics device. For example, one commercially available system contains eTag reporters that are fluorescent labels with unique and well-defined electrophoretic mobilities; each label is coupled to biological or chemical probes via cleavable linkages. The distinct mobility address of each eTag reporter allows mixtures of these tags to be rapidly deconvoluted and quantitated by capillary electrophoresis. This system allows concurrent gene expression, protein expression, and protein function analyses from the same sample Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety.

A biochip can include components for a microfluidic or nanofluidic assay. A microfluidic device can be used for isolating or analyzing a vesicle, such as determining a bio-signature of a vesicle. Microfluidic systems allow for the miniaturization and compartmentalization of one or more processes for isolating, capturing or detecting a vesicle, detecting a bio-signature, and other processes. The microfluidic devices can use one or more detection reagents in at least one aspect of the system, and such a detection reagent can be used to detect one or more biomarkers of a vesicle. In one embodiment, the device detects a biomarker on the isolated or bound vesicle. Various probes, antibodies, proteins, or other binding agents can be used to detect a biomarker within the microfluidic system. The detection agents may be immobilized in different compartments of the microfluidic device or be entered into a hybridization or detection reaction through various channels of the device.

A vesicle in a microfluidic device may be lysed and its contents detected within the microfluidic device, such as proteins or nucleic acids, e.g., DNA or RNA such as miRNA or mRNA. The nucleic acid may be amplified prior to detection, or directly detected, within the microfluidic device. Thus microfluidic system can also be used for multiplexing detection of various biomarkers.

Novel nanofabrication techniques are opening up the possibilities for biosensing applications that rely on fabrication of high-density, precision arrays, e.g., nucleotide-based chips and protein arrays otherwise know as heterogeneous nanoarrays. Nanofluidics allows a further reduction in the quantity of fluid analyte in a microchip to nanoliter levels, and the chips used here are referred to as nanochips. (See, e.g., Unger M et al., Biotechniques 1999; 27(5):1008-14, Kartalov E P et al., Biotechniques 2006; 40(1):85-90, each of which are herein incorporated by reference in their entireties.) Commercially available nanochips currently provide simple one step assays such as total cholesterol, total protein or glucose assays that can be run by combining sample and reagents, mixing and monitoring of the reaction. Gel-free analytical approaches based on liquid chromatography (LC) and nanoLC separations (Cutillas et al. Proteomics, 2005; 5:101-112 and Cutillas et al., Mol Cell Proteomics 2005; 4:1038-1051, each of which is herein incorporated by reference in its entirety) can be used in combination with the nanochips.

An array suitable for identifying a disease, condition, syndrome or physiological status can be included in a kit. A kit can include, as non-limiting examples, one or more reagents useful for preparing molecules for immobilization onto binding islands or areas of an array, reagents useful for detecting binding of a vesicle to immobilized molecules, and instructions for use.

Further provided herein is a rapid detection device that facilitates the detection of a particular bio-signature of vesicles in a biological sample. The device can integrate biological sample preparation with polymerase chain reaction (PCR) on a chip. The device can facilitate the detection of a particular bio-signature of a vesicle in a biological sample, and an example is provided as described in Pipper et al., Angewandte Chemie, 47(21), p. 3900-3904 (2008), which is herein incorporated by reference in its entirety. The bio-signature of the vesicle can be incorporated using micro-/nano-electrochemical system (MEMS/NEMS) sensors and oral fluid for diagnostic applications as described in Li et al., Adv Dent Res 18(1): 3-5 (2005), which is herein incorporated by reference in its entirety.

As an alternative to planar arrays, assays using particles, such as bead based assays as described herein, can be used in combination with flow cytometry. Multiparametric assays or other high throughput detection assays using bead coatings with cognate ligands and reporter molecules with specific activities consistent with high sensitivity automation can be used. In a bead based assay system, a binding agent for a vesicle, such as a capture agent (e.g. capture antibody), can be immobilized on an addressable microsphere. Each binding agent for each individual binding assay can be coupled to a distinct type of microsphere (i.e., microbead) and the assay reaction takes place on the surface of the microsphere, such as depicted in FIG. 4B. A binding agent for a vesicle can be a capture antibody is coupled to a bead. Dyed microspheres with discrete fluorescence intensities are loaded separately with their appropriate binding agent or capture probes. The different bead sets carrying different binding agents can be pooled as necessary to generate custom bead arrays. Bead arrays are then incubated with the sample in a single reaction vessel to perform the assay. Examples of microfluidic devices that may be used, or adapted for use with vesicles, include but are not limited to those described herein.

Product formation of the biomarker with an immobilized capture molecule or binding agent can be detected with a fluorescence based reporter system (see for example, FIG. 4A-B). The biomarker can either be labeled directly by a fluorophore or detected by a second fluorescently labeled capture biomolecule. The signal intensities derived from captured biomarkers can be measured in a flow cytometer. The flow cytometer can first identify each microsphere by its individual color code. For example, distinct beads can be dyed with discrete fluorescence intensities such that each bead with a different intensity has a different binding agent. The beads can be labeled or dyed with at least 2 different labels or dyes. In some embodiments, the beads are labeled with at least 3, 4, 5, 6, 7, 8, 9, or 10 different labels. The beads with more than one label or dye can also have various ratios and combinations of the labels or dyes. The beads can be labeled or dyed externally or may have intrinsic fluorescence or signaling labels.

The amount of captured biomarkers on each individual bead can be measured by the second color fluorescence specific for the bound target. This allows multiplexed quantitation of multiple targets from a single sample within the same experiment. Sensitivity, reliability and accuracy are compared or can be improved to standard microtiter ELISA procedures. An advantage of a bead-based system is the individual coupling of the capture biomolecule or binding agent for a vesicle to distinct microspheres provides multiplexing capabilities. For example, as depicted in FIG. 4C, a combination of 5 different biomarkers to be detected (detected by antibodies to antigens such as CD63, CD9, CD81, B7H3, and EpCam) and 20 biomarkers for which to capture a vesicle, (using capture antibodies, such as antibodies to CD9, PSCA, TNFR, CD63, B7H3, MFG-E8, EpCam, Rab, CD81, STEAP, PCSA, PSMA, 5T4, and CD24) can result in approximately 100 combinations to be detected. As shown in FIG. 4C as “EpCam 2×,” “CD63 2×,” multiple antibodies to a single target can be used to probe detection against various epitopes. In another example, multiplex analysis comprises capturing a vesicle using a binding agent to CD24 and detecting the captured vesicle using a binding agent for CD9, CD63, and/or CD81. The captured vesicles can be detected using a detection agent such as an antibody. The detection agents can be labeled directly or indirectly, as described herein.

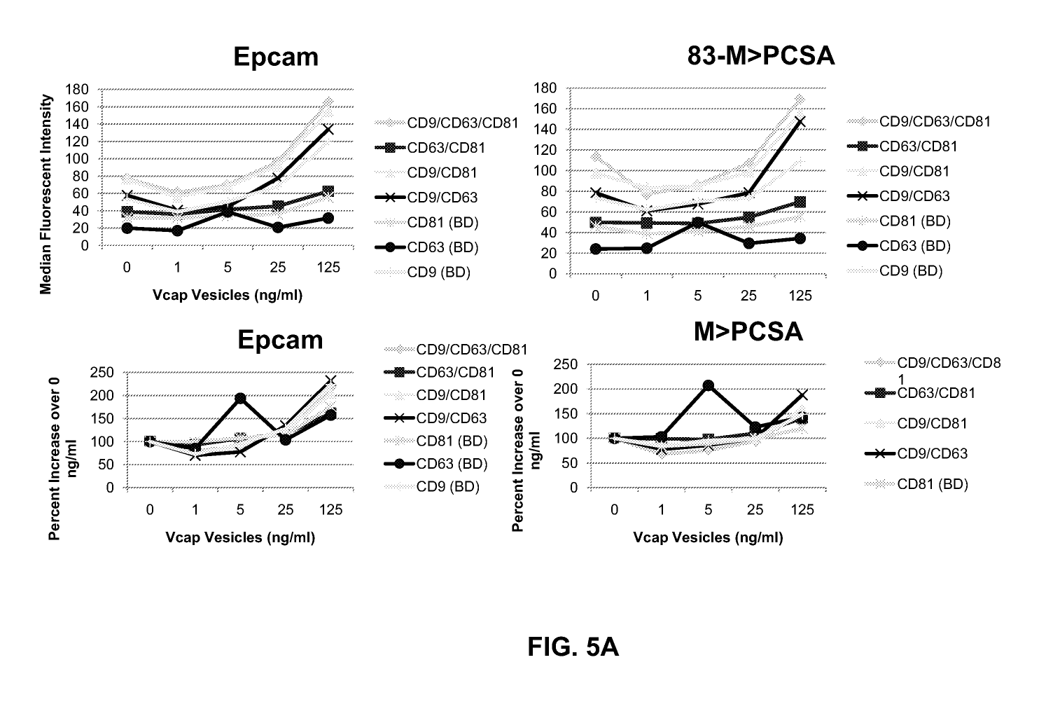

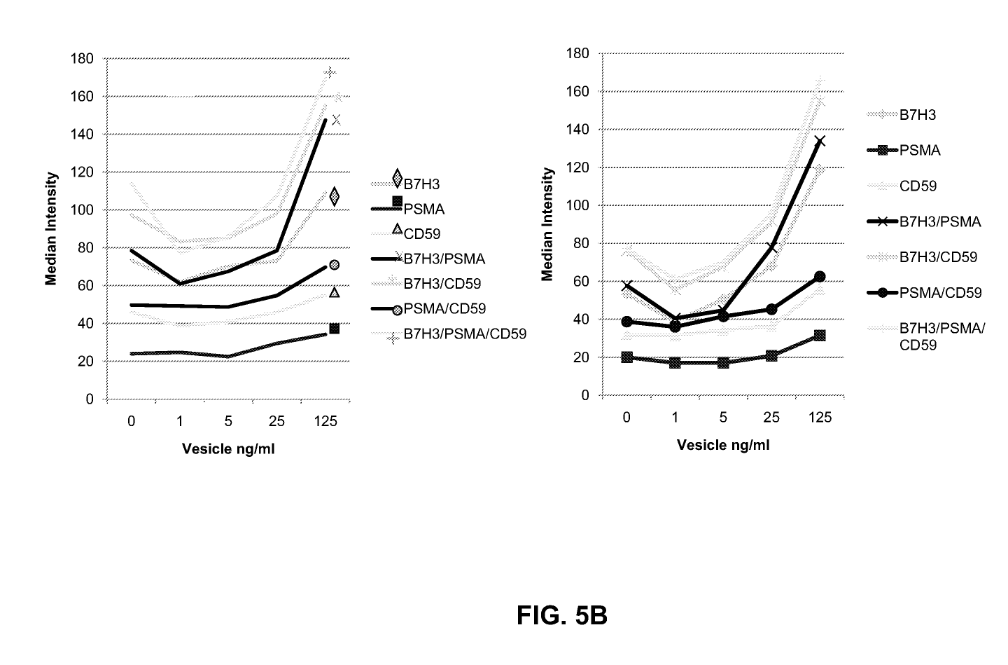

Multiplexing of at least 2, 3, 4, 5, 6, 7, 8, 9, 10, 11, 12, 13, 14, 15, 16, 17, 18, 19, 20, 25, 50, 75 or 100 different biomarkers may be performed. For example, an assay of a heterogeneous population of vesicles can be performed with a plurality of particles that are differentially labeled. There can be at least 2, 3, 4, 5, 6, 7, 8, 9, 10, 11, 12, 13, 14, 15, 16, 17, 18, 19, 20, 25, 50, 75 or 100 differentially labeled particles. The particles may be externally labeled, such as with a tag, or they may be intrinsically labeled. Each differentially labeled particle can be coupled to a capture agent, such as a binding agent, for a vesicle, resulting in capture of a vesicle. The multiple capture agents can be selected to characterize a phenotype of interest, including capture agents against general vesicle biomarkers, cell-of-origin specific biomarkers, and disease biomarkers. One or more biomarkers of the captured vesicle can then be detected by a plurality of binding agents. The binding agent can be directly labeled to facilitate detection. Alternatively, the binding agent is labeled by a secondary agent. For example, the binding agent may be an antibody for a biomarker on the vesicle. The binding agent is linked to biotin. A secondary agent comprises streptavidin linked to a reporter and can be added to detect the biomarker. In some embodiments, the captured vesicle is assayed for at least 2, 3, 4, 5, 6, 7, 8, 9, 10, 11, 12, 13, 14, 15, 16, 17, 18, 19, 20, 25, 50, 75 or 100 different biomarkers. For example, as depicted in FIG. 5, multiple detectors, i.e. detection of multiple biomarkers of a captured vesicle or population of vesicles, can increase the signal obtained, permitted increased sensitivity, specificity, or both, and the use of smaller amounts of samples.

An immunoassay based method or sandwich assay can also be used to detect a biomarker of a vesicle. An example includes ELISA. A binding agent or capture agent can be bound to a well. For example an antibody to an antigen of a vesicle can be attached to a well. A biomarker on the captured vesicle can be detected based on the methods described herein. FIG. 4A shows an illustrative schematic for a sandwich-type of immunoassay. The capture antibody can be against a vesicle antigen of interest, e.g., a general vesicle biomarker, a cell-of-origin marker, or a disease marker. In the figure, the captured vesicles are detected using fluorescently labeled antibodies against vesicle antigens of interest. Multiple capture antibodies can be used, e.g., in distinguishable addresses on an array or different wells of an immunoassay plate. The detection antibodies can be against the same antigen as the capture antibody, or can be directed against other markers. The capture antibodies can be substituted with alternate binding agents, such as tethered aptamers or lectins, and/or the detector antibodies can be similarly substituted, e.g., with detectable (e.g., labeled) aptamers, lectins or other binding proteins or entities. In an embodiment, one or more capture agents to a general vesicle biomarker, a cell-of-origin marker, and/or a disease marker are used along with detection agents against general vesicle biomarker, such as tetraspanin molecules including without limitation one or more of CD9, CD63 and CD81.

FIG. 4D presents an illustrative schematic for analyzing vesicles according to the methods of the invention. Capture agents are used to capture vesicles, detectors are used to detect the captured vesicles, and the level or presence of the captured and detected antibodies is used to characterize a phenotype. Capture agents, detectors and characterizing phenotypes can be any of those described herein. For example, capture agents include antibodies or aptamers tethered to a substrate that recognize a vesicle antigen of interest, detectors include labeled antibodies or aptamers to a vesicle antigen of interest, and characterizing a phenotype includes a diagnosis, prognosis, or theranosis of a disease. In the scheme shown in FIG. 4D i), a population of vesicles is captured with one or more capture agents against general vesicle biomarkers (400). The captured vesicles are then labeled with detectors against cell-of-origin biomarkers (401) and/or disease specific biomarkers (402). If only cell-of-origin detectors are used (401), the biosignature used to characterize the phenotype (403) can include the general vesicle markers (400) and the cell-of-origin biomarkers (401). If only disease detectors are used (402), the biosignature used to characterize the phenotype (403) can include the general vesicle markers (400) and the disease biomarkers (402). Alternately, detectors are used to detect both cell-of-origin biomarkers (401) and disease specific biomarkers (402). In this case, the biosignature used to characterize the phenotype (403) can include the general vesicle markers (400), the cell-of-origin biomarkers (401) and the disease biomarkers (402). The biomarkers combinations are selected to characterize the phenotype of interest and can be selected from the biomarkers and phenotypes described herein.

In the scheme shown in FIG. 4D ii), a population of vesicles is captured with one or more capture agents against cell-of-origin biomarkers (410) and/or disease biomarkers (411). The captured vesicles are then detected using detectors against general vesicle biomarkers (412). If only cell-of-origin capture agents are used (410), the biosignature used to characterize the phenotype (413) can include the cell-of-origin biomarkers (410) and the general vesicle markers (412). If only disease biomarker capture agents are used (411), the biosignature used to characterize the phenotype (413) can include the disease biomarkers (411) and the general vesicle biomarkers (412). Alternately, capture agents to one or more cell-of-origin biomarkers (410) and one or more disease specific biomarkers (411) are used to capture vesicles. In this case, the biosignature used to characterize the phenotype (413) can include the cell-of-origin biomarkers (410), the disease biomarkers (411), and the general vesicle markers (413). The biomarkers combinations are selected to characterize the phenotype of interest and can be selected from the biomarkers and phenotypes described herein.

Biomarkers comprising vesicle payload can be analyzed to characterize a phenotype. Payload comprises the biological entities contained within a vesicle membrane. These entities include without limitation nucleic acids, e.g., mRNA, microRNA, or DNA fragments; protein, e.g., soluble and membrane associated proteins; carbohydrates; lipids; metabolites; and various small molecules, e.g., hormones. The payload can be part of the cellular milieu that is encapsulated as a vesicle is formed in the cellular environment. In some embodiments of the invention, the payload is analyzed in addition to detecting vesicle surface antigens. Specific populations of vesicles can be captured as described above then the payload in the captured vesicles can be used to characterize a phenotype. For example, vesicles captured on a substrate can be further isolated to assess the payload therein. Alternately, the vesicles in a sample are detected and sorted without capture. The vesicles so detected can be further isolated to assess the payload therein. In an embodiment, vesicle populations are sorted by flow cytometry and the payload in the sorted vesicles is analyzed. In the scheme shown in FIG. 4E iii), a population of vesicles is captured and/or detected (430) using one or more of cell-of-origin biomarkers (420), disease biomarkers (421), and general vesicle markers (422). The payload of the isolated vesicles is assessed (423). A biosignature detected within the payload can be used to characterize a phenotype (424). In a non-limiting example, a vesicle population can be analyzed in a plasma sample from a patient using antibodies against one or more vesicle antigens of interest. The antibodies can be capture antibodies which are tethered to a substrate to isolate a desired vesicle population. Alternately, the antibodies can be directly labeled and the labeled vesicles isolated by sorting with flow cytometry. The presence or level of microRNA or mRNA extracted from the isolated vesicle population can be used to detect a biosignature. The biosignature is then used to diagnose, prognose or theranose the patient.

In other embodiments, vesicle payload is analyzed in a vesicle population without first capturing or detected subpopulations of vesicles. For example, vesicles can be generally isolated from a sample using centrifugation, filtration, chromatography, or other techniques as described herein. The payload of the isolated vesicles can be analyzed thereafter to detect a biosignature and characterize a phenotype. In the scheme shown in FIG. 4E iv), a population of vesicles is isolated (430) and the payload of the isolated vesicles is assessed (431). A biosignature detected within the payload can be used to characterize a phenotype (432). In a non-limiting example, a vesicle population is isolated from a plasma sample from a patient using size exclusion and membrane filtration. The presence or level of microRNA or mRNA extracted from the vesicle population is used to detect a biosignature. The biosignature is then used to diagnose, prognose or theranose the patient.

A peptide or protein biomarker can be analyzed by mass spectrometry or flow cytometry. Proteomic analysis of a vesicle may be carried out by immunocytochemical staining, Western blotting, electrophoresis, SDS-PAGE, chromatography, x-ray crystallography or other protein analysis techniques in accordance with procedures well known in the art. In other embodiments, the protein bio-signature of a vesicle may be analyzed using 2 D differential gel electrophoresis as described in, Chromy et al. J Proteome Res, 2004; 3:1120-1127, which is herein incorporated by reference in its entirety, or with liquid chromatography mass spectrometry as described in Zhang et al. Mol Cell Proteomics, 2005; 4:144-155, which is herein incorporated by reference in its entirety. A vesicle may be subjected to activity-based protein profiling described for example, in Berger et al., Am J Pharmacogenomics, 2004; 4:371-381, which is in incorporated by reference in its entirety. In other embodiments, a vesicle may be profiled using nanospray liquid chromatography-tandem mass spectrometry as described in Pisitkun et al., Proc Natl Acad Sci USA, 2004; 101:13368-13373, which is herein incorporated by reference in its entirety. In another embodiment, the vesicle may be profiled using tandem mass spectrometry (MS) such as liquid chromatography/MS/MS (LC-MS/MS) using for example a LTQ and LTQ-FT ion trap mass spectrometer. Protein identification can be determined and relative quantitation can be assessed by comparing spectral counts as described in Smalley et al., J Proteome Res, 2008; 7:2088-2096, which is herein incorporated by reference in its entirety.

Protein expression of a vesicle can also be identified. The analysis can optionally follow the isolation of specific vesicles using capture agents to capture populations of interest. In an embodiment, immunocytochemical staining is used to analyze protein expression within a vesicle. The vesicles can be resuspended in buffer, centrifuged at 100×g for example, for 3 minutes using a cytocentrifuge on adhesive slides in preparation for immunocytochemical staining. The cytospins can be air-dried overnight and stored at −80° C. until staining. Slides can then be fixed and blocked with serum-free blocking reagent. The slides can then be incubated with a specific antibody to detect the expression of a protein of interest. In some embodiments, the vesicles are not purified, isolated or concentrated prior to protein expression analysis.

A vesicle, such as isolated cell-of-origin specific vesicle, can be characterized by analysis of a metabolite marker or metabolite, which can also form a bio-signature for a vesicle. Various metabolite-oriented approaches have been described such as metabolite target analyses, metabolite profiling, or metabolic fingerprinting, see for example, Denkert et al., Molecular Cancer 2008; 7: 4598-4617, Ellis et al., Analyst 2006; 8: 875-885, Kuhn et al., Clinical Cancer Research 2007; 24: 7401-7406, Fiehn O., Comp Funct Genomics 2001; 2:155-168, Fancy et al., Rapid Commun Mass Spectrom 20(15): 2271-80 (2006), Lindon et al., Pharm Res, 23(6): 1075-88 (2006), Holmes et al., Anal Chem. 2007 Apr. 1; 79(7):2629-40. Epub 2007 Feb. 27. Erratum in: Anal Chem. 2008 Aug. 1; 80(15):6142-3, Stanley et al., Anal Biochem. 2005 Aug. 15; 343(2): 195-202, Lehtimäki et al., J Biol Chem. 2003 Nov. 14; 278(46):45915-23, each of which is herein incorporated by reference in its entirety.

Peptides from a vesicle can be analyzed by systems described in Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety. This system can generate sensitive molecular fingerprints of proteins present in a body fluid as well as in vesicles. Commercial applications which include the use of chromatography/mass spectroscopy and reference libraries of all stable metabolites in the human body, for example Paradigm Genetic's Human Metabolome Project, may be used to determine the metabolite bio-signature of vesicles, such as isolated cell-of-origin specific vesicles. Other methods for analyzing a metabolic profile can include methods and devices described in U.S. Pat. No. 6,683,455 (Metabometrix), U.S. Patent Application Publication Nos. 20070003965 and 20070004044 (Biocrates Life Science), each of which is herein incorporated by reference in its entirety. Other proteomic profiling techniques are described in Kennedy, Toxicol Lett 120:379-384 (2001), Berven et al., Curr Pharm Biotechnol 7(3): 147-58 (2006), Conrads et al., Expert Rev Proteomics 2(5): 693-703, Decramer et al., World J Urol 25(5): 457-65 (2007), Decramer et al., Mol Cell Proteomics 7(10): 1850-62 (2008), Decramer et al., Contrib Nephrol, 160: 127-41 (2008), Diamandis, J Proteome Res 5(9): 2079-82 (2006), Immler et al., Proteomics 6(10): 2947-58 (2006), Khan et al., J Proteome Res 5(10): 2824-38 (2006), Kumar et al., Biomarkers 11(5): 385-405 (2006), Noble et al., Breast Cancer Res Treat 104(2): 191-6 (2007), Omenn, Dis Markers 20(3): 131-4 (2004), Powell et al., Expert Rev Proteomics 3(1): 63-74 (2006), Rai et al., Arch Pathol Lab Med, 126(12): 1518-26 (2002), Ramstrom et al., Proteomics, 3(2): 184-90 (2003), Tammen et al., Breast Cancer Res Treat, 79(1): 83-93 (2003), Theodorescu et al., Lancet Oncol, 7(3): 230-40 (2006), or Zurbig et al., Electrophoresis, 27(11): 2111-25 (2006).

For analysis of mRNAs, miRNAs or other small RNAs, the total RNA can be first isolated from a vesicle using any other known methods for isolating nucleic acids such as methods described in U.S. Patent Application Publication No. 2008132694, which is herein incorporated by reference in its entirety. These include, but are not limited to, kits for performing membrane based RNA purification, which are commercially available. Generally, kits are available for the small-scale (30 mg or less) preparation of RNA from cells and tissues, for the medium scale (250 mg tissue) preparation of RNA from cells and tissues, and for the large scale (1 g maximum) preparation of RNA from cells and tissues. Other commercially available kits for effective isolation of small RNA-containing total RNA are available.

Alternatively, RNA can be isolated using the method described in U.S. Pat. No. 7,267,950, which is herein incorporated by reference in its entirety. U.S. Pat. No. 7,267,950 describes a method of extracting RNA from biological systems (cells, cell fragments, organelles, tissues, organs, or organisms) in which a solution containing RNA is contacted with a substrate to which RNA can bind and RNA is withdrawn from the substrate by applying negative pressure. Alternatively, RNA may be isolated using the method described in U.S. Patent Application No. 20050059024, which is herein incorporated by reference in its entirety, which describes the isolation of small RNA molecules. Other methods are described in U.S. Patent Application No. 20050208510, 20050277121, 20070238118, each of which is incorporated by reference in its entirety.

In one embodiment, mRNA expression analysis can be carried out on mRNAs from a vesicle isolated from a sample. In some embodiments, the vesicle is a cell-of-origin specific vesicle. An expression pattern generated from a vesicle can be indicative of a given disease state, disease stage, therapy related signature, or physiological condition.

In one embodiment, once the total RNA has been isolated, cDNA can be synthesized and either qRT-PCR assays (e.g. Applied Biosystem's Taqman® assays) for specific mRNA targets can be performed according to manufacturer's protocol, or an expression microarray can be performed to look at highly multiplexed sets of expression markers in one experiment. Methods for establishing gene expression profiles include determining the amount of RNA that is produced by a gene that can code for a protein or peptide. This can be accomplished by quantitative reverse transcriptase PCR (qRT-PCR), competitive RT-PCR, real time RT-PCR, differential display RT-PCR, Northern Blot analysis or other related tests. While it is possible to conduct these techniques using individual PCR reactions, it is also possible to amplify complementary DNA (cDNA) or complementary RNA (cRNA) produced from mRNA and analyze it via microarray.

The level of a miRNA product in a sample can be measured using any technique that is suitable for detecting mRNA expression levels in a biological sample, including but not limited to Northern blot analysis, RT-PCR, qRT-PCR, in situ hybridization or microarray analysis. For example, using gene specific primers and target cDNA, qRT-PCR enables sensitive and quantitative miRNA measurements of either a small number of target miRNAs (via singleplex and multiplex analysis) or the platform can be adopted to conduct high throughput measurements using 96-well or 384-well plate formats. See for example, Ross J S et al, Oncologist. 2008 May; 13(5):477-93, which is herein incorporated by reference in its entirety. A number of different array configurations and methods for microarray production are known to those of skill in the art and are described in U.S. patents such as: U.S. Pat. No. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; or 5,700,637; each of which is herein incorporated by reference in its entirety. Other methods of profiling miRNAs are described in Taylor et al., Gynecol Oncol. 2008 July; 110(1):13-21, Gilad et al, PLoS ONE. 2008 Sep. 5; 3(9):e3148, Lee et al., Annu Rev Pathol. 2008 Sep. 25 and Mitchell et al, Proc Natl Acad Sci USA. 2008 Jul. 29; 105(30):10513-8, Shen R et al, BMC Genomics. 2004 Dec. 14; 5(1):94, Mina L et al, Breast Cancer Res Treat. 2007 June; 103(2):197-208, Zhang L et al, Proc Natl Acad Sci USA. 2008 May 13; 105(19):7004-9, Ross J S et al, Oncologist. 2008 May; 13(5):477-93, Schetter A J et al, JAMA. 2008 Jan. 30; 299(4):425-36, Staudt L M, N Engl J Med 2003; 348:1777-85, Mulligan G et al, Blood. 2007 Apr. 15; 109(8):3177-88. Epub 2006 Dec. 21, McLendon R et al, Nature. 2008 Oct. 23; 455(7216):1061-8, and U.S. Pat. Nos. 5,538,848, 5,723,591, 5,876,930, 6,030,787, 6,258,569, and 5,804,375, each of which is herein incorporated by reference.

Microarray technology allows for the measurement of the steady-state mRNA or miRNA levels of thousands of transcripts or miRNAs simultaneously thereby presenting a powerful tool for identifying effects such as the onset, arrest, or modulation of uncontrolled cell proliferation. Two microarray technologies, such as cDNA arrays and oligonucleotide arrays can be used. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA or miRNA, expressed in the sample cells. A large number of such techniques are available and useful. Methods for determining gene expression can be found in U.S. Pat. No. 6,271,002 to Linsley, et al.; U.S. Pat. No. 6,218,122 to Friend, et al.; U.S. Pat. No. 6,218,114 to Peck et al.; or U.S. Pat. No. 6,004,755 to Wang, et al., each of which is herein incorporated by reference in its entirety.

Analysis of an expression level can be conducted by comparing such intensities. This can be performed by generating a ratio matrix of the expression intensities of genes in a test sample versus those in a control sample. The control sample may be used as a reference, and different references to account for age, ethnicity and sex may be used. Different references can be used for different conditions or diseases, as well as different stages of diseases or conditions, as well as for determining therapeutic efficacy.

For instance, the gene expression intensities of mRNA or miRNAs isolated from vesicles derived from a diseased tissue can be compared with the expression intensities generated from vesicles isolated from normal tissue of the same type (e.g., diseased breast tissue sample versus. normal breast tissue sample). A ratio of these expression intensities indicates the fold-change in gene expression between the test and control samples. Alternatively, if vesicles are not normally present in from normal tissues (e.g. breast) then absolute quantitation methods, as is known in the art, can be used to define the number of miRNA molecules present without the requirement of miRNA or mRNA isolated from vesicles derived from normal tissue.

Gene expression profiles can also be displayed in a number of ways. A common method is to arrange raw fluorescence intensities or ratio matrix into a graphical dendogram where columns indicate test samples and rows indicate genes. The data is arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data.

mRNAs or miRNAs that are considered differentially expressed can be either over expressed or under expressed in patients with a disease relative to disease free individuals. Over and under expression are relative terms meaning that a detectable difference (beyond the contribution of noise in the system used to measure it) is found in the amount of expression of the mRNAs or miRNAs relative to some baseline. In this case, the baseline is the measured mRNA/miRNA expression of a non-diseased individual. The mRNA/miRNA of interest in the diseased cells can then be either over or under expressed relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis includes the determination of disease/status issues such as determining the likelihood of relapse or metastasis and therapy monitoring. In therapy monitoring, clinical judgments are made regarding the effect of a given course of therapy by comparing the expression of genes over time to determine whether the mRNA/miRNA expression profiles have changed or are changing to patterns more consistent with normal tissue.

Levels of over and under expression are distinguished based on fold changes of the intensity measurements of hybridized microarray probes. A 2× difference is preferred for making such distinctions or a p-value less than 0.05. That is, before an mRNA/miRNA is the to be differentially expressed in diseased/relapsing versus normal/non-relapsing cells, the diseased cell is found to yield at least 2 times more, or 2 times less intensity than the normal cells. The greater the fold difference, the more preferred is use of the gene as a diagnostic or prognostic tool. mRNA/miRNAs selected for the expression profiles of the instant invention have expression levels that result in the generation of a signal that is distinguishable from those of the normal or non-modulated genes by an amount that exceeds background using clinical laboratory instrumentation.

Statistical values can be used to confidently distinguish modulated from non-modulated mRNA/miRNA and noise. Statistical tests find the mRNA/miRNA most significantly different between diverse groups of samples. The Student's t-test is an example of a robust statistical test that can be used to find significant differences between two groups. The lower the p-value, the more compelling the evidence that the gene shows a difference between the different groups. Nevertheless, since microarrays measure more than one mRNA/miRNA at a time, tens of thousands of statistical tests may be performed at one time. Because of this, one is unlikely to see small p-values just by chance and adjustments for this using a Sidak correction as well as a randomization/permutation experiment can be made. A p-value less than 0.05 by the t-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

In one embodiment, a method of generating a posterior probability score to enable diagnostic, prognostic, therapy-related, or physiological state specific bio-signature scores can be arrived at by obtaining mRNA or miRNA (biomarker) expression data from a statistically significant number of patient vesicles, such as vesicles; applying linear discrimination analysis to the data to obtain selected biomarkers; and applying weighted expression levels to the selected biomarkers with discriminate function factor to obtain a prediction model that can be applied as a posterior probability score. Other analytical tools can also be used to answer the same question such as, logistic regression and neural network approaches.

For instance, the following can be used for linear discriminant analysis:

where,
I(pₛi_{d})=The log base 2 intensity of the probe set enclosed in parenthesis. d(cp)=The discriminant function for the disease positive class d(C_{N})=The discriminant function for the disease negative class
P(_{CP})=The posterior p-value for the disease positive class
P(_{CN})=The posterior p-value for the disease negative class
Numerous other well-known methods of pattern recognition are available. The following references provide some examples: Weighted Voting: Golub et al. (1999); Support Vector Machines: Su et al. (2001); and Ramaswamy et al. (2001); K-nearest Neighbors: Ramaswamy (2001); and Correlation Coefficients: van't Veer et al. (2002), all of which are herein incorporated by reference in their entireties.

A bio-signature portfolio, further described below, can be established such that the combination of biomarkers in the portfolio exhibit improved sensitivity and specificity relative to individual biomarkers or randomly selected combinations of biomarkers. In one embodiment, the sensitivity of the bio-signature portfolio can be reflected in the fold differences, for example, exhibited by a transcript's expression in the diseased state relative to the normal state. Specificity can be reflected in statistical measurements of the correlation of the signaling of transcript expression with the condition of interest. For example, standard deviation can be a used as such a measurement. In considering a group of biomarkers for inclusion in a bio-signature portfolio, a small standard deviation in expression measurements correlates with greater specificity. Other measurements of variation such as correlation coefficients can also be used in this capacity.

Another parameter that can be used to select mRNA/miRNA that generate a signal that is greater than that of the non-modulated mRNA/miRNA or noise is the use of a measurement of absolute signal difference. The signal generated by the modulated mRNA/miRNA expression is at least 20% different than those of the normal or non-modulated gene (on an absolute basis). It is even more preferred that such mRNA/miRNA produce expression patterns that are at least 30% different than those of normal or non-modulated mRNA/miRNA.

MiRNA can also be detected and measured by amplification from a biological sample and measured using methods described in U.S. Pat. No. 7,250,496, U.S. Application Publication Nos. 20070292878, 20070042380 or 20050222399 and references cited therein, each of which is herein incorporated by reference in its entirety.

Peptide nucleic acids (PNAs) which are a new class of synthetic nucleic acid analogs in which the phosphate—sugar polynucleotide backbone is replaced by a flexible pseudo-peptide polymer may be utilized in analysis of bio-signatures of vesicles. PNAs are capable of hybridizing with high affinity and specificity to complementary RNA and DNA sequences and are highly resistant to degradation by nucleases and proteinases. Peptide nucleic acids (PNAs) are an attractive new class of probes with applications in cytogenetics for the rapid in situ identification of human chromosomes and the detection of copy number variation (CNV). Multicolor peptide nucleic acid-fluorescence in situ hybridization (PNA-FISH) protocols have been described for the identification of several human CNV-related disorders and infectious diseases. PNAs can also be utilized as molecular diagnostic tools to non-invasively measure oncogene mRNAs with tumor targeted radionuclide-PNA-peptide chimeras. Methods of using PNAs are described further in Pellestor F et al, Curr Pharm Des. 2008; 14(24):2439-44, Tian X et al, Ann N Y Acad Sci. 2005 November; 1059: 106-44, Paulasova P and Pellestor F, Annales de Génétique, 47 (2004) 349-358, Stender H. Expert Rev Mol Diagn. 2003 September; 3(5):649-55. Review, Vigneault et al., Nature Methods, 5(9), 777-779 (2008), each reference is herein incorporated by reference in its entirety. These methods can be used to screen the genetic materials isolated from a vesicle. When applying these techniques to a cell-of-origin specific vesicle, they can be used to identify a given molecular signal that directly pertains to the cell of origin.

Mutational analysis may be carried out for mRNAs and DNA that are identified from a vesicle. For mutational analysis of a target or biomarker that is of RNA origin, the RNA (mRNA, miRNA or other) can be reverse transcribed into cDNA and subsequently sequenced or assayed, such as for known SNPs (by Taqman SNP assays, for example) or single nucleotide mutations, as well as using sequencing to look for insertions or deletions to determine mutations present in the cell-of-origin. Multiplexed ligation dependent probe amplification (MLPA) could alternatively be used for the purpose of identifying CNV in small and specific areas of interest. For example, once the total RNA has been obtained from isolated colon cancer-specific vesicles, cDNA can be synthesized and primers specific for exons 2 and 3 of the KRAS gene can be used to amplify these two exons containing codons 12, 13 and 61 of the KRAS gene. The same primers used for PCR amplification can be used for Big Dye Terminator sequence analysis on the ABI 3730 to identify mutations in exons 2 and 3 of KRAS. Mutations in these codons are known to confer resistance to drugs such as Cetuximab and Panitumimab. Methods of conducting mutational analysis are described in Maheswaran S et al, Jul. 2, 2008 (10.1056/NEJMoa0800668) and Orita, M et al, PNAS 1989, (86): 2766-70, each of which is herein incorporated by reference in its entirety.

Other methods of conducting mutational analysis can include miRNA sequencing. Applications for identifying and profiling miRNAs can be done by cloning techniques and the use of capillary DNA sequencing or “next-generation” sequencing technologies. The new sequencing technologies currently available allow the identification of low-abundance miRNAs or those exhibiting modest expression differences between samples, which may not be detected by hybridization-based methods. Such new sequencing technologies include the massively parallel signature sequencing (MPSS) methodology described in Nakano et al. 2006, Nucleic Acids Res. 2006; 34:D731-D735. doi: 10.1093/nar/gkj077, the Roche/454 platform described in Margulies et al. 2005, Nature. 2005; 437:376-380 or the Illumina sequencing platform described in Berezikov et al. Nat. Genet. 2006b; 38:1375-1377, each of which is incorporated by reference in its entirety.

Additional methods to determine a bio-signature includes assaying a biomarker by allele-specific PCR, which includes specific primers to amplify and discriminate between two alleles of a gene simultaneously, single-strand conformation polymorphism (SSCP), which involves the electrophoretic separation of single-stranded nucleic acids based on subtle differences in sequence, and DNA and RNA aptamers. DNA and RNA aptamers are short oligonucleotide sequences that can be selected from random pools based on their ability to bind a particular molecule with high affinity. Methods of using aptamers are described in Ulrich H et al, Comb Chem High Throughput Screen. 2006 September; 9(8):619-32, Ferreira C S et al, Anal Bioanal Chem. 2008 February; 390(4):1039-50, Ferreira C S et al, Tumour Biol. 2006; 27(6):289-301, each of which is herein incorporated by reference in its entirety.

Biomarkers can also be detected using fluorescence in situ hybridization (FISH). Methods of using FISH to detect and localize specific DNA sequences, localize specific mRNAs within tissue samples or identify chromosomal abnormalities are described in Shaffer D R et al, Clin Cancer Res. 2007 Apr. 1; 13(7):2023-9, Cappuzo F et al, Journal of Thoracic Oncology, Volume 2, Number 5, May 2007, Moroni M et al, Lancet Oncol. 2005 May; 6(5):279-86, each of which is herein incorporated by reference in its entirety.

An illustrative schematic for analyzing a population of vesicles for their payload is presented in FIG. 4E.

A bio-signature of a vesicle can comprise a binding agent for the vesicle. The binding agent can be a DNA, RNA, aptamer, monoclonal antibody, polyclonal antibody, Fabs, Fab′, single chain antibody, synthetic antibody, aptamer (DNA/RNA), peptoid, zDNA, peptide nucleic acid (PNA), locked nucleic acid (LNA), lectin, synthetic or naturally occurring chemical compounds (including but not limited to drugs and labeling reagents).

A binding agent can used to isolate or detect a vesicle by binding to a component of the vesicle, as described above. The binding agent can be used to detect a vesicle, such as for detecting a cell-of-origin specific vesicle. A binding agent or multiple binding agents can themselves form a binding agent profile that provides a bio-signature for a vesicle. One or more binding agents can be selected from Table 2. For example, if a vesicle population is detected or isolated using two, three or four binding agents in a differential detection or isolation of a vesicle from a heterogeneous population of vesicles, the particular binding agent profile for the vesicle population provides a bio-signature for the particular vesicle population.

As an illustrative example, a vesicle for characterizing prostate cancer can be detected with one or more binding agents including, but not limited to, PSA, PSMA, PCSA, PSCA, B7H3, EpCam, TMPRSS2, mAB 5D4, XPSM-A9, XPSM-A10, Galectin-3, E-selectin, Galectin-1, or E4 (IgG2a kappa), or any combination thereof.

The binding agent can also be for a general vesicle biomarker, such as a “housekeeping protein” or antigen. The biomarker can be CD9, CD63, or CD81. For example, the binding agent can be an antibody for CD9, CD63, or CD81. The binding agent can also be for other proteins, such as for prostate specific or cancer specific vesicles. The binding agent can be for PCSA, PSMA, EpCam, B7H3, or STEAP. For example, the binding agent can be an antibody for PCSA, PSMA, EpCam, B7H3, or STEAP.

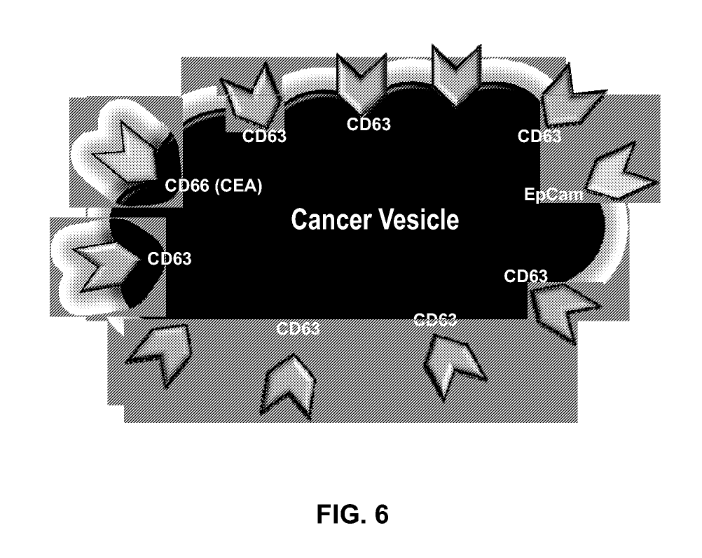

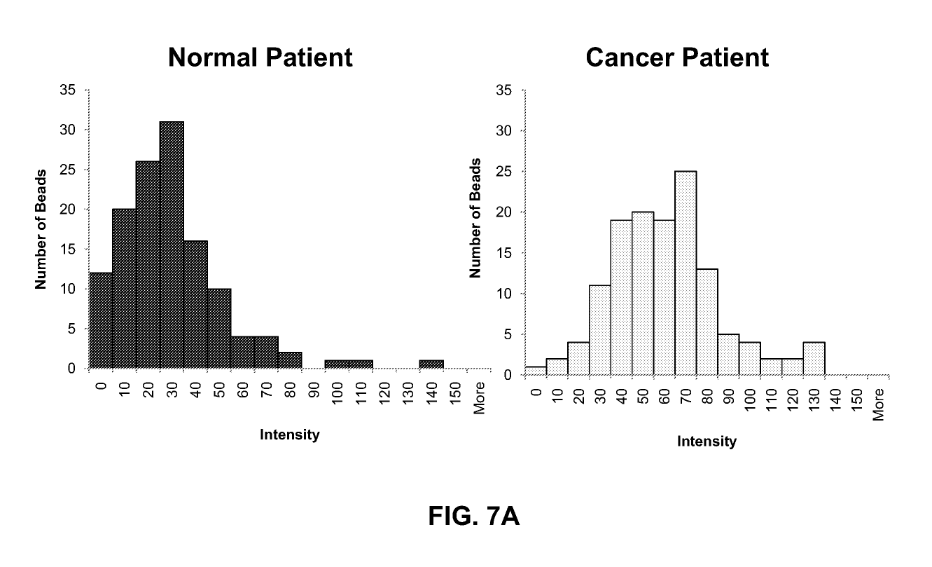

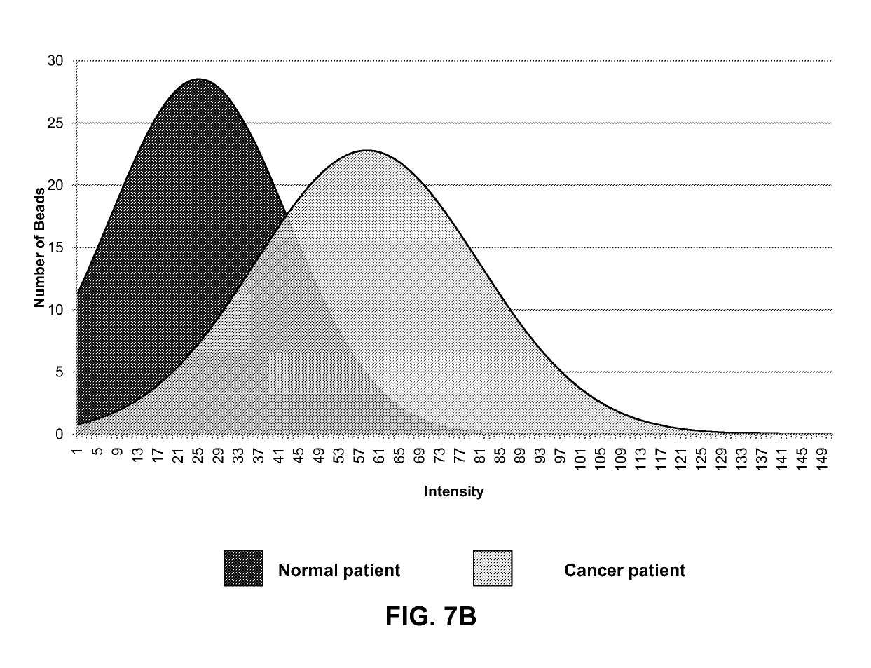

Various proteins are not typically distributed evenly or uniformly on a vesicle shell. See, e.g., FIG. 6, which illustrates a schematic of protein expression patterns. Vesicle-specific proteins are typically more common, while cancer-specific proteins are less common. In some embodiments, capture of a vesicle is accomplished using a more common, less cancer-specific protein, such as one or more housekeeping proteins or antigen or general vesicle antigen (e.g., a tetraspanin), and one or more cancer-specific biomarkers and/or one or more cell-of-origin specific biomarkers is used in the detection phase. In another embodiment, one or more cancer-specific biomarkers and/or one or more cell-of-origin specific biomarkers are used for capture, and one or more housekeeping proteins or antigen or general vesicle antigen (e.g., a tetraspanin) is used for detection. In addition, the same biomarker can be used for both capture and detection.

Additional cellular binding partners or binding agents may be identified by any conventional methods known in the art, or as described herein, and may additionally be used as a diagnostic, prognostic or therapy-related marker.

### Phenotypes

Analysis of a vesicle from a subject can be used to characterize a phenotype. A phenotype can be any observable characteristic or trait of a subject, such as a disease or condition, a disease stage or condition stage, susceptibility to a disease or condition, prognosis of a disease stage or condition, a physiological state, or response to therapeutics. A phenotype can result from a subject's gene expression as well as the influence of environmental factors and the interactions between the two, as well as from epigenetic modifications to nucleic acid sequences.

A phenotype in a subject can be characterized by obtaining a biological sample from said subject and analyzing one or more vesicles from the sample. For example, characterizing a phenotype for a subject or individual may include detecting a disease or condition (including pre-symptomatic early stage detecting), determining the prognosis, diagnosis, or theranosis of a disease or condition, or determining the stage or progression of a disease or condition. Characterizing a phenotype can also include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation.

For example, the phenotype can comprise a tumor, neoplasm, or cancer. A cancer detected or assessed by products or processes described herein includes, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma. The phenotype may also be a premalignant condition, such as Barrett's Esophagus.

The cancer can comprise, without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor. The methods of the invention can be used to characterize these and other cancers. Thus, characterizing a phenotype can be providing a diagnosis, prognosis or theranosis of one of the cancers disclosed herein.

The phenotype can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

The phenotype can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event.

The phenotype can also be a neurological disease, such as Multiple Sclerosis (MS), Parkinson's Disease (PD), Alzheimer's Disease (AD), schizophrenia, bipolar disorder, depression, autism, Prion Disease, Picks disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain.

The phenotype may also be an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. Viral proteins, such as HIV or HCV-like particles can be assessed in a vesicle, to characterize a viral condition.

The phenotype can also be a perinatal or pregnancy related condition (e.g. preeclampsia or preterm birth), metabolic disease or condition, such as a metabolic disease or condition associated with iron metabolism. For example, hepcidin can be assayed in a vesicle to characterize an iron deficiency. The metabolic disease or condition can also be diabetes, inflammation, or a perinatal condition.

In some embodiments, the phenotype is prostate cancer. For example, the presence or level of vesicles with a bio-signature can be used to diagnose, prognose or theranose the cancer. As described above, a bio-signature for prostate cancer can comprise one or more binding agents associated with prostate cancer (for example, as shown in Table 2), and one or more additional biomarkers. For example, a bio-signature for prostate cancer can comprise a binding agent to PSA, PSMA, PCSA, TMPRSS2, mAB 5D4, XPSM-A9, XPSM-A10, Galectin-3, E-selectin, Galectin-1, E4 (IgG2a kappa), or any combination thereof, with one or more additional biomarkers, such as one or more miRNA, one or more DNA, one or more additional peptide, protein, or antigen associated with prostate cancer, such as, but not limited to, those disclosed in U.S. patent application Ser. No. 12/591,226.

A bio-signature for prostate cancer can comprise an antigen associated with prostate cancer (for example, as shown in Table 1), and one or more additional biomarkers, such as those disclosed in U.S. patent application Ser. No. 12/591,226. A bio-signature for prostate cancer can comprise one or more antigens associated with prostate cancer, such as, but not limited to, KIA1, intact fibronectin, PSA, EZH2 (Enhancer of zeste homolog 2), TMPRSS2, FASLG, TNFSF10, PSMA, PCSA, NGEP, IL-7RI, CSCR4, CysLT1R, TRPM8, Kv1.3, TRPV6, TRPM8, PSGR, MISIIR, or any combination thereof. A biosignature for prostate cancer can also comprise one of more vesicle antigens selected from PSMA, PCSA, B7-H3, IL 6, OPG-13 (OPG), IL6R, PA2G4, EZH2, RUNX2, and SERPINB3. The bio-signature for prostate cancer can comprise one or more of the aforementioned antigens and one or more additional biomarkers, such as, but not limited to miRNA, mRNA, DNA, or any combination thereof.

A bio-signature for prostate cancer can also comprise one or more antigens associated with prostate cancer, such as, but not limited to, KIA1, intact fibronectin, PSA, TMPRSS2, FASLG, TNFSF10, PSMA, PCSA, NGEP, IL-7RI, CSCR4, CysLT1R, TRPM8, Kv1.3, TRPV6, TRPM8, PSGR, MISIIR, or any combination thereof, and one or more miRNA biomarkers, such as, but not limited to, miR-202, miR-210, miR-296, miR-320, miR-370, miR-373, miR-498, miR-503, miR-184, miR-198, miR-302c, miR-345, miR-491, miR-513, miR-32, miR-182, miR-31, miR-26a-1/2, miR-200c, miR-375, miR-196a-1/2, miR-370, miR-425, miR-425, miR-194-1/2, miR-181a-1/2, miR-34b, let-7i, miR-188, miR-25, miR-106b, miR-449, miR-99b, miR-93, miR-92-1/2, miR-125a, miR-141, let-7a, let-7b, let-7c, let-7d, let-7g, miR-16, miR-23a, miR-23b, miR-26a, miR-92, miR-99a, miR-103, miR-125a, miR-125b, miR-143, miR-145, miR-195, miR-199, miR-221, miR-222, miR-497, let-7f, miR-19b, miR-22, miR-26b, miR-27a, miR-27b, miR-29a, miR-29b, miR-30_{—}5p, miR-30c, miR-100, miR-141, miR-148a, miR-205, miR-520h, miR-494, miR-490, miR-133a-1, miR-1-2, miR-218-2, miR-220, miR-128a, miR-221, miR-499, miR-329, miR-340, miR-345, miR-410, miR-126, miR-205, miR-7-1/2, miR-145, miR-34a, miR-487, or let-7b, or any combination thereof.

Furthermore, the miRNA for a prostate cancer bio-signature can be a miRNA that interacts with PFKFB3, RHAMM (HMMR), cDNA FLJ42103, ASPM, CENPF, NCAPG, Androgen Receptor, EGFR, HSP90, SPARC, DNMT3B, GART, MGMT, SSTR3, TOP2B, or any combination thereof. The miRNA can also be miR-9, miR-629, miR-141, miR-671-3p, miR-491, miR-182, miR-125a-3p, miR-324-5p, miR-148B, miR-222, or any combination thereof.

The bio-signature for prostate cancer can comprise one or more antigens associated with prostate cancer, such as, but not limited to, KIA1, intact fibronectin, PSA, EZH2 (Enhancer of zeste homolog 2), TMPRSS2, FASLG, TNESF10, PSMA, PCSA, NGEP, IL-7RI, CSCR4, CysLT1R, TRPM8, Kv1.3, TRPV6, TRPM8, PSGR, MISIIR, B7-H3, IL 6, OPG-13 (OPG), IL6R, PA2G4, RUNX2, or any combination thereof, and one or more additional biomarkers such as, but not limited to, the aforementioned miRNAs, mRNAs (such as, but not limited to, AR or PCA3), snoRNA (such as, but not limited to, U50) or any combination thereof.

The bio-signature can also comprise one or more gene fusions, such as ACSL3-ETV1, C15ORF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4.

A vesicle can be isolated, assayed, or both, for one or more miRNA and one or more antigens associated with prostate cancer to provide a diagnostic, prognostic or theranostic profile, such as the stage of the cancer, the efficacy of the cancer, or other characteristics of the cancer. Alternatively, the vesicle can be directly assayed from a sample, such that the vesicle is not purified or concentrated prior to assaying for one or more miRNA or antigens associated with prostate cancer.

A bio-signature for prostate cancer can be used to assess the efficacy of a therapy. For example, biomarkers that are elevated in PCa can be monitored before and after a treatment. A reduction in the level of the biomarker post-treatment can indicate that the treatment is efficacious. The same bio-signature can be monitored overtime, e.g., to detect recurrence or relapse post-treatment.

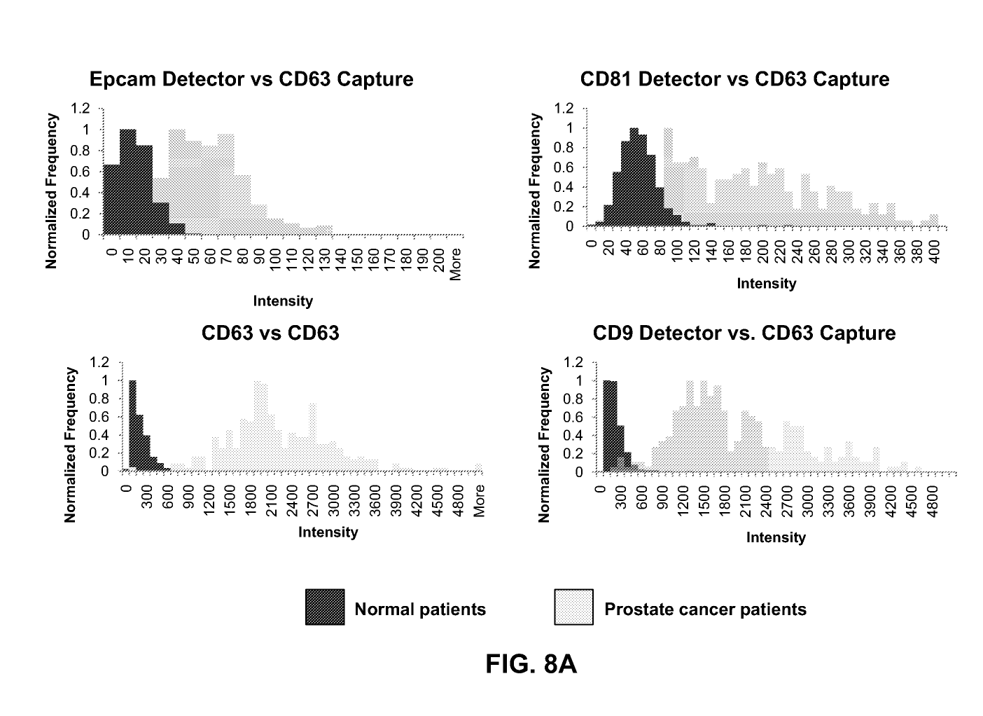

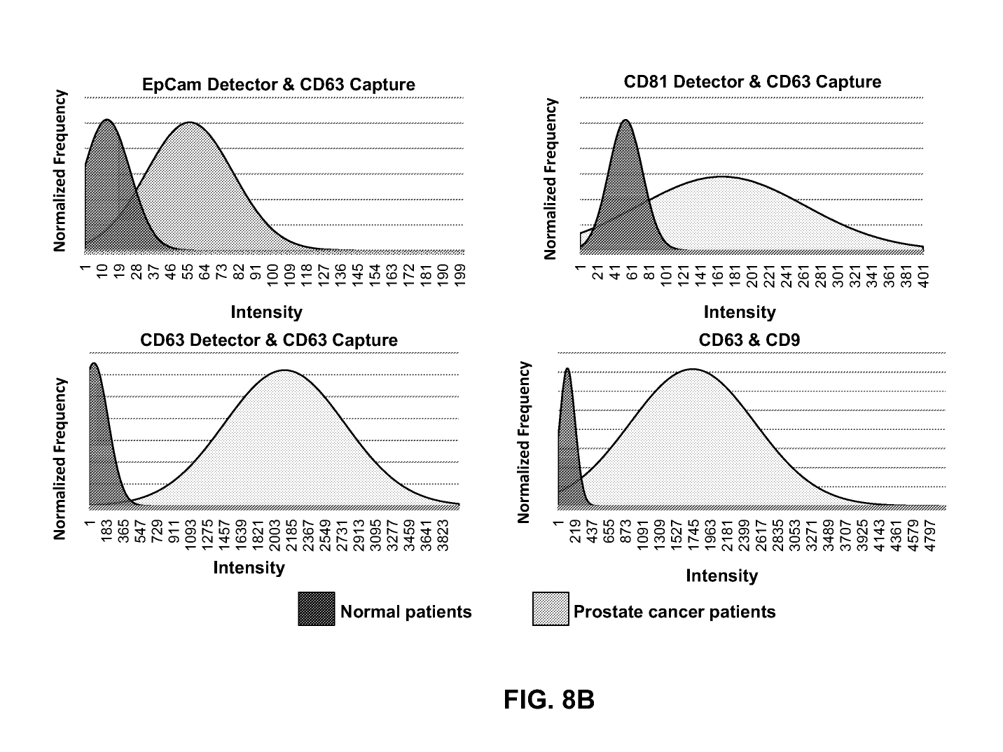

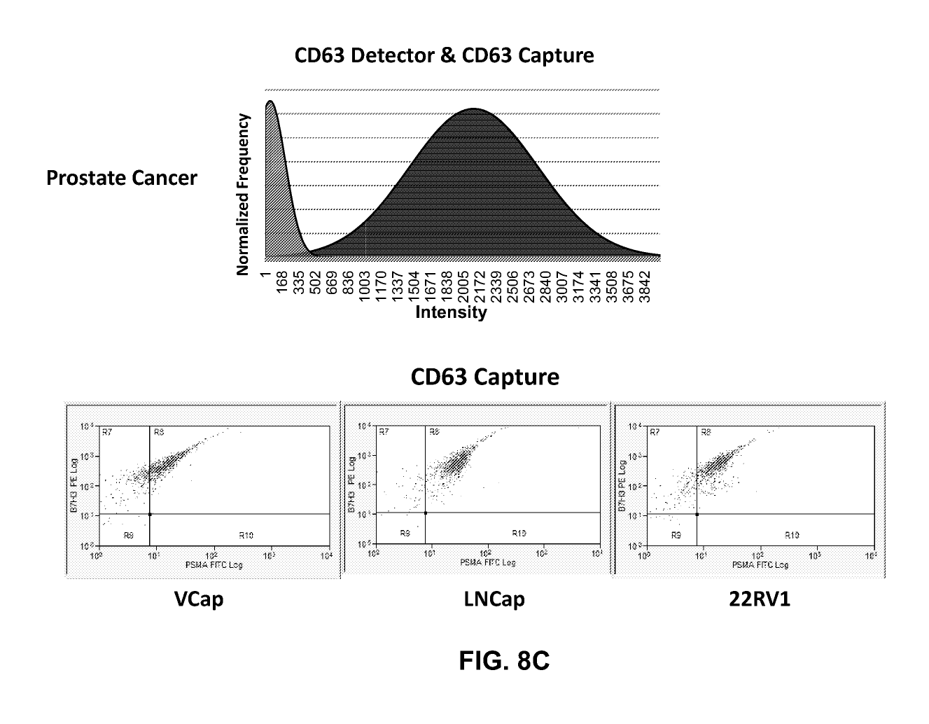

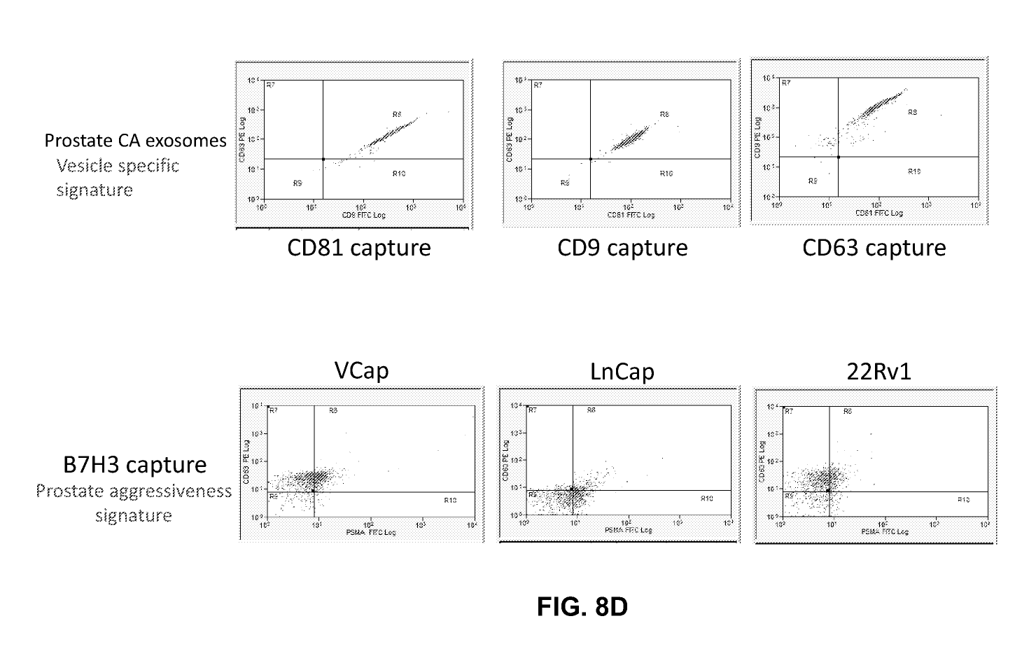

As depicted in FIG. 8, a prostate cancer bio-signature can comprise assaying EpCam, CD63, CD81, CD9, or any combination thereof, of a vesicle. The prostate cancer bio-signature can comprise detection of EpCam, CD9, CD63, CD81, PCSA or any combination thereof. For example, the prostate cancer bio-signature can comprise EpCam, CD9, CD63 and CD81 or PCSA, CD9, CD63 and CD81 (see for example, FIG. 5A). The prostate cancer bio-signature can also comprise PCSA, PSMA, B7H3, or any combination thereof (see for example, FIG. 5B). In one embodiment, the biosignature comprises PMSA and one or more tetraspanins, e.g., CD9, CD63 and/or CD81. In another embodiment, the biosignature comprises PCSA and one or more tetraspanins, e.g., CD9, CD63 and/or CD81. In these embodiments, PMSA or PSCA can be used to capture vesicles and the one or more tetraspanins can be used for detection. In some embodiments, PMSA, PSCA and/or B7H3 are used to capture the vesicles and CD9, CD63 and/or CD81 are used to detect the vesicles. Capture and detection can further comprise the use of lectins.

Furthermore, assessing a plurality of biomarkers can provide increased sensitivity, specificity, or signal intensity, as compared to assessing less than a plurality of biomarkers. For example, assessing PSMA and B7H3 can provide increased sensitivity in detection as compared to assessing PSMA or B7H3 alone. Assessing CD9 and CD63 can provide increased sensitivity in detection as compared to assessing CD9 or CD63 alone. In one embodiment, one or more of the following biomarkers are detected: EpCam, CD9, PCSA, CD63, CD81, PSMA, B7H3, PSCA, ICAM, STEAP, and EGFR.

Prostate cancer can also be characterized based on meeting at least 1, 2, 3, 4, 5, 6, 7, 8, 9, or 10 criteria. For example, a number of different criteria can be used: 1) if the amount of vesicles in a sample from a subject is higher than a reference value; 2) if the amount of prostate cell derived vesicles is higher than a reference value; and 3) if the amount of vesicles with one or more cancer specific biomarkers is higher than a reference value, the subject is diagnosed with prostate cancer. The method can further include a quality control measure.

In another embodiment, one or more bio-signatures of a vesicle are used for the diagnosis between normal prostate and prostate cancer, or between normal prostate, BPH and PCa. Any appropriate biomarker disclosed herein can be used to distinguish PCa. In some embodiments, one or more general capture agents to a biomarker (or capture biomarker, a biomarker that is detected or bound by a capture agent) can be used to capture one or more vesicles from a sample from a subject.

Prostate specific biomarkers can be used to identify prostate specific vesicles. Cancer biomarkers can be used to identify cancer specific vesicles. In some embodiments, one or more of CD9, CD81 and CD63 are used as capture biomarkers. In some embodiments, PCSA is used as a prostate biomarker. In some embodiments, the one or more cancer biomarkers comprise one or more of EpCam and B7H3.

In some embodiments, the method of identifying prostate cancer in a subject comprises: (a) capturing a population of vesicles in a sample from the subject using a capture agent; (b) determining a level of one or more cancer biomarkers in the population of vesicles; (c) determining a level of one or more prostate biomarkers in the population of vesicles; and (d) identifying the subject as having prostate cancer if the level of the one or more cancer biomarkers and the level of one or more prostate biomarkers meet a predetermined threshold value. In some embodiments, the capture agent comprises one or more binding agents for CD9, CD81 and CD63. In some embodiments, the one or more prostate biomarkers comprises PCSA. In some embodiments, the one or more prostate biomarkers comprises PSMA. In some embodiments, the one or more cancer biomarkers comprise one or more of EpCam and B7H3. In some embodiments, the predetermined threshold value comprises a measured value of a detectable label. For example, the detectable label can be a fluorescent moiety and the value can be a luminescence value of the moiety.

The prostate cancer can be characterizing using one or more processes disclosed herein with at least 60, 61, 62, 63, 64, 65, 66, 67, 68, 69, or 70% sensitivity. The prostate cancer can be characterized with at least 80, 81, 82, 83, 84, 85, 86, or 87% sensitivity. For example, the prostate cancer can be characterized with at least 87.1, 87.2, 87.3, 87.4, 87.5, 87.6, 87.7, 87.8, 87.9, 88.0, or 89% sensitivity, such as with at least 90% sensitivity, such as at least 91, 92, 93, 94, 95, 96, 97, 98, 99 or 100% sensitivity.

The prostate cancer of a subject can also be characterized with at least 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

The prostate cancer can also be characterized with at least 70% sensitivity and at least 80, 90, 95, 99, or 100% specificity; at least 80% sensitivity and at least 80, 85, 90, 95, 99, or 100% specificity; at least 85% sensitivity and at least 80, 85, 90, 95, 99, or 100% specificity; at least 86% sensitivity and at least 80, 85, 90, 95, 99, or 100% specificity; at least 87% sensitivity and at least 80, 85, 90, 95, 99, or 100% specificity; at least 88% sensitivity and at least 80, 85, 90, 95, 99, or 100% specificity; at least 89% sensitivity and at least 80, 85, 90, 95, 99, or 100% specificity; at least 90% sensitivity and at least 80, 85, 90, 95, 99, or 100% specificity; at least 95% sensitivity and at least 80, 85, 90, 95, 99, or 100% specificity; at least 99% sensitivity and at least 80, 85, 90, 95, 99, or 100% specificity; or at least 100% sensitivity and at least 80, 85, 90, 95, 99, or 100% specificity.

In some embodiments, the prostate cancer is characterized with at least 70, 71, 72, 73, 74, 75, 76, 77, 78, 79, 80, 81, 82, 83, 84, 85, 86, 87, 88, 89, 90, 91, 92, 93, 94, 95, 96, or 97% accuracy, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% accuracy.

In some embodiments, the prostate cancer is characterized with an AUC of at least 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, or 0.97, such as with at least 0.971, 0.972, 0.973, 0.974, 0.975, 0.976, 0.977, 0.978, 0.978, 0.979, 0.980, 0.981, 0.982, 0.983, 0.984, 0.985, 0.986, 0.987, 0.988, 0.989, 0.99, 0.991, 0.992, 0.993, 0.994, 0.995, 0.996, 0.997, 0.998, 0.999 or 1.00.

Furthermore, the confidence level for determining the specificity, sensitivity, accuracy and/or AUC can be determined with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% confidence.

### Compositions

Also provided herein are compositions of one or more vesicles. The vesicle can be cell-of-origin specific, such as derived from a cancer cell, such as a lung, pancreas, stomach, intestine, bladder, kidney, ovary, testis, skin, colorectal, breast, prostate, brain, esophagus, or liver cancer cell.

The composition can be stored and archived, such as in a bio-fluid bank and retrieved for analysis as necessary. Alternatively, the composition can be analyzed immediately after being collected and placed in a composition comprising the preservation buffer.

In some embodiments, the composition comprises a vesicle and a preservation buffer. The preservation buffer is useful for the stabilization of a vesicle, such as by preserving the structural integrity of the vesicle and the antigenic sites and biomarkers of the biomarkers, such as proteins, nucleic acids, and other vesicle components, for a useful period of time. Thus, the preservation buffer can prevent or slow the degradation or destabilization of a vesicle. A vesicle not present in a composition with a preservation buffer can degrade more rapidly as compared to a vesicle present with a preservation buffer. For example, the preservation buffer can prevent or slow the degradation of the vesicle that can typically occur at room temperature. The vesicle present in a composition comprising a preservation buffer can be stabilized for longer than a vesicle not present in a preservation buffer. For example, the vesicle present in a composition comprising a preservation buffer can be stabilized for at least about 1.5, 2, 3, 4, 5, 6, 7, 8, 9, 10, 11, 12, 13, 14, 15, 16, 17, 18, 19, 20 times longer than a vesicle not present in a preservation buffer. The comparison in time can be performed by comparing vesicle samples where all conditions, except for the buffer in which the vesicle is present, are the same. For example, the temperature and preparation of the vesicles are the same, except for the buffer in which the vesicles being compared are stored.

The degradation or stabilization effect of the preservation buffer on the vesicle can be determined by comparing the structural integrity of the vesicle, such as by comparing a vesicle collected or stored in a preservation buffer as compared to a vesicle not stored in a preservation buffer, at various time points, temperatures, or both. For example, a protein biomarker can be detected in a vesicle not stored in a preservation buffer up until 5 hours after being isolated. However, a protein biomarker can be detected in a vesicle stored in a preservation buffer more than 5 hours after being isolated. In another embodiment, a protein biomarker can be detected in a vesicle not stored in a preservation buffer of the vesicle is stored at 4° C. after being isolated, but not if stored at room temperature. However, a protein biomarker can be detected in a vesicle stored in a preservation buffer when stored at room temperature.

In some embodiments, the preservation buffer can prevent degradation of a vesicle for at least about 2, 4, 6, 8, 10, 12, 24, 36, 48, 60, 72, 84, or 96 hours. In yet other embodiments, the preservation buffer can prevent degradation at warmer temperatures, such as greater than about −80° C., −20° C., 0° C., 4° C., 10° C., 15° C., 20° C., 21° C., 22° C., 23° C., 24° C., or 25° C. In some embodiments, preservation buffer can prevent degradation of a vesicle at room temperature, such as about 25° C. The preservation buffer can prevent degradation at room temperature, for at least about 2, 4, 6, 8, 10, 12, 24, 36, 48, 60, 72, 84, or 96 hours.

The preservation buffer can comprise a fixative, such as diazolidinyl urea (DU), imidazolidinyl urea (IDU), dimethylol-5,5-dimethylhydantoin, dimethylol urea, 2-bromo-2-nitropropane-1,3-diol, 5-hydroxymethoxymethyl-1-aza-3,7-dioxabicyclo(3.3.0)octane and 5-hydroxymethyl-1-aza-3,7-dioxabicyclo(3.3.0)octane and 5-hydroxypoly[methyleneoxy]methyl-1-aza-3,7-dioxabicyclo(3.3.0)octane, sodium hydroxymethyl glycinate and mixtures thereof. In some embodiments, the preservation buffer comprises from about 1 to about 20 percent, about 2 to about 15 percent, about 3 to about 10 percent, or about 4 to about 6 percent, by weight imidazolidinyl urea. In some embodiments, the preservation buffer comprises about 1, 2, 3, 4, 5, 6, 7, 8, 9, 10, 11, 12, 13, 14, 15, 16, 17, 18, 19, or 20 percent by weight imidazolidinyl urea.

The preservation buffer can comprise a mixture of imidazolidinyl urea and diazolidinyl urea. The total concentration of imidazolidinyl urea and diazolidinyl urea can be from about 1 to 20 percent, about 2 to about 15 percent, about 3 to about 10 percent, or about 4 to about 10 percent by weight. In some embodiments, the weight ratio of imidazolidinyl urea to diazolidinyl urea is from about 100:1 to about 1:100, about 50:1 to about 1:50, about 20:1 to about 1:20, or about 10:1 to about 1:10.

The preservation buffer can also comprise a protease inhibitor, such as phenylmethylsulfonyl fluoride, AEBSF lysine, or any combination thereof. Other protease inhibitors can also be used.

Furthermore, the preservation buffer comprises an additive selected from the group consisting of polyethylene glycol (PEG), ethylenediaminetetraacetic acid (EDTA), phosphate buffered saline and mixtures thereof. For example, the preservation buffer comprises about 0.001 to about 0.2 percent by weight EDTA. In some embodiments, the preservation buffer can comprise up to about 1 percent by weight PEG. In one embodiment, the preservation buffer comprises 0.3% phosphate buffered saline and ethylene diaminetetraacetic acid, 0.3% polyethylene glycol and 3% imidazolidinyl urea.

The invention further provides a composition comprising a vesicle and a lectin. In some embodiments, the composition can further comprise a preservation buffer. The lectin can bind a vesicle proteoglycan or a fragment thereof. For example, lectin can binds high mannose glycoproteins present on a vesicle. The lectin can be, but not limited to, Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), or Griffonia (Bandeiraea) Simplicifolia Lectin II (GS-II).

The lectin can be attached to a substrate, such as disclosed hereon. The substrate can be a planar substrate or a particle. The substrate can be, but not limited to, agarose, aminocelite, resins, silica, polysaccharide, plastic or proteins (e.g. gelatin). For example, the silica can be glass beads, sand, and diatomaceous earth. The polysaccharide can be selected from the group consisting of dextran, cellulose and agarose. The plastic can be selected from the group consisting of polystyrenes, polysuflones, polyesters, polyurethanes, polyacrylates and their activated and native amino and carboxyl derivatives. The lectin can be attached to a substrate by a linker, such as a cleavable linker. The linker can be selected from the group consisting of gluteraldehyde, C2 to C18 dicarboxylates, diamines, dialdehydes, dihalides, and mixtures thereof.

The composition can further comprise a non-lectin binding agent. The non-lectin binding agent can bind a vesicle component. The non-lectin binding agent can be a nucleic acid (such as DNA or RNA), a monoclonal antibody, a polyclonal antibody, a Fab, a Fab′, a single chain antibody, a synthetic antibody, an aptamer (DNA/RNA), a peptoid, a zDNA, a peptide nucleic acids (PNA), a locked nucleic acids (LNA), a synthetic chemical compound, a naturally occurring chemical compound, a dendrimers, or any combinations thereof. For example, the non-lectin binding agent can be an antibody, such as an antibody that binds a tumor antigen.

The compositions disclosed herein can further comprise a label. For example, a label can be attached to component of the composition, such as a lectin or a non-lectin binding agent. The composition can comprise one or more labels. For example, a lectin can have 2 or more labels attached. A non-lectin binding agent can also have 2 or more labels attached. Alternatively, a composition can comprise a lectin and a non-lectin binding agent, wherein the lectin has a label and the non-lectin has a label that differs from that of the lectin. The lectin and non-lectin binding agent can be labelled with different combination of labels.

The label can be, but not limited to, a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal, a nanoparticle, a quantum dot, a gold particle, a silver particle or a radioactive label. For example, the label can be a radioisotope (radionuclides), such as ³H, ¹¹C, ¹⁴C, ¹⁸F, ³²F, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. The label can be a fluorescent label, such as a rare earth chelate (europium chelate), fluorescein type, such as, but not limited to, FITC, 5-carboxyfluorescein, 6-carboxy fluorescein; a rhodamine type, such as, but not limited to, TAMRA; dansyl; Lissamine; cyanines; phycoerythrins; Texas Red; and analogs thereof.

For example, various enzyme-substrate labels are available or disclosed (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

The label can be directly or indirectly attached to the lectin, non-lectin binding agent or both. For example, the label can be directly linked or conjugated to the binding agent. For example, a lectin can be directly labeled, such as commercially available (e.g. Molecular Probes from Invitrogen). A label can be attached to a binding agent, such as an antibody through biotin-streptavidin. Alternatively, the binding agent such as an antibody is not labeled, but is later contacted with a second antibody that is labeled after the first antibody is bound to an antigen of interest.

In another embodiment, various enzyme-substrate labels are available or disclosed and can be used (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Labels may include fluorescein or its derivatives, such as fluorescein isothiocyanate (FITC), Oregon Green, Tokyo Green, SNAFL, carboxynaphthofluorescein (CFSE), Carboxyfluorescein diacetate succinimidyl ester (CFDA-SE), DyLight 488, Alexa Fluor 488, green fluorescent protein (GFP), phycoerythrin (PE), Peridinin Chlorophyll protein (PerCP), PE-Alexa Fluor 700, PE-Cy5 (TRI-COLOR), PE-Cy5.5, PE-Alexa Fluor 750, PE-Cy7, allophycocyanin (APC), APC-Cy7, and derivatives thereof.

The invention further provides a composition comprising a plurality of vesicles, such as those described herein. For example, the composition can comprise a substantially enriched population of vesicles, wherein the enriched population of vesicles comprises vesicles with a substantially identical glycosylation pattern, such as the content, amount, or both, of sugars present.

The glycosylation pattern of a vesicle may comprise N- or O-glycosylation of any proteineous moiety, wherein the addition of one or more sugar molecules may be at the amide nitrogen of asparagine or the hydroxyl oxygen of hydroxylysine, hydroxyproline, serine, or threonine, respectively. The glycosylation pattern may be characterized by the level or type of sugar molecules or saccharides, such as monosaccharides, disaccharides, polysaccharides or oligosaccarhides. For example, the sugar molecules may be trioses, tetrososes, pentoses, hexoses, heptoses, octoses, nonoses, or derivatives thereof, such as deoxy sugars, such as deoxyhexoses; N- or O-substituted derivatives, such as sialic acid; or sugars with amino groups. The sugar molecules may include, but not be limited to, galactose (Gal), glucose (Glc), mannose (Man), N-acetylneuraminic acid (NeuAc), fucose (Fuc), N-Acetylgalactoseamine (GalNAc), N-Acetylglucosamine (GlcNAc); and Xylose (Xyl). The sugar molecules may be linked to other sugar molecules via α or β linkage.

The glycosylation pattern can be detected using agents that specifically or preferentially recognize or bind specific sugar molecules or the proteinaceous moieties that are associated or modified with the specific sugar molecules. Agents may include, but not be limited to, lectins, such as those described herein. For example, Lens culimaris agglutinin-A (LCA) is a chemical that specifically binds to proteins modified with fucose; wheat germ agglutinin (WGA), which has preferential binding to N-acetylglucosamine; concanavalin A (Con A), which recognizes α-linked mannose; and Griffonia (Bandeiraea) Simplicifolia Lectin II (GS-II), which binds to α- or β-linked N-acetylglucosamine residues.

The agents may be detected directly or indirectly. For example, the agents may be conjugated to a label, such as described herein. Any detectable label can be used, such as a fluorescent label. Detections methods known in the arts, such as flow cytometry may be used to detect and/or separate labeled cells. For example, detection and/or separation may be by fluorescence activate cell sorting (FACS). Labels may include fluorescein or its derivatives, such as fluorescein isothiocyanate (FITC), Oregon Green, Tokyo Green, SNAFL, carboxynaphthofluorescein (CFSE), Carboxyfluorescein diacetate succinimidyl ester (CFDA-SE), DyLight 488, Alexa Fluor 488, green fluorescent protein (GFP), phycoerythrin (PE), Peridinin Chlorophyll protein (PerCP), PE-Alexa Fluor 700, PE-Cy5 (TRI-COLOR), PE-Cy5.5, PE-Alexa Fluor 750, PE-Cy7, allophycocyanin (APC), APC-Cy7, and derivatives thereof. The aforementioned labels may also be used to analyze the glycosylation patterns of glycoproteins. Alternatively, the agents may be detected directly, for example, by using antibodies to detect the agents, such as by Western blotting and other methods well known in the arts.

Other methods to analyze the glycosylation pattern may include compositional analysis of different types of sugars, such as neutral sugars and sugars with amino groups. For example, neutral sugars, such as galactose, mannose, fucose or the like, and sugars with amino groups, such as N-acetylglucosamine or the like, and an acidic sugar, such as sialic acid or the like can be analyzed. The compositional ratio can be analyzed by releasing neutral sugars or amino sugars by acid hydrolysis of the sugar chain. Methods man include, but not be limited to, a method using a sugar composition analyzer (BioLC) manufactured by Dionex. The BioLC is an apparatus for analyzing sugar composition by HPAEC-PAD (high performance anion-exchange chromatography-pulsed amperometric detection) method (Rocklin et al., J. Lig. Chromatogr. 6(9), 1577-1590 (1983)). The compositional ratio can also be analyzed by a fluorescence labeling method using 2-aminopyridine (PA). Specifically, the compositional ratio can be calculated by fluorescence-labeling an acid-hydrolyzed sample with 2-aminopyridine in accordance with a known method (Kondo et al., Agric. Biol. Chem., 55(1), 283-284 (1991)) and carrying out HPLC analysis.

The glycosylation pattern can also be analyzed by a two-dimensional sugar chain mapping method (Tomiya et al., Anal. Biochem., 171, 73-80 (1988); Biochemical Experimentation Method 23—Method for Studying Glycoprotein Sugar Chains (Gakkai Shuppan Center), edited by Reiko Takahashi (1989)). The two-dimensional sugar chain mapping method is a method in which the sugar chain structure is estimated, for example, by plotting the retention time or eluting position of the sugar chain by reverse phase chromatography as the X axis and the retention time or eluting position of the sugar chain by a normal phase chromatography as the Y axis, and comparing the results with those of known sugar chains.

The sugar chain can be released by hydrazinolysis and then fluorescence labeling of the sugar chain with 2-aminopyridine (Hase et al., J. Biochem., 95, 1973203 (1984)) is carried out. The sugar chain is separated from an excess PA reagent and the like by gel filtration and subjected to reverse phase chromatography. Subsequently, each peak of the fractionated sugar chain is analyzed by normal phase chromatography. Based on these results, the sugar chain structure can be estimated by plotting the spots on a two-dimensional sugar chain map and comparing them with those of sugar chain standards (manufactured by Takara Shuzo) or a reference (Tomiya et al., Anal. Biochem., 171, 73-80 (1988)).

In addition, the glycosylation pattern can be analyzed by mass spectrometry, such as MALDI-TOF-MS or the like, of each sugar chain.

The composition can be enriched for a population of vesicles with substantially identical glycosylation pattern, such that the vesicles in the population have a sugar content that is at least 30, 40, 50, 60, 70, 80, 90, 95 or 99% identical. For example, each vesicle in the population can have the same type of sugars (e.g. all appropriate vesicles, e.g., exosomes, have fucose and glucose), same amount of each sugar or a plurality of sugars, same type of sugar chain structure, or any combination thereof. The population of vesicles may have a glycosylation pattern that differs from that of another population of vesicles. For example, the glycosylation pattern for a population of vesicles from a sample obtained from a subject without cancer can differ from the glycosylation pattern for a population of vesicles from a sample obtained from a subject with cancer.

The vesicles with a substantially identical glycosylation pattern can comprise at least about 30, 40, 50, 60, 70, 80, 90, 95, or 99% of the total vesicle population of the composition. In some embodiments, a composition comprising a substantially enriched population of vesicles comprises at least 2, 3, 4, 5, 10, 20, 25, 50, 100, 250, 500, or 1000 times the concentration of a vesicle with specific glycosylation pattern as compared to a concentration of the vesicle in a biological sample from which the composition was derived. In yet other embodiments, the composition can further comprise a second enriched population of vesicles.

Furthermore, the vesicles with a specific glycosylation pattern that is substantially identical can be cell-of-origin specific vesicles, such as derived from the same type of cell. The cell-of-origin can be a tumor or cancer cell. The cell-of-origin can be a lung, pancreas, stomach, intestine, bladder, kidney, ovary, testis, skin, colorectal, breast, prostate, brain, esophagus, liver, placenta, or fetal cell. An isolated vesicle of prostatic origin can comprise the biomarkers PCSA and/or PSMA. An isolated vesicle of prostate cancer origin can comprise the biomarkers PCSA and/or PSMA, along with EpCam and/or B7H3. The isolated vesicles can further comprise one or more tetraspanins such as CD9, CD63 and/or CD81. In some embodiments, an isolated vesicle of prostate cancer origin comprises the surface markers PCSA and/or PSMA, EpCam and/or B7H3, and CD9, CD63 and/or CD81. The isolated vesicle can display all seven of these markers.

### EXAMPLES

### Example 1

### Purification of Vesicles from Prostate Cancer Cell Lines

Prostate cancer cell lines are cultured for 3-4 days in culture media containing 20% FBS (fetal bovine serum) and 1% P/S/G. The cells are then pre-spun for 10 minutes at 400×g at 4° C. The supernatant is kept and centrifuged for 20 minutes at 2000×g at 4. The supernatant containing vesicles can be concentrated using a Millipore Centricon Plus-70 (Cat #UFC710008 Fisher).

The Centricon is pre washed with 30 mls of PBS at 1000×g for 3 minutes at room temperature. Next, 15-70 mls of the pre-spun cell culture supernatant is poured into the Concentrate Cup and is centrifuged in a Swing Bucket Adapter (Fisher Cat #75-008-144) for 30 minutes at 1000×g at room temperature.

The flow through in the Collection Cup is poured off. The volume in the Concentrate Cup is brought back up to 60 mls with any additional supernatant. The Concentrate Cup is centrifuged for 30 minutes at 1000×g at room temperature to concentrate the cell supernatant.

The Concentrate Cup is washed by adding 70 mls of PBS and centrifuged for 30-60 minutes at 1000×g until approximately 2 mls remains. The vesicles are removed from the filter by inverting the concentrate into the small sample cup and centrifuge for 1 minute at 4° C. The volume is brought up to 25 mls with PBS. The vesicles are now concentrated and are added to a 30% Sucrose Cushion.

To make a cushion, 4 mls of Tris/30% Sucrose/D2O solution (30 g protease-free sucrose, 2.4 g Tris base, 50 ml D2O, adjust pH to 7.4 with 10N NCL drops, adjust volume to 100 mls with D2O, sterilize by passing thru a 0.22-um filter) is loaded to the bottom of a 30 ml V bottom thin walled Ultracentrifuge tube. The diluted 25 mls of concentrated vesicles is gently added above the sucrose cushion without disturbing the interface and is centrifuged for 75 minutes at 100,000×g at 4° C. The ˜25 mls above the sucrose cushion is carefully removed with a 10 ml pipet and the ˜3.5 mls of vesicles is collected with a fine tip transfer pipet (SAMCO 233) and transferred to a fresh ultracentrifuge tube, where 30 mls PBS is added. The tube is centrifuged for 70 minutes at 100,000×g at 4° C. The supernatant is poured off carefully. The pellet is resuspended in 200 ul PBS and can be stored at 4° C. or used for assays. A BCA assay (1:2) can be used to determine protein content and Western blotting or electron micrography can be used to determine vesicle purification.

### Example 2

### Purification of Vesicles from VCaP and 22Rv1

Vesicles from Vertebral-Cancer of the Prostate (VCaP) and 22Rv1, a human prostate carcinoma cell line, derived from a human prostatic carcinoma xenograft (CWR22R) were collected by ultracentrifugation by first diluting plasma with an equal volume of PBS (1 ml). The diluted fluid was transferred to a 15 ml falcon tube and centrifuged 30 minutes at 2000×g 4° C. The supernatant (−2 mls) was transferred to an ultracentrifuge tube 5.0 ml PA thinwall tube (Sorvall #03127) and centrifuged at 12,000×g, 4° C. for 45 minutes.

The supernatant (˜2 mls) was transferred to a new 5.0 ml ultracentrifuge tubes and filled to maximum volume with addition of 2.5 mls PBS and centrifuged for 90 minutes at 110,000×g, 4° C. The supernatant was poured off without disturbing the pellet and the pellet resuspended with 1 ml PBS. The tube was filled to maximum volume with addition of 4.5 ml of PBS and centrifuged at 110,000×g, 4° C. for 70 minutes.

The supernatant was poured off without disturbing the pellet and an additional 1 ml of PBS was added to wash the pellet. The volume was increased to maximum volume with the addition of 4.5 mls of PBS and centrifuged at 110,000×g for 70 minutes at 4° C. The supernatant was removed with P-1000 pipette until ˜100 μl of PBS was in the bottom of the tube. The ˜90 μl remaining was removed with P-200 pipette and the pellet collected with the ˜10 it of PBS remaining by gently pipetting using a P-20 pipette into the microcentrifuge tube. The residual pellet was washed from the bottom of a dry tube with an additional 5 μl of fresh PBS and collected into microcentrifuge tube and suspended in phosphate buffered saline (PBS) to a concentration of 500 μg/ml.

### Example 3

### Plasma Collection and Vesicle Purification

Blood is collected via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

For storage, approximately 1 ml of the plasma (supernatant) is aliquoted to a cryovials, placed in dry ice to freeze them and stored in −80° C. Before vesicle purification, if samples were stored at −80° C., samples are thawed in a cold water bath for 5 minutes. The samples are mixed end over end by hand to dissipate insoluble material.

In a first prespin, the plasma is diluted with an equal volume of PBS (example, approximately 2 ml of plasma is diluted with 2 ml of PBS). The diluted fluid is transferred to a 15 ml Falcon tube and centrifuged for 30 minutes at 2000×g at 4° C.

For a second prespin, the supernatant (approximately 4 mls) is carefully transferred to a 50 ml Falcon tube and centrifuged at 12,000×g at 4° C. for 45 minutes in a Sorval.

In the isolation step, the supernatant (approximately 2 mls) is carefully transferred to a 5.0 ml ultracentrifuge PA thinwall tube (Sorvall #03127) using a P1000 pipette and filled to maximum volume with an additional 0.5 mls of PBS. The tube is centrifuged for 90 minutes at 110,000×g at 4° C.

In the first wash, the supernatant is poured off without disturbing the pellet. The pellet is resuspended or washed with 1 ml PBS and the tube is filled to maximum volume with an additional 4.5 ml of PBS. The tube is centrifuged at 110,000×g at 4° C. for 70 minutes. A second wash is performed by repeating the same steps.

The vesicles are collected by removing the supernatant with P-1000 pipette until approximately 100 μl of PBS is in the bottom of the tube. Approximately 90 μl of the PBS is removed and discarded with P-200 pipette. The pellet and remaining PBS is collected by gentle pipetting using a P-20 pipette. The residual pellet is washed from the bottom of the dry tube with an additional 5 μl of fresh PBS and collected into a microcentrifuge tube.

### Example 4

### Analysis of Vesicles Using Antibody-Coupled Microspheres and Directly Conjugated Antibodies

This example demonstrates the use of particles coupled to an antibody, where the antibody captures the vesicles (see for example, FIG. 4B). An antibody, the detector antibody, is directly coupled to a label, and is used to detect a biomarker on the captured vesicle.

First, an antibody-coupled microsphere set is selected (Luminex, Austin, Tex.). The microsphere set can comprise various antibodies, and thus allows multiplexing. The microspheres are resuspended by vortex and sonication for approximately 20 seconds. A Working Microsphere Mixture is prepared by diluting the coupled microsphere stocks to a final concentration of 100 microspheres of each set/μL in Startblock (Pierce (37538)). (Note: 50 μL of Working Microsphere Mixture is required for each well.) Either PBS-1% BSA or PBS-BN (PBS, 1% BSA, 0.05% Azide, pH 7.4) may be used as Assay Buffer.

A 1.2 μm Millipore filter plate is pre-wet with 100 μl/well of PBS-1% BSA (Sigma (P3688-10PAK+0.05% NaAzide (S8032))) and aspirated by vacuum manifold. An aliquot of 50 μl of the Working Microsphere Mixture is dispensed into the appropriate wells of the filter plate (Millipore Multiscreen HTS (MSBVN1250)). A 50 μl aliquot of standard or sample is dispensed into to the appropriate wells. The filter plate is covered and incubated for 60 minutes at room temperature on a plate shaker. The plate is covered with a sealer, placed on the orbital shaker and set to 900 for 15-30 seconds to re-suspend the beads. Following that the speed is set to 550 for the duration of the incubation.

The supernatant is aspirated by vacuum manifold (less than 5 inches Hg in all aspiration steps). Each well is washed twice with 100 μl of PBS-1% BSA (Sigma (P3688-10PAK+0.05% NaAzide (S8032))) and is aspirated by vacuum manifold. The microspheres are resuspended in 50 μL of PBS-1% BSA (Sigma (P3688-10PAK+0.05% NaAzide (S8032))). The PE conjugated detection antibody is diluted to 4 μg/mL (or appropriate concentration) in PBS-1% BSA (Sigma (P3688-10PAK+0.05% NaAzide (S8032))). (Note: 50 μL of diluted detection antibody is required for each reaction.) A 50 μl aliquot of the diluted detection antibody is added to each well. The filter plate is covered and incubated for 60 minutes at room temperature on a plate shaker. The filter plate is covered with a sealer, placed on the orbital shaker and set to 900 for 15-30 seconds to re-suspend the beads. Following that the speed is set to 550 for the duration of the incubation. The supernatant is aspirated by vacuum manifold. The wells are washed twice with 100 μl of PBS-1% BSA (Sigma (P3688-10PAK+0.05% NaAzide (S8032))) and aspirated by vacuum manifold. The microspheres are resuspended in 100 μl of PBS-1% BSA (Sigma (P3688-10PAK+0.05% NaAzide (S8032))). The microspheres are analyzed on a Luminex analyzer according to the system manual.

### Example 5

### Analysis of Vesicles Using Antibody-Coupled Microspheres and Biotinylated Antibody

This example demonstrates the use of particles coupled to an antibody, where the antibody captures the vesicles. An antibody, the detector antibody, is biotinylated. A label coupled to streptavidin is used to detect the biomarker.

First, the appropriate antibody-coupled microsphere set is selected (Luminex, Austin, Tex.). The microspheres are resuspended by vortex and sonication for approximately 20 seconds. A Working Microsphere Mixture is prepared by diluting the coupled microsphere stocks to a final concentration of 50 microspheres of each set/μL in Startblock (Pierce (37538)). (Note: 50 μl of Working Microsphere Mixture is required for each well.) Beads in Start Block should be blocked for 30 minutes and no more than 1 hour.

A 1.2 μm Millipore filter plate is pre-wet with 100 μl/well of PBS-1% BSA+Azide (PBS-BN) ((Sigma (P3688-10PAK+0.05% NaAzide (S8032))) and is aspirated by vacuum manifold. A 50 μl aliquot of the Working Microsphere Mixture is dispensed into the appropriate wells of the filter plate (Millipore Multiscreen HTS (MSBVN1250)). A 50 μl aliquot of standard or sample is dispensed to the appropriate wells. The filter plate is covered with a seal and is incubated for 60 minutes at room temperature on a plate shaker. The covered filter plate is placed on the orbital shaker and set to 900 for 15-30 seconds to re-suspend the beads. Following that, the speed is set to 550 for the duration of the incubation.

The supernatant is aspirated by a vacuum manifold (less than 5 inches Hg in all aspiration steps). Aspiration can be done with the Pall vacuum manifold. The valve is place in the full off position when the plate is placed on the manifold. To aspirate slowly, the valve is opened to draw the fluid from the wells, which takes approximately 3 seconds for the 100 μl of sample and beads to be fully aspirated from the well. Once the sample drains, the purge button on the manifold is pressed to release residual vacuum pressure from the plate.

Each well is washed twice with 100 μl of PBS-1% BSA+Azide (PBS-BN) (Sigma (P3688-10PAK+0.05% NaAzide (S8032))) and is aspirated by vacuum manifold. The microspheres are resuspended in 50 μl of PBS-1% BSA+Azide (PBS-BN) ((Sigma (P3688-10PAK+0.05% NaAzide (S8032))).

The biotinylated detection antibody is diluted to 4 μg/mL in PBS-1% BSA+Azide (PBS-BN) (Sigma (P3688-10PAK+0.05% NaAzide (S8032))). (Note: 50 μl of diluted detection antibody is required for each reaction.) A 50 μl aliquot of the diluted detection antibody is added to each well.

The filter plate is covered and incubated with shaking as described above. The supernatant is aspirated by vacuum manifold as described above. The wells are washed and resuspended with PBS-BN as described above.

The streptavidin-R-phycoerythrin reporter (Molecular Probes 1 mg/ml is diluted to 4 μg/mL in PBS-1% BSA+Azide (PBS-BN). 50 μl of diluted streptavidin-R-phycoerythrin is required for each reaction. A 50 μl aliquot of the diluted streptavidin-R-phycoerythrin is added to each well.

The filter plate is covered and incubated with shaking as described above. The supernatant is aspirated by vacuum manifold as described above.

Each well is washed twice with 100 μl of PBS-1% BSA+Azide (PBS-BN) ((Sigma (P3688-10PAK+0.05% NaAzide (S8032))) and is aspirated by vacuum manifold. The microspheres are resuspended in 100 μl of PBS-1% BSA+Azide (PBS-BN) and analyzed on the Luminex analyzer according to the system manual.

### Example 6

### Reference Values for Prostate Cancer

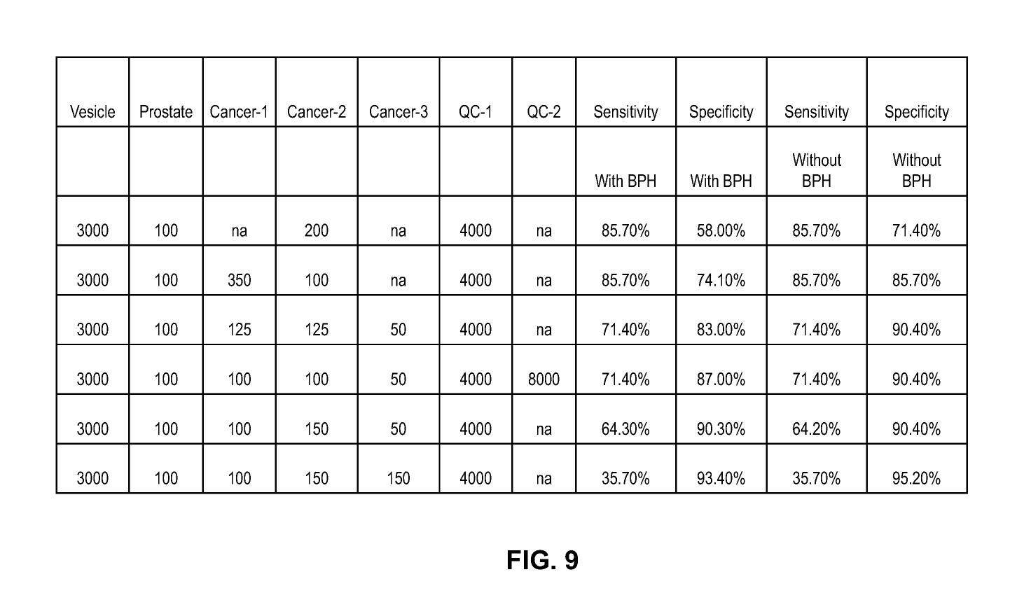

Fourteen stage 3 prostate cancer subjects, eleven benign prostate hyperplasia (BPH) samples, and 15 normal samples were tested. Vesicle samples were obtained using methods as described in Example 3 and used in multiplexing assays, such as described in Examples 4 and 5. The samples were analyzed to determine four criteria 1) if the sample has overexpressed vesicles, 2) if the sample has overexpressed prostate vesicles, 3) if the sample has overexpressed cancer vesicles, and 4) if the sample is reliable. If the sample met all four criteria, the categorization of the sample as positive for prostate cancer had varying sensitivities and specificities, depending on the different bio-signatures present for a sample as described below (Cancer-1, Cancer-2, and Cancer-3, FIG. 9). The four criteria were as follows:

Vesicle Overexpression

The mean fluorescence intensities (MFIs) for a sample in three assays were averaged to determine a value for the sample. Each assay used a different capture antibody. The first used a CD9 capture antibody, the second a CD81 capture antibody, and the third a CD63 antibody. The same combination of detection antibodies was used for each assay, antibodies for CD9, CD81, and CD63. If the average value obtained for the three assays was greater than 3000, the sample was categorized as having overexpressed vesicles (FIG. 9, Vesicle).

Prostate Vesicle Overexpression

The MFIs for a sample in two assays were averaged to determine a value for the sample. Each assay used a different capture antibody. The first used a PCSA capture antibody and the second used a PSMA capture antibody. The same combination of detection antibodies was used for each assay, antibodies for CD9, CD81, and CD63. If the average value obtained for the two assays was greater than 100, the sample was categorized as having prostate vesicles overexpressed (FIG. 9, Prostate).

Cancer Vesicle Overexpression

Three different cancer bio-signatures were used to determine if cancer vesicles were overexpressed in a sample. The first, Cancer-1, used an EpCam capture antibody and detection antibodies for CD81, CD9, and CD63. The second, Cancer-2, used a CD9 capture antibody with detection antibodies for EpCam and B7H3. If the MFI value of a sample for any two of the three cancer bio-signatures was above a reference value, the sample was categorized as having overexpressed cancer (see FIG. 9, Cancer-1, Cancer-2, Cancer-3).

Reliability of Sample

Two quality control measures, QC-1 and QC-2, were determined for each sample. If the sample met one of them, the sample was categorized as reliable.

For QC-1, the sum of all the MFIs of 7 assays was determined. Each of the 7 assays used detection antibodies for CD59 and PSMA. The capture antibody used for each assay was CD63, CD81, PCSA, PSMA, STEAP, B7H3, and EpCam. If the sum was greater than 4000, the sample was not reliable and not included.

For QC-2, the sum of all the MFIs of 5 assays was determined. Each of the 5 assays used detection antibodies for CD9, CD81 and CD63. The capture antibody used for each assay was PCSA, PSMA, STEAP, B7H3, and EpCam. If the sum was greater than 8000, the sample was not reliable and not included.

The sensitivity and specificity for samples with BPH and without BPH samples after a sample met the criteria as described herein, are shown in FIG. 9.

### Example 7

### Determining Bio-Signatures for Prostate Cancer Using Multiplexing

The samples obtained using methods as described in Example 1-3 are used in multiplexing assays as described in Examples 4 and 5. The detection antibodies used are CD63, CD9, CD81, B7H3 and EpCam. The capture antibodies used are CD9, PSCA, TNFR, CD63 (2 antibodies), B7H3, MFG-E8, EpCam (2 antibodies), CD63, Rab, CD81, STEAP, PCSA, PSMA, 5T4, Rab IgG (control) and IgG (control), resulting in 100 combinations to be screened (FIG. 4C).

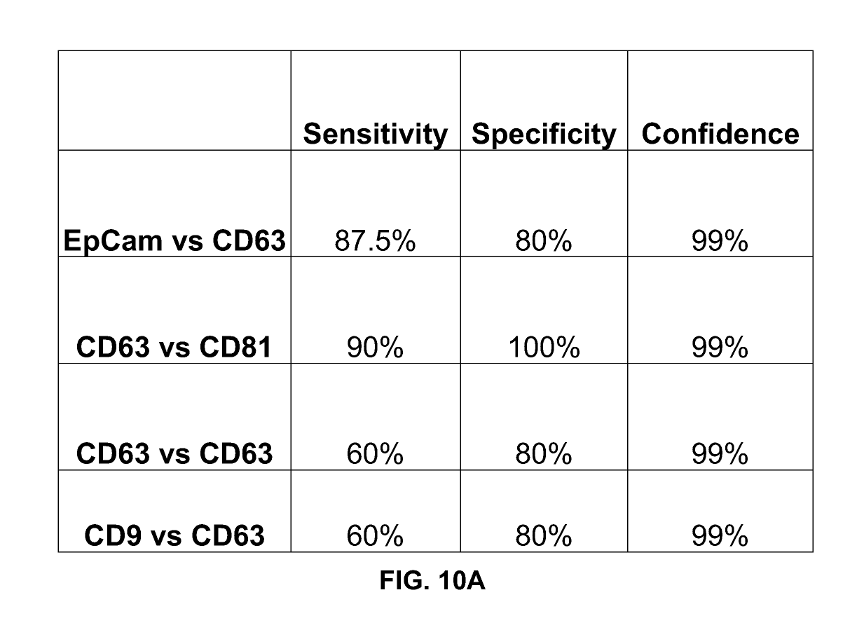

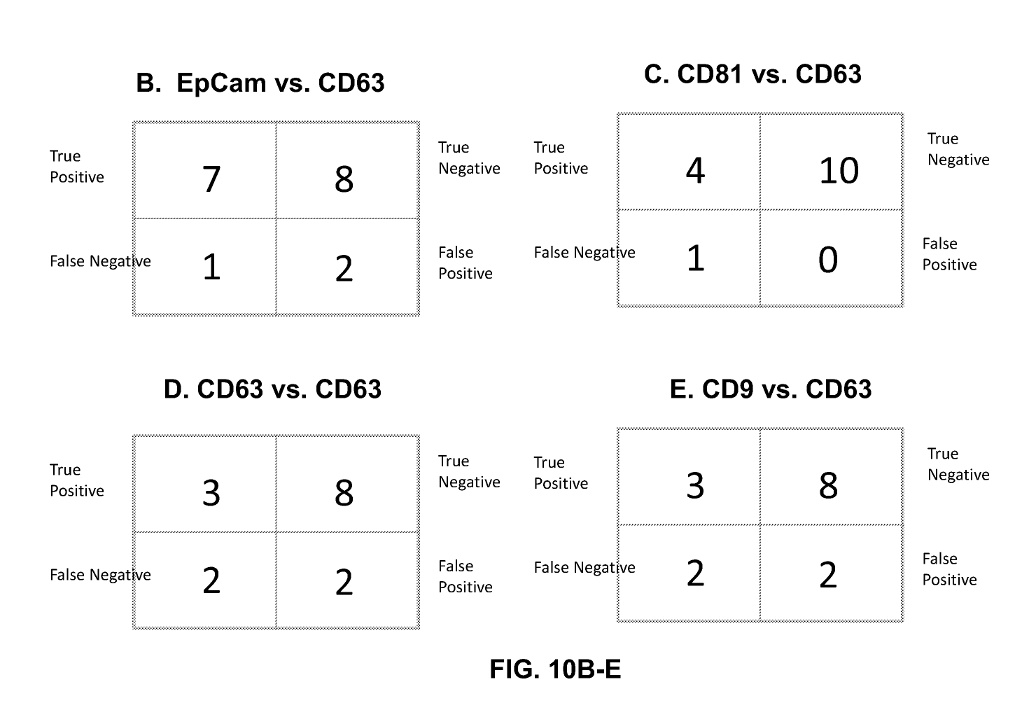

Ten prostate cancer patients and 12 normal control patients were screened. The results are depicted in FIG. 8 and FIG. 5A. FIG. 5B depicts the results of using PCSA capture antibodies (FIG. 5B, left graph) or EpCam capture antibodies (FIG. 5B, right graph), and detection using one or more detector antibodies. The sensitivity and specificity of the different combinations is depicted in FIG. 10.

### Example 8

### Capture of Vesicles Using Magnetic Beads

Vesicles isolated as described in Example 2 are used. Approximately 40 ul of the vesicles are incubated with approximately 5 μg (˜50 μl) of EpCam antibody coated Dynal beads (Invitrogen, Carlsbad, Calif.) and 50 μl of Starting Block. The vesicles and beads are incubated with shaking for 2 hours at 45° C. in a shaking incubator. The tube containing the Dynal beads is placed on the magnetic separator for 1 minute and the supernatant removed. The beads are washed twice and the supernatant removed each time. Wash beads twice, discarding the supernatant each time.

### Example 9

### Vesicle PCa Assay/Test

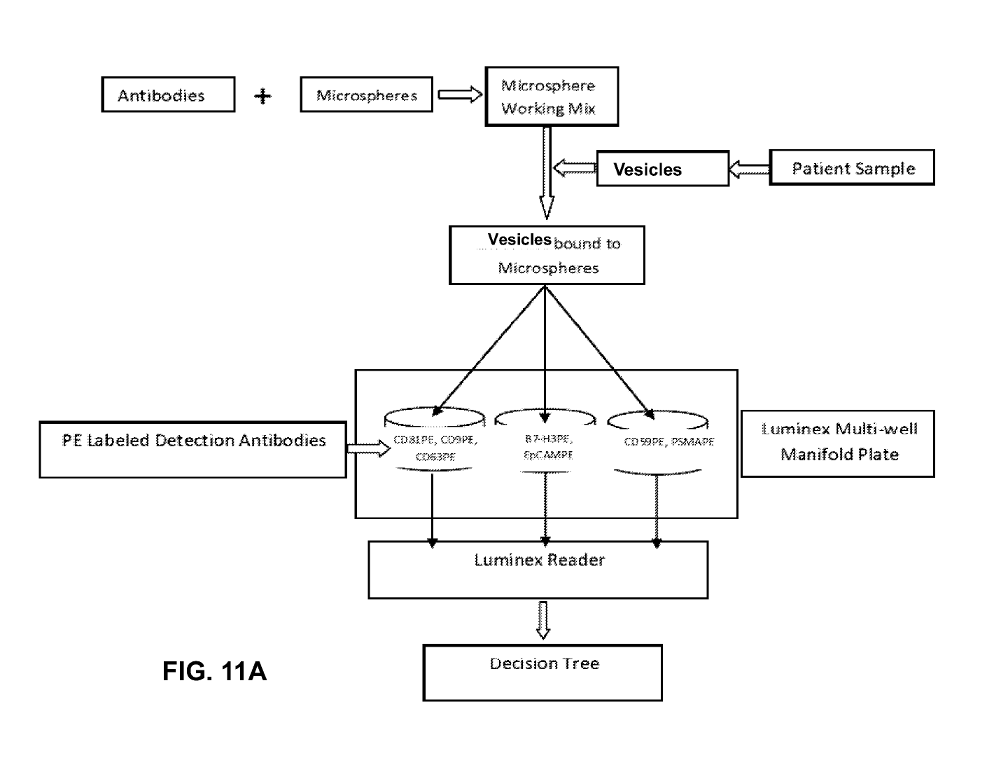

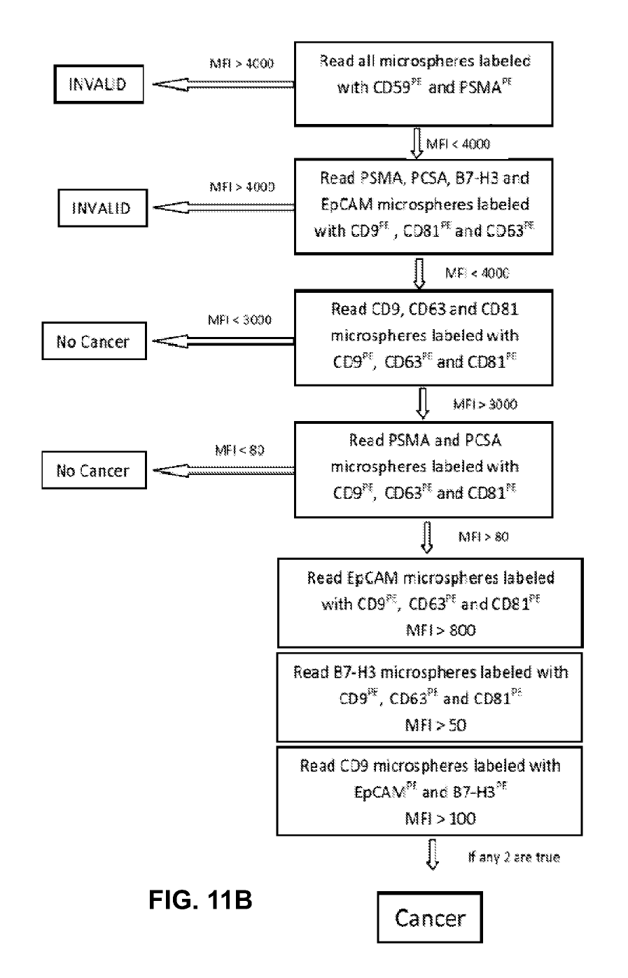

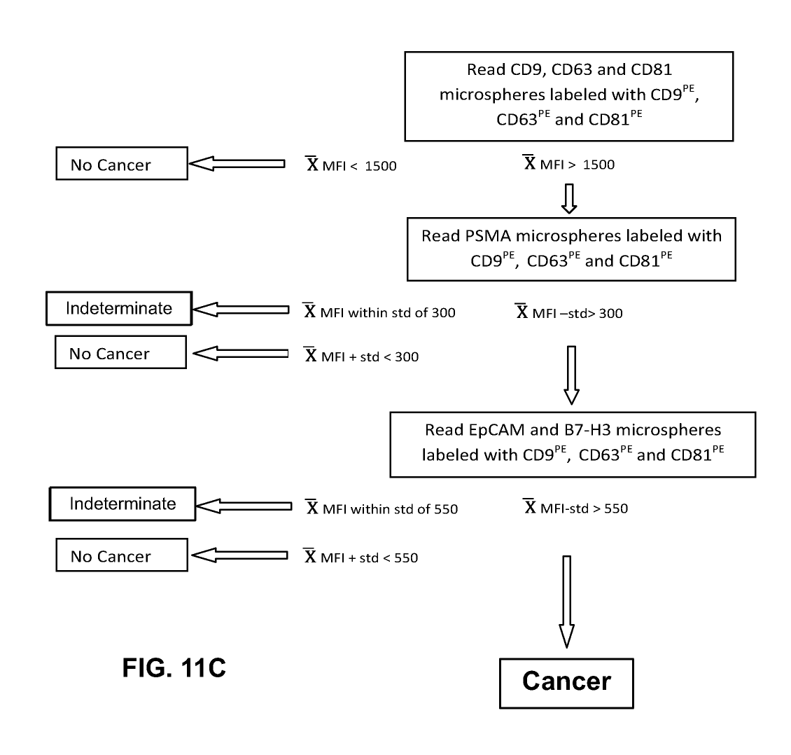

In this example, the vesicle (e.g., exosome, microvesicle, etc) PCa test is a microsphere based immunoassay for the detection of a set of protein biomarkers present on the vesicles from plasma of patients with prostate cancer. The test employs specific antibodies to the following protein biomarkers: CD9, CD59, CD63, CD81, PSMA, PCSA, B7H3 and EpCAM (FIG. 11A). After capture of the vesicles by antibody coated microspheres, phycoerythrin-labeled antibodies are used for the detection of vesicle specific biomarkers. Depending on the level of binding of these antibodies to the vesicles from a patient's plasma a determination of the presence or absence of prostate cancer is made.

Vesicles are isolated as described in Example 1.

Microspheres

Specific antibodies are conjugated to microspheres (Luminex) after which the microspheres are combined to make a Microsphere Master Mix consisting of L100-C105-01; L100-C115-01; L100-C119-01; L100-C120-01; L100-C122-01; L100-C124-01; L100-C135-01; and L100-C175-01. xMAP® Classification Calibration Microspheres L100-CAL1 (Luminex) are used as instrument calibration reagents for the Luminex LX200 instrument. xMAP® Reporter Calibration Microspheres L100-CAL2 (Luminex) are used as instrument reporter calibration reagents for the Luminex LX200 instrument. xMAP® Classification Control Microspheres L100-CON1 (Luminex) are used as instrument control reagents for the Luminex LX200 instrument. xMAP Reporter Control Microspheres L100-CON2 (Luminex) and are used as reporter control reagents for the Luminex LX200 instrument.

Capture Antibodies

The following antibodies are used to coat Luminex microspheres for use in capturing certain populations of vesicles by binding to their respective protein targets on the vesicles in this Example: a. Mouse anti-human CD9 monoclonal antibody is an IgG2b used to coat microsphere L100-C105 to make *EPCLMACD9-C105; b. Mouse anti-human PSMA monoclonal antibody is an IgG1 used to coat microsphere L100-C115 to make EPCLMAPSMA-C115; c. Mouse anti-human PCSA monoclonal antibody is an IgG1 used to coat microsphere L100-C119 to make EPCLMAPCSA-C119; d. Mouse anti-human CD63 monoclonal antibody is an IgG1 used to coat microsphere L100-C120 to make EPCLMACD63-C120; e. Mouse anti-human CD81 monoclonal antibody is an IgG1 used to coat microsphere L100-C124 to make EPCLMACD81-C124; f. Goat anti-human B7-H3 polyclonal antibody is an IgG purified antibody used to coat microsphere L100-C125 to make EPCLGAB7-H3-C125; and g. Mouse anti-human EpCAM monoclonal antibody is an IgG2b purified antibody used to coat microsphere L100-C175 to make EPCLMAEpCAM-C175.

Detection Antibodies

The following phycoerythrin (PE) labeled antibodies are used as detection probes in this assay: a. EPCLMACD81PE: Mouse anti-human CD81 PE labeled antibody is an IgG1 antibody used to detect CD81 on captured vesicles; b. EPCLMACD9PE: Mouse anti-human CD9 PE labeled antibody is an IgG1 antibody used to detect CD9 on captured vesicles; c. EPCLMACD63PE: Mouse anti-human CD63 PE labeled antibody is an IgG1 antibody used to detect CD63 on captured vesicles; d. EPCLMAEpCAMPE: Mouse anti-human EpCAM PE labeled antibody is an IgG1 antibody used to detect EpCAM on captured vesicles; e. EPCLMAPSMAPE: Mouse anti-human PSMA PE labeled antibody is an IgG1 antibody used to detect PSMA on captured vesicles; f. EPCLMACD59PE: Mouse anti-human CD59 PE labeled antibody is an IgG1 antibody used to detect CD59 on captured vesicles; and g. EPCLMAB7-H3PE: Mouse anti-human B7-H3 PE labeled antibody is an IgG1 antibody used to detect B7-H3 on captured vesicles.

Reagent Preparation

Antibody Purification:

The following antibodies in Table 3 are received from vendors and purified and adjusted to the desired working concentrations according to the following protocol.

TABLE 3
Antibodies for PCa Assay

| Antibody | Use |
|---|---|
| EPCLMACD9 | Coating of microspheres for vesicle capture |
| EPCLMACD63 | Coating of microspheres for vesicle capture |
| EPCLMACD81 | Coating of microspheres for vesicle capture |
| EPCLMAPSMA | Coating of microspheres for vesicle capture |
| EPCLGAB7-H3 | Coating of microspheres for vesicle capture |
| EPCLMAEpCAM | Coating of microspheres for vesicle capture |
| EPCLMAPCSA | Coating of microspheres for vesicle capture |
| EPCLMACD81PE | PE coated antibody for vesicle biomarker |
| | detection |
| EPCLMACD9PE | PE coated antibody for vesicle biomarker |
| | detection |
| EPCLMACD63PE | PE coated antibody for vesicle biomarker |
| | detection |
| EPCLMAEpCAMPE | PE coated antibody for vesicle biomarker |
| | detection |
| EPCLMAPSMAPE | PE coated antibody for vesicle biomarker |
| | detection |
| EPCLMACD59PE | PE coated antibody for vesicle biomarker |
| | detection |
| EPCLMAB7-H3PE | PE coated antibody for vesicle biomarker |
| | detection |

Antibody Purification Protocol: Antibodies are purified using Protein G resin from Pierce (Protein G spin kit, prod #89979). Micro-chromatography columns made from filtered P-200 tips are used for purification.

One hundred μl of Protein G resin is loaded with 100 μl buffer from the Pierce kit to each micro column. After waiting a few minutes to allow the resin to settle down, air pressure is applied with a P-200 Pipettman to drain buffer when needed, ensuring the column is not let to dry. The column is equilibrated with 0.6 ml of Binding Buffer (pH 7.4, 100 mM Phosphate Buffer, 150 mM NaCl; (Pierce, Prod #89979). An antibody is applied to the column (<1 mg of antibody is loaded on the column). The column is washed with 1.5 ml of Binding Buffer. Five tubes (1.5 ml micro centrifuge tubes) are prepared and 10 μl of neutralization solution (Pierce, Prod #89979) is applied to each tube. The antibody is eluted with the elution buffer from the kit to each of the five tubes, 100 ul for each tube (for a total of 500 μl). The relative absorbance of each fraction is measured at 280 nm using Nanodrop (Thermo scientific, Nanodrop 1000 spectrophotometer). The fractions with highest OD reading are selected for downstream usage. The samples are dialyzed against 0.25 liters PBS buffer using Pierce Slide-A-Lyzer Dialysis Cassette (Pierce, prod 66333, 3 KDa cut off). The buffer is exchanged every 2 hours for minimum three exchanges at 4° C. with continuous stirring. The dialyzed samples are then transferred to 1.5 ml microcentifuge tubes, and can be labeled and stored at 4° C. (short term) or −20° C. (long term).

Microsphere Working Mix Assembly:

A microsphere working mix MWM101 includes the first four rows of antibody, microsphere and coated microsphere of Table 4.

TABLE 4
Antibody-Microsphere Combinations

| Antibody | Microsphere | Coated Microsphere |
|---|---|---|
| EPCLMACD9 | L100-C105 | EPCLMACD9-C105 |
| EPCLMACD63 | L100-C120 | EPCLMACD63-C120 |
| EPCLMACD81 | L100-C124 | EPCLMACD81-C124 |
| EPCLMAPSMA | L100-C115 | EPCLMAPSMA-C115 |
| EPCLGAB7-H3 | L100-C125 | EPCLGAB7-H3-C125 |
| bEPCLMAEpCAM | L100-C175 | EPCLMAEpCAM-C175 |
| EPCLMAPCSA | L100-C119 | EPCLMAPCSA-C119 |

Microspheres are coated with their respective antibodies as listed above according to the following protocol.

Protocol for Two-Step Carbodiimide Coupling of Protein to Carboxylated Microspheres:

The microspheres should be protected from prolonged exposure to light throughout this procedure. The stock uncoupled microspheres are resuspended according to the instructions described in the Product Information Sheet provided with the microspheres (xMAP technologies, MicroPlex TM Microspheres). Five×106 of the stock microspheres are transferred to a USA Scientific 1.5 ml microcentrifuge tube. The stock microspheres are pelleted by microcentrifugation at ≧8000×g for 1-2 minutes at room temperature. The supernatant is removed and the pelleted microspheres are resuspended in 100 μl of dH2O by vortex and sonication for approximately 20 seconds. The microspheres are pelleted by microcentrifugation at ≧8000×g for 1-2 minutes at room temperature. The supernatant is removed and the washed microspheres are resuspended in 80 μl of 100 mM Monobasic Sodium Phosphate, pH 6.2 by vortex and sonication (Branson 1510, Branson ULTrasonics Corp.) for approximately 20 seconds. Ten μl of 50 mg/ml Sulfo-NHS (Thermo Scientific, Cat #24500) (diluted in dH2O) is added to the microspheres and is mixed gently by vortex. Ten μl of 50 mg/ml EDC (Thermo Scientific, Cat #25952-53-8) (diluted in dH2O) is added to the microspheres and gently mixed by vortexing. The microspheres are incubated for 20 minutes at room temperature with gentle mixing by vortex at 10 minute intervals. The activated microspheres are pelleted by microcentrifugation at ≧8000×g for 1-2 minutes at room temperature. The supernatant is removed and the microspheres are resuspended in 250 μl of 50 mM MES, pH 5.0 (MES, Sigma, Cat #M2933) by vortex and sonication for approximately 20 seconds. (Only PBS-1% BSA+Azide (PBS-BN) ((Sigma (P3688-10PAK+0.05% NaAzide (S8032))) should be used as assay buffer as well as wash buffer). The microspheres are then pelleted by microcentrifugation at ≧8000×g for 1-2 minutes at room temperature.

The supernatant is removed and the microspheres are resuspended in 250 μl of 50 mM MES, pH 5.0 (MES, Sigma, Cat #M2933) by vortex and sonication for approximately 20 seconds. (Only PBS-1% BSA+Azide (PBS-BN) ((Sigma (P3688-10PAK+0.05% NaAzide (S8032))) should be used as assay buffer as well as wash buffer). The microspheres are then pelleted by microcentrifugation at ≧8000×g for 1-2 minutes at room temperature, thus completing two washes with 50 mM MES, pH 5.0.

The supernatant is removed and the activated and washed microspheres are resuspended in 100 μl of 50 mM MES, pH 5.0 by vortex and sonication for approximately 20 seconds. Protein in the amount of 125, 25, 5 or 1 μg is added to the resuspended microspheres. (Note: Titration in the 1 to 125 μg range can be performed to determine the optimal amount of protein per specific coupling reaction). The total volume is brought up to 500 μl with 50 mM MES, pH 5.0. The coupling reaction is mixed by vortex and is incubated for 2 hours with mixing (by rotating on Labquake rotator, Barnstead) at room temperature. The coupled microspheres are pelleted by microcentrifugation at ≧8000×g for 1-2 minutes at room temperature. The supernatant is removed and the pelleted microspheres are resuspended in 500 μL of PBS-TBN by vortex and sonication for approximately 20 seconds. (Concentrations can be optimized for specific reagents, assay conditions, level of multiplexing, etc. in use).

The microspheres are incubated for 30 minutes with mixing (by rotating on Labquake rotator, Barnstead) at room temperature. The coupled microspheres are pelleted by microcentrifugation at ≧8000×g for 1-2 minutes at room temperature. The supernatant is removed and the microspheres are resuspended in 1 ml of PBS-TBN by vortex and sonication for approximately 20 seconds. (Each time there is the addition of samples, detector antibody or SA-PE the plate is covered with a sealer and light blocker (such as aluminum foil), placed on the orbital shaker and set to 900 for 15-30 seconds to re-suspend the beads. Following that the speed should be set to 550 for the duration of the incubation).

The microspheres are pelleted by microcentrifugation at ≧8000×g for 1-2 minutes. The supernatant is removed and the microspheres are resuspended in 1 ml of PBS-TBN by vortex and sonication for approximately 20 seconds. The microspheres are pelleted by microcentrifugation at ≧8000×g for 1-2 minutes (resulting in a total of two washes with 1 ml PBS-TBN).

Protocol for Microsphere Assay:

For multiple phycoerythrin detector antibody, the preparation is as described in Example 4. One hundred μl is analyzed on the Luminex analyzer (Luminex 200, xMAP technologies) according to the system manual. (High PMT setting).

Decision Tree:

A decision tree (FIG. 11B) using the results from the Luminex assay to determine if a subject has cancer. Threshold limits on the MFI is established and samples classified according to the result of MFI scores for the antibodies, to classify if a sample is PCa positive. FIG. 11C shows a decision tree in which a sample is classified as indeterminate if the MFI is within the standard deviation of the predetermined threshold. For validation, the sum of the MFI signal from PCSA, EpCAM and B7-H3 must be greater than 200 or the test is a ‘No Test’ meaning no result can be obtained.

Results:

See Examples that follow.

### Example 10

### Detection of Prostate Cancer

High quality training set samples were obtained from commercial suppliers. The samples comprised plasma from 42 normal prostate, 42 PCa and 15 BPH patients. The PCa samples included 4 stage III and the remainder state II. The samples were blinded until all laboratory work was completed.

The vesicles from the samples were obtained by filtration to eliminate particles greater than 1.5 microns, followed by column concentration and purification using hollow fiber membrane tubes. The samples were analyzed using a multiplexed bead-based assay system.

Antibodies to the following proteins were analyzed:
a. General Vesicle (MV) markers: CD9, CD81, and CD63
b. Prostate MV markers: PCSA
c. Cancer-Associated MV markers: EpCam and B7H3

Samples were required to pass a quality test as follows: if multiplexed fluorescence intensity (MFI) PSCA+MFI B7H3+MFI EpCam<200 then sample fails due to lack of signal above background. In the training set, six samples (three normals and three prostate cancers) did not achieve an adequate quality score and were excluded. An upper limit on the MFI was also established as follows: if MFI of EpCam is >6300 then test is over the upper limit score and samples are deemed not cancer (i.e., “negative” for purposes of the test).

The samples were classified according to the result of MFI scores for the six antibodies to the training set proteins, wherein the following conditions must be met to classified as PCa positive:
a. Average MFI of General MV markers>1500
b. PCSA MFI>300
c. B7H3 MFI>550
d. EpCam MFI 550-6300

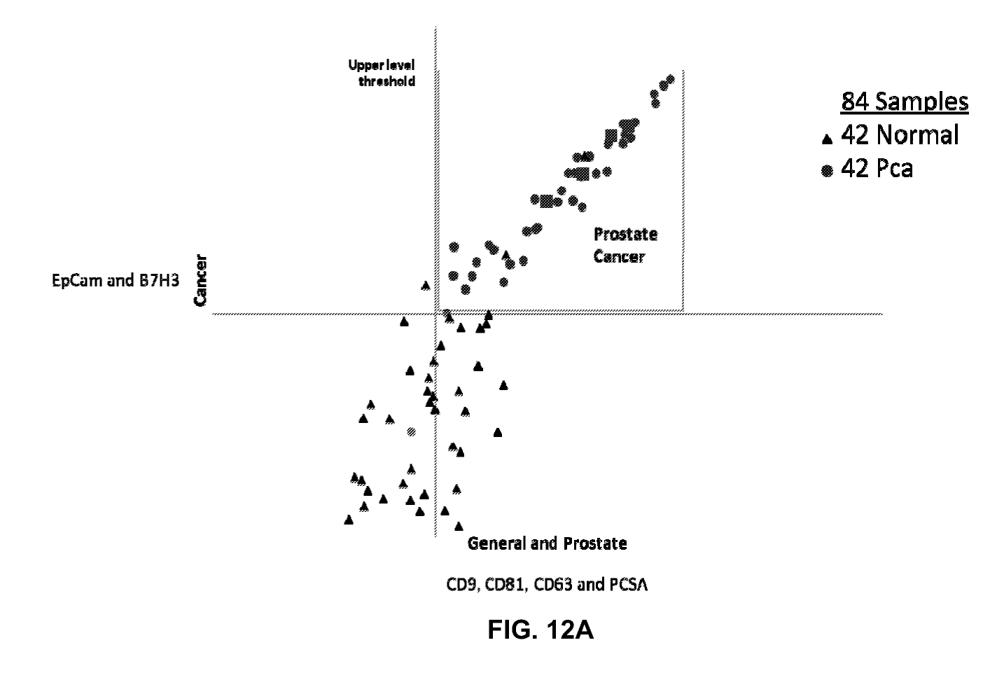

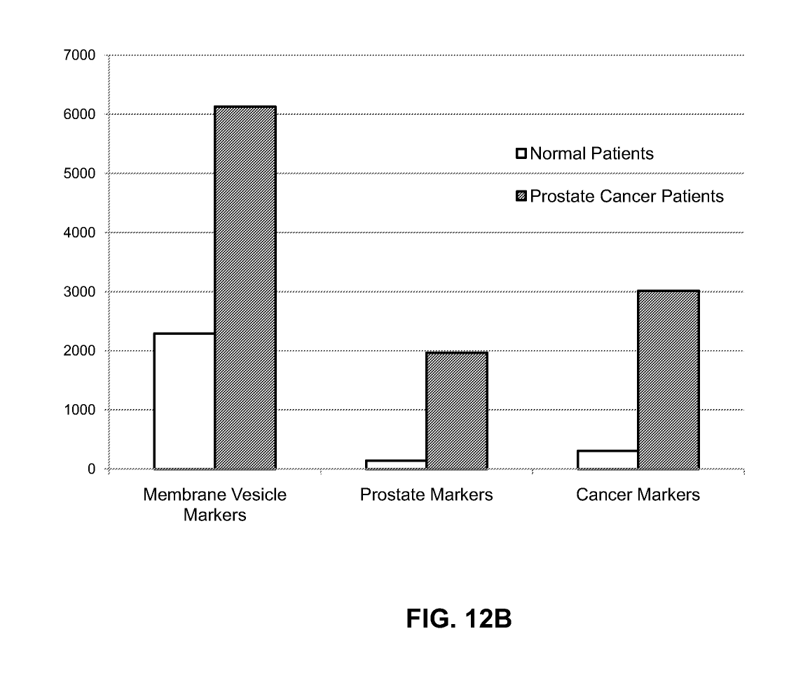

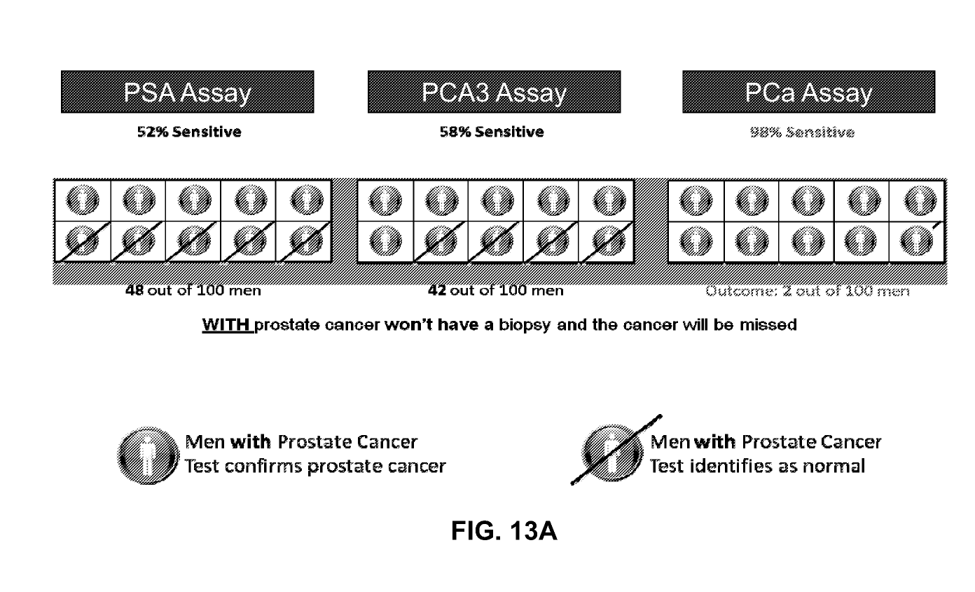

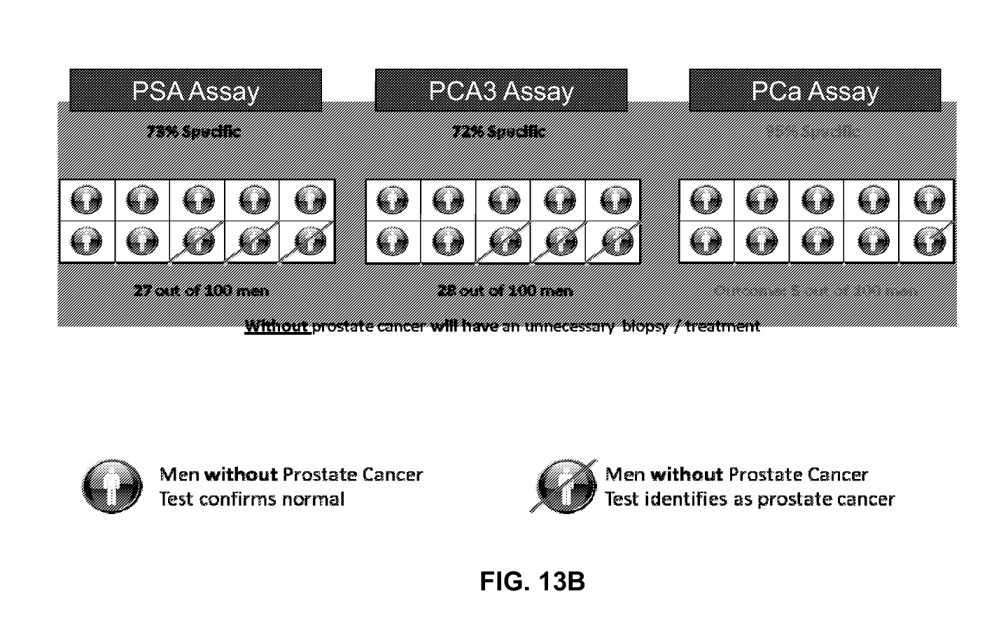

Using the 84 normal and PCa training data samples, the test was found to be 98% sensitive and 95% specific for PCa vs normal samples. See FIG. 12A. The increased MFI of the PCa samples compared to normals is shown in FIG. 12B. The sensitivity and specificity of the test compared to conventional PSA and PCA3 are presented in FIG. 13A and FIG. 13B, respectively. Compared to PSA and PCA3 testing, the PCa Test presented in this Example can result in saving 220 men without PCa in every 1000 normal men screened from having an unnecessary biopsy.

### Example 11

### Differentiating BPH from PCa

BPH is a common cause of elevated PSA levels. PSA can only indicated whether there is something wrong with the prostate, but it cannot effectively differentiate between BPH and PCa. PCA3, a transcript found to be overexpressed by prostate cancer cells, is thought to be slightly more specific for PCa, but this depends on the cutoffs used for PSA and PCA3, as well as the populations studied.

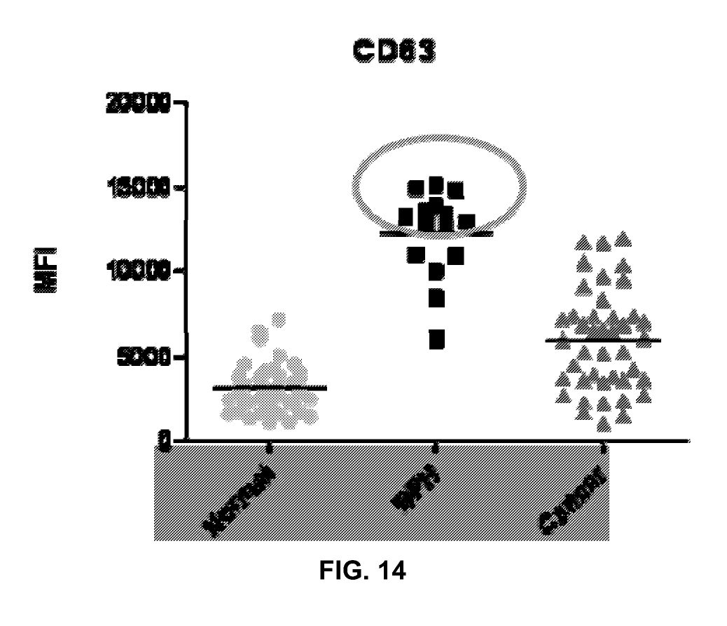

BPH can be characterized by vesicle (MV) analysis. Examining the samples described in Example 10, ten out of the 15 BPH samples (67%) have higher levels of CD63+ vesicles than the PCa samples, including the stage IIIs. See FIG. 14. Also, 14 out of 15 BPH (93%) have higher levels of CD63+ vesicles than the normals. This indicates that an inflammation-specific signature that differs from cancer may be used in differentiating BPH from PCa.

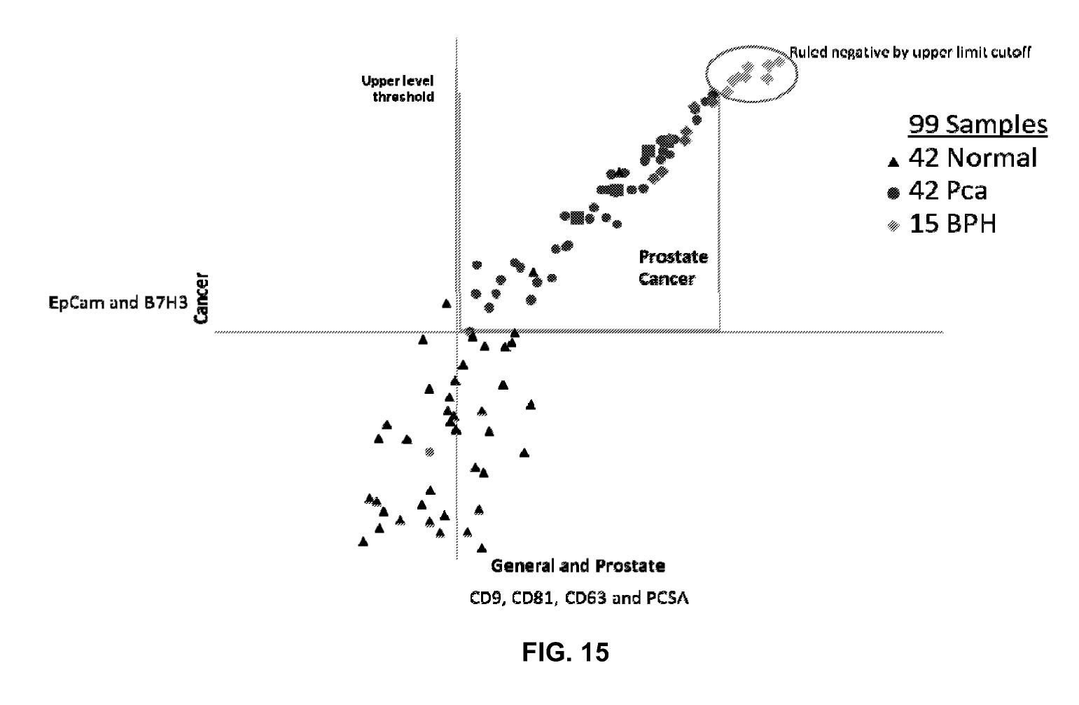

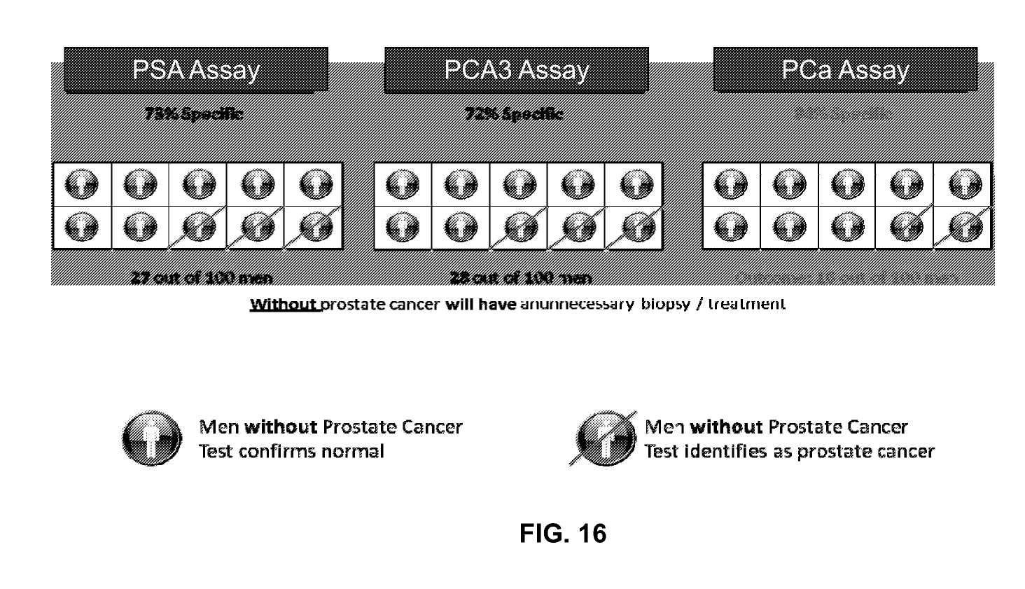

The PCa test from Example 10 was repeated including the 15 BPH samples. Using all 99 samples, the test was 98% sensitive and 84% specific. See FIG. 15. Thus, the test provides a 15% improvement over PSA. Performance values for PSA and PCA3 are commonly reported for settings without BPH in their cohorts, nevertheless, the vesicle test of the invention still outperforms conventional testing even when BPH was included. See FIG. 16. In this setting, the PCa test of the invention results in saving 110 men in every 1000 men without PCa screened from having an unnecessary biopsy as compared to PSA testing. And of those men biopsied due to a positive result from the assay, most will have something wrong with their prostate because the test performs well at identifying normal men (i.e., 95% specific in that population, see Example 10).

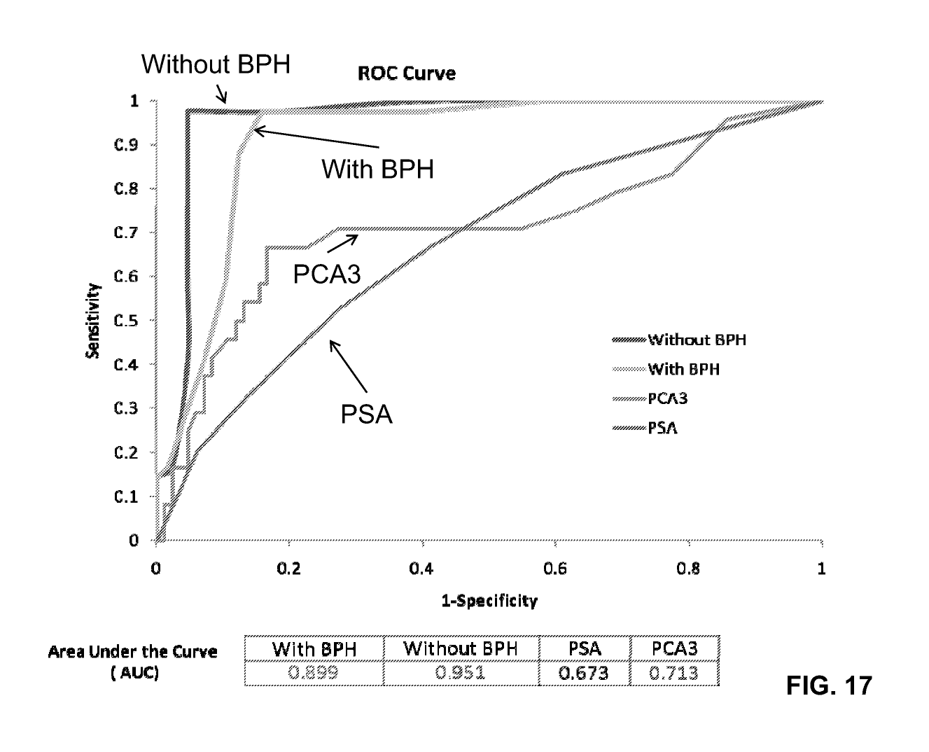

FIG. 17 presents ROC curve analysis of the vesicle assays of the invention versus conventional testing. When the ROC curve climbs rapidly towards upper left hand corner of the graph, the true positive rate is high and the false positive rate (1−specificity) is low. The AUC comparison shown in FIG. 16 shows that the test of the invention is much more likely to correctly classify a sample than conventional PSA or PCA3 testing.

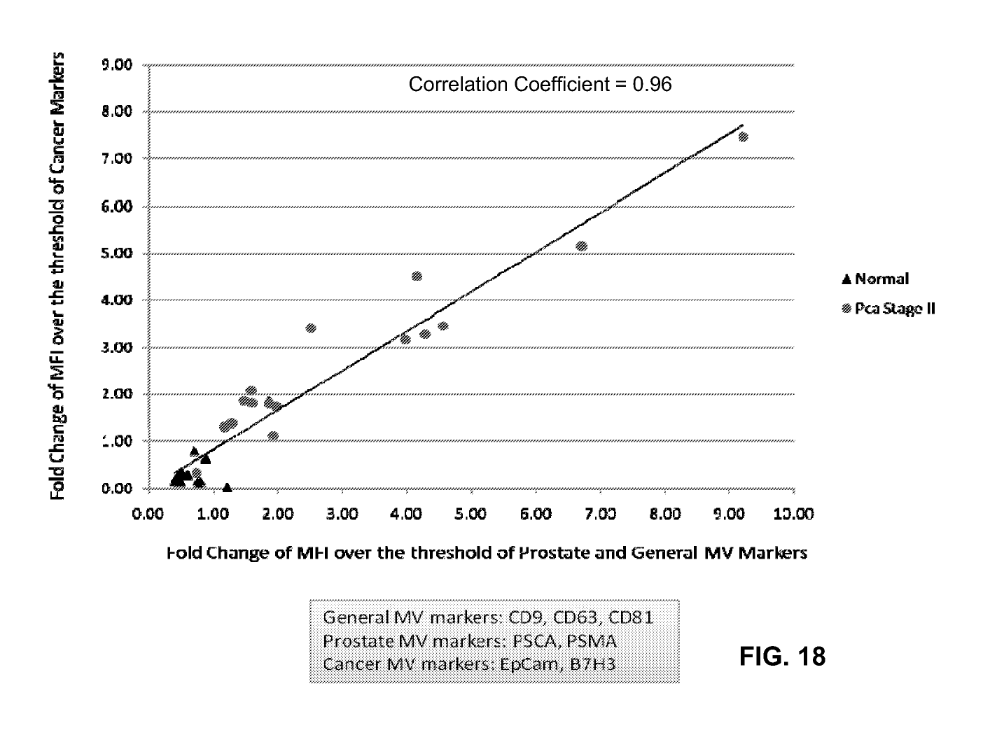

FIG. 18 shows that there is a correlation between general vesicle (MV) levels, levels of prostate-specific MVs and MVs with cancer markers, indicating these markers are correlated in the subject populations. Such cancer specific markers can be further used to differentiate between BPH and PCa. In the figure, General MV markers include CD9, CD63 and CD81; Prostate MV markers include PCSA and PSMA; and Cancer MV markers include EpCam and B7H3. Testing of PCa samples without the vesicle capture markers revealed sensitivity and specificity values nearly the same as those with the general MV markers were used. Similarly, detection of cancer without using B7H3 only leads to minimal reduction in performance. These data reveal that the markers of the invention can be substituted and tested in various configurations to still achieve optimal assay performance.

### Example 12

### Vesicle Bio-Signature for Prostate Cancer

This Example provides a multiplexed vesicle-based diagnostic platform that can identify specific and sensitive disease biosignatures. The platform was used to identify a blood based vesicle biosignature for prostate cancer (PCa). Current tests rely on increased levels of either PSA in the blood or elevated levels of the PCA3 transcript in urine. Unfortunately, PSA levels can also be elevated due to confounding conditions like benign prostate hyperplasia (BPH) and prostatitis, thereby reducing specificity of the marker. PCA3, while appearing to be prostate cancer specific, only offers moderate benefit over the performance of PSA, and requires a digital rectal exam to obtain a suitable specimen for analysis. The invention provides for screening and diagnosis of PCa with biomarkers that are both specific and sensitive and that can be surveyed from the blood or urine.

The present invention provides a method using a multiplexed diagnostic platform for quantifying and profiling vesicles in plasma. The method was used to develop a vesicle-derived biosignature comprising 7 different surface membrane protein biomarkers. These biomarkers include proteins specific to: vesicles generally (CD9, CD81, and CD63), vesicles from prostate epithelial cells (PSMA and PCSA), and tumor-associated vesicles (EpCam and B7H3).

The vesicle-specific PCa biosignature was compared between 29 PCa patients and 31 age-matched healthy male controls from the general population. The blood-based vesicle assay correctly identified PCa patients with a sensitivity of 83% and specificity of 90%, with an area under the curve (AUC)=0.881. Additionally, when patients with BPH (n=15) were included, the sensitivity was 83% and the specificity of the test remained significant at 85%, AUC=0.844. Further analysis revealed that 2 of the vesicle-associated markers (PCSA and B7H3) showed significant differences between stage II and stage III disease using a Kolmogorov-Smirnov test (p=0.009, 0.0271).

The present invention used a vesicle-based platform that is highly specific and sensitive to identify a plasma-based vesicle biosignature and an assay able to accurately differentiate prostate cancer from normal samples. The vesicle-derived biosignature patient profile can distinguish PCa from both normal and BPH and allow PCa progression and therapeutic monitoring to be analyzed through a simple blood test.

### Example 13

### Vesicle PCa Assay/Test

In this example, the vesicle PCa test is a microsphere based immunoassay for the detection of a set of protein biomarkers present on the vesicles from plasma of patients with prostate cancer. The test is performed similarly to that of Example 9 with modifications indicated below.

The test uses a multiplexed immunoassay designed to detect circulating microvesicles. The test uses PCSA, PSMA and B7H3 to capture the microvesicles present in patient samples such as plasma and uses CD9, CD81, and CD63 to detect the captured microvesicles. The output of this assay is the median fluorescent intensity (MFI) that results from the antibody capture and fluorescently labeled antibody detection of microvesicles that contain both the individual capture protein and the detector proteins on the microvesicle. A sample is “POSITIVE” by this test if the MFI levels of PSMA or PCSA, and B7H3 protein-containing microvesicles are above the empirically determined threshold. A sample is determined to be “NEGATIVE” if any one of these two microvesicle capture categories exhibit an MFI level that is below the empirically determined threshold. Alternatively, a result of “INDETERMINATE” will be reported if the sample MFI fails to clearly produce a positive or negative result due to MFI values not meeting certain thresholds or the replicate data showed too much statistical variation. A “NON-EVALUABLE” interpretation for this test indicates that this patient sample contained inadequate microvesicle quality for analysis. See Example 14 for a method to determine the empirically derived threshold values.

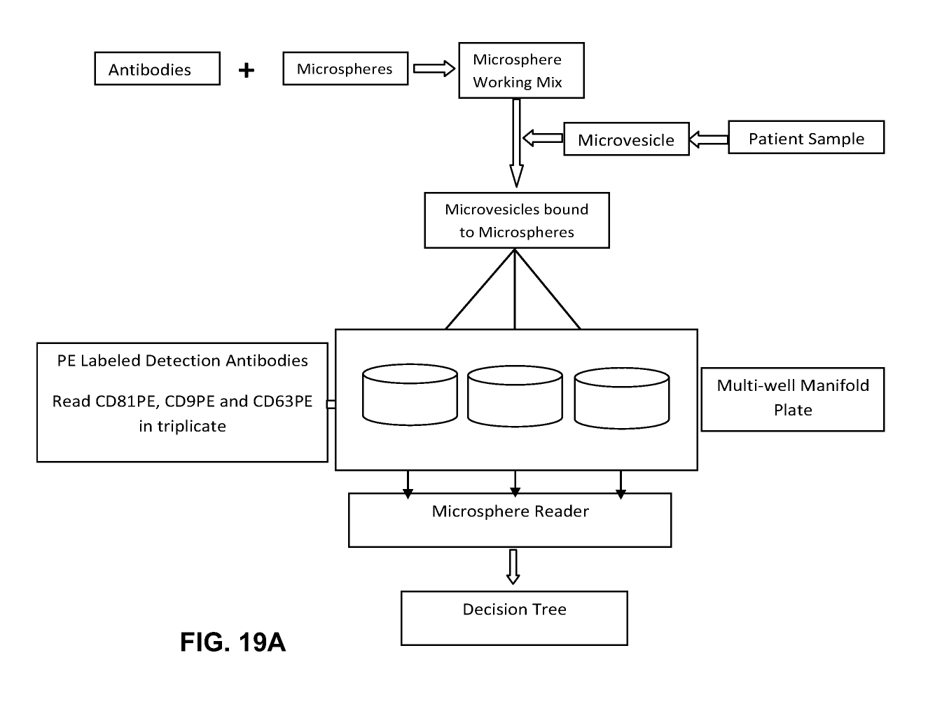

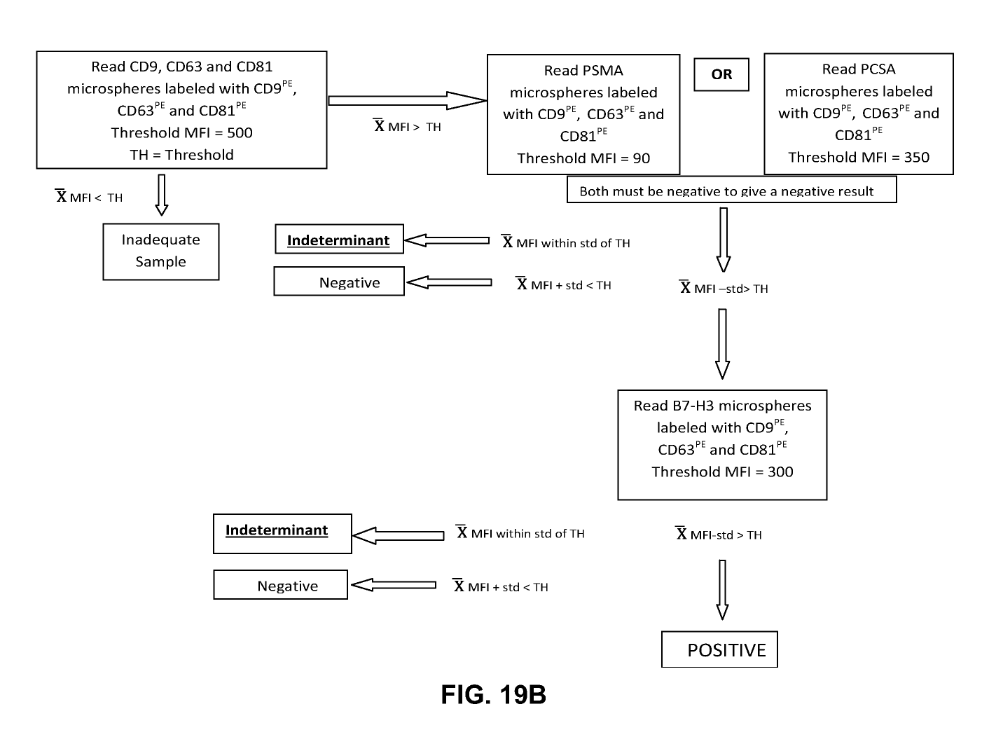

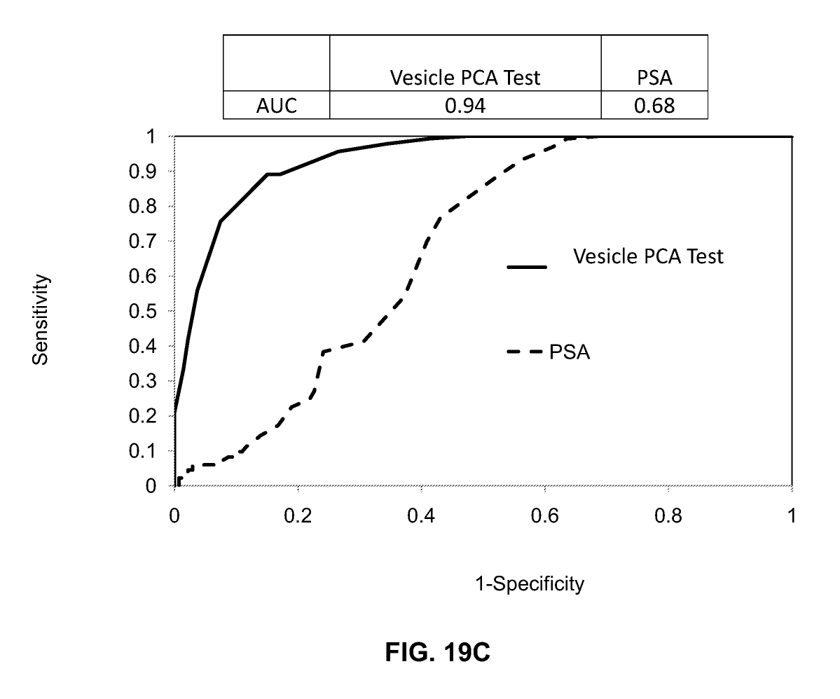

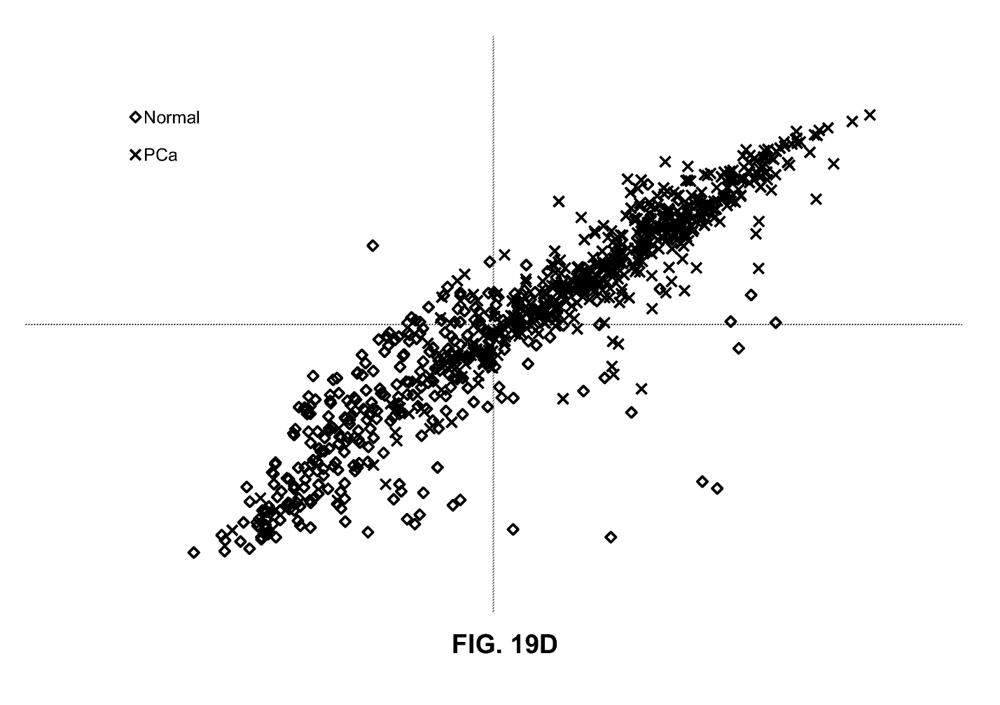

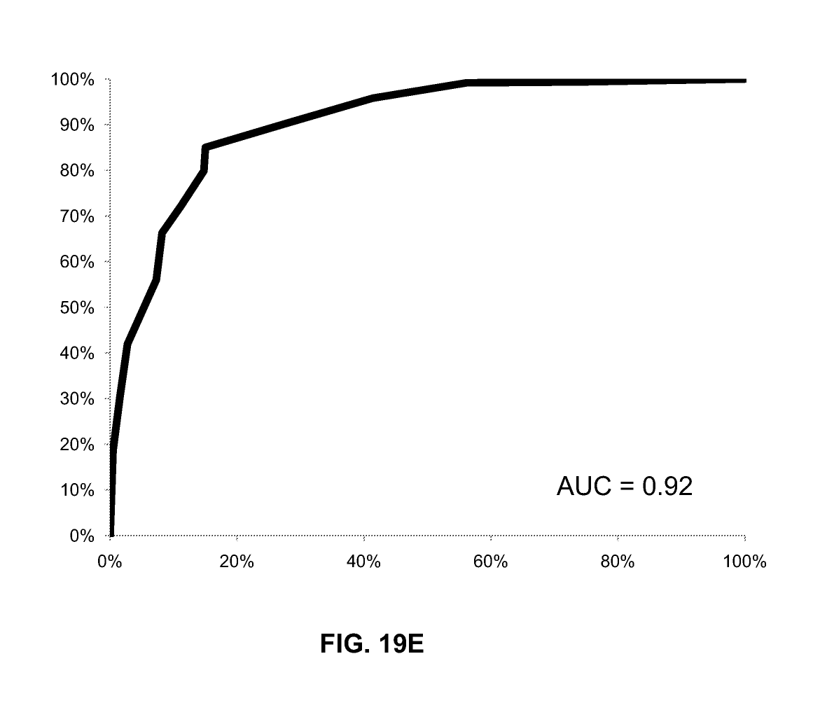

The test employs specific antibodies to the following protein biomarkers: CD9, CD59, CD63, CD81, PSMA, PCSA, and B7H3 (FIG. 19A-B). Decision rules are set to determine if a sample is called positive, negative or indeterminate, as outlined in Table 5 and FIG. 19B. For a sample to be called positive the replicates must exceed all four of the MFI cutoffs determined for the tetraspanin markers (CD9, CD63, CD81), prostate markers (PSMA or PCSA), and B7H3. Samples are called indeterminate if both of the three replicates from PSMA and PCSA or any of the three replicates from B7H3 antibodies span the cutoff MFI value. Samples are called negative if there is at least one of the tetraspanin markers (CD9, CD63, and CD81), prostate markers (PSMA or PCSA), B7H3 that fall below the MFI cutoffs.

TABLE 5
MFI Parameter for Each Capture Antibody

| Tetraspanin Markers | Prostate Markers | | Result |
|---|---|---|---|
| (CD9, CD63, CD81) | (PSMA, PCSA) | B7H3 | Determination |
| Average of all | All replicates from | All replicates from | If all 3 are true, |
| replicates from the | either of the two | B7H3 have a MFI >300 | then the sample is |
| three tetraspanins have | prostate markers have | | called Positive |
| a MFI >500 | a MFI >350 for PCSA | | |
| | and >90 for PSMA | | |
| | Both replicate sets | Any replicates | If either are true, |
| | from either prostate | from B7H3 have | then the sample is |
| | marker have values | values both above | called |
| | both above and below | and below a MFI = 300 | indeterminate |
| | a MFI = 350 for | | |
| | PCSA and =90 for | | |
| | PSMA | | |
| All replicates from the | All replicates from | All replicates from | If any of the 3 are |
| three tetraspanins have | either of the two | B7H3 have a MFI <300 | true, then the |
| a MFI <500 | prostate markers have | | sample is called |
| | a MFI <350 for PCSA | | Negative, given the |
| | and <90 for PSMA | | sample doesn't |
| | | | qualify as |
| | | | indeterminate |

The vesicle PCa test was compared to elevated PSA on a cohort of 296 patients with or without PCa as confirmed by biopsy. An ROC curve of the results is shown in FIG. 19C. As shown, the area under the curve (AUC) for the vesicle PCa test was 0.94 whereas the AUC for elevated PSA on the same samples was only 0.68. The PCa samples were likely found due to a high PSA value. Thus this population is skewed in favor of PSA, accounting for the higher AUC than is observed in a true clinical setting.

The vesicle PCa test was further performed on a cohort of 933 patient plasma samples. Results are shown in FIG. 19D and are summarized in Table 6:

TABLE 6
Performance of vesicle PCa test on 933 patient cohort

| | |
|---|---|
| True Positive | 409 |
| True Negative | 307 |
| False Positive | 50 |
| False Negative | 72 |
| Non-evaluable | 63 |
| Indeterminate | 32 |
| Total | 933 |
| Sensitivity | 85% |
| Specificity | 86% |
| Accuracy | 85% |
| Non-evaluable Rate | 8% |
| Indeterminate Rate | 5% |

As shown in Table 6, the vesicle PCa test achieved an 85% sensitivity level at a 86% specificity level, for an accuracy of 85%. In contrast, PSA at a sensitivity of 85% had a specificity of about 55%, and PSA at a specificity of 86% had a sensitivity of about 5%. FIG. 19C. About 12% of the 933 samples were non-evaluable or indeterminate. Samples from the patients could be recollected and re-evaluated. FIG. 19E shows an ROC curve corresponding to the data shown in FIG. 19D. The vesicle PCa test had an AUC of 0.92 for the 933 samples.

### Example 14

### Threshold Calculations

It is common to set a threshold level for a biomarker, wherein values above or below the threshold signify differential results, e.g., positive versus negative results. For example, the standard for PSA is a threshold of 4 ng/ml of PSA in serum. PSA levels below this threshold are considered normal whereas PSA values above this threshold may indicate a problem with the prostate, e.g., BPH or PCa. The threshold can be adjusted to favor enhanced sensitivity versus specificity. In the case of PSA, a lower threshold would detect more cancers, and thus increase sensitivity, but would concomitantly increase the number of false positives, and thus decrease specificity. Similarly, a higher threshold would detect fewer cancers, and thus decrease sensitivity, but would concomitantly decrease the number of false positives, and thus increase specificity.

In Examples 9-13, threshold MFI values are set for the vesicle biomarkers to construct a test for detecting PCa. This Example provides an approach to determining the threshold values. To this end, cluster analysis was used to determine if there were PCa positive and negative populations that could be separated based on MFI threshold values that result in the desired level of sensitivity.

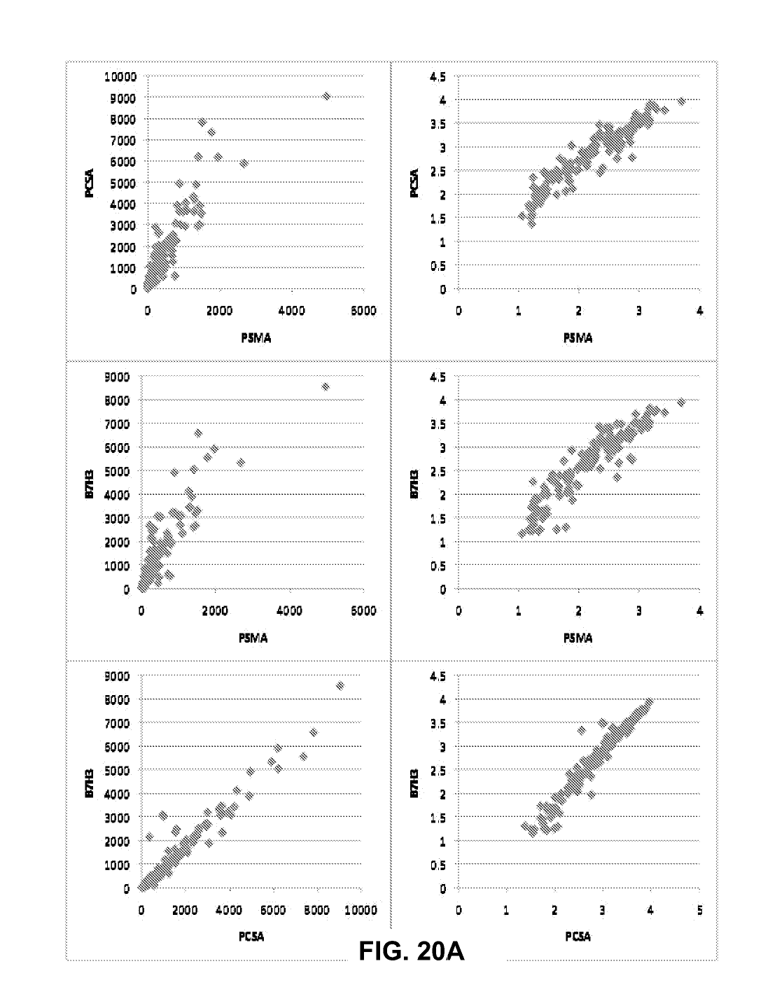

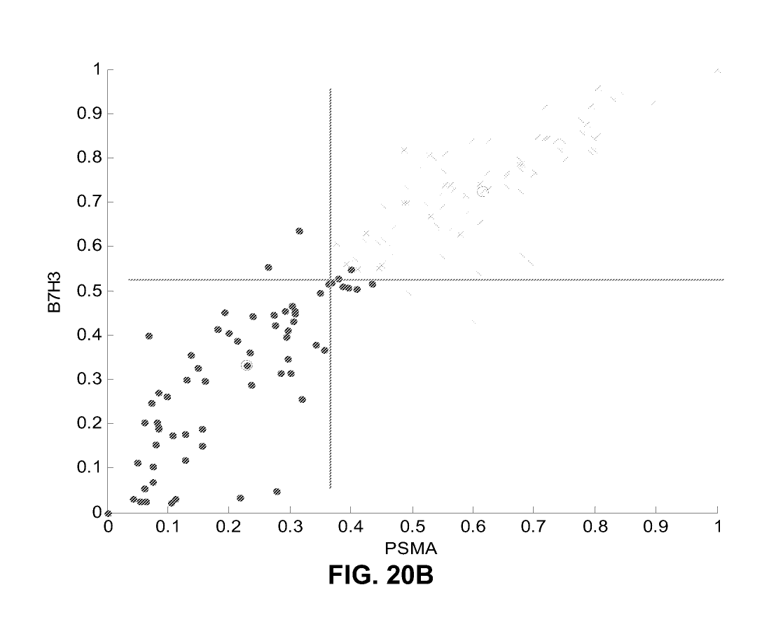

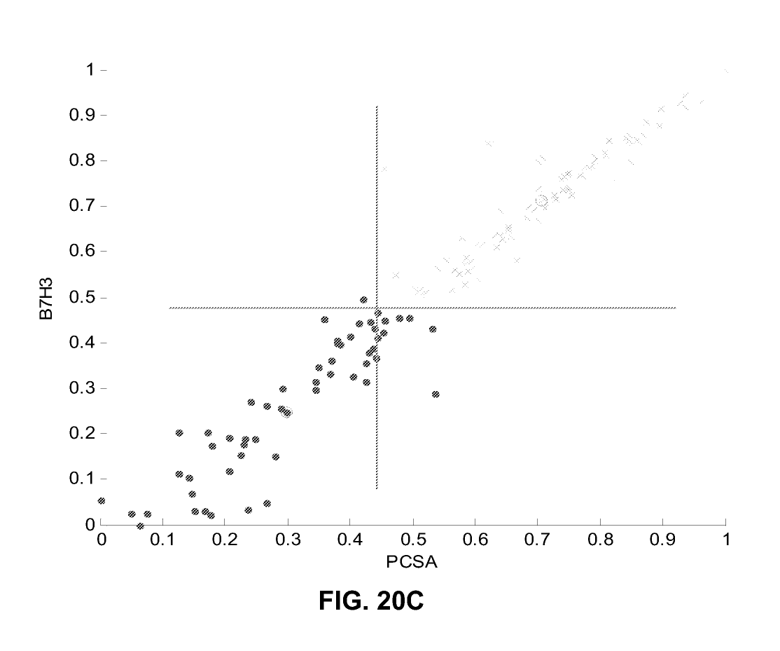

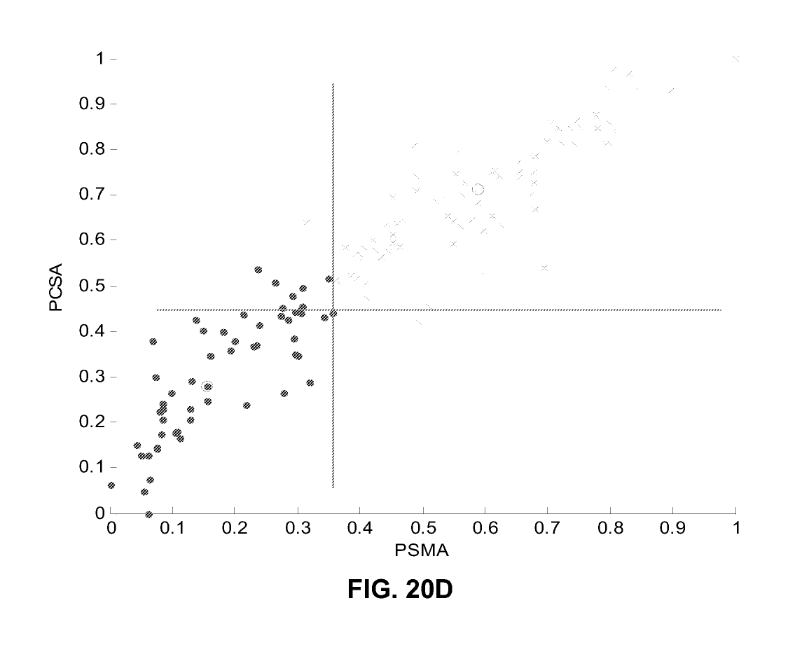

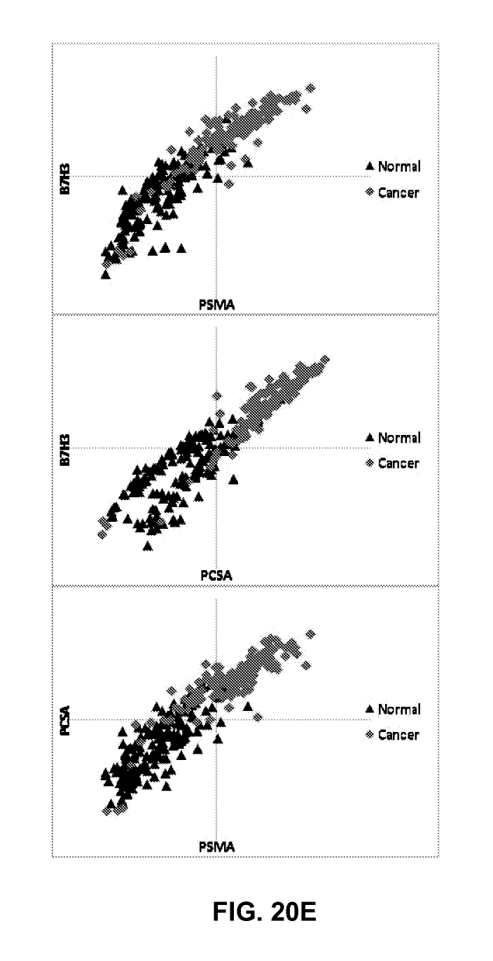

Fluorescence intensity values are exponentially distributed, thus prior to performing the clustering analysis, the data was logarithmically transformed. The resulting data set was subjected to traditional hard clustering methods. The hard clustering implemented here uses defined Euclidean distance parameter to determine if a data point belongs to a particular cluster. The algorithm used allocates each data point to one of c clusters to minimize the within-cluster sum of squares:

$${\sum }_{i = 1}^{c} {\sum }_{k ∈ {A}_{i}} {‖ {x}_{k} - {v}_{i} ‖}_{2}$$

where Aᵢ is a set of objects (data points) in the i-th cluster and vᵢ is the mean for those points over cluster i. This equation denotes a Euclidian distance norm. The data from 149 samples was used to determine the clusters. The raw data was logarithmically transformed so that it was uniformly distributed. The data was then normalized by subtracting the minimum value and dividing by the maximum. Plots of PSMA vs B7H3, PCSA vs B7H3, and PSMA vs PCSA both before and after transformation are shown in FIG. 20A.

Each possible combination of markers was analyzed, PSMA vs B7H3, PCSA vs PSMA, and PCSA vs B7H3 and thresholds were determined to optimally separate the populations identified. Horizontal and vertical lines where found that best separated the two clusters. The point where the line crossed the axis was used to define the cutoff, which required first that the value be denormalized, then the antilog taken. This resulted in cutoffs of 90 and 300 for each PSMA vs B7H3 respectively, as shown in shown in FIG. 20B.

For PCSA vs B7H3, the two clusters found are shown in FIG. 20C. Horizontal and vertical lines where found that best separated the two clusters. The point that the line crossed the axis was used to define the cutoff, which required first that the value be denormalized, then the antilog taken. This resulted in cutoffs of 430 and 300 for each PCSA vs B7H3 respectively.

For PSMA vs PCSA, the two clusters found are shown in FIG. 20D. Horizontal and vertical lines where found that best separated the two clusters. The point that the line crossed the axis was used to define the cutoff, which required first that the value be denormalized, then the antilog taken. This resulted in cutoffs of 85 and 350 for each PSMA vs PCSA respectively.

Sensitivity and specificity were calculated for all combinations of thresholds found with the cluster analysis. There was no change in sensitivity or specificity with values of 85 or 90 for PSMA, thus 90 was chosen to use as the cutoff. There was no change in sensitivity with thresholds of 430 or 350 for PCSA, though specificity decreased by 0.3% with the change. Since this is an insignificant change, a value of 350 was chosen for the PCSA cutoff so as to err on the side of higher sensitivity. Both clusters had the same threshold of 300 for B7H3, so this value was used. The resulting sensitivity and specificity with these threshold values was 92.7% and 81.8% respectively.

These thresholds were applied to the larger set of data containing 313 samples, and resulted in a sensitivity of 92.8% and a specificity of 78.7%. See FIG. 20E.

The thresholds in this Example were determined in a fashion that was independent of whether the samples were from normal or cancer patients. Since the thresholds perform well at separating the two populations, it is likely that there are in fact two separate underlying populations due to differences in the biology of the specimens. This difference is highly correlated to the presence or absence of prostate cancer, and thus serves as a good recommendation for the performance of a biopsy.

### Example 15

### Preparation of Lectin-Affinity Matrix

Cyanogen bromide (CNBr) activated agarose is used for direct coupling according to Cuatrecasas, et al (Cuatracasas et al. Proc Natl Acad Sci USA 61(2): 636-643, 1968). 1 ml of GNA at a concentration of 10 mg/ml in 0.1M NaHCO₃ pH 9.5 is added to 1 ml CNBr activated agarose (Sigma, St. Louis, Mo.) and is allowed to react overnight in the cold. When the reaction is complete, unreacted materials are aspirated and the lectin coupled agarose washed extensively with sterile cold PBS. The lectin agarose affinity matrix is then stored cold until ready for use. Alternatively, GNA agarose is available commercially from Vector Labs (Burlingame, Calif.)

A lectin affinity matrix using GNA covalently coupled to glass beads via Schiff's base and reduction with cyanoborohydride is prepared by first preparing a silica lectin affinity matrix using a modification of the method of Hermanson (Hermanson. Bioconjugate Techniques: 785, 1996). GNA lectin is dissolved to a final protein concentration of 10 mg/ml in 0.1M sodium borate pH 9.5 and added to aldehyde derivatized silica glass beads (BioConnexant, Austin Tex.). The reaction is performed at alkaline pH (or about pH 7-9) and is done at a 2-4 fold excess of GNA over coupling sites. To this mixture 10 μl 5M NaCNBH₃ in 1N NaOH (Aldrich, St Louis, Mo.) is added per ml of coupling reaction and the mixture allowed to react for 2 hours at room temperature. At the end of the reaction, remaining unreacted aldehyde on the glass surfaces are capped with 20 μl 3M ethanolamine pH 9.5 per ml of reaction. After 15 minutes at room temperature, the reaction solution is decanted and the unbound proteins and reagents are removed by washing extensively in PBS. The matrix is then stored cold until ready for use until ready for use.

A preparation of GNA covalently coupled to aminocelite using glutaraldehyde is prepared by first preparing aminocelite by reacting celite (silicate containing diatomaceous earth) overnight in a 5% aqueous solution of aminopropyl triethoxysilane. The aminated celite is washed free of excess reagent with water and ethanol and is dried overnight to yield an off white powder. One gram of the powder is then suspended in 5 ml 5% glutaraldehyde (Sigma) for 30 minutes. Excess glutaraldehyde is then removed by filtration and washing with water until no detectable aldehyde remains in the wash using Schiff's reagent. The filter cake is then resuspended in 5 ml of Sigma borohydride coupling buffer containing 2-3 mg/ml GNA and the reaction proceeds overnight at room temperature. At the end of the reaction, unreacted GNA is washed off and the unreacted aldehyde is aminated with ethanolamine. After final washing in sterile PBS, the material is stored cold until ready for use.

### Example 16

### Preparation of a Cartridge

A slurry of particulate immobilized GNA on agarose beads or celite in sterile PBS buffer is pumped into the outside compartment of a hollow-fiber dialysis column using a syringe. The Microkros polyethersulfone hollow-fiber dialysis cartridge is equipped with Luer fittings (200μ ID×240μ OD, pore diameter 200-500 nm, ˜0.5 ml internal volume) obtained from Spectrum Labs (Rancho Dominguez, Calif.). Cartridges containing the lectin affinity resin are equilibrated with 5-10 column volumes sterile PBS.

### Example 17

### Isolation of Vesicles Using Lectin Capture

Blood is collected from a patient via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

A cartridge comprising a porous membrane allows vesicles to flow freely through the membrane while extracellular proteins, larger membrane fragments, platelets and other non-vesicle bodies are bound and/or entrapped or prevented from flowing through. The cartridge is described in Example 16.

The flow through that passes through the membrane enters a column or matrix that comprises one or more binding agents that selectively binds the one or more vesicles present in the sample. The column or matrix is a lectin-affinity matrix as described in Example 15. The vesicles are collected or captured by this column, are washed and then are analyzed for the desired biomarkers using techniques as described herein. Generally, surface protein biomarkers are detected using antibody or aptamers which are bound to a substrate or labeled (see, e.g., FIG. 4A-4E), payload protein biomarkers are detected using immunoassays, and microRNA and mRNA are detected and quantitated by RT-PCR. The biomarker or bio-signature is then used to characterize a phenotype, such as for the diagnostic, prognostic, monitoring or theranostic purposes for a disease.

### Example 18

### Storage of Vesicles

Blood is collected from a patient via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

A cartridge comprising a porous membrane allows vesicles flow freely through the membrane while extracellular proteins, larger membrane fragments, platelets and other non-vesicle bodies are bound and/or entrapped or prevented from flowing through.

The flow through that passes through them membrane enters a column or matrix that comprises one or more binding agents that selectively binds the one or more vesicles present in the sample. The column or matrix can be a lectin-affinity matrix as described in Example 15. The vesicles are collected or captured by this column, are washed and eluted. The eluted vesicles are then collected in a CellSave Preservation Tube (Veridex, LLC, Raritan, N.J.) and stored for future use.

### Example 19

### Identifying Vesicle Subpopulations

This Example identifies various vesicle subpopulations by their particular surface protein topography. Plasma-derived vesicle RNA content of each subpopulation was characterized for association with a cancer phenotype. The protein topography and RNA content of vesicles found in plasma from patients with cancer, benign prostatic hyperplasia (BPH), and unaffected individuals were profiled to characterize and identify the vesicle subpopulations that are indicative of a given disease state. The biosignatures of these vesicle subpopulations can be used to develop a diagnostic platform to aid in the screening and diagnosis of various cancers. Plasma-derived vesicles are separated using flow cytometry (FACS) and cell sorting techniques into protein-specific subpopulations by using membrane-specific protein biomarkers (e.g. EpCam). Vesicles from prostate cancer (PCa) patients had the highest percentage of vesicles labeled with EpCam, PSMA and CD9 compared to vesicles from normal, BPH and colorectal cancer (CRC) patients. Additionally, for each subpopulation of vesicles separated by FACS, quantitative expression profiling of miRs was used to identify expression signatures specific to cancer patients.

The RNA content of various subpopulations of vesicles, defined by their membrane protein biosignature, can be unique. In a vesicle subpopulation with proteins CD9 and CD81 on the surface, miR 141 is significantly overexpressed in vesicles from prostate cancer (PCa) patient plasma compared to vesicles derived from normal plasma. miR 9 was significantly overexpressed in vesicles from BPH plasma in EpCam and PSMA vesicles when compared to vesicles of the same subpopulation isolated from normal and PCa plasma, thereby providing a signature to separate BPH and PCa samples. miR 491 was overexpressed in EpCam expressing vesicles derived from colon cancer plasma compared to normal and PCa.

### Example 20

### Vesicle Bio-Signature for Colorectal Cancer

Although colonoscopy is the gold standard to screen and identify colorectal cancer (CRC), it is estimated half of patients who are recommended for colonoscopy are not compliant. Often the lack of compliance is because many perceive a colonoscopy as an uncomfortable and invasive procedure. An ideal first step toward increasing participation in preventive strategies would be the development of a less invasive diagnostic test to identify those patients that have a blood-based biosignature indicative of the need for detection and biopsy by colonoscopy. This strategy would result in cancers being identified earlier and prevent disease-free individuals from undergoing an unnecessary invasive procedure. Current blood-based tests rely on increased levels of either carcinoembryonic antigen (CEA) or carbohydrate antigenic determinant (CA 19-9). Unfortunately, CEA and CA 19-9 are neither organ-specific nor tumor-specific.

The present invention provides a vesicle-based platform to identify patients with CRC using a vesicle-based biosignature derived from plasma samples. The vesicles comprise exosomes, which are endosome-derived vesicles between 40-100 nm in diameter that are secreted by most cell types, including tumor cells. The present invention provides a vesicle-specific assay that can diagnose CRC from surface membrane protein biosignatures on vesicles derived from peripheral blood of patients with CRC.

Biosignatures were derived from vesicles isolated from plasma of patients with and without CRC. Vesicle surface proteins (CD9, CD81, CD63, EpCam, EGFR, and STEAP) were used in a multiplexed microsphere assay to capture and detect vesicles as described herein. The quantity of vesicles with significant concentrations of these surface proteins lead to the development of a vesicle-specific biosignature that differentiated CRC samples from normal.

Vesicles present in blood plasma of CRC patients provide a signature by which CRC can be diagnosed as early as histological grade 1. The biosignature comprises different vesicle surface membrane protein markers, which include both general vesicle and cancer-specific proteins. Measurement of the vesicle biosignature in plasma differentiated patients with CRC (n=20) diagnosed by biopsy from individuals from the general population (n=20) with a sensitivity of 85% and specificity of 85%. The CRC samples analyzed were comprised of AJCC/UICC stage I (n=10), IIA (n=6), and IIIB (n=4).

Biosignatures identified in vesicles derived from the blood of patients with CRC provide a sensitive and specific test that can assist physicians screen, diagnose and treat patients with CRC.

### Example 21

### Vesicle Biosignatures from Cell Lines and Patient Samples

To develop a vesicle-specific protein and RNA signature to identify prostate cancer (PCa), RNA and surface membrane protein profiles of vesicles with flow cytometry (F ACS) of vesicles derived from four prostate cancer cell lines, VCaP, 22Rvl, LNCaP and DU145 were determined. The vesicle biosignatures identified from the cell lines were then measured in plasma vesicles isolated from PCa patients.

There was variability between each of the four cell line vesicle-specific mRNA expression levels and vesicle surface protein content. The mRNA vesicle biosignatures identified in the four cell lines were not found in the vesicles from plasma samples of patients with PCa. Using a combination of antibodies for B7H3, PSMA and CD63, a flow cytometry protein signature was identified from all prostate cancer cell line vesicle population that defined a specific subpopulation of vesicles containing all 3 proteins on their surface. This same vesicle subpopulation was not found in vesicles derived from patients with prostate cancer. Additionally, vesicle-specific mRNA expression of two mRNA transcripts often found to be overexpressed in PCa, STEAPI and SPINK1, was consistently identified in vesicles derived from prostate cancer cell lines VCaP and 22Rvl, but were only found in the plasma derived vesicles from one out of eight patients with PCa.

Patient samples are useful to identify disease-specific vesicle biosignatures. The signatures obtained in cell lines may sometimes be used to identify individuals with or without prostate cancer.

### Example 22

### Vesicle PCa Test with Lectin Isolation

Plasma samples are collected from a cohort of subjects with prostate cancer (PCa) or without prostate cancer, with status confirmed by prostate biopsy. Blood is collected from each subject via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

A cartridge comprising a porous membrane allows vesicles to flow freely through the membrane while extracellular proteins, larger membrane fragments, platelets and other non-vesicle bodies are bound and/or entrapped or prevented from flowing through. The flow through that passes through the membrane enters a GNA lectin-affinity matrix as described in Examples 15 and 16. The vesicles captured by the lectin-affinity matrix are washed and resuspended. Analysis of the vesicles is performed in Example 9 using the modifications presented in Example 13. Briefly, the lectin-captured vesicles are assessed for the surface markers PCSA, PSMA, B7H3, CD9, CD63 and CD81 using bead based immunoassay methodology. The MFI levels of detected microvesicles are compared between the prostate cancer and non-prostate cancer subjects. MFI thresholds are constructed as in Examples 13-14 that are determined to optimally detect PCa.

A blood sample is extracted from a subject with an elevated PSA level above 4 ng/ml and/or a suspicious digital rectal exam, but with a negative biopsy. Vesicles in the sample are assessed using the lectin-affinity isolation and surface marker detection as described in this Example. A diagnosis of PCa is provided.

### Example 22

### Vesicle CRC Test with Lectin Isolation

Plasma samples are collected from a cohort of subjects with colorectal cancer (CRC) or without colorectal cancer, with status confirmed by colonoscopy. Blood is collected from each subject via standard veinpuncture in a 7 ml K2-EDTA tube. The sample is spun at 400 g for 10 minutes in a 4° C. centrifuge to separate plasma from blood cells (SORVALL Legend RT+ centrifuge). The supernatant (plasma) is transferred by careful pipetting to 15 ml Falcon centrifuge tubes. The plasma is spun at 2,000 g for 20 minutes and the supernatant is collected.

A cartridge comprising a porous membrane allows vesicles to flow freely through the membrane while extracellular proteins, larger membrane fragments, platelets and other non-vesicle bodies are bound and/or entrapped or prevented from flowing through. The flow through that passes through the membrane enters a GNA lectin-affinity matrix as described in Examples 15 and 16. The vesicles captured by the lectin-affinity matrix are washed and resuspended. Analysis of the vesicles is performed in Example 9 using the biomarkers presented in Example 20. Briefly, the lectin-captured vesicles are assessed for the surface markers CD9, CD81, CD63, EpCam, EGFR, and STEAP using bead based immunoassay methodology. The MFI levels of detected microvesicles are compared between the CRC and non-CRC subjects. MFI thresholds are constructed using an approach as in Example 14 that are determined to optimally detect CRC.

A blood sample is extracted from a subject at age 50 who is reluctant to undergo a colonoscopy. Vesicles in the sample are assessed using the lectin-affinity isolation and surface marker detection as described in this Example. A suspicious result for CRC indicates that the subject should undergo a confirmatory colonoscopy.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A method for detecting a presence or level of one or more vesicle in a biological sample obtained from a subject comprising:
i) contacting the biological sample with one or more lectin;
ii) contacting the biological sample with one or more non-lectin binding agent, wherein the one or more non-lectin binding agent binds a microvesicle surface antigen; and
iii) detecting a presence or level of one or more vesicle recognized by the one or more lectin and the one or more non-lectin binding agent.

3. (canceled)

4. (canceled)

5. The method of claim 2, further comprising characterizing a cancer based on the detected presence or level of the one or more vesicle.

6. (canceled)

7. The method of claim 5, wherein the characterizing comprises a diagnosis, prognosis, determination of drug efficacy, monitoring the status of the subject's response or resistance to a treatment or selection of a treatment for the cancer.

8. (canceled)

9. (canceled)

10. The method of claim 5, wherein the cancer comprises prostate cancer.

11. The method of claim 5, wherein characterizing the cancer comprises comparing the detected presence or level of the one or more vesicle to one or more reference values.

12. (canceled)

13. (canceled)

14. (canceled)

15. (canceled)

16. The method of claim 2, wherein the the one or more non-lectin binding agent comprises one or more binding agent to one or more general vesicle biomarker, and one or more binding agent to one or more cell-of-origin biomarker and/or one or more binding agent to one or more disease specific biomarker.

17. The method of claim 16, wherein the one or more general vesicle biomarker comprises one or more of CD63, CD9, CD81, CD82, CD37, CD53, and Rab-5b.

18. The method of claim 2, wherein the detected presence or level of the one or more vesicle comprises a presence or level of one or more of CD9, CD63 and CD81; a presence or level of one or more of PSMA and PCSA; and a presence or level of one or more of B7H3 and EpCam.

19. (canceled)

20. The method of claim 2, further comprising detecting a nucleic acid associated with the one or more vesicle.

21. The method of claim 17, wherein the nucleic acid is detected using microarray analysis, PCR, hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), image cytometry, qRT-PCR, real-time PCR, PCR, flow cytometry, or a combination thereof.

22. The method of claim 2, wherein the one or more lectin comprises Galanthus nivalis agglutinin (GNA), Narcissus pseudonarcissus agglutinin (NPA), cyanovirin (CVN), Lens culimaris agglutinin-A (LCA), wheat germ agglutinin (WGA), concanavalin A (Con A), Griffonia (Bandeiraea) Simplicifolia Lectin II (GS-II), or a combination thereof.

23. The method of claim 2, wherein the one or more lectin is bound to a substrate.

24. The method of claim 23, wherein the substrate is a planar substrate or a particle.

25. The method of claim 23, further comprising releasing the one or more vesicle from the substrate prior to step ii).

26. The method of claim 2, further comprising passing the biological sample through one or more porous membranes.

27. (canceled)

28. (canceled)

29. The method of claim 2, wherein the one or more non-lectin binding agent comprises one or more binding agent selected from the group consisting of DNA, RNA, monoclonal antibodies, polyclonal antibodies, Fabs, Fab′, single chain antibodies, synthetic antibodies, DNA aptamers, RNA aptamers, peptoids, zDNA, peptide nucleic acids (PNAs), locked nucleic acids (LNAs), synthetic occurring chemical compounds, naturally occurring chemical compounds, dendrimers, and combinations thereof.

30. The method of claim 2, wherein the one or more vesicle is isolated prior to step ii) by size exclusion chromatography, density gradient centrifugation, differential centrifugation, nanomembrane ultrafiltration, or combinations thereof.

31. (canceled)

32. (canceled)

33. (canceled)

34. (canceled)

35. The method of claim 2, wherein the one or more vesicle is derived from a tumor or cancer cell.

36. The method of claim 35, wherein the tumor or cancer cell is a lung, pancreas, stomach, intestine, bladder, kidney, ovary, testis, skin, colorectal, breast, prostate, brain, esophagus, liver, placenta, or fetal cell.

37. The method of claim 2, wherein the biological sample comprises a bodily fluid.

38. The method of claim 37, wherein the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, or umbilical cord blood.

39. (canceled)

40. (canceled)

41. (canceled)

42. (canceled)

43. (canceled)

44. (canceled)

45. (canceled)

46. The method of claim 2, wherein the biological sample comprises peripheral blood, sera, and/or plasma.

47. The method of claim 46, further comprising removal of highly abundant proteins from the biological sample.

48. The method of claim 47, wherein the highly abundant proteins comprise one or more of albumin, IgG, α1-antitrpsin, IgA, IgM, transferrin, haptoglobin, α1-acid glycoprotein, α2-macroglobin, apolipoprotein A-I, apolipoprotein A-II and fibrinogen.

49. The method of claim 47, wherein the highly abundant proteins are removed prior to step i).

50. The method of claim 47, wherein the highly abundant proteins are removed prior to step ii).

51. The method of claim 2, wherein detecting the presence or level of one or more vesicle recognized by the one or more lectin and the one or more non-lectin binding agent comprises detecting the presence or level of one or more intact microvesicles.

52. The method of claim 2, wherein the one or more non-lectin binding agent comprises two non-lectin binding agents that bind two different microvesicle surface antigens.
